SEQUENCE LISTING (95 sequences)

SEQ ID NO: 1 - protein, 102 aa, Artificial Sequence
  synthetic
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 2 - protein, 35 aa, Artificial Sequence
  synthetic
IKIATRKYLGKQNVYDIGVERDHNFALKNGFIASN

SEQ ID NO: 3 - protein, 106 aa, Artificial Sequence
  synthetic
CLAGDTLITLADGRRVPIRELVSQQNFSVWALNPQTYRLERARVSRAFCTGIKPVYRLTTRLGRSIRATANHRFLTPQGWKRVDELQPGDYLALPRRIPTASTPTL

SEQ ID NO: 4 - protein, 51 aa, Artificial Sequence
  synthetic
AAACPELRQLAQSDVYWDPIVSIEPDGVEEVFDLTVPGPHNFVANDIIAHN

SEQ ID NO: 5 - protein, 102 aa, Artificial Sequence
  synthetic
CLSYDTEILTVEYGILPIGKIVEKRIECTVYSVDNNGNIYTQPVAQWHDRGEQEVFEYCLEDGSLIRATKDHKFMTVDGQMMPIDEIFERELDLMRVDNLPN

SEQ ID NO: 6 - protein, 35 aa, Artificial Sequence
  synthetic
VKVIGRRSLGVQRIFDIGLPQYHNFLLANGAIAAN

SEQ ID NO: 7 - protein, 101 aa, Artificial Sequence
  synthetic
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 8 - protein, 35 aa, Artificial Sequence
  synthetic
VKIISRKSLGTQNVYDIGVEKDHNFLLKNGLVASN

SEQ ID NO: 9 - protein, 102 aa, Artificial Sequence; identical to SEQ ID NO: 1
  synthetic

SEQ ID NO: 10 - protein, 35 aa, Artificial Sequence; identical to SEQ ID NO: 2
  synthetic

SEQ ID NO: 11 - protein, 101 aa, Artificial Sequence; identical to SEQ ID NO: 7
  synthetic

SEQ ID NO: 12 - protein, 52 aa, Artificial Sequence
  synthetic
KRTADGSEFESPKKKRKVKIISRKSLGTQNVYDIGVEKDHNFLLKNGLVASN

SEQ ID NO: 13 - protein, 123 aa, Artificial Sequence
  synthetic
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 14 - protein, 36 aa, Artificial Sequence
  synthetic
VKVIGRRSLGVQRIFDIGLPQDHNFLLANGAIAANC

SEQ ID NO: 15 - protein, 261 aa, Artificial Sequence
  synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKDYKDHDGDYKDHDIDYKDDDDK

SEQ ID NO: 16 - protein, 2293 aa, Artificial Sequence
  synthetic
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQDDYKDHDGDYKDHDIDYKDDD

SEQ ID NO: 17 - protein, 2501 aa, Artificial Sequence
  synthetic
MPPNINWKEIMKVDPDDLPRQEELADNLLISLSKVEVNELKSEKQENVIHLFRITQSLMKMKAQEVELALEEVEKAGEEQAKFENQLKTKVMKLENELEMAQQSAGGRDTRFLRNEICQLEKQLEQKDRELEDMEKELEKEKKVNEQLALRNEEAENENSKLRRENKRLKKKNEQLCQDIIDYQKQIDSQKETLLSRRGEDSDYRSQLSKKNYELIQYLDEIQTLTEANEKIEVQNQEMRKNLEESVQEMEKMTDEYNRMKAIVHQTDNVIDQLKKENDHYQLQVQELTDLLKSKNEEDDPIMVAVNAKVEEWKLILSSKDDEIIEYQQMLHNLREKLKNAQLDADKSNVMALQQGIQERDSQIKMLTEQVEQYTKEMEKNTCIIEDLKNELQRNKGASTLSQQTHMKIQSTLDILKEKTKEAERTAELAEADAREKDKELVEALKRLKDYESGVYGLEDAVVEIKNCKNQIKIRDREIEILTKEINKLELKISDFLDENEALRERVGLEPKTMIDLTEFRNSKHLKQQQYRAENQILLKEIESLEEERLDLKKKIRQMAQERGKRSATSGLTTEDLNLTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEVESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLDQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKTTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIYDYKDHDGDYKDHDIDYKDDDDK

SEQ ID NO: 18 - protein, 2351 aa, Artificial Sequence
  synthetic
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 19 - protein, 159 aa, Artificial Sequence
  synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 20 - protein, 105 aa, Artificial Sequence
  synthetic
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLP

SEQ ID NO: 21 - protein, 100 aa, Artificial Sequence
  synthetic
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNL

SEQ ID NO: 22 - protein, 67 aa, Artificial Sequence
  synthetic
CLSTEILTVEYGLPIGKIVICVYSVDGYTQAQWHDRGEQEVEYEDGSIRATDHFTDQLIEIFRLDLN

SEQ ID NO: 23 - protein, 100 aa, Artificial Sequence
  synthetic
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENI

SEQ ID NO: 24 - protein, 36 aa, Artificial Sequence
  synthetic
MIKIATRKYLGKQNVYDIGVERDHNFALKNGFIASN

SEQ ID NO: 25 - protein, 19 aa, Artificial Sequence
  synthetic
MKRLGQDIGDHNFLNGIAN

SEQ ID NO: 26 - protein, 36 aa, Artificial Sequence
  synthetic
MVKVIGRRSLGVQRIFDIGLPQDHNFLLANGAIAAN

SEQ ID NO: 27 - DNA, 477 bp, Artificial Sequence
  synthetic
atcagcctgatcgccgccctggccgtggactacgtgatcggcatggagaacgccatgccctggaacctgcccgccgacctggcctggttcaagaggaacaccctgaacaagcccgtgatcatgggcaggcacacctgggagagcatcggcaggcccctgcccggcaggaagaacatcatcctgagcagccagcccagcaccgacgacagggtgacctgggtgaagagcgtggacgaggccatcgccgcctgcggcgacgtgcccgagatcatggtgatcggcggcggcagggtgatcgagcagttcctgcccaaggcccagaagctgtacctgacccacatcgacgccgaggtggagggcgacacccacttccccgactacgagcccgacgactgggagagcgtgttcagcgagttccacgacgccgacgcccagaacagccacagctactgcttcgagatcctggagaggaggtga

SEQ ID NO: 28 - protein, 159 aa, Artificial Sequence
  synthetic
MISLIAALAVDRVIGMENAMPWNLPADLAWFKRNTLDKPVIMGRHTWESIGRPLPGRKNIILSSQPGTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 29 - protein, 159 aa, Artificial Sequence; identical to SEQ ID NO: 19
  synthetic

SEQ ID NO: 30 - DNA, 315 bp, Artificial Sequence
  synthetic
atcagcctgatcgccgccctggccgtggactacgtgatcggcatggagaacgccatgccctggaacctgcccgccgacctggcctggttcaagaggaacaccctgaacaagcccgtgatcatgggcaggcacacctgggagagcatcggcaggcccctgcccggcaggaagaacatcatcctgagcagccagcccagcaccgacgacagggtgacctgggtgaagagcgtggacgaggccatcgccgcctgcggcgacgtgcccgagatcatggtgatcggcggcggcagggtgatcgagcagttcctgccctga

SEQ ID NO: 31 - protein, 105 aa, Artificial Sequence
  synthetic
MISLIAALAVDRVIGMENAMPWNLPADLAWFKRNTLDKPVIMGRHTWESIGRPLPGRKNIILSSQPGTDDRVTWVKSVDEAIAACGDVPEIMVIGGGRVYEQFLP

SEQ ID NO: 32 - protein, 105 aa, Artificial Sequence; identical to SEQ ID NO: 20
  synthetic

SEQ ID NO: 33 - DNA, 2665 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcctgagctacgagaccgagatcctgaccgtggagtacggcctgctgcccatcggcaagatcgtggagaagcggatcgagtgcaccgtgtacagcgtggacaacaacggcaacatctacacccagcccgtggcccagtggcacgaccggggcgagcaggaggtgttcgagtactgcctggaggacggcagcctgatccgggccaccaaggaccacaagttcatgaccgtggacggccagatgctgcccatcgacgagatcttcgagcgggagctggacctgatgcgggtggacaacctgcccaacgactacaaagaccatgacggtgattataaagatcatgacatcgactacaaggatgacgatgacaagtgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 34 - DNA, 108 bp, Artificial Sequence
  synthetic
atgatcaagatcgccacccggaagtacctgggcaagcagaacgtgtacgacatcggcgtggagcgggaccacaacttcgccctgaagaacggcttcatcgccagcaat

SEQ ID NO: 35 - DNA, 507 bp, Artificial Sequence
  synthetic
tgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 36 - DNA, 583 bp, Artificial Sequence
  synthetic
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 37 - DNA, 301 bp, Artificial Sequence
  synthetic
ctagtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggcaagggcgc

SEQ ID NO: 38 - DNA, 477 bp, Artificial Sequence; identical to SEQ ID NO: 27
  synthetic

SEQ ID NO: 39 - DNA, 315 bp, Artificial Sequence; identical to SEQ ID NO: 30
  synthetic

SEQ ID NO: 40 - DNA, 318 bp, Artificial Sequence
  synthetic
tgcctggccggcgacaccctgatcaccctggccgacggcaggagggtgcccatcagggagctggtgagccagcagaacttcagcgtgtgggccctgaacccccagacctacaggctggagagggccagggtgagcagggccttctgcaccggcatcaagcccgtgtacaggctgaccaccaggctgggcaggagcatcagggccaccgccaaccacaggttcctgaccccccagggctggaagagggtggacgagctgcagcccggcgactacctggccctgcccaggaggatccccaccgccagcacccccaccctg

SEQ ID NO: 41 - DNA, 156 bp, Artificial Sequence
  synthetic
atggccgccgcctgccccgagctgaggcagctggcccagagcgacgtgtactgggaccccatcgtgagcatcgagcccgacggcgtggaggaggtgttcgacctgaccgtgcccggcccccacaacttcgtggccaacgacatcatcgcccacaac

SEQ ID NO: 42 - DNA, 306 bp, Artificial Sequence
  synthetic
tgcctgagctacgacaccgagatcctgaccgtggagtacggcatcctgcccatcggcaagatcgtggagaagaggatcgagtgcaccgtgtacagcgtggacaacaacggcaacatctacacccagcccgtggcccagtggcacgacaggggcgagcaggaggtgttcgagtactgcctggaggacggcagcctgatcagggccaccaaggaccacaagttcatgaccgtggacggccagatgatgcccatcgacgagatcttcgagagggagctggacctgatgagggtggacaacctgcccaac

SEQ ID NO: 43 - DNA, 108 bp, Artificial Sequence
  synthetic
atggtgaaggtgatcggcaggaggagcctgggcgtgcagaggatcttcgacatcggcctgccccagtaccacaacttcctgctggccaacggcgccatcgccgccaac

SEQ ID NO: 44 - DNA, 270 bp, Artificial Sequence
  synthetic
ctagcgttgacattgattattgactagtacggtaaatggcccgcctggctgatgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaactagagaacccactgcttactggcttctcgagattccaccatggcgc

SEQ ID NO: 45 - DNA, 3225 bp, Artificial Sequence
  synthetic
atgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatctcctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagagttagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacat

SEQ ID NO: 46 - DNA, 59 bp, Artificial Sequence
  synthetic
aattcaataaaagatctttattttcattagatctgtgtgttggttttttgtgtgcggcc

SEQ ID NO: 47 - DNA, 4212 bp, Artificial Sequence
  synthetic
tgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaactagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttac

SEQ ID NO: 48 - DNA, 3822 bp, Artificial Sequence
  synthetic
atgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatctcctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagagttagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagttt

SEQ ID NO: 49 - DNA, 3615 bp, Artificial Sequence
  synthetic
agtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaactagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttac

SEQ ID NO: 50 - DNA, 3822 bp, Artificial Sequence; identical to SEQ ID NO: 48
  synthetic

SEQ ID NO: 51 - DNA, 3615 bp, Artificial Sequence; identical to SEQ ID NO: 49
  synthetic

SEQ ID NO: 52 - DNA, 2784 bp, Artificial Sequence
  synthetic
atgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatctcctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagagttagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtt

SEQ ID NO: 53 - DNA, 1635 bp, Artificial Sequence
  synthetic
tgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaact

SEQ ID NO: 54 - DNA, 3018 bp, Artificial Sequence
  synthetic
tgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaactagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttac

SEQ ID NO: 55 - DNA, 1356 bp, Artificial Sequence
  synthetic
atgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatctcctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagagttagttgaggctctgaagaggttaaaagattatgaa

SEQ ID NO: 56 - DNA, 3063 bp, Artificial Sequence
  synthetic
tcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcgaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaact

SEQ ID NO: 57 - DNA, 1356 bp, Artificial Sequence; identical to SEQ ID NO: 55
  synthetic

SEQ ID NO: 58 - DNA, 3063 bp, Artificial Sequence; identical to SEQ ID NO: 56
  synthetic

SEQ ID NO: 59 - DNA, 3018 bp, Artificial Sequence; identical to SEQ ID NO: 54
  synthetic

SEQ ID NO: 60 - DNA, 4908 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggcaagggcgcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgcctgagctacgagaccgagatcctgaccgtggagtacggcctgctgcccatcggcaagatcgtggagaagcggatcgagtgcaccgtgtacagcgtggacaacaacggcaacatctacacccagcccgtggcccagtggcacgaccggggcgagcaggaggtgttcgagtactgcctggaggacggcagcctgatccgggccaccaaggaccacaagttcatgaccgtggacggccagatgctgcccatcgacgagatcttcgagcgggagctggacctgatgcgggtggacaacctgcccaacgactacaaagaccatgacggtgattataaagatcatgacatcgactacaaggatgacgatgacaagtgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 61 - DNA, 4634 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggcaagggcgcggccgcatgatcaagatcgccacccggaagtacctgggcaagcagaacgtgtacgacatcggcgtggagcgggaccacaacttcgccctgaagaacggcttcatcgccagcaattgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggacgactacaaagaccatgacggtgattataaagatcatgacatcgactacaaggatgacgatgacaagtgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 62 - DNA, 4875 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagcgttgacattgattattgactagtacggtaaatggcccgcctggctgatgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaactagagaacccactgcttactggcttctcgagattccaccatggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgcctgagctacgagaccgagatcctgaccgtggagtacggcctgctgcccatcggcaagatcgtggagaagcggatcgagtgcaccgtgtacagcgtggacaacaacggcaacatctacacccagcccgtggcccagtggcacgaccggggcgagcaggaggtgttcgagtactgcctggaggacggcagcctgatccgggccaccaaggaccacaagttcatgaccgtggacggccagatgctgcccatcgacgagatcttcgagcgggagctggacctgatgcgggtggacaacctgcccaacgactacaaagaccatgacggtgattataaagatcatgacatcgactacaaggatgacgatgacaagtgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 63 - DNA, 4602 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagcgttgacattgattattgactagtacggtaaatggcccgcctggctgatgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaactagagaacccactgcttactggcttctcgagattccaccatggcggccgccatgatcaagatcgccacccggaagtacctgggcaagcagaacgtgtacgacatcggcgtggagcgggaccacaacttcgccctgaagaacggcttcatcgccagcaattgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggacgactacaaagaccatgacggtgattataaagatcatgacatcgactacaaggatgacgatgacaagtgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 64 - DNA, 4955 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagcctagtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggcaagggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgcctgagctacgagaccgagatcctgaccgtggagtacggcctgctgcccatcggcaagatcgtggagaagcggatcgagtgcaccgtgtacagcgtggacaacaacggcaacatctacacccagcccgtggcccagtggcacgaccggggcgagcaggaggtgttcgagtactgcctggaggacggcagcctgatccgggccaccaaggaccacaagttcatgaccgtggacggccagatgctgcccatcgacgagatcttcgagcgggagctggacctgatgcgggtggacaacctgcccaacgactacaaagaccatgacggtgattataaagatcatgacatcgactacaaggatgacgatgacaagatcagcctgatcgccgccctggccgtggactacgtgatcggcatggagaacgccatgccctggaacctgcccgccgacctggcctggttcaagaggaacaccctgaacaagcccgtgatcatgggcaggcacacctgggagagcatcggcaggcccctgcccggcaggaagaacatcatcctgagcagccagcccagcaccgacgacagggtgacctgggtgaagagcgtggacgaggccatcgccgcctgcggcgacgtgcccgagatcatggtgatcggcggcggcagggtgatcgagcagttcctgccctgattcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccaattgaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 65 - DNA, 4929 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagcgttgacattgattattgactagtacggtaaatggcccgcctggctgatgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaactagagaacccactgcttactggcttctcgagattccaccatggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagtgcctgagctacgagaccgagatcctgaccgtggagtacggcctgctgcccatcggcaagatcgtggagaagcggatcgagtgcaccgtgtacagcgtggacaacaacggcaacatctacacccagcccgtggcccagtggcacgaccggggcgagcaggaggtgttcgagtactgcctggaggacggcagcctgatccgggccaccaaggaccacaagttcatgaccgtggacggccagatgctgcccatcgacgagatcttcgagcgggagctggacctgatgcgggtggacaacctgcccaacgactacaaagaccatgacggtgattataaagatcatgacatcgactacaaggatgacgatgacaagtgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 66 - DNA, 4548 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagcgttgacattgattattgactagtacggtaaatggcccgcctggctgatgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaactagagaacccactgcttactggcttctcgagattccaccatggcggccgccatgatcaagatcgccacccggaagtacctgggcaagcagaacgtgtacgacatcggcgtggagcgggaccacaacttcgccctgaagaacggcttcatcgccagcaatagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggacgactacaaagaccatgacggtgattataaagatcatgacatcgactacaaggatgacgatgacaagtgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 67 - DNA, 4695 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagcgttgacattgattattgactagtacggtaaatggcccgcctggctgatgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaactagagaacccactgcttactggcttctcgagattccaccatggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctgcctgagctacgacaccgagatcctgaccgtggagtacggcatcctgcccatcggcaagatcgtggagaagaggatcgagtgcaccgtgtacagcgtggacaacaacggcaacatctacacccagcccgtggcccagtggcacgacaggggcgagcaggaggtgttcgagtactgcctggaggacggcagcctgatcagggccaccaaggaccacaagttcatgaccgtggacggccagatgatgcccatcgacgagatcttcgagagggagctggacctgatgagggtggacaacctgcccaacgactacaaagaccatgacggtgattataaagatcatgacatcgactacaaggatgacgatgacaagtgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 68 - DNA, 4782 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagcgttgacattgattattgactagtacggtaaatggcccgcctggctgatgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaactagagaacccactgcttactggcttctcgagattccaccatggcggccgccatggtgaaggtgatcggcaggaggagcctgggcgtgcagaggatcttcgacatcggcctgccccagtaccacaacttcctgctggccaacggcgccatcgccgccaactctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggacgactacaaagaccatgacggtgattataaagatcatgacatcgactacaaggatgacgatgacaagtgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 69 - DNA, 1983 bp, Artificial Sequence
  synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctctagtagcacagtgtctggcatgtagcaggaactaaaataatggcagtgattaatgttatgatatgcagacacaacacagcaagataagatgcaatgtaccttctgggtcaaaccaccctggccactcctccccgatacccagggttgatgtgcttgaattagacaggattaaaggcttactggagctggaagccttgccccaactcaggagtttagccccagaccttctgtccaccagcgcggccgaccggccaagggcgaattctgcagatatccatcacactggcatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 70 - DNA, 251 bp, Artificial Sequence
  synthetic
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 71 - DNA, 57 bp, Artificial Sequence
  synthetic
atgcaaatagagctctccacctgcttctttctgtgccttttgcgattctgctttagt

SEQ ID NO: 72 - DNA, 3876 bp, Artificial Sequence
  synthetic
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgca

SEQ ID NO: 73 - DNA, 3120 bp, Artificial Sequence
  synthetic
tgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 74 - DNA, 2892 bp, Artificial Sequence
  synthetic
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgta

SEQ ID NO: 75 - DNA, 4104 bp, Artificial Sequence
  synthetic
tcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 76 - protein, 7 aa, Artificial Sequence
  synthetic
GVQCFSR

SEQ ID NO: 77 - protein, 22 aa, Artificial Sequence
  synthetic
LPVPWPTLVTTLTYGVQCFSRY

SEQ ID NO: 78 - protein, 16 aa, Artificial Sequence
  synthetic
PTLVTTLTYGVQCFSR

SEQ ID NO: 79 - protein, 9 aa, Artificial Sequence
  synthetic
TYGVQCFSR

SEQ ID NO: 80 - protein, 8 aa, Artificial Sequence
  synthetic
YGVQCFSR

SEQ ID NO: 81 - protein, 6 aa, Artificial Sequence
  synthetic
VQCFSR

SEQ ID NO: 82 - protein, 5 aa, Artificial Sequence
  synthetic
QCFSR

SEQ ID NO: 83 - protein, 6 aa, Artificial Sequence
  synthetic
KNCFGT

SEQ ID NO: 84 - protein, 8 aa, Artificial Sequence
  synthetic
KNCFGTGL

SEQ ID NO: 85 - protein, 9 aa, Artificial Sequence
  synthetic
KNCFGTGLY

SEQ ID NO: 86 - protein, 10 aa, Artificial Sequence
  synthetic
FLKNCFGTGL

SEQ ID NO: 87 - protein, 11 aa, Artificial Sequence
  synthetic
KNCFGTGLYLT

SEQ ID NO: 88 - protein, 12 aa, Artificial Sequence
  synthetic
KNCFGTGLYLTL

SEQ ID NO: 89 - protein, 13 aa, Artificial Sequence
  synthetic
LYCSGTPLFLKNC

SEQ ID NO: 90 - protein, 13 aa, Artificial Sequence
  synthetic
YCSGTPLFLKNCF

SEQ ID NO: 91 - protein, 14 aa, Artificial Sequence
  synthetic
KNCFGTGLYLTLVR

SEQ ID NO: 92 - protein, 16 aa, Artificial Sequence
  synthetic
KNCFGTGLYLTLVRKM

SEQ ID NO: 93 - protein, 29 aa, Artificial Sequence
  synthetic
IAIIAQGRLYCSGTPLFLKNCFGTGLYLT

SEQ ID NO: 94 - protein, 36 aa, Artificial Sequence
  synthetic
QGRLYCSGTPLFLKNCFGTGLYLTLVRKMKNIQSQR

SEQ ID NO: 95 - protein, 40 aa, Artificial Sequence
  synthetic
GTPLFLKNCFGTGLYLTLVRKMKNIQSQRKGSEGTCSCSS

(19) United States
(12) Patent Application Publication
(10) Pub. No.: US 2021/0371878 A1
(43) Pub. Date: Dec. 2, 2021

(54) INTEIN PROTEINS AND USES THEREOF

(71) Applicant: FONDAZIONE TELETHON, Roma (IT)

(72) Inventors: Alberto AURICCHIO, Pozzuoli (IT); Ivana TRAPANI, Pozzuoli (IT); Patrizia TORNABENE, Pozzuoli (IT)

(21) Appl. No.: 17/285,356

(22) PCT Filed: Oct. 15, 2019
(86) PCT No.: PCT/EP2019/078020

§ 371 Date: Apr. 14, 2021

(30) Foreign Application Priority Data
Oct. 15, 2018 (EP) 18200490.3
Apr. 12, 2019 (EP) 19169116.1

(51) Int. Cl. C12N 15/86; A61P 27/02; A61P 7/04

(52) U.S. Cl. CPC C12N 15/86; C12N 2750/14143; A61P 7/04; A61P 27/02

(57) ABSTRACT

The present invention relates to constructs, vectors, relative host cells and pharmaceutical compositions which allow an effective gene therapy, in particular of genes larger than 5 Kb.

### TECHNICAL FIELD

The present invention relates to constructs, vectors, relative host cells and pharmaceutical compositions which allow an effective gene therapy, in particular for diseases due to mutations in genes with a coding sequence (CDS) larger than 5 kb.

### BACKGROUND OF THE INVENTION

Gene therapy with adeno-associated viral (AAV) vectors is safe and effective in humans. AAV-based gene therapy products have been approved in recent years both in USA and Europe for inherited metabolic and blinding diseases, whilst clinical trials for AAV-based gene therapy approaches for diseases in different therapeutic areas ranging from ophthalmology to hematology to musculoskeletal and metabolic disorders, are ever increasing.

However, the limit of AAV vectors cargo capacity prevents development of AAV-based therapies for diseases due to mutations in genes with a coding sequence (CDS) larger than 5 kb (herein referred to also as large genes).

Genetic diseases due to mutations in large genes (listed in Table 1 below) include, among others, Duchenne muscular dystrophy due to mutations in the DMD gene, cystic fibrosis due to mutations in CFTR gene, hemophilia A due to mutations in F8 gene, dysferlinopathies due to mutations in the DYSF gene, Polycystic kidney disease due to mutation in PKD gene, Wilson's disease due to mutation in ATP7B gene, Huntington's disease due to mutation in HTTgene, Niemann-Pick type C due to mutation in NPC1 gene.

TABLE 1
Genetic diseases due to mutations in large genes

| DISEASE | GENE | CDS | | Accession number |
|---|---|---|---|---|
| Duchenne muscular dystrophy | DMD | 11 | Kb | NM_000109 |
| cystic fibrosis | CFTR | 4.4 | Kb | NM_000492 |
| hemophilia A | F8 | 7 | Kb | NM_000132 |
| dysferlinopathies | DYSF | 6.2 | Kb | NM_001130455 |
| Polycystic kidney disease | PKD1 | 12.9 | Kb | NM_000296 |
| Wilson's disease | ATP7B | 4.4 | Kb | NM_000053 |
| Huntington's disease | HTT | 9.4 | Kb | NM_002111 |
| Niemann-Pick type C | NPC1 | 3.8 | Kb | NM_000271 |

Furthermore, several inherited retinal degenerations (IRDs) are due to mutations in large genes, as listed table 2 below. IRDs affect ˜1 in 3000 people in Europe and the United States (58).

Among the most frequent and severe IRDs are retinitis pigmentosa (RP), Leber congenital amaurosis (LCA), and Stargardt disease (STGD), which are most often inherited as monogenic conditions, with an overall global prevalence of 1/2,000 (1), and are a major cause of blindness worldwide. The majority of mutations causing IRDs occur in genes expressed in neuronal photoreceptors (PR), rods and/or cones in the retina (2).

Gene therapy holds great promise for the treatment of IRDs. The first adeno-associated viral (AAV) vector-based gene therapy product for an inherited form of blindness was approved in December 2017 (3). In addition, a number of other AAV-based products are currently under clinical development for gene therapy of rare and common forms of blindness (4). While it is now well established that AAV represents, to date, the most efficient gene therapy vehicle for the retina (4,5) its limited cargo capacity has hampered its use for conditions that require delivery of DNA sequences that exceed 5 kb in size (6) which include not only the transgene but also the cis regulatory elements that are necessary for its expression.

Examples of disease genes exceeding 5 kb in size are summarized in table 2 below.

TABLE 2
Disease genes exceeding 5 kb in size

| DISEASE | GENE | CDS | | Accession number | EXPRESSION |
|---|---|---|---|---|---|
| Stargardt Disease and | ABCA4 | 6.8 | Kb | NM_000350 | rod&cone PRs |
| ABCA4-associated diseases | | | | | |
| Usher 1B | MYO7A | 6.7 | Kb | NM_000260 | RPE and PRs |
| Leber Congenital | CEP290 | 7.5 | Kb | NM_025114 | mainly PRs |
| Amaurosis10 | | | | | (pan retinal) |
| Usher1D, Nonsyndromic | CDH23 | 10.1 | Kb | NM_001171930 | PRs |
| deafness, autosomal | | | | | |
| recessive (DFNB12) | | | | | |
| Retinitis Pigmentosa | EYS | 9.4 | Kb | NM_001142800 | PR ECM |
| Usher 2A | USH2a | 15.6 | Kb | NM_007123 | rod&cone PRs |
| Usher 2C | ADGRV1 | 18.0 | Kb | NM_032119 | mainly PRs |
| Alstrom Syndrome | ALMS1 | 12.5 | Kb | NM_015120 | rod&cone PRs |

Stargardt disease (STGD; MIM #248200) is the most common form of inherited macular degeneration caused by mutations in the ABCA4 gene (CDS: 6822 bp), which encodes the all-trans retinal transporter located in the PR outer segment (7); Usher syndrome type IB (USH11B; MIM #276900) is the most severe form of RP and deafness caused by mutations in the MYO7A gene (CDS: 6648 bp) (8) encoding the unconventional MYO7A, an actin-based motor expressed in both PR and RPE within the retina (9-11).

Cone-rod dystrophy type 3, fundus flavimaculatus, age-related macular degeneration type 2, Early-onset severe retinal dystrophy, and Retinitis pigmentosa type 19 are also associated with ABCA4 mutations (herein referred to as ABCA4-associated diseases).

The inventors and others have shown that this limitation can be overcome by using either dual (up to 9 kb) (6, 12, 13) or triple (up to 14 kb) (14) AAV vectors, each containing fragments of the coding sequence (CDS) of the large transgene expression cassette. Dual and triple AAV vectors exploit concatemerization and recombination of AAV genomes to reconstitute the full-length genomes in cells co-infected by multiple AAV vectors. However, the efficiency of transgene expression achieved with either dual or triple AAV vectors in photoreceptors, which are the main therapeutic targets for most inherited retinal diseases, is lower than that achieved with single AAV vectors (6, 14, 15). This might be due to the various limiting steps required for efficient transduction, including proper DNA concatemer formation, stability of the heterogeneous mRNA and splicing efficiency across the junctions of the vectors.

The present inventors have shown in WO2014/170480 and Colella et al (15) dual AAV vectors which reconstitute a large gene by either splicing (trans-splicing), homologous recombination (overlapping), or a combination of the two (hybrid), finding that dual trans-splicing and hybrid vectors to be particularly efficient for treatment of inherited retinal degenerations. Furthermore, Maddalena et al. (14) demonstrated a triple AAV vector approach for genes up to 14 kb. However, the efficiency of transgene expression achieved with either dual or triple AAV vectors is lower than that achieved with single AAV vectors (6, 13, 14). This might be due to the various limiting steps required for efficient transduction, including: proper DNA concatemer formation, stability of the heterogeneous mRNA and splicing efficiency across the junctions of the vectors. Further, the triple AAV vector strategy yields levels of gene expression below the threshold needed for a therapeutic approach.

Therefore, there is still the need for constructs and vectors that can be exploited to reconstitute large gene expression for an effective gene therapy.

The inventors have now found that delivery of multiple AAV vectors each encoding one of the fragments of either reporter or large therapeutic proteins flanked by short split-inteins results in protein trans-splicing and full-length protein reconstitution both in vitro and in vivo.

Inteins are genetic elements transcribed and translated within a host protein from which they self-excise similarly to a protein intron, without leaving amino acid modifications in the final protein product, in the absence of energy supply, exogenous host-specific proteases or co-factors (16, 17, 27, 28). Intein activity is context-dependent, with certain peptide sequences surrounding their ligation junction (called N- and C-exteins) that are required for efficient trans-splicing to occur, of which the most important is an amino acid containing a thiol or hydroxyl group (i.e., Cys, Ser or Thr) as first residue in the C-extein (18). Split-inteins are a subset of inteins that are expressed as two separate polypeptides at the ends of two host proteins, and catalyze their trans-splicing resulting in the generation of a single larger polypeptide (19). Inteins, including split-inteins, are widely used in biotechnological applications that include protein purification and labeling steps (19, 20), as well as the reconstitution of the widely used CRISPR/Cas9 genome editing nuclease (21, 22).

Several attempts have been made at exploiting intein-based protein splicing to reconstitute expression of therapeutic genes including the Factor VIII gene, wherein the Synechocystis sp (Ssp) DnaB intein-fused heavy and light chain genes of Factor VIII were demonstrated to lead to reconstitution of Factor VIII in cell culture and in animal models (23, 24). Similarly, a highly functional form of the dystrophin gene was expressed in vitro and in vivo, wherein the 6.3-kb Becker dystrophin gene was split onto two AAV vectors and each half was fused to split inteins obtained from the Synechocystis sp. PCC 6803 (Ssp) DnaB intein or the Rhodothermus marinus (Rma) DnaB intein (25). Further, split-intein (namely N. punctiforme DnaE split inteins)-mediated protein trans-splicing strategy was reported to reconstitute the large pore-forming subunit of L-type calcium channels from two separate fragments in heart cells, (26). U.S. Pat. No. 6,544,786 further reports the use of split inteins to deliver a dystrophin minigene.

The present inventors took advantage of the intrinsic ability of split-inteins to mediate protein trans-splicing to reconstitute large full-length proteins following their fragmentation into either two or three split-intein-flanked polypeptides, whose coding sequences fit into single AAV vectors.

The present invention therefore implements cellular large protein reconstitution by providing to a target cell two or more fragments of said large protein fused to split inteins to promote intein-mediated trans-splicing and reconstitute the functional protein.

### SUMMARY OF THE INVENTION

The present invention provides gene therapy with AAV vectors for diseases due to mutations of genes, in particular of genes with coding regions exceeding 5 kb.

Based on the findings that protein trans-splicing mediated by split-inteins is used by single cell organisms to reconstitute proteins, the inventors have constructed multiple AAV vectors each encoding one of the fragments of either reporter or large therapeutic proteins flanked by short split-inteins, resulting in protein trans-splicing and full-length protein reconstitution in vitro and in vivo.

Advantageously, the AAV-based protein trans-splicing-mediated reconstitution of disease proteins achieved by the present invention afforded expression of larger amounts of target proteins than AAV-based methods for large proteins known in the art. This is probably due to the overcoming of various limiting steps required for efficient transduction of dual vector-based systems including: proper DNA concatemer formation, stability of the heterogeneous mRNA and splicing efficiency across the junctions of the vectors.

The present invention provides a vector system to express a coding sequence in a cell, said coding sequence consisting of a first portion (CDS1), a second portion (CDS2) and optionally a third portion (CDS3), said vector system comprising:
a) a first vector comprising:
said first portion of said coding sequence (CDS1),
a first intein nucleotide sequence coding for a N-Intein, said sequence being located at the 3′ end of CDS1; and
b) a second vector comprising:
said second portion of said coding sequence (CDS2),
a second intein nucleotide sequence coding for a C-Intein, said sequence being located at the 5′ end of CDS2;
   wherein when the first vector and the second vector are inserted in a cell, the protein product of the coding sequence is produced by protein splicing;
   or said vector system comprising:
a′) a first vector comprising:
said first portion of said coding sequence (CDS1),
a first intein nucleotide sequence coding for a first N-Intein, said sequence being located at the 3′ end of CDS1; and
b′) a second vector comprising:
said second portion of said coding sequence (CDS2),
a second intein nucleotide sequence coding for a first C-Intein, said sequence being located at the 5′ end of CDS2;
a third intein nucleotide sequence coding for a second N-Intein, said sequence being located at the 3′ end of CDS2; and
c′) a third vector comprising:
said third portion of said coding sequence (CDS3)
a fourth intein nucleotide sequence coding for a second C-Intein, said sequence being located at the 5′ end of CDS3
   wherein the first intein nucleotide sequence is different from the third intein nucleotide sequence and the second intein sequence is different from the fourth intein nucleotide sequence, wherein when the first vector, the second vector, the third vector are inserted in a cell, the protein product of the coding sequence is produced by protein trans-splicing.

Preferably in the vector system the first intein, the second intein, the third intein and the fourth intein encodes for a split intein, preferably said split intein has a maximum length of 150 amino acids, more preferably said split intein is a DnaE or DnaB intein.

According to the present invention, an intein is a segment of a protein that is able to excise itself and join the remaining portions (the exteins) with a peptide bond in a process known as protein splicing. The segments are called “intein” for internal protein sequence, and “extein” for external protein sequence, with upstream exteins termed “N-exteins” and downstream exteins called “C-exteins”, the upstream intein called “N-Intein” and the downstream intein called “C-Intein”.”

Therefore, in the context of the present invention, an N-Intein is an intein fragment located at the N-terminus of (and fused with) the first polypeptide and a C-Intein is an intein fragment located at the C-terminus of (and fused with) the second polypeptide, wherein upon expression of the two polypeptides, the two intein fragments undergo protein trans-splicing and are joined to form a full intein, and the two polypeptides are joined, wherein when the two polypeptides form a full length protein, said full length protein is reconstituted.

According to the present invention, the first intein sequence is an N-intein sequence and the second intein sequence is a C-Intein sequence, wherein said N-Intein and said C-Intein are preferably derived from the same intein or split intein gene. Alternatively, said N-Intein and said C-Intein derive from two different intein genes which are able to undergo the trans-splicing reaction naturally or are modified to do so. Accordingly, the same gene may be the from the same organism or from different organisms. For instance, widely used split inteins derive from the DnaE gene from different organisms. According to the present invention, when the coding sequence of the protein of interest is split into two portions, the N-intein coding sequence is fused in frame with the sequence coding for the N-terminal portion of the protein of interest; the C-Intein coding sequence is fused in frame with the sequence coding for the C-terminal portion of the sequence of interest. Upon expression of the two precursor fusion proteins, the inteins undergo autocatalytic excision and form a ligated extein, eg the reconstituted protein of interest.

According to the present invention, the coding sequence of the protein of interest may be split into three portions. Accordingly, the first intein sequence is an N-intein sequence and the second intein sequence is a C-Intein sequence, wherein the first intein coding sequence is fused in frame at the C-terminus to the sequence coding for the N-portion of the protein of interest, and the second intein coding sequence is fused in frame at the N-terminus of the sequence coding for the middle portion of the protein of interest. Accordingly, said N-Intein and said C-Intein are preferably derived from the same intein or split intein gene. Alternatively, said N-Intein and said C-Intein derive from two different intein genes which are able to undergo the trans-splicing reaction naturally or are modified to do so. Accordingly, the same gene may be the from the same organism or from different organisms. Within the present configuration, the third intein is an N-Intein coding sequence fused in frame to the sequence coding for the C-terminus of the middle portion of the protein of interest, and the fourth intein is a C-Intein coding sequence fused in frame to the sequence coding for the N-terminus of the C-portion of the protein of interest. Accordingly, said third and fourth inteins are preferably derived from the same intein or split intein gene. Alternatively, said N-Intein and said C-Intein derive from two different intein genes which are able to undergo the trans-splicing reaction naturally or are modified to do so. Accordingly, the same gene may be the from the same organism or from different organisms. Within the scope of the present invention, said first and second inteins and said third and fourth inteins derive from different intein genes and the first intein binds selectively the second intein, while the third intein binds selectively the fourth intein.

In the present invention when the first vector, the second vector and optionally the third vector are inserted in a cell, a least two fusion proteins or three fusion proteins are formed and when contacting said two fusion proteins or three fusion proteins, the protein product of the coding sequence is produced. The step of contacting is performed under conditions that permit binding of the N-intein to the C-intein.

In the present invention when the first vector, the second vector and the third vector are inserted in a cell, three independent polypeptides are produced, and full-length protein is produced via trans-splicing. Pivotal to the development of the three AAV intein vectors has been the use of different inteins, i.e. DnaE and DnaB, which do not cross-react thus preventing improper trans-splicing between the polypeptides produced by the first and the third vector.

According to preferred embodiments of the present invention, a vector system to express the coding sequence of a gene of interest in a cell comprise two vectors, each vector comprising a portion of said coding sequence flanked by an intein sequence, wherein the 5′end of said coding sequence is flanked at the 3′ terminus by the sequence of an N-intein, and the 3′ end of the coding sequence of the gene of interest is flanked by the sequence of a C-Intein, such that when both vectors are expressed in a cell, two fusion proteins are produced and the full length protein of interest is generated as a result of a spontaneous trans-splicing reaction.

According to a further preferred embodiment of the invention, the vector system to express the coding sequence of a gene of interest in a cell comprises three vectors, each vector comprising a portion of said coding sequence flanked by an intein sequence, wherein the coding sequence is divided in three portions such that the 5′end of said coding sequence is flanked at the 3′ terminus by the sequence of a first N-intein; the middle portion of said coding sequence is flanked at the 5′ terminus by a first C-Intein, and at the 3′ terminus with a second N-Intein; the 3′ portion of said coding sequence is flanked at the 5′ terminus by a second C-Intein, such that when all three vectors are expressed in a cell, three fusion proteins are produced, and the full length protein of interest is generated as a result of a spontaneous trans-splicing reaction wherein the first N-Intein reacts with the first C-Intein and the second N-Intein reacts with the second C-Intein.

Split inteins of the invention may be encoded by one gene which is then engineered to encode two separate intein fragments, eg split inteins; alternatively, naturally occurring split inteins are encoded by two separate genes; for instance in cyanobacteria, DnaE, the catalytic subunit α of DNA polymerase III, is encoded by two separate genes, dnaE-n and dnaE-c. Preferred inteins within the present invention are inteins which derive from intein proteins (eg mini inteins) or split inteins which form intein proteins via trans-splicing reaction, which are 150 aa long or less.

Split inteins of the invention may be 100% identical, 98%, 80%, 75%, 70%, 65%, 60%, 55%, 50% identical to naturally occurring inteins or to SEQ ID No. 1 to 14 (homologs), wherein said inteins retain the ability to undergo trans-splicing reactions. Within the scope of the present invention are fragments or variants of naturally occurring or modified inteins which retain trans-splicing activity.

Conveniently, split inteins of the invention may be derived from the same gene isolated from different organisms. Preferred intein genes are Dna B and Dna E.

In a preferred embodiment, the intein of the invention is a split intein derived from the DnaE gene (eg DNA polymerase III subunit alpha) from cyanobacteria including Nostoc punctiforme (Npu) Synechocystis sp. PCC6803 (Ssp), Fischerella sp. PCC 9605, Scytonema tolypothrichoides, Cyanobacteria bacterium SW_9_47_5, Nodularia spumigena, Nostoc flagelliforme, Crocosphaera watsonii WH 8502, Chroococcidiopsis cubana CCALA 043, Trichodesmium erythraeum; preferably, the intein of the invention is derived from Dna E gene isolated from Nostoc puntiforme or Synechocystis sp. PCC6803.

In a further preferred embodiment, the intein of the invention is a split intein derived from the DnaB gene from cyanobacteria including R. marinus (Rma), Synechocystis sp. PC6803 (Ssp), Porphyra purpurea chloroplast (Ppu) which are described for instance in (59).

Preferably,
the first intein nucleotide sequence encodes for an intein selected from the group consisting of: SEQ ID No 1, 3, 5, 7, 9, 11, 13 or a variant thereof or a fragment thereof or an homolog thereof;
the second intein nucleotide sequence encodes for an intein selected from the group consisting of: SEQ ID No 2, 4, 6, 8, 10, 12, 14 or a variant thereof or a fragment thereof or an homolog thereof;
the third intein nucleotide sequence encodes for an intein selected from the group consisting of: SEQ ID No1, 3, 5, 7, 9, 11, 13 or a variant thereof or a fragment thereof or an homolog thereof;
the fourth intein nucleotide sequence encodes for an intein selected from the group consisting of: SEQ ID No2, 4, 6, 8, 10, 12, 14 or a variant thereof or a fragment thereof or an homolog thereof.

Preferably, wherein when the first or third intein is SEQ ID 1, the second or fourth is SEQ ID 2; or when the first or third intein is SEQ ID 3, the second or fourth intein is SEQ ID 4; or when the first or third intein is SEQ ID 5, the second or fourth is SEQ ID 6; or when the first or third intein is SEQ ID 7, the second or fourth is SEQ ID 8; or when the first or third intein is SEQ ID 9, the second or fourth is SEQ ID 10; or when the first or third intein is SEQ ID 11, the second or fourth is SEQ ID 12.

Preferably when the first intein is SEQ ID 1 and the second intein is SEQ ID 2, the third intein is not SEQ ID 1 and the fourth intein is not SEQ ID 2; preferably when the first intein is SEQ ID 3 and the second intein is SEQ ID 4, the third intein is not SEQ ID 3 and the fourth intein is not SEQ ID 4; preferably when the first intein is SEQ ID 5 and the second intein is SEQ ID 6, the third intein is not SEQ ID 5 and the fourth intein is not SEQ ID 6; preferably when the first intein is SEQ ID 7 and the second intein is SEQ ID 8, the third intein is not SEQ ID 7 and the fourth intein is not SEQ ID 8; preferably when the first intein is SEQ ID 9 and the second intein is SEQ ID 10, the third intein is not SEQ ID 9 and the fourth intein is not SEQ ID 10; preferably when the first intein is SEQ ID 11 and the second intein is SEQ ID 12, the third intein is not SEQ ID 11 and the fourth intein is not SEQ ID 12.

In a particular embodiment, the first intein is SEQ ID 1, the second intein is SEQ ID 2, the third intein is SEQ ID 3, the fourth Intein is SEQ ID 4; or, the first intein is SEQ ID 5, the second intein is SEQ ID 6, the third intein is SEQ ID 3 and the fourth Intein is SEQ ID 4.

In a preferred embodiment the first vector, the second vector and the third vector further comprise a promoter sequence operably linked to the 5′end portion of said first portion of the coding sequence (CDS1) or of said second portion of the coding sequence (CDS2) or of said third portion of the coding sequence (CDS3).

Preferred promoters are ubiquitous, artificial, or tissue specific promoters, including fragments and variants thereof retaining a transcription promoter activity. Particularly preferred promoters are photoreceptor-specific promoters including photoreceptor-specific human G protein-coupled receptor kinase 1 (GRK1), Interphotoreceptor retinoid binding protein promoter (IRBP), Rhodopsin promoter (RHO), vitelliform macular dystrophy 2 promoter (VMD2), Rhodopsin kinase promoter (RK); Further particularly preferred promoters are muscle-specific promoters including MCK, MYODI; liver-specific promoters including thyroxine binding globulin (TBG), hybrid liver-specific promoter (HLP) (67); neuron-specific promoters including hSYN1, CaMKlla; kidney-specific promoters including Ksp-cadherin16, NKCC2. Ubiquitous promoters according to the present invention are for instance the ubiquitous cytomegalovirus (CMV)(32) and short CMV (33) promoters More preferred promoters within the scope of the present invention are GRK1, TBG, CaMKlla, Ksp-cadherin16.

In a still preferred embodiment the first vector, the second vector and the third vector further comprise a 5′-terminal repeat (5′-TR) nucleotide sequence and a 3′-terminal repeat (3′-TR) nucleotide sequence, preferably the 5′-TR is a 5′-inverted terminal repeat (5′-ITR) nucleotide sequence and the 3′-TR is a 3′-inverted terminal repeat (3′-ITR) nucleotide sequence.

In a still preferred embodiment the first vector, the second vector and the third vector further comprise a poly-adenylation signal nucleotide sequence.

In a still preferred embodiment the coding sequence is split into the first portion, the second portion and optionally the third portion, at a position consisting of a nucleophile amino acid which does not fall within a structural domain or a functional domain of the encoded protein product, wherein the nucleophile amino acid is selected from serine, threonine, or cysteine.

Preferably at least one of the first vector, the second vector and the third vector further comprises at least one enhancer or regulatory nucleotide sequence, operably linked to the coding sequence.

Preferred enhancer or regulatory nucleotide sequence are the [symbol] -globin IgG chimeric intron, the Woodchuck hepatitis virus Post-transcriptional Regulatory Element.

Optionally, at least one of the first vector, the second vector and the third vector further comprises at least one degradation signal to decrease the stability of the reconstituted intein protein.

Preferably, said degradation signal is a CL1 degron or a PB29 degron. More preferably said degradation signal is ecDHFR or a fragment thereof, preferably the ecDHFR degradation signal is a variant DHFR that functions as internal degron as described herein. Most preferably the fragment retains the degradation property of ecDHFR, preferably the property of a variant DHFR that functions as internal degron preferably the fragment is mini ecDHFR wherein the mini ecDHFR is a variant that functions as internal degron.

Preferably the coding sequence encodes a protein able to correct a pathological state or disorder, preferably the disorder is a retinal degeneration, a metabolic disorder, a blood disorder, a neurodegenerative disorder, hearing loss, channelopathy, lung disease, myopathy, heart disease, muscular dystrophy.

Still preferably the coding sequence encodes a protein able to correct a pathological state or disorder, preferably the disorder is a retinal degeneration, preferably the retinal degeneration is inherited, preferably the pathology or disease is selected from the group consisting of: retinitis pigmentosa (RP), Leber congenital amaurosis (LCA), Stargardt disease (STGD), Usher disease (USH), Alstrom syndrome, congenital stationary night blindness (CSNB), macular dystrophy, occult macular dystrophy, a disease caused by a mutation in the ABCA4 gene. More preferably the coding sequence is the coding sequence of a gene selected from the group consisting of: ABCA4, MYO7A, CEP290, CDH23, EYS, PCDH15, CACNA1, SNRNP200, RP1, PRPF8, RP1L1, ALMS1, USH2A, GPR98, HMCN1 or a fragment thereof or an ortholog thereof or a minigene thereof with a coding sequence exceeding 5kb in length, i.e. a minimal gene fragment that includes one or more exons and the regulatory elements necessary for the gene to express itself in the same way as a wild type gene fragment.

Yet preferably the coding sequence encodes a protein able to correct muscular dystrophy, such as Duchenne muscular dystrophy, cystic fibrosis, hemophilia A, Wilson disease, Phenylketonuria, dysferlinopathies, Rett's syndrome, Polycystic kidney disease, Niemann-Pick type C, Huntington's disease.

More preferably the coding sequence is the coding sequence of a gene selected from the group consisting of: ABCA4, MYO7A, CEP290, CDH23, EYS, PCDH15, CACNA1, SNRNP200, RP1, PRPF8, RP1L1, ALMS1, USH2A, GPR98, HMCN1 or a fragment thereof or an ortholog thereof or a minigene thereof with a coding sequence exceeding 5kb in length, i.e., a minimal gene fragment that includes one or more and the control regions necessary for the gene to express itself in the same way as a wild type gene fragment.

Still preferably the coding sequence is the coding sequence of a gene selected from the group consisting of: DMD, CFTR, F8, ATP7B, PAH, DYSF, MECP2, PKD, NPC1, HTT or a fragment thereof or an ortholog thereof or a minigene thereof thereof with a coding sequence exceeding 5kb in length, i.e., a minimal gene fragment that includes one or more and the regulatory elements necessary for the gene to express itself in the same way as a wild type gene fragment.

In a particularly preferred embodiment of the invention, the coding sequence encodes the ABCA4 gene. Preferably, said coding sequence is split at a nucleotide corresponding to aa Cys1150, Ser1168, Ser 1090 of said ABCA4 protein, and a split intein is inserted at the split point.

In a further preferred embodiment, the coding sequence encodes the CEP290 gene.

Preferably, said coding sequence is split at a nucleotide corresponding to aa Cys1076; Ser1275. More preferably, said coding sequence is split at a nucleotide sequence corresponding to aa Cys 929 and 1474; Ser 453 and Cys 1474 of said CEP290 protein, and two split inteins are inserted at the split points.

EGFP
SEQ ID No. 15
The first amino acid of the c-extein is highlighted whitin the sequence.Split
Cys.71 (bold)
MVSKGEELFTGVVPILVELDGDVNGHKFSVSGEGEGDATYGKLTLKFICTTGKLPVPWPTLVTTLTYGVQ [symbol] FSRYPDHMKQHD
FFKSAMPEGYVQERTIFFKDDGNYKTRAEVKFEGDTLVNRIELKGIDFKEDGNILGHKLEYNYNSHNVYIMADKQKNGIKVNFKI
RHNIEDGSVQLADHYQQNTPIGDGPVLLPDNHYLSTQSALSKDPNEKRDHMVLLEFVTAAGITLGMDELYKDYKDHDGDYKD
HDIDYKDDDDK*
ABCA4
SEQ ID No. 16
The first amino acid of the c-extein is highlighted whitin the sequence.
Split set1 Cys.1150 (bold)
Split set2 Ser.1168 (underlined)
Split set3 Ser.1090 (italic)
MGFVRQIQLLLWKNWTLRKRQKIRFVVELVWPLSLFLVLIWLRNANPLYSHHECHFPNKAMPSAGMLP
WLQGIFCNVNNPCFQSPTPGESPGIVSNYNNSILARVYRDFQELLMNAPESQHLGRIWTELHILSQFMDT
LRTHPERIAGRGIRIRDILKDEETLTLFLIKNIGLSDSVVYLLINSQVRPEQFAHGVPDLALKDIACSEALLERFII
FSQRRGAKTVRYALCSLSQGTLQWIEDTLYANVDFFKLFRVLPTLLDSRSQGINLRSWGGILSDMSPRIQE
FIHRPSMQDLLWVTRPLMQNGGPETFTKLMGILSDLLCGYPEGGGSRVLSFNWYEDNNYKAFLGIDSTR
KDPIYSYDRRTTSFCNALIQSLESNPLTKIAWRAAKPLLMGKILYTPDSPAARRILKNANSTFEELEHVRKLV
KAWEEVGPQIWYFFDNSTQMNMIRDTLGNPTVKDFLNRQLGEEGITAEAILNFLYKGPRESQADDMAN
FDWRDIFNITDRTLRLVNQYLECLVLDKFESYNDETQLTQRALSLLEENMFWAGVVFPDMYPWTSSLPP
HVKYKIRMDIDVVEKTNKIKDRYWDSGPRADPVEDFRYIWGGFAYLQDMVEQGITRSQVQAEAPVGIYL
QQMPYPCFVDDSFMIILNRCFPIFMVLAWIYSVSMTVKSIVLEKELRLKETLKNQGVSNAVIWCTWFLDS
FSIMSMSIFLLTIFIMHGRILHYSDPFILFLFLLAFSTATIMLCFLLSTFFSKASLAAACSGVIYFTLYLPHILCFA
WQDRMTAELKKAVSLLSPVAFGFGTEYLVRFEEQGLGLQWSNIGNSPTEGDEFSFLLSMQMMLLDAAV
YGLLAWYLDQVFPGDYGTPLPWYFLLQESYWLGGEGCSTREERALEKTEPLTEETEDPEHPEGIHDSFFER
EHPGWVPGVCVKNLVKIFEPCGRPAVDRLNITFYENQITAFLGHNGAGKTTTLSILTGLLPPTSGTVLVGG
RDIETSLDAVRQSLGMCPQHNILFHHLTVAEHMLFYAQLKGKSQEEAQLEMEAMLEDTGLHHKRNEEA
QDLSGGMQRKLSVAIAFVGDAKVVILDEPTSGVDPYSRRSIWDLLLKYRSGRTIIMSTHHMDEADLLGDRI
AIIAQGRLYCSGTPLFLKNCFGTGLYLTLVRKMKNIQSQRKGSEGTCSCSSKGFSTTCPAHVDDLTPEQVLD
GDVNELMDVVLHHVPEAKLVECIGQELIFLLPNKNFKHRAYASLFRELEETLADLGLSSFGISDTPLEEIFLKV
TEDSDSGPLFAGGAQQKRENVNPRHPCLGPREKAGQTPQDSNVCSPGAPAAHPEGQPPPEPECPGPQL
NTGTQLVLQHVQALLVKRFQHTIRSHKDFLAQIVLPATFVFLALMLSIVIPPFGEYPALTLHPWIYGQQYTF
FSMDEPGSEQFTVLADVLLNKPGFGNRCLKEGWLPEYPCGNSTPWKTPSVSPNITQLFQKQKWTQVNP
SPSCRCSTREKLTMLPECPEGAGGLPPPQRTQRSTEILQDLTDRNISDFLVKTYPALIRSSLKSKFWVNEQR
YGGISIGGKLPVVPITGEALVGFLSDLGRIMNVSGGPITREASKEIPDFLKHLETEDNIKVWFNNKGWHAL
VSFLNVAHNAILRASLPKDRSPEEYGITVISQPLNLTKEQLSEITVLTTSVDAVVAICVIFSMSFVPASFVLYLI
QERVNKSKHLQFISGVSPTTYWVTNFLWDIMNYSVSAGLVVGIFIGFQKKAYTSPENLPALVALLLLYGW
AVIPMMYPASFLFDVPSTAYVALSCANLFIGINSSAITFILELFENNRTLLRFNAVLRKLLIVFPHFCLGRGLID
LALSQAVTDVYARFGEEHSANPFHWDLIGKNLFAMVVEGVVYFLLTLLVQRHFFLSQWIAEPTKEPIVDE
DDDVAEERQRIITGGNKTDILRLHELTKIYPGTSSPAVDRLCVGVRPGECFGLLGVNGAGKTTTFKMLTGD
TTVTSGDATVAGKSILTNISEVHQNMGYCPQFDAIDELLTGREHLYLYARLRGVPAEEIEKVANWSIKSLGL
TVYADCLAGTYSGGNKRKLSTAIALIGCPPLVLLDEPTTGMDPQARRMLWNVIVSIIREGRAVVLTSHSME
ECEALCTRLAIMVKGAFRCMGTIQHLKSKFGDGYIVTMKIKSPKDDLLPDLNPVEQFFQGNFPGSVQRER
HYNMLQFQVSSSSLARIFQLLLSHKDSLLIEEYSVTQTTLDQVFVNFAKQQTESHDLPLHPRAAGASRQAQ
DDYKDHDGDYKDHDIDYKDDD*
CEP290
SEQ ID No. 17
The first amino acid of the c-extein is highlighted whitin the sequence.
Split set1 Cys.1076 (bold)
Split set2-3 Ser.1275 (underlined)
Split set4 Cys.929 and Cys.1474(italic)
Split set5 Ser.453 and Cys.1474 (double underlined)
MPPNINWKEIMKVDPDDLPRQEELADNLLISLSKVEVNELKSEKQENVIHLFRITQSLMKMKAQEVELAL
EEVEKAGEEQAKFENQLKTKVMKLENELEMAQQSAGGRDTRFLRNEICQLEKQLEQKDRELEDMEKELE
KEKKVNEQLALRNEEAENENSKLRRENKRLKKKNEQLCQDIIDYQKQIDSQKETLLSRRGEDSDYRSQLSK
KNYELIQYLDEIQTLTEANEKIEVQNQEMRKNLEESVQEMEKMTDEYNRM KAIVHQTDNVIDQLKKEND
HYQLQVQELTDLLKSKNEEDDPIMVAVNAKVEEWKLILSSKDDEIIEYQQMLHNLREKLKNAQLDADKSN
VMALQQGIQERDSQIKMLTEQVEQYTKEMEKNTCIIEDLKNELQRNKGASTLSQQTHMKIQSTLDILKEK
TKEAERTAELAEADAREKDKELVEALKRLKDYESGVYGLEDAVVEIKNCKNQIKIRDREIEILTKEINKLELKIS
DFLDENEALRERVGLEPKTMIDLTEFRNSKHLKQQQYRAENQILLKEIESLEEERLDLKKKIRQMAQERGKR
SATSGLTTEDLNLTENISQGDRISERKLDLLSLKNMSEAQSKNEFLSRELIEKERDLERSRTVIAKFQNKLKEL
VEENKQLEEGMKEILQAIKEMQKDPDVKGGETSLIIPSLERLVNAIESKNAEGIFDASLHLKAQVDQLTGR
NEELRQELRESRKEAINYSQQLAKANLKIDHLEKETSLLRQSEGSNVVFKGIDLPDGIAPSSASIINSQNEYLI
HLLQELENKEKKLKNLEDSLEDYNRKFAVIRHQQSLLYKEYLSEKETWKTESKTIKEEKRKLEDQVQQDAIK
VKEYNNLLNALQMDSDEMKKILAENSRKITVLQVNEKSLIRQYTTLVELERQLRKENEKQKNELLSMEAEV
CEKIGCLQRFKEMAIFKIAALQKVVDNSVSLSELELANKQYNELTAKYRDILQKDNMLVQRTSNLEHLECE
NISLKEQVESINKELEITKEKLHTIEQAWEQETKLGNESSMDKAKKSITNSDIVSISKKITMLEMKELNERQR
AEHCQKMYEHLRTSLKQMEERNFELETKFAELTKINLDAQKVEQMLRDELADSVSKAVSDADRQRILELE
KNEMELKVEVSKLREISDIARRQVEILNAQQQSRDKEVESLRMQLLDYQAQSDEKSLIAKLHQHNVSLQLS
EATALGKLESITSKLQKMEAYNLRLEQKLDEKEQALYYARLEGRNRAKHLRQTIQSLRRQFSGALPLAQQE
KFSKTMIQLQNDKLKIMQEMKNSQQEHRNMENKTLEMELKLKGLEELISTLKDTKGAQKVINWHMKIEE
LRLQELKLNRELVKDKEEIKYLNNIISEYERTISSLEEEIVQQNKFHEERQMAWDQREVDLERQLDIFDRQQ
NEILNAAQKFEEATGSIPDPSLPLPNQLEIALRKIKENIRIILETRAT [symbol] KSLEEKLKEKESALRLAEQNILSRDKVI
NELRLRLPATAEREKLIAELGRKEMEPKSHHTLKIAHQTIANMQARLNQKEEVLKKYQRLLEKAREEQREIV
KKHEEDLHILHHRLELQADSSLNKFKQTAWDLMKQSPTPVPTNKHFIRLAEMEQTVAEQDDSLSSLLVKL
KKVSQDLERQREITELKVKEFENIKLQLQENHEDEVKKVKAEVEDLKYLLDQSQKESQCLKSELQAQKEAN
SRAPTTTMRNLVERLKSQLALKEKQQKALSRALLELRAEMTAAAEERIISATSQKEAHLNVQQIVDRHTRE
LKTQVEDLNENLLKLKEALKTSKNRENSLTDNLNDLNNELQKKQKAYNKILREKEEIDQENDELKRQIKRLT
SGLQGKPLTDNKQSLIEELQRKVKKLENQLEGKVEEVDLKPMKEKNAKEELIRWEEGKKWQAKIEGIRNK
LKEKEGEVFTLTKQLNTLKDLFAKADKEKLTLQRKLKTTGMTVDQVLGIRALESEKELEELKKRNLDLENDIL
YMRAHQALPRDSVVEDLHLQNRYLQEKLHALEKQFSKDTYSKPSISGIESDDHCQREQELQKENLKLSSEN
IELKFQLEQANKDLPRLKNQVRDLKEMCEFLKKEKAEVQRKLGHVRGSGRSGKTIPELEKTIGLMKKVVEK
VQRENEQLKKASGILTSEKMANIEQENEKLKAELEKLKAHLGHQLSMHYESKTKGTEKIIAENERLRKELKK
ETDAAEKLRIAKNNLEILNEKMTVQLEETGKRLQFAESRGPQLEGADSKSWKSIVVTRMYETKLKELETDIA
KKNQSITDLKQLVKEATEREQKVNKYNEDLEQQIKILKHVPEGAETEQGLKRELQVLRLANHQLDKEKAELI
HQIEANKDQSGAESTIPDADQLKEKIKDLETQLKMSDLEKQHLKEEIKKLKKELENFDPSFFEEIEDLKYNYK
EEVKKNILLEEKVKKLSEQLGVELTSPVAASEEFEDEEESPVNFPIYDYKDHDGDYKDHDIDYKDDDDK*
F8
SEQ ID No. 18
The first amino acid of the c-extein is highlighted whitin the
sequence.Split set1 Cys.1312 (undeline) set Split set2 Ser.984 (bold)
MQIELSTCFFLCLLRFCFSATRRYYLGAVELSWDYMQSDLGELPVDARFPPRVPKSFPFNTSVVYKKTLFVE
FTDHLFNIAKPRPPWMGLLGPTIQAEVYDTVVITLKNMASHPVSLHAVGVSYWKASEGAEYDDQTSQRE
KEDDKVFPGGSHTYVWQVLKENGPMASDPLCLTYSYLSHVDLVKDLNSGLIGALLVCREGSLAKEKTQTL
HKFILLFAVFDEGKSWHSETKNSLMQDRDAASARAWPKMHTVNGYVNRSLPGLIGCHRKSVYWHVIG
MGTTPEVHSIFLEGHTFLVRNHRQASLEISPITFLTAQTLLMDLGQFLLFCHISSHQHDGMEAYVKVDSCP
EEPQLRMKNNEEAEDYDDDLTDSEMDVVRFDDDNSPSFIQIRSVAKKHPKTWVHYIAAEEEDWDYAPL
VLAPDDRSYKSQYLNNGPQRIGRKYKKVRFMAYTDETFKTREAIQHESGILGPLLYGEVGDTLLIIFKNQAS
RPYNIYPHGITDVRPLYSRRLPKGVKHLKDFPILPGEIFKYKWTVTVEDGPTKSDPRCLTRYYSSFVNMERD
LASGLIGPLLICYKESVDQRGNQIMSDKRNVILFSVFDENRSWYLTENIQRFLPNPAGVQLEDPEFQASNI
MHSINGYVFDSLQLSVCLHEVAYWYILSIGAQTDFLSVFFSGYTFKHKMVYEDTLTLFPFSGETVFMSMEN
PGLWILGCHNSDFRNRGMTALLKVSSCDKNTGDYYEDSYEDISAYLLSKNNAIEPRSFSQNSRHPSTRQK
QFNATTIPENDIEKTDPWFAHRTPMPKIQNVSSSDLLMLLRQSPTPHGLSLSDLQEAKYETFSDDPSPGAI
DSNNSLSEMTHFRPQLHHSGDMVFTPESGLQLRLNEKLGTTAATELKKLDFKVSSTSNNLISTIPSDNLAA
GTDNTSSLGPPSMPVHYDSQLDTTLFGKKSSPLTESGGPLSLSEENNDSKLLESGLMNSQESSWGKNVSS
TESGRLFKGKRAHGPALLTKDNALFKVSISLLKTNKTSNNSATNRKTHIDGPSLLIENSPSVWQNILESDTEF
KKVTPLIHDRMLMDKNATALRLNHMSNKTTSSKNMEMVQQKKEGPIPPDAQNPDMSFFKMLFLPESA
RWIQRTHGKNSLNSGQGPSPKQLVSLGPEKSVEGQNFLSEKNKVVVGKGEFTKDVGLKEMVFPSSRNLF
LTNLDNLHENNTHNQEKKIQEEIEKKETLIQENVVLPQIHTVTGTKNFMKNLFLLSTRQNVEGSYDGAYAP
VLQDFRSLNDSTNRTKKHTAHFSKKGEEENLEGLGNQTKQIVEKYACTTRISPNTSQQNFVTQRSKRALK
QFRLPLEETELEKRIIVDDTSTQWSKNMKHLTPSTLTQIDYNEKEKGAITQSPLSDCLTRSHSIPQANRSPLP
IAKVSSFPSIRPIYLTRVLFQDNSSHLPAASYRKKDSGVQESSHFLQGAKKNNLSLAILTLEMTGDQREVGSL
GTSATNSVTYKKVENTVLPKPDLPKTSGKVELLPKVHIYQKDLFPTETSNGSPGHLDLVEGSLLQGTEGAIK
WNEANRPGKVPFLRVATESSAKTPSKLLDPLAWDNHYGTQIPKEEWKSQEKSPEKTAFKKKDTILSLNAC
ESNHAIAAINEGQNKPEIEVTWAKQGRTERLCSQNPPVLKRHQREITRTTLQSDQEEIDYDDTISVEMKKE
DFDIYDEDENQSPRSFQKKTRHYFIAAVERLWDYGMSSSPHVLRNRAQSGSVPQFKKVVFQEFTDGSFT
QPLYRGELNEHLGLLGPYIRAEVEDNIMVTFRNQASRPYSFYSSLISYEEDQRQGAEPRKNFVKPNETKTYF
WKVQHHMAPTKDEFDCKAWAYFSDVDLEKDVHSGLIGPLLVCHTNTLNPAHGRQVTVQEFALFFTIFDE
TKSWYFTENMERNCRAPCNIQMEDPTFKENYRFHAINGYIMDTLPGLVMAQDQRIRWYLLSMGSNENI
HSIHFSGHVFTVRKKEEYKMALYNLYPGVFETVEMLPSKAGIWRVECLIGEHLHAGMSTLFLVYSNKCQT
PLGMASGHIRDFQITASGQYGQWAPKLARLHYSGSINAWSTKEPFSWIKVDLLAPMIIHGIKTQGARQKF
SSLYISQFIIMYSLDGKKWQTYRGNSTGTLMVFFGNVDSSGIKHNIFNPPIIARYIRLHPTHYSIRSTLRMEL
MGCDLNSCSMPLGMESKAISDAQITASSYFTNMFATWSPSKARLHLQGRSNAWRPQVNNPKEWLQV
DFQKTMKVTGVTTQGVKSLLTSMYVKEFLISSSQDGHQWTLFFQNGKVKVFQGNQDSFTPVVNSLDPP
LLTRYLRIHPQSWVHQIALRMEVLGCEAQDLY*
ecDHFR
SEQ ID No. 19
MISLIAALAVDYVIGMENAMPWNLPADLAWFKRNTLNKPVIMGRHTWESIGRPLPGRKNI
ILSSQPSTDDRVTWVKSVDEAIAACGDVPEIMVIGGGRVIEQFLPKAQKLYLTHIDAEVE
GDTHFPDYEPDDWESVFSEFHDADAQNSHSYCFEILERR*
mini ecDHFR
SEQ ID No. 20
MISLIAALAVDYVIGMENAMPWNLPADLAWFKRNTLNKPVIMGRHTWESIGRPLPGRKNI
ILSSQPSTDDRVTWVKSVDEAIAACGDVPElMVIGGGRVIEQFLP*

In a preferred embodiment, the vector system of the invention comprises:
a) a first vector comprising in a 5′-3′ direction:
a 5′-inverted terminal repeat (5′-ITR) sequence;
a promoter sequence;
a 5′ end portion of a coding sequence (CDS1), said 5′end portion being operably linked to and under control of said promoter;
a first intein nucleotide sequence coding for a N-Intein; and
a 3′-inverted terminal repeat (3′-ITR) sequence; and
b) a second vector comprising in a 5′-3′ direction:
a 5′-inverted terminal repeat (5′-ITR) sequence;
a promoter sequence;
a second intein nucleotide sequence coding for a C-Intein;
a 3′end portion of the coding sequence (CDS2); and
a 3′-inverted terminal repeat (3′-ITR) sequence;
   or comprises:
a′) a first vector comprising in a 5′-3′ direction:
a 5′-inverted terminal repeat (5′-ITR) sequence;
a promoter sequence;
a 5′ end portion of a coding sequence (CDS1′), said 5′end portion being operably linked to and under control of said promoter;
a first intein nucleotide sequence coding for a first N-Intein; and
a 3′-inverted terminal repeat (3′-ITR) sequence; and
b′) a second vector comprising in a 5′-3′ direction:
a 5′-inverted terminal repeat (5′-ITR) sequence;
a promoter sequence;
a second intein nucleotide sequence coding for a first C-Intein;
the second portion of the coding sequence (CDS2′); and
a third intein nucleotide sequence coding for a second N-intein;
a 3′-inverted terminal repeat (3′-ITR) sequence; and
c′) a third vector comprising in a 5′-3′ direction:
a 5′-inverted terminal repeat (5′-ITR) sequence;
a promoter sequence;
a fourth intein nucleotide sequence coding for a second C-Intein;
the third portion of the coding sequence (CDS3′); and
a 3′-inverted terminal repeat (3′-ITR) sequence.

Preferably said first, second and third vector are independently a viral vector, preferably an adeno viral vector or adeno-associated viral (AAV) vector, preferably said first, second and third adeno-associated viral (AAV) vectors are selected from the same or different AAV serotypes, preferably the serotype is selected from the serotype 2, the serotype 8, the serotype 5, the serotype 7 or the serotype 9, serotype 7m8, serotype sh10; serotype 2(quad Y-F).

The present invention also provides a host cell transformed with the vector system as defined above.

Preferably the vector system or the host cell are for medical use, preferably for use in gene therapy, preferably for use in the treatment and/or prevention of a pathology or disease characterized by a retinal degeneration, a metabolic disorder, a blood disorder, a neurodegenerative disorder, hearing loss, channelopathy, lung disease, myopathy, heart disease, muscular dystrophy.

Preferably the retinal degeneration is inherited, preferably the pathology or disease is selected from the group consisting of: retinitis pigmentosa (RP), Leber congenital amaurosis (LCA), Stargardt disease (STGD), Usher disease (USH), Alstrom syndrome, congenital stationary night blindness (CSNB), macular dystrophy, occult macular dystrophy, a disease caused by a mutation in the ABCA4 gene.

Preferably the vector system or the host cell is for use in the prevention and/or treatment of Duchenne muscular dystrophy, cystic fibrosis, hemophilia A, Wilson disease, Phenylketonuria, dysferlinopathies, Rett's syndrome, Polycystic kidney disease, Niemann-Pick type C, Huntington's disease.

The present invention also provides a pharmaceutical composition comprising the vector system or the host cell of the invention and pharmaceutically acceptable vehicle.

### DETAILED DESCRIPTION OF THE INVENTION

### Brief Description of the Drawings

FIG. 1 AAV intein reconstitute EGFP both in vitro and in mouse and pig retina at levels that are higher than dual AAV and up to those achieved with a single AAV.

(A) Schematic representation of AAV intein-mediated protein trans-splicing. ITR: AAV2 inverted terminal repeats; CDS: coding sequence; [symbol] : 3× flag tag; PolyA: polyadenylation signal.

(B) Western blot (WB) analysis of lysates from HEK293 transfected with either full-length or AAV intein CMV-EGFP plasmids. pEGFP: full-length EGFP plasmid; pAAV I+II: AAV-EGFP I+II intein plasmids; pAAV I: single AAV-EGFP I intein plasmid; pAAV II: single AAV-EGFP II intein plasmid; Neg: untransfected cells. The arrows indicate both the full-length EGFP protein (EGFP), the N- and C-terminal halves of the EGFP protein (B and A, respectively), and the reconstituted intein excised from the full-length EGFP protein (C). The WB are representative of n=3 independent experiments.

(C) WB analysis of lysates from HEK293 infected with either single, intein or dual AAV2/2-CMV-EGFP vectors. The WB are representative of n=5 independent experiments.

(D) Retinal cryosections from C57BL/6J mice injected subretinally with AAV2/8-CMV-EGFP intein vectors. Scale bar: 50 μm. RPE: retinal pigment epithelium; OS: outer segments; ONL: outer nuclear layer.

(E-F) Retinal cryosections from either C57BL/6J mice (E) or Large White pigs (F) injected subretinally with either single, intein or dual AAV2/8-GRK1-EGFP vectors. Scale bar: 50 μm (E); 200 am (F). OS: outer segment; ONL: outer nuclear layer.

(G) Fluorescence analysis of retinal organoids infected with AAV2/2-GRK1-EGFP-intein vectors at 293 days of culture. Scale bar: 100 μm.

FIG. 2 Optimization of AAV intein allows proper reconstitution of the large ABCA4 and CEP290 proteins.

(A-B) Western blot (WB) analysis of lysates from HEK293 transfected with different sets of either AAV-shCMV-ABCA4 or -CEP290 intein plasmids (set 1 and set 5, respectively). A schematic representation of the various sets used is depicted in FIG. 16. The WB are representative of n=3 independent experiments.

(C-D) Representative images of immunofluorescence analysis of HeLa cells transfected with either AAV-shCMV-ABCA4 (C) or AAV-shCMV-CEP290 (D) intein plasmids. pABCA4 (C) or pCEP290 (D): plasmid including the full-length expression cassette; pAAV intein: AAV-intein plasmids (either Set 1 in C or Set 5 in D); I+II+III: AAV I+II+III intein plasmids; I+II: AAV I+II intein plasmids; I+III: AAV I+III intein plasmids; II+III: AAV I+III intein plasmids; I: single AAV I intein plasmid; II: single AAV II intein plasmid; III: single AAV III intein plasmid; Neg: untransfected cells.

Cells were stained for 3×FLAG and either VAP-B (endoplasmic reticulum marker) and TGN46 (Trans-Golgi network marker) in C, or acetylated tubulin (marker of microtubules) in D. White arrows point at cells shown at higher magnification in FIG. 18.

FIG. 3 AAV intein reconstitute the large ABCA4 and CEP290 proteins more efficiently than dual AAV vectors.

Western blot (WB) analysis of lysates from HEK293 cells infected with either dual or intein AAV2/2-shCMV-ABCA4 (A) or -CEP290 (B) vectors.

AAV intein: AAV-ABCA4 (set 1, A) or -CEP290 (set 5, B) intein vectors; I+II+III: AAV I+II+III intein vectors; I+II: AAV I+II intein vectors; I+III: AAV I+III intein vectors; II+III: AAV II+III intein vectors; I: single AAV I intein vector; II: single AAV II intein vector; III: single AAV III intein vector; dual AAV: dual AAV vectors; Neg: AAV-EGFP vectors.

(A) The arrows indicate the full-length ABCA4 protein and A: protein product derived from AAV I; B: protein product derived from AAV II. * protein product with a potentially different post-translational modification.

(B) The arrows indicate the full-length CEP290 protein and A: protein product derived from AAV II+III; B: protein product derived from AAV I+II; C: protein product derived from AAV II; D: protein product derived from AAV III; E: protein product derived from AAV I. The WB are representative of n=3 independent experiments

FIG. 4 AAV intein reconstitute large proteins in mouse, pig and human photoreceptors to therapeutic levels.

(A-C) Western blot (WB) analysis of retinal lysates from either wild-type mice (A, B) or Large White pigs (C) injected with either dual or intein AAV2/8-GRK1-ABCA4 (A, C) or -CEP290 (B) vectors (set 1 and set 5, respectively). AAV intein: AAV intein vectors; Dual AAV: dual AAV vectors; Neg: either AAV-EGFP vectors or PBS.

(D) WB analysis of lysates from human iPSCs-derived 3D retinal organoids infected with AAV2/2-GRK1-ABCA4 intein vectors. AAV intein: AAV-ABCA4 intein vectors; Neg: not infected organoids; −/−: organoids derived from STGD1 patients.

(A, C, D) The arrows indicate the full-length ABCA4 protein (ABCA4) and A: protein product derived from AAV I; B: protein product derived from AAV II. * protein product with a potentially different post translational modification.

(B) The arrows indicate both the full-length CEP290 protein (CEP290); A: protein product derived from AAV II+III and D: protein product derived from AAV III.

FIG. 5 Subretinal administration of AAV intein improves the retinal phenotype of mouse models of inherited retinal degenerations.

(A) Quantification of the mean area occupied by lipofuscin in the RPE of Abca4^{-/-} mice treated with AAV intein. Each dot represents the mean value measured for each eye. The mean value of the lipofuscin area for each group is indicated in the graph. +/+ or +/−: control injected Abca4^{+/+} or ^{+/-} eyes (PBS); ^{-/-}: negative control injected Abca4^{-/-} eyes (AAV I ABCA4 or AAV II ABCA4 or PBS); ^{-/-} AAV intein: Abca4^{-/-} eyes injected with AAV intein vectors (set 1). * ANOVA p value <0.05; *** ANOVA p value <0.001.

(B) Representative images of retinal sections from wild-type uninjected and rd16 mice either injected subretinally with AAV2/8-GRK1-CEP290 intein vectors (AAV intein, set 5) or injected with negative controls (Neg; i.e. AAV I+II or AAV II+III or PBS). Scale bar: 25 μm. The thickness of the ONL measured in each image is indicated by the vertical black line. RPE: retinal pigment epithelium; ONL: outer nuclear layer; INL: inner nuclear layer; GCL: ganglion cell layer.

(C) Representative images of eyes from wild-type uninjected and rd16 mice either injected subretinally with AAV2/8-GRK1-CEP290 intein vectors (AAV intein, set 5) or injected with negative controls (Neg; i.e. AAV I+II or AAV II+III or PBS). White circles define pupils.

FIG. 6 Schematic representation of protein trans-splicing-mediated reconstitution of a large protein.

The coding sequence (CDS) of a large gene is split in two halves (5′ and 3′), flanked by the inverted terminal repeats (ITR), which are separately packaged into two AAV capsids. Upon co-transduction of the same cell, different mechanisms are explored to reconstitute full-length protein expression through joining of the two halves at protein level. The 5′-vector includes the 5′ CDS, 5′intein (n-intein) and the degron, while the 3′-vector includes the 3′CDS and 3′intein (c-intein); both vectors include the promoter and the polyA. Pairing of the two half polypeptides is mediated via inteins self-recognition; subsequent intein self-excision from the host protein results in full-length protein reconstitution. The degron, now embedded within the excised intein, it's rapidly ubiquitinated and degraded by the proteasome.

FIG. 7 In vitro EGFP expression from AAV intein vectors with and without degradation signal.

Western blot (WB) analysis of lysates from HEK293 cells transfected with AAV intein plasmids either containing ecDHFR (+) or not (−). The arrows indicate the full-length EGFP protein (EGFP), the excised intein containing the degron (DnaE+ecDHFR) or not (DnaE).

FIG. 8 In vitro ABCA4 expression from AAV intein vectors with and without degradation signal.

Western blot (WB) analysis of lysates from HEK293 cells transfected with AAV intein plasmids either containing ecDHFR (+) or not (−). The arrows indicate the full-length ABCA4 protein (ABCA4), the excised intein containing the degron (DnaE+ecDHFR) or not (DnaE).

FIG. 9 Intein DnaE-ecDHFR expression is TMP-dependent.

Western blot (WB) analysis of lysates from HEK293 cells transfected with AAV_ABCA4 intein plasmids either containing ecDHFR (pAAV intein+ecDHFR) or not (pAAV intein) and treated with increased dose of Trimetrophin (from 1 to 50 [symbol] m). The arrows indicate the excised intein containing the degron (DnaE+ecDHFR) or not (DnaE).

FIG. 10 In vitro EGFP expression from AAV intein vectors with and without degradation signal.

Western blot (WB) analysis of lysates from HEK293 cells transfected with AAV intein plasmids either containing mini ecDHFR (+) or not (−). The arrows indicate the full-length EGFP protein (EGFP), the excised intein containing the degron (DnaE+mini ecDHFR) or not (DnaE).

FIG. 11. In vitro ABCA4 expression from AAV intein vectors with and without degradation signal.

Western blot (WB) analysis of lysates from HEK293 cells transfected with AAV intein plasmids either containing mini ecDHFR (+) or not (−). The arrows indicate the full-length ABCA4 protein (ABCA4), the excised intein containing the degron (DnaE+mini ecDHFR) or not (DnaE).

FIG. 12 EGFP fluorescence in HEK293 cells transfected with AAV I+II but not single AAV I or AAV II intein plasmids.

Fluorescence analysis of HEK293 cells transfected with either full-length or intein CMV-EGFP plasmids. pEGFP: plasmid including the full-length EGFP expression cassette; pAAV I+II: AAV I+II intein plasmids; pAAV I: single AAV I intein plasmid; pAAV II: single AAV II intein plasmid; Neg: untransfected cells. Scale bar: 100 μm.

FIG. 13 Intein relative to full-length protein varies across species.

Western blot (WB) analysis of lysates from HEK293 cells (A), C57BL/6J mice (B) and Large White pig retinas (C) infected with either AAV-CMV-EGFP (A) or AAV-GRK1-EGFP intein vectors (B-C). AAV intein: cells infected (A) or eyes injected (B, C) with AAV intein vectors; Neg: not infected cells (A) or eyes injected with PBS (B, C). The arrows indicate both the full-length EGFP protein (EGFP) and the excised intein (DnaE).

FIG. 14 Characterization of human iPSCs-derived 3D retinal organoids.

(A) Light microscopy analysis of retinal organoids at 183 days of culture.

(B) Immunofluorescence analysis with antibodies directed to mature photoreceptor markers. Scale bar: 100 μm.

(C) Fluorescence analysis of retinal organoids infected with both AAV2/2-CMV-EGFP and AAV2/2-IRBP-DsRed vectors. Scale bar: 100 μm.

(D) Outer segment-like structures were observed which protrude from the surface of retinal organoids at 230 days of culture. The inset shows the presence of outer segment (OS)-like structures with radial architecture. NR: neural retina; RPE: retinal pigment epithelium.

(E) Scanning electron microscopy analysis reveals the presence of inner segments (IS), connecting cilia (CC) and outer segment (OS)-like structures. Scale bar: 4 μm.

(F) Electron microscopy analysis reveals the presence of the outer limiting membrane (*), centriole (C), basal bodies (BB), connecting cilia (CC) and sketches of outer segments (OS). The inset shows the presence of disorganized membranous discs in the OS. Scale bar: 500 nm.

D: days of culture.

FIG. 15 Low intein relative to full-length protein in human 3D retinal organoids.

Western blot (WB) analysis of lysates from human iPSCs-derived 3D retinal organoids infected with AAV2/2-GRK1-EGFP intein vectors. AAV intein: AAV intein vectors; Neg: not infected organoids. The arrows indicate both the full-length EGFP protein (EGFP) and the excised intein (DnaE).

FIG. 16 Schematic representation of the various sets of AAV-ABCA4 and -CEP290 intein.

(A) AAV-ABCA4-intein constructs. (Set 1-2 as exemplified by construct) n-DnaE: n-intein from DnaE of Npu; c-DnaE: c-intein from DnaE of Npu; (Set 3) n-mDnaE: n-intein from mutated DnaE of Npu (mNpu); c-mDnaE: c-intein from DnaE of mNpu.

(B) AAV-CEP290-intein cosntructs. (Set 1) n-DnaE: n-intein from DnaE of Npu; c-DnaE: c-intein from DnaE of Npu; shPolyA: short synthetic polyA; (Set 2) n-DnaE: n-intein from DnaE of mNpu; c-DnaE: c-intein from DnaE of mNpu; (Set 3) n-mDnaE: n-intein from DnaE of mNpu; c-mDnaE: c-intein from DnaE of mNpu; (Set 4) n-DnaE: n-intein from DnaE of Npu; c-DnaE: c-intein from DnaE of Npu between AAV I and AAV II; n-DnaB: N-intein from DnaB of Rhodothermus marinus (Rma); c-DnaB: c-intein from DnaE of Rma between AAV II and AAV III; wpre: Woodchuck hepatitis virus Posttranscriptional Regulatory Element. (Set 5) n-mDnaE: n-intein from DnaE of mNpu; c-mDnaE: c-intein from DnaE of mNpu between AAV I [symbol] and AAV II; n-DnaB: n-intein from DnaB of Rhodothermus marinus (Rma); c-DnaB: c-intein from DnaE of Rma between AAV II and AAV II; wpre: Woodchuck hepatitis virus Posttranscriptional Regulatory Element. (A-B) ITR: AAV2 inverted terminal repeats; : 3× flag tag; Promoter: short CMV for the in vitro experiments and the human G-protein coupled receptor (GRK1) promoter for the in vivo experiments; PolyA: simian virus 40 polyadenylation signal (for ABCA4, A) and bovine growth hormone polyadenylation signal (for CEP290, B). Amino acids at the splitting points of each set are depicted in the figure. Predicted proteins molecular weights are depicted below each AAV vector.

FIG. 17 Combination of heterologous N- and C-inteins does not result in detectable EGFP protein reconstitution in vitro.

Fluorescence analysis of HEK293 cells transfected with either full-length or intein AAV-CMV-EGFP plasmids. N+C-DnaE: AAV I+II fused to inteins from DnaE; N+C-DnaB: AAV I+II fused to inteins from DnaB; N+C-mDnaE: AAV I+II fused to split-inteins from mDnaE; N-DnaE+C-DnaB: AAV I fused to n-intein from DnaE and AAV II fused to c-intein from DnaB; N-DnaB+C-DnaE: AAV I fused to n-intein from DnaB and AAV II fused to c-intein from DnaE; N-mDnaE+C-DnaB: AAV I fused to n-intein from mDnaE and AAV II fused to c-intein from DnaB; N-DnaB+C-mDnaE: AAV I fused to n-intein from DnaB and AAV II fused to c-intein from mDnaE; pEGFP: plasmid including the full-length EGFP expression cassette; Neg: untransfected cells. Scale bar: 100 μm.

FIG. 18 CEP290 aligns along microtubules.

Magnification of single cells from FIG. 2D. Immunofluorescence analysis of HeLa cells transfected either with a plasmid including the full-length CEP290 expression cassette (pCEP290) or with CEP290 intein plasmids (set 5, pAAV I+II+III). Cells were stained for 3×FLAG and acetylated tubulin (marker of microtubules). Scale bar: 50 μm.

Western blot (WB) analysis of lysates from HEK293 cells transfected with either full-length or AAV intein plasmids encoding for either short-CMV-ABCA4 (set 1, A) or -CEP290 (set 5, B).

(A) pABCA4: full-length ABCA4 expression cassette; Set 1: ABCA4 (Cys.1150)-intein plasmids.

(B) pCEP290: full-length CEP290 expression cassette; Set 5: CEP290 (Ser.453 and Cys.1474)-intein plasmids.

Neg: AAV EGFP plasmids. The WB are representative of n=3 independent experiments.

FIG. 19 Transfection of AAV intein plasmids reconstitutes ABCA4 and CEP290 proteins at lower amounts than transfection of single plasmids with full-length expression cassettes.

Western blot (WB) analysis of lysates from HEK293 cells transfected with either full-length or AAV intein plasmids encoding for either short-CMV-ABCA4 (A) or -CEP290 (B). (A) pABCA4: full-length ABCA4 expression cassette; Set 1: ABCA4 (Cys.1150)-intein plasmids. (B) pCEP290: full-length CEP290 expression cassette; Set 5: CEP290 (Ser.453 and Cys.1474)-intein plasmids. Neg: AAV EGFP plasmids. The WB are representative of n=3 independent experiments.

FIG. 20 Subretinal delivery of AAV intein vectors results in ABCA4 expression in the mouse retina.

Western blot (WB) analysis of retinal lysates from wild-type mice injected with either dual or intein AAV2/8-GRK1-ABCA4 vectors (set 1). AAV intein: AAV intein vectors; Dual AAV: dual AAV vectors; Neg: AAV-EGFP vectors.

FIG. 21 AAV intein reconstitute about 10% of endogenous Abca4.

Western blot (WB) analysis of retinal lysates from either Abca4^{+/-} or Abca4^{-/-} mice injected with AAV2/8-GRK1-ABCA4 intein vectors (set 1). mAbca4: Abca4^{+/-} retina; AAV intein: AAV intein-injected retina; Neg: not injected retina. Retinal lysates from Abca4+/− loaded on Gel #2 and #3 are the same. The percentage of AAV intein ABCA4 expression relative to endogenous is depicted below each lane.

FIG. 22 AAV intein reconstitute full-length ABCA4 protein in human retinal organoids.

Western blot analysis of lysates from human iPSCs-derived 3D retinal organoids infected with AAV2/2-GRK1-ABCA4 intein vectors (set 1). AAV intein: AAV intein vectors; Neg: not infected organoids. −/−: organoids derived from STGD1 patients; +/+: organoids derived from healthy donors.

FIG. 23 Subretinal administration of AAV intein vectors results in reduction of lipofuscin accumulation in Abca4−/− mice.

Representative pictures of transmission electron microscopy analysis showing lipofuscin granules in the RPE of wild-type and Abca4^{-/-} mice injected with either negative control (Neg) or AAV intein vectors (set 1). The white arrows indicate lipofuscin granules; M: mitochondria.

FIG. 24 Subretinal delivery of AAV intein vectors in mice does not modify the ONL thickness.

Spectral domain optical coherence tomogram analysis of C57BL/6J mice eyes injected subretinally with either AAV intein vectors, unrelated AAV vectors (AAV neg) or PBS. The black bars represent eyes at 6 months post-injection with AAV-ABCA4 intein vectors (set 1), and their corresponding controls; the white bars represent eyes at 4.5 months post-injection with AAV-CEP290 intein vectors (set 5), and their corresponding controls. Data are represented as mean±s.e. The mean values are indicated above the corresponding bar.

FIG. 25. AAV intein vectors could deliver the full-length wild type F8

A) Schematic representation of a single AAV B-domain deleted variant 3 Factor VIII (F8-V3) and AAV F8 intein vectors.

The coding sequence of the F8 gene is split into two halves (5′ and 3′ F8), flanked by the inverted terminal repeats (ITR), which are separately packaged into two AAV capsids. The 5′-vector includes the 5′ F8 and 5′ intein (n-DnaE) while the 3′-vector includes the 3′ F8 and 3′ intein (c-DnaE); both vectors include the HLP promoter and the synthetic polyA. V3, variant 3; SS, signal sequence.

B) F8 intein are properly packaged into AAV capsids with defined vector genomes unlike the single oversize AAV F8-V3.

Southern blot analysis of the vectors genome integrity with a probe specific to the HLP promoter showed truncated products in the oversize AAV F8-V3 that were not present in the AAV F8 intein vectors. Neg, negative control.

C) AAV F8 intein vectors show slight correction of the bleeding phenotype of hemophilia A knockout mice at 8 weeks post injection.

aPTT analysis of blood plasma samples of hemophilia A knockout mice at 8 weeks post injection with AAV F8 intein (both splitting points) show slight phenotypic correction compared to the PBS-injected control group. aPTT, activated partial thromboplastin time.

Gene Therapy

During the past decade, gene therapy has been applied to the treatment of disease in hundreds of clinical trials. Various tools have been developed to deliver genes into human cells; among them, genetically engineered viruses, including adeno-associated viruses, are currently amongst the most popular tool for gene delivery. Most of the systems contain vectors that are capable of accommodating genes of interest and helper cells that can provide the viral structural proteins and enzymes to allow for the generation of vector-containing infectious viral particles. Adeno-associated virus is a family of viruses that differs in nucleotide and amino acid sequence, genome structure, pathogenicity, and host range. This diversity provides opportunities to use viruses with different biological characteristics to develop different therapeutic applications. As with any delivery tool, the efficiency, the ability to target certain tissue or cell type, the expression of the gene of interest, and the safety of Adeno-associated virus-based systems are important for successful application of gene therapy. Significant efforts have been dedicated to these areas of research in recent years. Various modifications have been made to Adeno-associated virus-based vectors and helper cells to alter gene expression, target delivery, improve viral titers, and increase safety. The present invention represents an improvement in this design process in that it acts to efficiently deliver genes of interest with a size exceeding the limit cargo for a single adeno-associated virus-based vector. Viruses are logical tools for gene delivery. They replicate inside cells and therefore have evolved mechanisms to enter the cells and use the cellular machinery to express their genes. The concept of virus-based gene delivery is to engineer the virus so that it can express the gene of interest. Depending on the specific application and the type of virus, most viral vectors contain mutations that hamper their ability to replicate freely as wild-type viruses in the host. Viruses from several different families have been modified to generate viral vectors for gene delivery. These viruses include retroviruses, lentivirus, adenoviruses, adeno-associated viruses, herpes simplex viruses, picornaviruses, and alphaviruses. The present invention preferably employs adeno-associated viruses. Therefore, virus-based vectors for gene delivery include without limitations adenoviral vectors, adeno-associated viral (AAV) vectors, pseudotyped AAV vectors, herpes viral vectors, retroviral vectors, lentiviral vectors, baculoviral vectors.

An ideal adeno-associated virus-based vector for gene delivery must be efficient, cell-specific, regulated, and safe. The efficiency of delivery is important because it can determine the efficacy of the therapy. Current efforts are aimed at achieving cell-type-specific infection and gene expression with adeno-associated viral vectors. In addition, adeno-associated viral vectors are being developed to regulate the expression of the gene of interest, since the therapy may require long-lasting or regulated expression. Safety is a major issue for viral gene delivery because most viruses are either pathogens or have a pathogenic potential.

Adeno-associated virus (AAV) is a small virus which infects humans and some other primate species. AAV is not currently known to cause disease and consequently the virus causes a very mild immune response. Gene therapy vectors using AAV can infect both dividing and quiescent cells and persist in an extrachromosomal state without integrating into the genome of the host cell. These features make AAV a very attractive candidate for creating viral vectors for gene therapy, and for the creation of isogenic human disease models.

Wild-type AAV has attracted considerable interest from gene therapy researchers due to a number of features. Chief amongst these is the virus's apparent lack of pathogenicity. It can also infect non-dividing cells and has the ability to stably integrate into the host cell genome at a specific site (designated AAVS1) in the human chromosome 19. The feature makes it somewhat more predictable than retroviruses, which present the threat of a random insertion and of mutagenesis, which is sometimes followed by development of a cancer. The AAV genome integrates most frequently into the site mentioned, while random incorporations into the genome take place with a negligible frequency. Development of AAVs as gene therapy vectors, however, has eliminated this integrative capacity by removal of the rep and cap from the DNA of the vector. The desired gene together with a promoter to drive transcription of the gene is inserted between the inverted terminal repeats (ITR) that aid in concatamer formation in the nucleus after the single-stranded vector DNA is converted by host cell DNA polymerase complexes into double-stranded DNA. AAV-based gene therapy vectors form episomal concatamers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency. AAVs also present very low immunogenicity, seemingly restricted to generation of neutralizing antibodies, while they induce no clearly defined cytotoxic response. This feature, along with the ability to infect quiescent cells present their dominance over adenoviruses as vectors for the human gene therapy.

AAV Genome, Transcriptome and Proteome

The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry.

ITR Sequences

The Inverted Terminal Repeat (ITR) sequences comprise 145 bases each. They were named so because of their symmetry, which was shown to be required for efficient multiplication of the AAV genome. Another property of these sequences is their ability to form a hairpin, which contributes to so-called self-priming that allows primase-independent synthesis of the second DNA strand. The ITRs were also shown to be required for both integration of the AAV DNA into the host cell genome (19th chromosome in humans) and rescue from it, as well as for efficient encapsidation of the AAV DNA combined with generation of a fully assembled, deoxyribonuclease-resistant AAV particles.

With regard to gene therapy, ITRs seem to be the only sequences required in cis next to the therapeutic gene: structural (cap) and packaging (rep) genes can be delivered in trans. With this assumption, many methods were established for efficient production of recombinant AAV (rAAV) vectors containing a reporter or therapeutic gene. However, it was also published that the ITRs are not the only elements required in cis for the effective replication and encapsidation. A few research groups have identified a sequence designated cis-acting Rep-dependent element (CARE) inside the coding sequence of the rep gene. CARE was shown to augment the replication and encapsidation when present in cis.

AAV Serotypes

To date, dozens of different AAV variants (serotypes) have been identified and classified (60). All of the known serotypes can infect cells from multiple diverse tissue types. Tissue specificity is determined by the capsid serotype and pseudotyping of AAV vectors to alter their tropism range will likely be important to their use in therapy. Pseudotyped AAV vectors are those which contain the genome of one AAV serotype in the capsid of a second AAV serotype; for example an AAV2/8 vector contains the AAV8 capsid and the AAV 2 genome (61). Such vectors are also known as chimeric vectors

Serotype 2

Serotype 2 (AAV2) has been the most extensively examined so far. AAV2 presents natural tropism towards skeletal muscles, neurons, vascular smooth muscle cells and hepatocytes. Three cell receptors have been described for AAV2: heparan sulfate proteoglycan (HSPG), aᵥβ₅ integrin and fibroblast growth factor receptor 1 (FGFR-1). The first functions as a primary receptor, while the latter two have a co-receptor activity and enable AAV to enter the cell by receptor-mediated endocytosis. These study results have been disputed by Qiu, Handa, et al.. HSPG functions as the primary receptor, though its abundance in the extracellular matrix can scavenge AAV particles and impair the infection efficiency.

Studies have shown that serotype 2 of the virus (AAV-2) apparently kills cancer cells without harming healthy ones. “Our results suggest that adeno-associated virus type 2, which infects the majority of the population but has no known ill effects, kills multiple types of cancer cells yet has no effect on healthy cells,” said Craig Meyers, a professor of immunology and microbiology at the Penn State College of Medicine in Pennsylvania. This could lead to a new anti-cancer agent.

Other Serotypes

Although AAV2 is the most popular serotype in various AAV-based research, it has been shown that other serotypes can be more effective as gene delivery vectors. For instance AAV6 appears much better in infecting airway epithelial cells, AAV7 presents very high transduction rate of murine skeletal muscle cells (similarly to AAV1 and AAV5), AAV8 is superb in transducing hepatocytes and photorecetors, AAV1 and 5 were shown to be very efficient in gene delivery to vascular endothelial cells. In the brain, most AAV serotypes show neuronal tropism, while AAV5 also transduces astrocytes. AAV6, a hybrid of AAV1 and AAV2, also shows lower immunogenicity than AAV2.

Serotypes can differ with the respect to the receptors they are bound to. For example AAV4 and AAV5 transduction can be inhibited by soluble sialic acids (of different form for each of these serotypes), and AAV5 was shown to enter cells via the platelet-derived growth factor receptor. Novel AAV variants such as quadruple tyrosine mutants or AAV 2/7m8 were shown to transduce the outer retina from the vitreous in small animal models (62, 63). Another AAV mutant named ShH10, an AAV6 variant with improved glial tropism after intravitreal administration (64). A further AAV mutant with particularly advantageous tropism for the retina is the AAV2 (quad Y-F) (65).

The gene delivery vehicles of the present invention may be administered to a patient. Said administration may be an “in vivo” administration or an “ex vivo” administration. A skilled worker would be able to determine appropriate dosage rates. The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors etc as described above.

Non-viral delivery systems include DNA transfection such as electroporation, lipid mediated transfection, compacted DNA-mediated transfection; liposomes, immunoliposomes, lipofectin, cationic facial amphiphiles (CFAs) and combinations thereof.

The delivery of one or more therapeutic genes by a vector system according to the present invention may be used alone or in combination with other treatments or components of the treatment.

Pharmaceutical Compositions

The present invention also provides a pharmaceutical composition for treating an individual by gene therapy, wherein the composition comprises a therapeutically effective amount of the vector/construct or host cell of the present invention comprising one or more deliverable therapeutic and/or diagnostic transgenes(s) or a viral particle produced by or obtained from same. The pharmaceutical composition may be for human or animal usage. Typically, a physician will determine the actual dosage which will be most suitable for an individual subject and it will vary with the age, weight and response of the particular individual. The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s), and other carrier agents that may aid or increase the viral entry into the target site (such as for example a lipid delivery system). Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents; preferably they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner.

A preferred formulation is where the vector system is administered topically in the conjunctival sac, or subconjunctivally, preferably administered from 1 to 10 times a day, preferably for 1 day to 6 months, preferably for 1 day to 30 days.

Preferred administration is administration into the anterior chamber, intravitreal injection, subretinal injection, parabulbar and/or retrobulbar injection, intrastromal corneal injection.

Preferably, the pharmaceutical composition of the invention is for topical ocular use and is therefore an ophthalmic composition.

The vector system according to the present invention can be administered by any convenient route, however the preferred route of administration is topically to the ocular surface and specially topically to the cornea. Even more preferred route is instillation into the conjunctival sac.

It is a specific object of the present invention, the use of the vector system for the production of an ophthalmic composition to be administered topically to the eye for medical use.

More generally, one preferred embodiment of the present invention is a composition formulated for topical application on a local, superficial or restricted area in the eye and/or the adnexa of the eye comprising the vector system optionally together with one or more pharmaceutically acceptable additives (such as diluents or carriers).

As used herein, the terms “vehicle”, “diluent”, “carrier” and “additive” are interchangeable.

The ophthalmic compositions of the invention may be in the form of solution, emulsion or suspension (collyrium), ointment, gel, aerosol, mist or liniment together comprising a pharmaceutically acceptable, eye tolerated and compatible with active principle ophthalmic carrier.

Also within the scope of the present invention are particular routes for ophthalmic administration for delayed release, e.g. as ocular erodible inserts or polymeric membrane “reservoir” systems to be located in the conjunctiva sac or in contact lenses.

The ophthalmic compositions of the invention may be administered topically, e.g., the composition is delivered and directly contacts the eye and/or the adnexa of the eye.

The pharmaceutical composition containing at least a vector system of the present invention may be prepared by any conventional technique, e.g. as described in Remington: The Science and Practice of Pharmacy 1995, edited by E. W. Martin, Mack Publishing Company, 19th edition, Easton, Pa.

In one embodiment the composition is formulated so it is a liquid, wherein the vector system may be in solution or in suspension. The composition may be formulated in any liquid form suitable for topical application such as eye-drops, artificial tears, eye washes, or contact lens adsorbents comprising a liquid carrier such as a cellulose ether (e.g. methylcellulose).

Preferably the liquid is an aqueous liquid. It is furthermore preferred that the liquid is sterile. Sterility may be conferred by any conventional method, for example filtration, irradiation or heating or by conducting the manufacturing process under aseptic conditions.

The liquid may comprise one or more lipophile vehicles.

In one embodiment of the present invention, the composition is formulated as an ointment. Preferably one carrier in the ointment may be a petrolatum carrier.

The pharmaceutical acceptable vehicles may in general be any conventionally used pharmaceutical acceptable vehicle, which should be selected according to the specific formulation, intended administration route etc. Furthermore, the pharmaceutical acceptable vehicle may be any accepted additive from FDAs “inactive ingredients list”, which for example is available on the internet address http://www.fda.gov/cder/drug/iig/default.htm.

At least one pharmaceutically acceptable diluents or carrier may be a buffer. For some purposes it is often desirable that the composition comprises a buffer, which is capable of buffering a solution to a pH in the range of 5 to 9, for example pH 5 to 6, pH 6 to 8 or pH 7 to 7.5.

However, in other embodiments of the invention the pharmaceutical composition may comprise no buffer at all or only micromolar amounts of buffer. The buffer may for example be selected from the group consisting of TRIS, acetate, glutamate, lactate, maleate, tartrate, phosphate, citrate, borate, carbonate, glycinate, histidine, glycine, succinate and triethanolamine buffer. Hence, the buffer may be K2HPO4, Na2HPO4 or sodium citrate.

In a preferred embodiment the buffer is a TRIS buffer. TRIS buffer is known under various other names for example tromethamine including tromethamine USP, THAM, Trizma, Trisamine, Tris amino and trometamol. The designation TRIS covers all the aforementioned designations.

The buffer may furthermore for example be selected from USP compatible buffers for parenteral use, in particular, when the pharmaceutical formulation is for parenteral use. For example, the buffer may be selected from the group consisting of monobasic acids such as acetic, benzoic, gluconic, glyceric and lactic, dibasic acids such as aconitic, adipic, ascorbic, carbonic, glutamic, malic, succinic and tartaric, polybasic acids such as citric and phosphoric and bases such as ammonia, diethanolamine, glycine, triethanolamine, and TRIS.

The compositions may contain preservatives such as thimerosal, chlorobutanol, benzalkonium chloride, or chlorhexidine, buffering agents such as phosphates, borates, carbonates and citrates, and thickening agents such as high molecular weight carboxy vinyl polymers such as the ones sold under the name of Carbopol which is a trademark of the B. F. Goodrich Chemical Company, hydroxymethylcellulose and polyvinyl alcohol, all in accordance with the prior art.

In some embodiments of the invention the pharmaceutically acceptable additives comprise a stabiliser. The stabiliser may for example be a detergent, an amino acid, a fatty acid, a polymer, a polyhydric alcohol, a metal ion, a reducing agent, a chelating agent or an antioxidant, however any other suitable stabiliser may also be used with the present invention. For example, the stabiliser may be selected from the group consisting of poloxamers, Tween-20, Tween-40, Tween-60, Tween-80, Brij, metal ions, amino acids, polyethylene glycol, Triton, and ascorbic acid.

Furthermore, the stabiliser may be selected from the group consisting of amino acids such as glycine, alanine, arginine, leucine, glutamic acid and aspartic acid, surfactants such as polysorbate 20, polysorbate 80 and poloxamer 407, fatty acids such as phosphatidyl choline ethanolamine and acethyltryptophanate, polymers such as polyethylene glycol and polyvinylpyrrolidone, polyhydric alcohol such as sorbitol, mannitol, glycerin, sucrose, glucose, propylene glycol, ethylene glycol, lactose and trehalose, antioxidants such as ascorbic acid, cysteine HCL, thioglycerol, thioglycolic acid, thiosorbitol and glutathione, reducing agents such as several thiols, chelating agents such as EDTA salts, gluthamic acid and aspartic acid.

The pharmaceutically acceptable additives may comprise one or more selected from the group consisting of isotonic salts, hypertonic salts, hypotonic salts, buffers and stabilisers.

In preferred embodiments other pharmaceutically excipients such as preservatives are present. In one embodiment said preservative is a parabene, such as but not limited to methyl parahydroxybenzoate or propyl parahydroxybenzoate.

In some embodiments of the invention the pharmaceutically acceptable additives comprise mucolytic agents (for example N-acetyl cysteine), hyaluronic acid, cyclodextrin, petroleum.

Exemplary compounds that may be incorporated in the pharmaceutical composition of the invention to facilitate and expedite transdermal delivery of topical compositions into ocular or adnexal tissues include, but are not limited to, alcohol (ethanol, propanol, and nonanol), fatty alcohol (lauryl alcohol), fatty acid (valeric acid, caproic acid and capric acid), fatty acid ester (isopropyl myristate and isopropyl n-hexanoate), alkyl ester (ethyl acetate and butyl acetate), polyol (propylene glycol, propanedione and hexanetriol), sulfoxide (dimethylsulfoxide and decylmethylsulfoxide), amide (urea, dimethylacetamide and pyrrolidone derivatives), surfactant (sodium lauryl sulfate, cetyltrimethylammonium bromide, polaxamers, spans, tweens, bile salts and lecithin), terpene (d-limonene, alpha-terpeneol, 1,8-cineole and menthone), and alkanone (N-heptane and N-nonane). Moreover, topically-administered compositions may comprise surface adhesion molecule modulating agents including, but not limited to, a cadherin antagonist, a selectin antagonist, and an integrin antagonist.

Also, the ophthalmic solution may contain a thickener such as hydroxymethylcellulose, hydroxyethylcellulose, hydroxypropylmethylcellulose, methylcellulose, polyvinylpyrrolidone, or the like, to improve the retention of the medicament in the conjunctival sac.

In an embodiment, the vector system for use according to the invention may be combined with ophthalmologically acceptable preservatives, surfactants, viscosity enhancers, penetration enhancers, buffers, sodium chloride and water to form aqueous, sterile, ophthalmic suspensions or solutions. The ophthalmic solution may further include an ophthalmologically acceptable surfactant to assist in dissolving the Vector system. Ophthalmic solution formulations may be prepared by dissolving the vector system in a physiologically acceptable isotonic aqueous buffer.

In order to prepare sterile ophthalmic ointment formulations, the vector system may be combined with a preservative in an appropriate vehicle, such as, mineral oil, liquid lanolin, or white petrolatum. Sterile ophthalmic gel formulations may be prepared by suspending the Vector system in a hydrophilic base prepared from the combination of, for example, carbopol-940, or the like, according to the published formulations for analogous ophthalmic preparations; preservatives and tonicity agents can be incorporated.

Preferably, the formulation of the present invention is an aqueous, non-irritating, ophthalmic composition for topical application to the eye comprising: a therapeutically effective amount of a vector system for topical treatment; a xanthine derivative being present in an amount between the amount of derivative soluble in the water of said composition and 0.05% by weight/volume of said composition which is effective to reduce the discomfort associated with the vector system upon topical application of said composition, said xanthine derivative being selected from the group consisting of theophylline, caffeine, theobromine and mixtures thereof; an ophthalmic preservative; and a buffer, to provide an isotonic, aqueous, nonirritating ophthalmic composition.

Drug Delivery Devices

In one embodiment, the invention comprises a drug-delivery device consisting of at least an vector system and a pharmaceutically compatible polymer. For example, the composition is incorporated into or coated onto said polymer. The composition is either chemically bound or physically entrapped by the polymer. The polymer is either hydrophobic or hydrophilic. The polymer device comprises multiple physical arrangements. Exemplary physical forms of the polymer device include, but are not limited to, a film, a scaffold, a chamber, a sphere, a microsphere, a stent, or other structure. The polymer device has internal and external surfaces. The device has one or more internal chambers. These chambers contain one or more compositions. The device contains polymers of one or more chemically-differentiable monomers. The subunits or monomers of the device polymerize in vitro or in vivo.

In a preferred embodiment, the invention comprises a device comprising a polymer and a bioactive composition incorporated into or onto said polymer, wherein said composition includes a vector system, and wherein said device is implanted or injected into an ocular surface tissue, an adnexal tissue in contact with an ocular surface tissue, a fluid-filled ocular or adnexal cavity, or an ocular or adnexal cavity.

Exemplary mucoadhesive polyanionic natural or semi-synthetic polymers from which the device may be formed include, but are not limited to, polygalacturonic acid, hyaluronic acid, carboxymethylamylose, carboxymethylchitin, chondroitin sulfate, heparin sulfate, and mesoglycan. In one embodiment, the device comprises a biocompatible polymer matrix that may optionally be biodegradable in whole or in part. A hydrogel is one example of a suitable polymer matrix material. Examples of materials which can form hydrogels include polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-.epsilon.-caprolactone, polyanhydrides; polyphosphazines, polyvinyl alcohols), poly(alkylene oxides) particularly poly(ethylene oxides), poly(allylamines)(PAM), poly(acrylates), modified styrene polymers such as poly(4-aminomethylstyrene), pluronic polyols, polyoxamers, poly(uronic acids), poly(vinylpyrrolidone) and copolymers of the above, including graft copolymers. In another embodiment, the scaffolds may be fabricated from a variety of synthetic polymers and naturally-occurring polymers such as, but not limited to, collagen, fibrin, hyaluronic acid, agarose, and laminin-rich gels.

One preferred material for the hydrogel is alginate or modified alginate material. Alginate molecules are comprised of (I-4)-linked β-D-mannuronic acid (M units) and a L-guluronic acid (G units) monomers which vary in proportion and sequential distribution along the polymer chain. Alginate polysaccharides are polyelectrolyte systems which have a strong affinity for divalent cations (e.g. Ca+2, Mg+2, Ba+2) and form stable hydrogels when exposed to these molecules.

The device is administered topically, subconjunctively, or in the episcleral space, subcutaneously, or intraductally. Specifically, the device is placed on or just below the surface of an ocular tissue. Alternatively, the device is placed inside a tear duct or gland. The composition incorporated into or onto the polymer is released or diffuses from the device.

In one embodiment the composition is incorporated into or coated onto a contact lens or drug delivery device, from which one or more molecules diffuse away from the lens or device or are released in a temporally-controlled manner. In this embodiment, the contact lens composition either remains on the ocular surface, e.g. if the lens is required for vision correction, or the contact lens dissolves as a function of time simultaneously releasing the composition into closely juxtaposed tissues. Similarly, the drug delivery device is optionally biodegradable or permanent in various embodiments.

For example, the composition is incorporated into or coated onto said lens. The composition is chemically bound or physically entrapped by the contact lens polymer. Alternatively, a colour additive is chemically bound or physically entrapped by the polymer composition that is released at the same rate as the therapeutic drug composition, such that changes in the intensity of the colour additive indicate changes in the amount or dose of therapeutic drug composition remaining bound or entrapped within the polymer. Alternatively, or in addition, an ultraviolet (UV) absorber is chemically bound or physically entrapped within the contact lens polymer. The contact lens is either hydrophobic or hydrophilic.

Exemplary materials used to fabricate a hydrophobic lens with means to deliver the compositions of the invention include, but are not limited to, amefocon A, amsilfocon A, aquilafocon A, arfocon A, cabufocon A, cabufocon B, carbosilfocon A, crilfocon A, crilfocon B, dimefocon A, enflufocon A, enflofocon B, erifocon A, flurofocon A, flusilfocon A, flusilfocon B, flusilfocon C, flusilfocon D, flusilfocon E, hexafocon A, hofocon A, hybufocon A, itabisfluorofocon A, itafluorofocon A, itafocon A, itafocon B, kolfocon A, kolfocon B, kolfocon C, kolfocon D, lotifocon A, lotifocon B, lotifocon C, melafocon A, migafocon A, nefocon A, nefocon B, nefocon C, onsifocon A, oprifocon A, oxyfluflocon A, paflufocon B, paflufocon C, paflufocon D, paflufocon E, paflufocon F, pasifocon A, pasifocon B, pasifocon C, pasifocon D, pasifocon E, pemufocon A, porofocon A, porofocon B, roflufocon A, roflufocon B, roflufocon C, roflufocon D, roflufocon E, rosilfocon A, satafocon A, siflufocon A, silafocon A, sterafocon A, sulfocon A, sulfocon B, telafocon A, tisilfocon A, tolofocon A, trifocon A, unifocon A, vinafocon A, and wilofocon A. Exemplary materials used to fabricate a hydrophilic lens with means to deliver the compositions of the invention include, but are not limited to, abafilcon A, acofilcon A, acofilcon B, acquafilcon A, alofilcon A, alphafilcon A, amfilcon A, astifilcon A, atlafilcon A, balafilcon A, bisfilcon A, bufilcon A, comfilcon A, crofilcon A, cyclofilcon A, darfilcon A, deltafilcon A, deltafilcon B, dimefilcon A, droxfilcon A, elastofilcon A, epsilfilcon A, esterifilcon A, etafilcon A, focofilcon A, galyfilcon A, genfilcon A, govafilcon A, hefilcon A, hefilcon B, hefilcon C, hilafilcon A, hilafilcon B, hioxifilcon A, hioxifilcon B, hioxifilcon C, hydrofilcon A, lenefilcon A, licryfilcon A, licryfilcon B, lidofilcon A, lidofilcon B, lotrafilcon A, lotrafilcon B, mafilcon A, mesafilcon A, methafilcon B, mipafilcon A, nelfilcon A, netrafilcon A, ocufilcon A, ocufilcon B, C, ocufilcon D, ocufilcon E, ofilcon A, omafilcon A, oxyfilcon A, pentafilcon A, perfilcon A, pevafilcon A, phemfilcon A, polymacon, senofilcon A, silafilcon A, siloxyfilcon A, surfilcon A, tefilcon A, tetrafilcon A, trilfilcon A, vifilcon A, vifilcon B, and xylofilcon A.

Within the scope of the invention are compositions formulated as a gel or gel-like substance, creme or viscous emulsions. It is preferred that said compositions comprise at least one gelling component, polymer or other suitable agent to enhance the viscosity of the composition. Any gelling component known to a person skilled in the art, which has no detrimental effect on the area being treated and is applicable in the formulation of compositions and pharmaceutical compositions for topical administration to the skin, eye or mucous can be used. For example, the gelling component may be selected from the group of: acrylic acids, carbomer, carboxypolymethylene, such materials sold by B. F. Goodrich under the trademark Carbopol (e.g. Carbopol 940), polyethylene-polypropyleneglycols, such materials sold by BASF under the trademark Poloxamer (e.g. Poloxamer 188), a cellulose derivative, for example hydroxypropyl cellulose, hydroxyethyl cellulose, hydroxyethylene cellulose, methyl cellulose, carboxymethyl cellulose, alginic acid-propylene glycol ester, polyvinylpyrrolidone, veegum (magnesium aluminum silicate), Pemulen, Simulgel (such as Simulgel 600, Simulgel EG, and simulgel NS), Capigel, Colafax, plasdones and the like and mixtures thereof.

A gel or gel-like substance according to the present invention comprises for example less than 10% w/w water, for example less than 20% w/w water, for example at least 20% w/w water, such as at least 30% w/w water, for example at least 40% w/w water, such as at least 50% w/w water, for example at least 75% w/w water, such as at least 90% w/w water, for example at least 95% w/w water. Preferably said water is deionised water.

Gel-like substances of the invention include a hydrogel, a colloidal gel formed as a dispersion in water or other aqueous medium. Thus, a hydrogel is formed upon formation of a colloid in which a dispersed phase (the colloid) has combined with a continuous phase (i.e. water) to produce a viscous jellylike product; for example, coagulated silicic acid. A hydrogel is a three-dimensional network of hydrophilic polymer chains that are crosslinked through either chemical or physical bonding. Because of the hydrophilic nature of the polymer chains, hydrogels absorb water and swell. The swelling process is the same as the dissolution of non-crosslinked hydrophilic polymers. By definition, water constitutes at least 10% of the total weight (or volume) of a hydrogel.

Examples of hydrogels include synthetic polymers such as polyhydroxy ethyl methacrylate, and chemically or physically crosslinked polyvinyl alcohol, polyacrylamide, poly(N-vinyl pyrrolidone), polyethylene oxide, and hydrolyzed polyacrylonitrile. Examples of hydrogels which are organic polymers include covalent or ionically crosslinked polysaccharide-based hydrogels such as the polyvalent metal salts of alginate, pectin, carboxymethyl cellulose, heparin, hyaluronate and hydrogels from chitin, chitosan, pullulan, gellan and xanthan. The particular hydrogels used in our experiment were a cellulose compound (i.e. hydroxypropylmethylcellulose [HPMC]) and a high molecular weight hyaluronic acid (HA).

Hyaluronic acid is a polysaccharide made by various body tissues. U.S. Pat. No. 5,166,331 discusses purification of different fractions of hyaluronic acid for use as a substitute for intraocular fluids and as a topical ophthalmic drug carrier. Other U.S. patent applications which discuss ocular uses of hyaluronic acid include Ser. Nos. 11/859,627; 11/952,927; 10/966,764; 11/741,366; and 11/039,192 Formulations of macromolecules for intraocular use are known, See eg U.S. patent application Ser. Nos. 11/370,301; 11/364,687; 60/721,600; 11/116,698 and 60/567,423; 11/695,527. Use of various active agents is a high viscosity hyaluronic acid is known. See eg U.S. patent application Ser. Nos. 10/966,764; 11/091,977; 11/354,415; 60/519,237; 60/530,062, and; Ser. No. 11/695,527.

Sustained release formulations as described in WO2010048086 are within the scope if the invention.

The man skilled in the art is well aware of the standard methods for incorporation of a polynucleotide or vector into a host cell, for example transfection, lipofection, electroporation, microinjection, viral infection, thermal shock, transformation after chemical permeabilisation of the membrane or cell fusion.

As used herein, the term “host cell or host cell genetically engineered” relates to host cells which have been transduced, transformed or transfected with the construct or with the vector described previously.

As representative examples of appropriate host cells, one can cites bacterial cells, such as E. coli, Streptomyces, Salmonella typhimurium, fungal cells such as yeast, insect cells such as Sf9, animal cells such as CHO or COS, plant cells, etc. The selection of an appropriate host is deemed to be within the scope of those skilled in the art from the teachings herein. Preferably, said host cell is an animal cell, and most preferably a human cell. The invention further provides a host cell comprising any of the recombinant expression vectors described herein. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α, E. coli cells, Chinese hamster ovarian cells, monkey VERO cells, COS cells, HEK293 cells, and the like.

In case of ex vivo gene therapy, a host cell may be a cell isolated from a patient, for instance a hematopoietic stem cells, which upon introduction of the transgene is reintroduced into said patient in need thereof.

AAV-Based Viral Delivery Systems

The construction of an AAV vector can be carried out following procedures and using techniques which are known to a person skilled in the art. The theory and practice for adeno-associated viral vector construction and use in therapy are illustrated in several scientific and patent publications (the following bibliography is herein incorporated by reference: Flotte T R. Adeno-associated virus-based gene therapy for inherited disorders. Pediatr Res. 2005 December; 58(6):1143-7; Goncalves M A. Adeno-associated virus: from defective virus to effective vector, Virol J. 2005 May 6; 2:43; Surace E M, Auricchio A. Adeno-associated viral vectors for retinal gene transfer. Prog Retin Eye Res. 2003 November; 22(6):705-19; Mandel R J, Manfredsson F P, Foust K D, Rising A, Reimsnider S, Nash K, Burger C. Recombinant adeno-associated viral vectors as therapeutic agents to treat neurological disorders. Mol Ther. 2006 March; 13(3):463-83).

Suitable administration forms of a pharmaceutical composition containing AAV vectors include, but are not limited to, injectable solutions or suspensions, eye lotions and ophthalmic ointment. In a preferred embodiment, the AAV vector is administered by intra-thecal injection. In a particularly preferred embodiment, the AAV vector is administered by subretinal injection, in the anterior chamber or in the retrobulbar space and intravitreal. Preferably the viral vectors are delivered via subretinal approach (as described in Bennicelli J, et al Mol Ther. 2008 Jan. 22; Reversal of Blindness in Animal Models of Leber Congenital Amaurosis Using Optimized AAV2-mediated Gene Transfer).

The doses of virus for use in therapy shall be determined on a case by case basis, depending on the administration route, the severity of the disease, the general conditions of the patients, and other clinical parameters. In general, suitable dosages will vary from 10⁸ to 10¹³ vg (vector genomes)/eye.

Inteins

An intein is a segment of a protein that is able to excise itself and join the remaining portions (the exteins) with a peptide bond in a process known as protein splicing. The segments are called “intein” for internal protein sequence, and “extein” for external protein sequence, with upstream exteins termed “N-exteins” and downstream exteins called “C-exteins.” The products of the protein splicing process are two stable proteins: the mature protein and the intein.

Inteins can also exist as two fragments encoded by two separately transcribed and translated genes, herein named “split-inteins”.

Inteins of the present invention include without limitations split inteins listed in the New England Biolabs Intein database, disclosed in (66).

Split inteins may be produced starting from inteins by first removing the homing endonuclease domain sequence to produce a mini intein. Said mini intein may then split at one or more sites designed through protein sequence alignments with inteins of known crystal structures to generate split inteins, assayed for trans-splicing activity according to protocols included in the present disclosure.

Split inteins may be further improved in desirable characteristics including activity, efficiency, generality, and stability through site-directed mutagenesis or modifications of the intein sequences based on rational design, and/or through directed evolution using methods like functional selection, phage display, and ribosome display.

An example of split inteins are the inteins derived from DnaE which is the catalytic subunit α of DNA polymerase III in cyanobacteria, encoded by two separate genes, dnaE-n and dnaE-c. The intein encoded by the dnaE-n gene is herein referred as “N-intein.” The intein encoded by the dnaE-c gene is herein referred as “C-intein”. Generally, the N-part of a split intein is referred to as “N-Intein” and the C-Part of a split intein is referred to as “C-Intein”. Split inteins self-associate and catalyze protein-splicing activity in trans (herein “trans-splicing”)

Further examples of split inteins of the present invention comprise intein of DnaE from Nostoc punctiforme (Npu) (27, 28)), indicated in the table 3 below as SEQ ID 1 coded by the Npu-DnaE-n nucleotide sequence, and SEQ ID 2 coded by the Npu-DnaE-c nucleotide sequence; the intein of DnaB from Rhodothermus marinus (Rma) (29) indicated in the table below as SEQ ID 4 coded by the Rma-DnaB-n nucleotide sequence and SEQ ID 5 coded by the Rma-DnaB-c nucleotide sequence; mutated N- and C-inteins wherein the N-Intein is from DnaE of Npu (SEQ IDs 5) and the C-Intein is from Synechocystis species strain PCC6803 (Ssp (SEQ ID 6), respectively (30); the Synechocystis species strain PCC6803 N-Intein and C-Intein are included as SEQ ID 13 and 14 respectively in the Table below. Other intein systems may also be used. For example, a synthetic fast intein based on the dnaE intein, the Cfa-N and Cfa-C intein pair, has been described (e.g., (31) and in WO 2017/132580, incorporated herein by reference). Additional Inteins have been described in U.S. Pat. No. 8,394,604, including Ssp GyrB intein, Ssp DnaX intein, Ter DnaE3 intein, Ter ThyX intein, and Cne Prp8 intein. Further inteins within the present invention are the inteins disclosed in WO2018071868, wherein the first pair of inteins is listed in the table below and named as SEQ ID 9 (N-Intein) and SEQ ID 10 (C-Intein); a second pair of inteins is listed, eg SEQ ID 11 and SEQ ID12.

Alternatively, the intein system may be a ligand-dependent intein which exhibits no or minimal protein splicing activity in the absence of ligand (e.g., small molecules such as 4-hydroxytamoxifen, peptides, proteins, polynucleotides, amino acids, and nucleotides).

Ligand-dependent inteins include for instance those described in U.S. 2014/0065711 A1, incorporated herein by reference.

TABLE 3
Examples of split inteins of the
present invention

| | SEQ | |
|---|---|---|
| | ID | |
| Intein | No. | Sequence |
| Npu- | 1 | CLSYETEILTVEYGLLPIGKIVEKRIECTV |
| DnaE-n | | YSVDNNGNIYTQPVAQWHDRGEQEVFEYCL |
| | | EDGSLIRATKDHKFMTVDGQMLPIDEIFER |
| | | ELDLMRVDNLPN |
| Npu- | 2 | IKIATRKYLGKQNVYDIGVERDHNFALKNG |
| DnaE-c | | FIASN |
| Rma- | 3 | CLAGDTLITLADGRRVPIRELVSQQNFSVW |
| DnaB-n | | ALNPQTYRLERARVSRAFCTGIKPVYRLTT |
| | | RLGRSIRATANHRFLTPQGWKRVDELQPGD |
| | | YLALPRRIPTASTPTL |
| Rma- | 4 | AAACPELRQLAQSDVYWDPIVSIEPDGVEE |
| DnaB-c | | VFDLTVPGPHNFVANDIIAHN |
| mNpu | 5 | CLSYDTEILTVEYGILPIGKIVEKRIECTV |
| DnaE-n | | YSVDNNGNIYTQPVAQWHDRGEQEVFEYCL |
| | | EDGSLIRATKDHKFMTVDGQMMPIDEIFER |
| | | ELDLMRVDNLPN |
| mNpu- | 6 | VKVIGRRSLGVQRIFDIGLPQYHNFLLANG |
| DnaE-c | | AIAAN |
| Cfa-n | 7 | CLSYDTEILTVEYGFLPIGKIVEERIECTV |
| | | YTVDKNGFVYTQPIAQWHNRGEQEVFEYCL |
| | | EDGSIIRATKDHKFMTTDGQMLPIDEIFER |
| | | GLDLKQVDGLP |
| Cfa-c | 8 | VKIISRKSLGTQNVYDIGVEKDHNFLLKNG |
| | | LVASN |
| N-intein | 9 | CLSYETEILTVEYGLLPIGKIVEKRIECTV |
| SEQ 351 | | YSVDNNGNIYTQPVAQWHDRGEQEVFEYCL |
| WO_2018_071868_ | | EDGSLIRATKDHKFMTVDGQMLPIDEIFER |
| 351 | | ELDLMRVDNLPN |
| C-Intein | 10 | IKIATRKYLGKQNVYDIGVERDHNFALKNG |
| SEQ 353 | | FIASN |
| N- Intein | 11 | CLSYDTEILTVEYGFLPIGKIVEERIECTV |
| SEQ 354 | | YTVDKNGFVYTQPIAQWHNRGEQEVFEYCL |
| | | EDGSIIRATKDHKFMTTDGQMLPIDEIFER |
| | | GLDLKQVDGLP |
| C-Intein | 12 | KRTADGSEFESPKKKRKVKIISRKSLGTQN |
| SEQ 357 | | VYDIGVEKDHNFLLKNGLVASN |
| Ssp DnaE- | 13 | CLSFGTEILTVEYGPLPIGKIVSEEINCSV |
| n | | YSVDPEGRVYTQAIAQWHDRGEQEVLEYEL |
| PCC6803 | | EDGSVIRATSDHRFLTTDYQLLAIEEIFAR |
| | | QLDLLTLENIKQTEEALDNHRLPFPLLDAG |
| | | TIK |
| Ssp DnaE- | 14 | VKVIGRRSLGVQRIFDIGLPQDHNFLLANG |
| c | | AIAANC |
| PCC6803 | | |

As described herein, within the scope of the present invention are inteins originated from the same gene from different organisms, retaining trans-splicing activity. As a non limiting example, the DNA-E split intein may be derived from split inteins the DnaE gene (eg DNA polymerase III subunit alpha) from cyanobacteria including Nostoc punctiforme (Npu) Synechocystis sp. PCC6803 (Ssp), Fischerella sp. PCC 9605, Scytonema tolypothrichoides, Cyanobacteria bacterium SW_9_47_5, Nodularia spumigena, Nostoc flagelliforme, Crocosphaera watsonii WH 8502, Chroococcidiopsis cubana CCALA 043, Trichodesmium erythraeum. As a further example, the DNA-B ssplit intein may be derived from the DnaB gene from cyanobacteria including R. marinus (Rma), Synechocystis sp. PC6803 (Ssp), Porphyra purpurea chloroplast (Ppu) which are described for instance in (59).

Hence, split inteins of the invention may be 100% identical, 98%, 80%, 75%, 70%, 65% 50% identical to naturally occurring inteins, wherein said inteins retain the ability to undergo trans-splicing reactions. Within the scope of the present invention are fragments of naturally occurring or modified inteins which retain trans-splicing activity.

See for instance the alignment between Npu (Nostoc puntiforme) DnaE and Synechocytis sp. PCC6803 N-Intein:

| Score | Identities | Positives | Gaps |
|---|---|---|---|
| 148 bits(373) | 68/100(68%) | 83/100(83%) | 0/100(0%) |

CLSYETEILTVEYGLLPIGKIVEKRIECTVYSVDNNGNIYTQPVAQWHDRGEQEVFEYCLEDGSLIRATKDHKFMTVDGQMLP
CLS+ TEILTVEYG LPIGKIV + I C+VYSVD G +YTQ +AQWHDRGEQEV EY EDGS+IRAT DH+F+T D Q+L
CLSFGTEILTVEYGPLPIGKIVSEEINCSVYSVDPEGRVYTQAIAQWHDRGEQEVLEYELEDGSVIRATSDHRFLTTDYQLLA
IDEIFERELDLMRVDNL SEQ ID No. 21
I+EIF R+LDL+ ++N SEQ ID No. 22
IEEIFARQLDLLTLENI SEQ ID No. 23

And the alignment between Npu (Nostoc puntiforme) DnaE and Synechocytis sp. PCC6803 C-Intein:

| Score | Identities | Positives | Gaps |
|---|---|---|---|
| 46.6 bits(109) | 19/36(53%) | 27/36(75%) | 0/36(0%) |

MIKIATRKYLGKQNVYDIGVERDHNFALKNGFIASN SEQ ID No. 24
M+K+ R+ LG Q ++DIG+ +DHNF L NG IA+N SEQ ID No. 25
MVKVIGRRSLGVQRIFDIGLPQDHNFLLANGAIAAN SEQ ID No. 26

Hence, within the scope of the present invention are also split inteins variants and fragments of the inteins of the invention retaining trans-splicing activity

Interestingly, it has been reported that inteins have conserved functional features that guarantee their splicing activity. In particular, four intein motifs have been identified (see below for their consensus sequence): Blocks A-H (Pietrokovski 1994 and Perler 1997) and Blocks N2 and N4 (Pietrokovski 1998). Intein Blocks A, N2, B, N4, F, and G are involved in protein splicing. Blocks C, D, E, H are in the endonuclease domain, which is absent from split inteins. Thus, split inteins retain conserved motifs that are essential to the trans-splicing activity. (Intein database, disclosed in [Perler, F. B. (2002). InBase, the Intein Database. Nucleic Acids Res. 30, 383-384.])

Although, no single residue is invariant, the Ser and Cys in Block A, the His in Block B, the His, Asn and Ser/Cys/Thr in Block G are the most conserved residues in the splicing motifs.

Alignment of the inteins of the present invention:

CLUSTAL W Alignment of all N-inteins listed:

| | |
|---|---|
| SEQ1 | CLSYETEILTVEYGLLPIGKIVEKRIECTVYSVDNNGNIYTQPVAQWHDRGEQEVFEYCL |
| SEQ9 | CLSYETEILTVEYGLLPIGKIVEKRIECTVYSVDNNGNIYTQPVAQWHDRGEQEVFEYCL |
| SEQ5 | CLSYDTEILTVEYGILPIGKIVEKRIECTVYSVDNNGNIYTQPVAQWHDRGEQEVFEYCL |
| SEQ7 | CLSYDTEILTVEYGFLPIGKIVEERIECTVYTVDKNGFVYTQPIAQWHNRGEQEVFEYCL |
| SEQ11 | CLSYDTEILTVEYGFLPIGKIVEERIECTVYTVDKNGFVYTQPIAQWHNRGEQEVFEYCL |
| SEQ13 | CLSFGTEILTVEYGPLPIGKIVSEEINCSVYSVDPEGRVYTQAIAQWHDRGEQEVLEYEL |
| SEQ3 | CLAGDTLITLADGRRVPIRELVSQQNFSVWALNPQTYRLERARVSRAFCTGIKPVYRLTT |
| | **: * * .: :** ::*.:. . : ::: . * : * . |
| SEQ1 | EDGSLIRATKDHKFMTVDGQMLPIDEIFERELDLMRVDNLPN------------------ |
| SEQ9 | EDGSLIRATKDHKFMTVDGQMLPIDEIFERELDLMRVDNLPN------------------ |
| SEQ5 | EDGSLIRATKDHKFMTVDGQMMPIDEIFERELDLMRVDNLPN------------------ |
| SEQ7 | EDGSIIRATKDHKFMTTDGQMLPIDEIFERGLDLKQVDGLP------------------- |
| SEQ11 | EDGSIIRATKDHKFMTTDGQMLPIDEIFERGLDLKQVDGLP------------------- |
| SEQ13 | EDGSVIRATSDHRFLTTDYQLLAIEEIFARQLDLLTLENIKQTEEALDNHRLPFPLLDAG |
| SEQ3 | RLGRSIRATANHRFLTPQGWKRVDELQPGDYLALPRRIPTASTPTL-------------- |
| | . * **** :*:*:* : : * * |
| SEQ1 | --- |
| SEQ9 | --- |
| SEQ5 | --- |
| SEQ7 | --- |
| SEQ11 | --- |
| SEQ13 | TIK |
| SEQ3 | --- |

CLUSTAL 2.1 multiple sequence alignment of all C-Inteins listed

| | |
|---|---|
| SEQ2 | -----------------MIKIATRKYLGKQNVYDIGVERDHNFALKNGFIASN- |
| SEQ10 | -----------------MIKIATRKYLGKQNVYDIGVERDHNFALKNGFIASN- |
| SEQ8 | ------------------VKIISRKSLGTQNVYDIGVEKDHNFLLKNGLVASN- |
| SEQ12 | MKRTADGSEFESPKKKRKVKIISRKSLGTQNVYDIGVEKDHNFLLKNGLVASN- |
| SEQ6 | ------------------VKVIGRRSLGVQRIFDIGLPQYHNFLLANGAIAAN- |
| SEQ14 | -----------------MVKVIGRRSLGVQRIFDIGLPQDHNFLLANGAIAANC |
| SEQ4 | -AAACPELRQLAQSDVYWDPIVSIEPDGVEEVFDLTVPGPHNFVAN-DIIAHN- |
| | : . * :.::*: : *** . :* * |

In summary, intein activity is context-dependent, with certain peptide sequences surrounding their ligation junction (called N- and C-exteins) that are required for efficient trans-splicing to occur, of which the most important is an amino acid containing a nucleophilic thiol or hydroxyl group (i.e., Cys, Ser or Thr) as first residue in the C-extein.

The present inventors have used intein-mediated protein-transplicing in order to reconstitute large proteins in vivo. Split inteins encoded by intein gene sequences are produced as precursor polypeptides, which through their structural complementation can reassemble and catalyze a protein trans-splicing reaction.

In the context of protein trans-splicing, the N-intein gene is fused in frame with the sequence coding for the N-terminal portion of the protein of interest; the C-Intein gene is fused in frame with the sequence coding for the C-terminal portion of the sequence of interest. Upon expression of the two precursor fusion proteins, the inteins undergo autocatalytic excision and form a ligated extein, eg the reconstituted protein of interest.

Hence, reconstitution of a protein of interest requires splitting said protein into two or three fragments, whose coding sequences are cloned separately into AAV vector, fused to a N- or C-Intein and under the control of a promoter. Splitting points for each protein are selected taking into account the amino acid requirement at the junction point (eg presence of an amino acid containing a nucleophilic thiol or hydroxyl group (i.e. Cys, Ser or Thr) as first residue in the C-extein, as well as preservation of the integrity of critical protein domains in order to favor proper protein folding and stability of each intein-polypeptide precursor polypeptide and the resulting reconstituted protein.

Of particular note, the present inventors have selected junction points within two proteins of interest: the protein ABCA4 is split at amino acid Cys1150, Ser1168, Ser 1090, and a split intein is inserted at the split point. The CEP290 protein is split at aa Cys1076, Ser1275, Cys 929 and 1474; Ser 453 and Cys 1474.

Degradation Signals

Regulated protein degradation protects cells from misfolded, aggregated, or otherwise abnormal proteins, and also controls the levels of proteins that evolved to be short-lived in vivo and is mediated largely by the ubiquitin (Ub)-proteasome system (UPS) and by autophagy-lysosome pathways, with molecular chaperones being a part of both systems. Degradation signals are features of proteins that make them targets of the protein degradation pathways, with the result of decreasing their half life. In particular, N-degrons and C-degrons are degradation signals whose main determinants are, respectively, the N-terminal and C-terminal residues of cellular proteins. N-degrons and C-degrons include, to varying extents, adjoining sequence motifs, and also internal lysine residues that function as polyubiquitylation sites.

Within the meaning of the present invention, internal degrons are defined as degradation signals located within a protein sequence neither at N-terminal nor at C-terminal and whose functionally essential elements do not include either N-terminal residues or C-terminal residues and mediate protein degradation.

The degron pathways comprise sets of proteolytic systems whose unifying feature is their ability to recognize proteins containing N- or C- or internal-degrons, thereby causing the degradation of these proteins by the 26S proteasome or autophagy.

E. coli dihydrofolate reductase (ecDHFR) is a 159-residue enzyme which catalyzes the reduction of dihydrofolate to tetrahydrofolate, a cofactor that is essential for several steps in prokaryotic primary metabolism. Numerous inhibitors of DHFR have been developed as drugs, and one such inhibitor, trimethoprim (TMP), inhibits ecDHFR much more potently than mammalian DHFR. This large therapeutic window renders TMP “biologically silent” in mammalian cells. The specificity of the ecDHFR-TMP interaction, coupled with the commercial availability and attractive pharmacological properties of TMP, makes this protein-ligand pair ideal for development as a degradation system. (69) Hence the presence of the DHFR aminoacid sequence preferably the ecDHFR aminoacid sequence, within a protein, functions as a target signal for the proteasome system resulting in protein degradation. In presence of TMP, said protein is stabilized.

Conveniently, ecDHFR derived degron signals carrying point putations developed by Iwamoto et al. include three amino acidic mutations, R12Y, Y100I and G67S (69) that confers functional activity (eg degradation of the fusion protein) only when placed at N-terminal or within an internal position.

Further improvements to the ecDHFR-derived degron were made by the present inventors who identified the shortest active peptide. Conveniently, a shorter sequence allows fitting longer coding sequences within the same AAV vector.

Within the present invention, the ecDHFR-derived degron was fused to the N-terminal of the Intein where it is inactive. Upon protein transplicing, the degron is located within the reconstituted Intein and mediates its degradation.

ecDHFR of the present invention are WT ecDHFR, mutant DHFR, full length ecDHFR, shorter scDHFR.

DHFR may be from 105 to 159 aa long, wherein the shortening occurs at the C-terminal end

ecDHFR E. Coli derived, wild type
Nucleotide sequence: (623 nt)
SEQ ID No. 27
Atcagcctgatcgccgccctggccgtggactacgtgatcggcatggagaac
gccatgccctggaacctgcccgccgacctggcctggttcaagaggaacacc
ctgaacaagcccgtgatcatgggcaggcacacctgggagagcatcggcagg
cccctgcccggcaggaagaacatcatcctgagcagccagcccagcaccgac
gacagggtgacctgggtgaagagcgtggacgaggccatcgccgcctgcggc
gacgtgcccgagatcatggtgatcggcggcggcagggtgatcgagcagttc
ctgcccaaggcccagaagctgtacctgacccacatcgacgccgaggtggag
ggcgacacccacttccccgactacgagcccgacgactgggagagcgtgttc
agcgagttccacgacgccgacgcccagaacagccacagctactgcttcgag
atcctggagaggaggtga
Aminoacid sequence:
159 aa- WT
SEQ ID No. 28
MISLIAALAVDRVIGMENAMPWNLPADLAWFKRNTLDKPVIMGRHTWESIG
RPLPGRKNIILSSQPGTDDRVTWVKSVDEAIAACGDVPEIMVIGGGRVYEQ
FLPKAQKLYLTHIDAEVEGDTHFPDYEPDDWESVFSEFHDADAQNSHSYCF
EILERR
ecDHFR E. Coli derived, Internal degron mutant
(159 aa)
mutation positions in bold-
SEQ ID No. 29
MISLIAALAVDYVIGMENAMPWNLPADLAWFKRNTLNKPVIMGRHTWESIG
RPLPGRKNIILSSQPSTDDRVTWVKSVDEAIAACGDVPEIMVIGGGRVIEQ
FLPKAQKLYLTHIDAEVEGDTHFPDYEPDDWESVFSEFHDADAQNSHSYCF
EILERR
ecDHFR E. Coli derived, wild type, minimum active
fragment
nucleotide sequence:
SEQ ID No. 30
atcagcctgatcgccgccctggccgtggactacgtgatcggcatggagaac
gccatgccctggaacctgcccgccgacctggcctggttcaagaggaacacc
ctgaacaagcccgtgatcatgggcaggcacacctgggagagcatcggcagg
cccctgcccggcaggaaaacatcatcctgagcagccagcccagcaccgacg
acagggtgacctgggtgaagagcgtggacgaggccatcgccgcctgcggcg
acgtgcccgagatcatggtgatcggcggcggcagggtgatcgagcagttcc
tgccctga
aminoacid sequence
SEQ ID No. 31
MISLIAALAVDRVIGMENAMPWNLPADLAWFKRNTLDKPVIMGRHTWESIG
RPLPGRKNIILSSQPGTDDRVTWVKSVDEAIAACGDVPEIMVIGGGRVYEQ
FLP
ecDHFR E. Coli derived, Internal degron mutant pe,
minimum active fragment (104 aa)
(mutation positions in bold)
SEQ ID No. 32
MISLIAALAVDYVIGMENAMPWNLPADLAWFKRNTLNKPVIMGRHTWESIG
RPLPGRKNIILSSQPSTDDRVTWVKSVDEAIAACGDVPEIMVIGGGRVIEQ
FLP

Sequences

Coding sequences of the invention may be operably linked to a promoter sequence optionally followed by an intron sequence, able to regulate the expression thereof in a mammalian cell, preferably a mammalian retinal cell, particularly photoreceptor cell, or a liver cell, a muscle cell, a cardiac cell, a neuronal cell, a kidney cell, an endothelial cell. Illustrative promoters include, without limitation, ubiquitous, artificial, or tissue specific promoters, including fragments and variants thereof retaining a transcription promoter activity, such as photoreceptor-specific promoters including photoreceptor-specific human G protein-coupled receptor kinase 1 (GRK1), Interphotoreceptor retinoid binding protein promoter (IRBP), Rhodopsin promoter (RHO), vitelliform macular dystrophy 2 promoter (VMD2), Rhodopsin kinase promoter (RK); muscle-specific promoters including MCK, MYODI; liver-specific promoters including thyroxine binding globulin (TBG), hybrid liver-specific promoter (HLP) (67); neuron-specific promoters including hSYN1, CaMKlla; kidney-specific promoters including Ksp-cadherin16, NKCC2. Ubiquitous promoters according to the present invention are for instance the ubiquitous cytomegalovirus (CMV)(32) and short CMV (33) promoters.

Optionally, the promoter sequence includes an enhancer sequence such as the [symbol] -globin IgG chimeric intron.

For the purposes of this invention, a coding sequence of EGFP (YP_009062989), ABCA4, and CEP290 which are preferably respectively selected from the sequences herein enclosed, or sequences encoding the same amino acid sequence due to the degeneracy of the genetic code, is functionally linked to a promoter sequence able to regulate the expression thereof in a mammalian retinal cell, particularly in photoreceptor cells.

Illustrative polyadenylation signals include, without limitations, the bovine growth hormone polyadenylation signal (bGHpA), the human beta globin polyadenylation signal or a short synthetic version (68), the SV40 polyadenylation signal, or other naturally occurring or artificial polyadenylation signal.

The present invention provides the use of a nucleotide sequence of a degradation signal in order to decrease the stability of the reconstituted intein protein. Conveniently, one or more sequence may be repeated in order to retain maximal effect.

Suitable degradation signals, according to the present invention include: (i) the short degron CL1, a C-terminal destabilizing peptide that shares structural similarities with misfolded proteins and is thus recognized by the ubiquitination system, (ii) ubiquitin, whose fusion at the N-terminal of a donor protein mediates both direct protein degradation or degradation via the N-end rule pathway, (iii) the N-terminal PB29 degron which is a 9 amino acid-long peptide which, similarly to the CL1 degron, is predicted to fold in structures that are recognized by enzymes of the ubiquitination pathway, variant ecDHFR and fragments thereof as described herein and in (69), particularly ecDHFR derived degron signals carrying point mutations which include three amino acidic mutations, R12Y, Y100I and G67S conferring functional activity (eg degradation of the fusion protein) only when placed at N-terminal or within an internal position

Exemplary degradation signals are described in WO 201613932, incorporated herein by reference.

As those skilled in the art can readily appreciate, there can be a number of variant sequences of a protein found in nature, in addition to those variants that can be artificially created by the skilled artisan in the lab. The polynucleotides and polypeptides of the subject invention encompasses those specifically exemplified herein, as well as any natural variants thereof, as well as any variants which can be created artificially, so long as those variants retain the desired functional activity. Also, within the scope of the subject invention are polypeptides which have the same amino acid sequences of a polypeptide exemplified herein except for amino acid substitutions, additions, or deletions within the sequence of the polypeptide, as long as these variant polypeptides retain substantially the same relevant functional activity as the polypeptides specifically exemplified herein. For example, conservative amino acid substitutions within a polypeptide which do not affect the function of the polypeptide would be within the scope of the subject invention. Thus, the polypeptides disclosed herein should be understood to include variants and fragments, as discussed above, of the specifically exemplified sequences. The subject invention further includes nucleotide sequences which encode the polypeptides disclosed herein. These nucleotide sequences can be readily constructed by those skilled in the art having the knowledge of the protein and amino acid sequences which are presented herein. As would be appreciated by one skilled in the art, the degeneracy of the genetic code enables the artisan to construct a variety of nucleotide sequences that encode a particular polypeptide or protein. The choice of a particular nucleotide sequence could depend, for example, upon the codon usage of a particular expression system or host cell. Polypeptides having substitution of amino acids other than those specifically exemplified in the subject polypeptides are also contemplated within the scope of the present invention. For example, non-natural amino acids can be substituted for the amino acids of a polypeptide of the invention, so long as the polypeptide having substituted amino acids retains substantially the same activity as the polypeptide in which amino acids have not been substituted. Examples of non-natural amino acids include, but are not limited to, ornithine, citrulline, hydroxyproline, homoserine, phenylglycine, taurine, iodotyrosine, 2,4-diaminobutyric acid, a-amino isobutyric acid, 4-aminobutyric acid, 2-amino butyric acid, γ-amino butyric acid, ε-amino hexanoic acid, 6-amino hexanoic acid, 2-amino isobutyiic acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form. Amino acids can be generally categorized in the following classes: non-polar, uncharged polar, basic, and acidic. Conservative substitutions whereby a polypeptide having an amino acid of one class is replaced with another amino acid of the same class fall within the scope of the subject invention so long as the polypeptide having the substitution still retains substantially the same biological activity as a polypeptide that does not have the substitution. Table 4 provides a listing of examples of amino acids belonging to each class.

TABLE 4
Listing of examples of amino acids belonging to each class

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

Also within the scope of the subject invention are polynucleotides which have the same nucleotide sequences of a polynucleotide exemplified herein except for nucleotide substitutions, additions, or deletions within the sequence of the polynucleotide, as long as these variant polynucleotides retain substantially the same relevant functional activity as the polynucleotides specifically exemplified herein (e.g., they encode a protein having the same amino acid sequence or the same functional activity as encoded by the exemplified polynucleotide). Thus, the polynucleotides disclosed herein should be understood to include variants and fragments, as discussed above, of the specifically exemplified sequences.

The subject invention also contemplates those polynucleotide molecules having sequences which are sufficiently homologous with the polynucleotide sequences of the invention so as to permit hybridization with that sequence under standard stringent conditions and standard methods (Maniatis, T. et al, 1982). Polynucleotides described herein can also be defined in terms of more particular identity and/or similarity ranges with those exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% or greater as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/N1H website.

Plasmids of the Invention

| | | | Size | | |
|---|---|---|---|---|---|
| | | | ITR-ITR | AAV serotype | |
| | Plasmid | Sets | (bp) | 2/2 | 2/8 |
| EGFP | pAAV2.1-CMV-5′ EGFP intein DnaB | | 2659 | | |
| | pAAV2.1-CMV-3′ EGFP intein DnaB | | 2704 | | |
| | pAAV2.1-CMV-5′ EGFP intein mDnaE | | 2557 | | |
| | pAAV2.1-CMV-3′ EGFP intein mDnaE | | 2656 | | |
| | pAAV2.1-CMV-5′ EGFP intein | | 2557 | X | X |
| | pAAV2.1-CMV-3′ EGFP intein | | 2656 | X | X |
| | pAAV2.1-CMV-5′ EGFP intein_ecDHFR | | 3031 | | |
| | pAAV2.1-CMV-5′ EGFP intein_mini ecDHFR | | 2869 | | |
| | pAAV2.1-GRK1-5′ EGFP intein | | 2090 | | X |
| | pAAV2.1-GRK1-3′ EGFP intein | | 2189 | | X |
| | pAAV2.1-TBG-5′ EGFP intein | | 2665 | | X |
| | pAAV2.1-TBG-3′ EGFP intein | | 2764 | | X |
| ABCA4 | pzac-CMV260-5′ ABCA4 intein | Set 1 | 4875 | X | |
| | pzac-CMV260-3′ABCA4 intein | | 4602 | X | |
| | pAAV2.1-CMV260-5′ ABCA4 intein_ecDHFR | Set 1 | 5086 | | |
| | pAAV2.1-CMV260-5′ ABCA4 intein_mini cDHFR | Set 1 | 4924 | | |
| | pzac-GRK1-5′ ABCA4 intein | Set 1 | 4908 | | X |
| | pzac-GRK1-3′ABCA4 intein | | 4634 | | X |
| | pAAV2.1-GRK1-5′ ABCA4 intein_ecDHFR | Set 1 | 5059 | | X |
| | pAAV2.1-GRK1-5′ ABCA4 intein_mini cDHFR | Set 1 | 4968 | | X |
| | pzac-CMV260-5′ ABCA4 intein | Set 2 | 4929 | | |
| | pzac-CMV260-3′ABCA4 intein | | 4548 | | |
| | pzac-CMV260-5′ ABCA4 intein | Set 3 | 4695 | | |
| | pzac-CMV260-3′ABCA4 intein | | 4782 | | |
| CEP290 | pAAV2.1-CMV260-5′ CEP290 intein | Set 1 | 4281 | | |
| | pAAV2.1-CMV260-3′ CEP290 intein | | 5070 | | |
| | pAAV2.1-CMV260-5′ CEP290 intein | Set 2 | 5051 | | |
| | pAAV2.1-CMV260-3′ CEP290 intein | | 4646 | | |
| | pAAV2.1-CMV260-5′ CEP290 intein | Set 3 | 5051 | | |
| | pAAV2.1-CMV260-3′ CEP290 intein | | 4646 | | |
| | pAAV2.1-CMV260-5′ CEP290 intein | Set 4 | 4631 | | |
| | pAAV2.1-CMV260-CEP290 body intein | | 3602 | | |
| | pAAV2.1-CMV260-3′ CEP290 intein | | 4586 | | |
| | pAAV2.1-CMV260-5′ CEP290 intein | Set 5 | 3074 | X | |
| | pAAV2.1-CMV260-CEP290 body intein | | 4906 | X | |
| | pAAV2.1-CMV260-3′ CEP290 intein | | 4586 | X | |
| | pAAV2.1-GRK1-5′ CEP290 intein | Set 5 | 3118 | | X |
| | pAAV2.1-GRK1-CEP290 body intein | | 4945 | | X |
| | pAAV2.1-GRK1-3′ CEP290 intein | | 4630 | | X |
| F8 | pAAV2.1_HLP_5′ F8 intein | Set 1 | 4919 | | X |
| | pAAV2.1_HLP_3′ F8 intein | | 3962 | | X |
| | pAAV2.1_HLP_5′ F8 intein | Set 2 | 3935 | | X |
| | pAAV2.1_HLP_ 3′ F8 intein | | 4946 | | X |

EGFP
p915_pAAV2.1-TBG-5′ EGFP intein (SEQ ID No. 33)

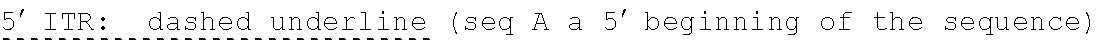

TBG promoter: bold (seq B)
5′ EGFP: underline (seq C)
N-intein Npu DnaE: double underline (seq D)
3xflag: italic (seq E)
WPRE: italic underline (seq F)
Bgh PolyA: bold underline (seq G)

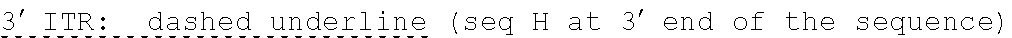

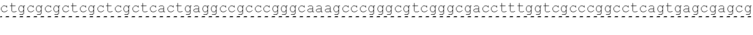

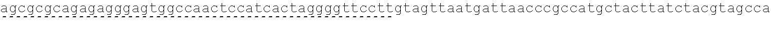

tgctctaggaagatcggaattcgcccttaagctagcaggttaatttttaaaaagcagtcaaaagtccaagtggcccttggcagcatt
tactctctctgtttgctctggttaataatctcaggagcacaaacattccagatccaggttaatttttaaaaagcagtcaaaagtcca
agtggcccttggcagcatttactctctctgtttgctctggttaataatctcaggagcacaaacattccagatccggcgcgccagggct
ggaagctacctttgacatcatttcctctgcgaatgcatgtataatttctacagaacctattagaaaggatcacccagcctctgctttt
gtacaactttcccttaaaaaactgccaattccactgctgtttggcccaatagtgagaactttttcctgctgcctcttggtgcttttgcct
atggcccctattctgcctgctgaagacactcttgccagcatggacttaaacccctccagctctgacaatcctctttctcttttgttttac
atgaagggtctggcagccaaagcaatcactcaaagttcaaaccttatcattttttgctttgttcctcttggccttggttttgtacatca
gctttgaaaataccatcccagggttaatgctggggttaatttataactaagagtgctctagttttgcaatacaggacatgctataaa
aatggaaagatgttgctttctgagagactgcagaagttggtcgtgaggcactgggcaggtaagtatcaaggttacaagacaggttt
aaggagaccaatagaaactgggcttgtcgagacagagaagactcttgcgtttctgataggcacctattggtcttactgacatccactt
tgcctttctctccacaggtgtccaggcggccgccatggtgagcaagggcgaggagctgttcaccggggtggtgcccatcctggtcga
gctggacggcgacgtaaacggccacaagttcagcgtgtccggcgagggcgagggcgatgccacctacggcaagctgaccctgaag
ttcatctgcaccaccggcaagctgcccgtgccctggcccaccctcgtgaccaccctgacctacggcgtgcagtgcctgagctacgag
accgagatcctgaccgtggagtacggcctgctgcccatcggcaagatcgtggagaagcggatcgagtgcaccgtgtacagcgtgga
caacaacggcaacatctacacccagcccgtggcccagtggcacgaccggggcgagcaggaggtgttcgagtactgcctggaggac
ggcagcctgatccgggccaccaaggaccacaagttcatgaccgtggacggccagatgctgcccatcgacgagatcttcgagcggga
gctggacctgatgcgggtggacaacctgcccaacgactocaaagaccargacggrgartataaagarcargacarcgactoca
aggatgacgatgacaagtgaaagcttggatccaataacctctggattacaaaatttgtgaaagattgactggtattcttaact
atgttgctccttttacgctatgtggatacgctgctttaatgcctttgtatcatgctattgcttcccgtatggctttcattttctcctccttg
tataaatcctggttgctgtctctttatgaggagttgtaggcccgttgtcaggcaacgtggcgtggtgtgcactgtgtttgctgacgc
aacccccactggttggggcattgccaccacctgtcagctcctttccgggactttcgctttccccctccctattgccacggcaggaact
catcgccgcctgccttgcccgctgctggacaggggctcggctgttgggcactgacaattcgtggtgttgtcaggggaagctgac
gtcctttccatggctgctcgcctgtgttgccacctggattctgcgcgggacgtccttctgctacgtcccttcggccctcaatccagcg
gaccttccttcccgcggcctgctgccggctctgcggcctcttccgcgtcttcgagatctgcctcgactgtgccttctagttgccagcca
tctgttgtttgcccctcccccgtgccttccttgaccctggaaggtgccactcccactgtcctttcctaataaaatgaggaaattgcatc
gcattgtctgagtaggtgtcattctattctggggggtggggtggggcaggacagcaagggggaggattgggaagacaatagcag
gcatgctggggactcgagttaagggcgaattcccgattaggatcttcctagagcatggctacgtagataagtagcatggcgggttaa

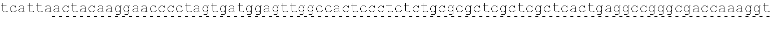

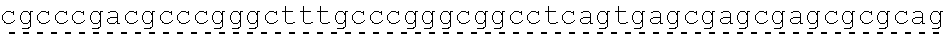

p917_pAAV2.1-TBG-3′ EGFP intein
5′ ITR (seq A)
TBG promoter (seq B)
C-intein Npu DnaE (seqI) SEQ ID NO. 34
atgatcaagatcgccacccggaagtacctgggcaagcagaacgtgtacgacatcggcgtggagcgggaccacaacttcgccctga
agaacggcttcatcgccagcaat
3′ EGFP (seq L) SEQ ID NO. 35
tgcttcagccgctaccccgaccacatgaagcagcacgacttcttcaagtccgccatgcccgaaggctacgtccaggagcgcaccatc
ttcttcaaggacgacggcaactacaagacccgcgccgaggtgaagttcgagggcgacaccctggtgaaccgcatcgagctgaagg
gcatcgacttcaaggaggacggcaacatcctggggcacaagctggagtacaactacaacagccacaacgtctatatcatggccgac
aagcagaagaacggcatcaaggtgaacttcaagatccgccacaacatcgaggacggcagcgtgcagctcgccgaccactaccagc
agaacacccccatcggcgacggccccgtgctgctgcccgacaaccactacctgagcacccagtccgccctgagcaaagaccccaac
gagaagcgcgatcacatggtcctgctggagttcgtgaccgccgccgggatcactctcggcatggacgagctgtacaag
3xflag (seq E)
WPRE (seq F)
Bgh PolyA (seq G)
3′ ITR (seq H)
p914_pAAV2.1-CMF-5′ EGFP intein
5′ ITR (seq A)
CMV promoter (seq M) SEQ. ID No. 36
tagttattaatagtaatcaattacggggtcattagttcatagcccatatatggagttccgcgttacataacttacggtaaatggcccgc
ctggctgaccgcccaacgacccccgcccattgacgtcaataatgacgtatgttcccatagtaacgccaatagggactttccattgacg
tcaatgggtggagtatttacggtaaactgcccacttggcagtacatcaagtgtatcatatgccaagtacgccccctattgacgtcaat
gacggtaaatggcccgcctggcattatgcccagtacatgaccttatgggactttcctacttggcagtacatctacgtattagtcatcgc
tattaccatggtgatgcggttttggcagtacatcaatgggcgtggatagcggtttgactcacggggatttccaagtctccaccccattg
acgtcaatgggagtttgttttggcaccaaaatcaacgggactttccaaaatgtcgtaacaactccgccccattgacgcaaatgggcg
gtaggcgtgtacggtgggaggtctatataagcagagctggtttagtgaaccgt
5' EGFP (seq C)
N-intein Npu DnaE (seq D)
3xflag (seq E)
WPRE (seq F)
Bgh PolyA (seq g)
3′ ITR (seq H)
p916_pAAV2.1-CMV-3′ EGFP intein
5′ ITR (seq A)
CMV promoter (secq M)
C-intein Npu DnaE (seq I)
3′ EGFP (seq L)
3xflag (seq E)
WPRE (seq F)
Bgh PolyA (seq G)
3′ ITR (seq H)
p932_pAAV2.1-GRK1-5′ EGFP intein
5′ ITR (seq A)
GRK1 promoter (seq N) SEQ ID No. 37
ctagtgggccccagaagcctggtggttgtttgtccttctcaggggaaaagtgaggcggccccttggaggaaggggccgggcagaat
gatctaatcggattccaagcagctcaggggattgtctttttctagcaccttcttgccactcctaagcgtcctccgtgaccccggctggga
tttagcctggtgctgtgtcagccccgggctcccaggggcttcccagtggtccccaggaaccctcgacagggccagggcgtctctctcg
tccagcaagggcagggacgggccacaggcaagggcgc
5′ EGFP (seq C)
N-intein Npu DnaE (seq D)
3xflag (seq E)
WPRE (seq F)
Bgh PolyA (seq G)
3′ ITR (seq H)
p933_pAAV2.1-GRK1-3′ EGFP intein
5′ ITR (seq A)
GRK1 promoter (seq N)
C-intein Npu DnaE (seq I)
3′ EGFP (seq L)
3xflag (seq E)
WPRE (seq F)
Bgh PolyA (seq G)
3′ ITR (seq H)
p36 pAAV2.1-CMV-5′ EGFP intein_ecDHFR
5′ ITR (seq A)
CMV promoter (seq M)
5′ EGFP (seq C)
N-intein Npu DnaE (seq D)
3xflag (seq E)
ecDHFR (seq 0) SEQ. ID No. 38
atcagcctgatcgccgccctggccgtggactacgtgatcggcatggagaacgccatgccctggaacctgcccgccgacctggcctgg
ttcaagaggaacaccctgaacaagcccgtgatcatgggcaggcacacctgggagagcatcggcaggcccctgcccggcaggaaga
acatcatcctgagcagccagcccagcaccgacgacagggtgacctgggtgaagagcgtggacgaggccatcgccgcctgcggcga
cgtgcccgagatcatggtgatcggcggcggcagggtgatcgagcagttcctgcccaaggcccagaagctgtacctgacccacatcg
acgccgaggtggagggcgacacccacttccccgactacgagcccgacgactgggagagcgtgttcagcgagttccacgacgccga
cgcccagaacagccacagctactgcttcgagatcctggagaggaggtga
WPRE (seq F)
Bgh PolyA (seq G)
3′ TR (seq H)
p37 pAAV2.1-CMV-5′ EGFP intein_mini ecDHFR
5′ ITR (seq A)
CMV promoter (seq M)
5′ EGFP (seq C)
N-intein Npu DnaE (seq D)
3xflag (seq E)
mini ecDHFR (seq P) SEQ. ID No. 39
atcagcctgatcgccgccctggccgtggactacgtgatcggcatggagaacgccatgccctggaacctgcccgccgacctggcctgg
ttcaagaggaacaccctgaacaagcccgtgatcatgggcaggcacacctgggagagcatcggcaggcccctgcccggcaggaaga
acatcatcctgagcagccagcccagcaccgacgacagggtgacctgggtgaagagcgtggacgaggccatcgccgcctgcggcga
cgtgcccgagatcatggtgatcggcggcggcagggtgatcgagcagttcctgccctga
WPRE (seq F)
Bgh PolyA (seq G)
3′ TR (seq H)
p902_pAAV2.1-CMF-5′ EGFP intein DnaB
5′ ITR (seq A)
CMV promoter (seq M)
5′ EGFP (seq C)
N-intein RmaDnaB (seq Q) SEQ. ID No. 40
tgcctggccggcgacaccctgatcaccctggccgacggcaggagggtgcccatcagggagctggtgagccagcagaacttcagcgt
gtgggccctgaacccccagacctacaggctggagagggccagggtgagcagggccttctgcaccggcatcaagcccgtgtacagg
ctgaccaccaggctgggcaggagcatcagggccaccgccaaccacaggttcctgaccccccagggctggaagagggtggacgagc
tgcagcccggcgactacctggccctgcccaggaggatccccaccgccagcacccccaccctg
N-intein Npu DnaE (seq D)
3xflag (seq E)
WPRE (seq F)
Bgh PolyA (seq G)
3′ ITR (seq H)
p903_pAAV2.1-CMV-3′ EGFP intein DnaB
5′ ITR (seq A)
CMV promoter (seq M)
C-intein Rma DnaB (seq R) SEQ. ID No. 41
atggccgccgcctgccccgagctgaggcagctggcccagagcgacgtgtactgggaccccatcgtgagcatcgagcccgacggcgt
ggaggaggtgttcgacctgaccgtgcccggcccccacaacttcgtggccaacgacatcatcgcccacaac
3′ EGFP (seq L)
3xflag (seq E)
WPRE (seq F)
Bgh PolyA (seq G)
3′ ITR (seq H)
p1256_pAAV2.1-CMV-5′ EGFP intein mDnaE
5′ ITR (seq A)
CMV promoter (seq M)
5′ EGFP (seq C)
N-intein mDnaE (seq S) SEQ. ID No. 42
tgcctgagctacgacaccgagatcctgaccgtggagtacggcatcctgcccatcggcaagatcgtggagaagaggatcgagtgcac
cgtgtacagcgtggacaacaacggcaacatctacacccagcccgtggcccagtggcacgacaggggcgagcaggaggtgttcgag
tactgcctggaggacggcagcctgatcagggccaccaaggaccacaagttcatgaccgtggacggccagatgatgcccatcgacg
agatcttcgagagggagctggacctgatgagggtggacaacctgcccaac
3xflag (seq E)
WPRE (seq F)
Bgh PolyA (seq G)
3′ ITR (seq H)
p1257 pAAV2.1-CMV-3′ EGFP intein mDnaE
5′ ITR (seq A)
CMV promoter (seq M)
C-intein mDnaE (seq T) SEQ. ID No. 43
atggtgaaggtgatcggcaggaggagcctgggcgtgcagaggatcttcgacatcggcctgccccagtaccacaacttcctgctggcc
aacggcgccatcgccgccaac
3′ EGFP (seq L)
3xflag (seq E)
WPRE (seq F)
Bgh PolyA (seq G)
3′ ITR (seq H)
CEP290
p1005 pAAV2.1-CMV260-5′ CEP290 intein (set 1)
5′ ITR (seq A)
CMV260 (seq U) SEQ ID No. 44
ctagcgttgacattgattattgactagtacggtaaatggcccgcctggctgatgactcacggggatttccaagtctccaccccattgac
gtcaatgggagtttgttttggcaccaaaatcaacgggactttccaaaatgtcgtaacaactccgccccattgacgcaaatgggcggt
aggcgtgtacggtgggaggtctatataagcagagctggtttagtgaactagagaacccactgcttactggcttctcgagattccacca
tggcgc
5′ CEP290: SEQ. ID No. 45
atgccacctaatataaactggaaagaaataatgaaagttgacccagatgacctgccccgtcaagaagaactggcagataatttatt
gatttccttatccaaggtggaagtaaatgagctaaaaagtgaaaagcaagaaaatgtgatacaccttttcagaattactcagtcact
aatgaagatgaaagctcaagaagtggagctggctttggaagaagtagaaaaagctggagaagaacaagcaaaatttgaaaatca
attaaaaactaaagtaatgaaactggaaaatgaactggagatggctcagcagtctgcaggtggacgagatactcggtttttacgta
atgaaatttgccaacttgaaaaacaattagaacaaaaagatagagaattggaggacatggaaaaggagttggagaaagagaaga
aagttaatgagcaattggctcttcgaaatgaggaggcagaaaatgaaaacagcaaattaagaagagagaacaaacgtctaaaga
aaaagaatgaacaactttgtcaggatattattgactaccagaaacaaatagattcacagaaagaaacacttttatcaagaagaggg
gaagacagtgactaccgatcacagttgtctaaaaaaaactatgagcttatccaatatcttgatgaaattcagactttaacagaagct
aatgagaaaattgaagttcagaatcaagaaatgagaaaaaatttagaagagtctgtacaggaaatggagaagatgactgatgaat
ataatagaatgaaagctattgtgcatcagacagataatgtaatagatcagttaaaaaaagaaaacgatcattatcaacttcaagtg
caggagcttacagatctcctgaaatcaaaaaatgaagaagatgatccaattatggtagctgtcaatgcaaaagtagaagaatgga
agctaattttgtcttctaaagatgatgaaattattgagtatcagcaaatgttacataacctaagggagaaacttaagaatgctcagct
tgatgctgataaaagtaatgttatggctctacagcagggtatacaggaacgagacagtcaaattaagatgctcaccgaacaagtag
aacaatatacaaaagaaatggaaaagaatacttgtattattgaagatttgaaaaatgagctccaaagaaacaaaggtgcttcaacc
ctttctcaacagactcatatgaaaattcagtcaacgttagacattttaaaagagaaaactaaagaggctgagagaacagctgaact
ggctgaggctgatgctagggaaaaggataaagagttagttgaggctctgaagaggttaaaagattatgaatcgggagtatatggtt
tagaagatgctgtcgttgaaataaagaattgtaaaaaccaaattaaaataagagatcgagagattgaaatattaacaaaggaaat
caataaacttgaattgaagatcagtgatttccttgatgaaaatgaggcacttagagagcgtgtgggccttgaaccaaagacaatgat
tgatttaactgaatttagaaatagcaaacacttaaaacagcagcagtacagagctgaaaaccagattcttttgaaagagattgaaa
gtctagaggaagaacgacttgatctgaaaaaaaaaattcgtcaaatggctcaagaaagaggaaaaagaagtgcaacttcaggatt
aaccactgaggacctgaacctaactgaaaacatttctcaaggagatagaataagtgaaagaaaattggatttattgagcctcaaaa
atatgagtgaagcacaatcaaagaatgaatttctttcaagagaactaattgaaaaagaaagagatttagaaaggagtaggacagt
gatagccaaatttcagaataaattaaaagaattagttgaagaaaataagcaacttgaagaaggtatgaaagaaatattgcaagca
attaaggaaatgcagaaagatcctgatgttaaaggaggagaaacatctctaattatccctagccttgaaagactagttaatgctata
gaatcaaagaatgcagaaggaatctttgatgcgagtctgcatttgaaagcccaagttgatcagcttaccggaagaaatgaagaatt
aagacaggagctcagggaatctcggaaagaggctataaattattcacagcagttggcaaaagctaatttaaagatagaccatcttg
aaaaagaaactagtcttttacgacaatcagaaggatcgaatgttgtttttaaaggaattgacttacctgatgggatagcaccatctag
tgccagtatcattaattctcagaatgaatatttaatacatttgttacaggaactagaaaataaagaaaaaaagttaaagaatttaga
agattctcttgaagattacaacagaaaatttgctgtaattcgtcatcaacaaagtttgttgtataaagaatacctaagtgaaaaggag
acctggaaaacagaatctaaaacaataaaagaggaaaagagaaaacttgaggatcaagtccaacaagatgctataaaagtaaaa
gaatataataatttgctcaatgctcttcagatggattcggatgaaatgaaaaaaatacttgcagaaaatagtaggaaaattactgttt
tgcaagtgaatgaaaaatcacttataaggcaatatacaaccttagtagaattggagcgacaacttagaaaagaaaatgagaagca
aaagaatgaattgttgtcaatggaggctgaagtttgtgaaaaaattgggtgtttgcaaagatttaaggaaatggccattttcaagatt
gcagctctccaaaaagttgtagataatagtgtttctttgtctgaactagaactggctaataaacagtacaatgaactgactgctaagt
acagggacatcttgcaaaaagataatatgcttgttcaaagaacaagtaacttggaacacctggagtgtgaaaacatctccttaaaa
gaacaagtggagtctataaataaagaactggagattaccaaggaaaaacttcacactattgaacaagcctgggaacaggaaacta
aattaggtaatgaatctagcatggataaggcaaagaaatcaataaccaacagtgacattgtttccatttcaaaaaaaataactatgc
tggaaatgaaggaattaaatgaaaggcagcgggctgaacat
N-intein DnaE (seq D)
3xflag (seq E)
shPolyA (seq V) SEQ. ID No. 46
aattcaataaaagatctttattttcattagatctgtgtgttggttttttgtgtgcggcc
3′ ITR (seq H)
p1093 pAAV2.1-CMV260-3′ CEP290 intein (set 1)
5′ ITR (seq A)
CMV260 (seq U)
3′ CEP290: SEQ. ID No. 47
tgtcaaaaaatgtatgaacacttacggacttcgttaaagcaaatggaggaacgtaattttgaattggaaaccaaatttgctgagctta
ccaaaatcaatttggatgcacagaaggtggaacagatgttaagagatgaattagctgatagtgtgagcaaggcagtaagtgatgct
gataggcaacggattctagaattagagaagaatgaaatggaactaaaagttgaagtgtcaaaactgagagagatttctgatattgc
cagaagacaagttgaaattttgaatgcacaacaacaatctagggacaaggaagtagagtccctcagaatgcaactgctagactatc
aggcacagtctgatgaaaagtcgctcattgccaagttgcaccaacataatgtctctcttcaactgagtgaggctactgctcttggtaa
gttggagtcaattacatctaaactgcagaagatggaggcctacaacttgcgcttagagcagaaacttgatgaaaaagaacaggctc
tctattatgctcgtttggagggaagaaacagagcaaaacatctgcgccaaacaattcagtctctacgacgacagtttagtggagcttt
acccttggcacaacaggaaaagttctccaaaacaatgattcaactacaaaatgacaaacttaagataatgcaagaaatgaaaaatt
ctcaacaagaacatagaaatatggagaacaaaacattggagatggaattaaaattaaagggcctggaagagttaataagcacttt
aaaggataccaaaggagcccaaaaggtaatcaactggcatatgaaaatagaagaacttcgtcttcaagaacttaaactaaatcgg
gaattagtcaaggataaagaagaaataaaatatttgaataacataatttctgaatatgaacgtacaatcagcagtcttgaagaaga
aattgtgcaacagaacaagtttcatgaagaaagacaaatggcctgggatcaaagagaagttgacctggaacgccaactagacattt
ttgaccgtcagcaaaatgaaatactaaatgcggcacaaaagtttgaagaagctacaggatcaatccctgaccctagtttgccccttc
caaatcaacttgagatcgctctaaggaaaattaaggagaacattcgaataattctagaaacacgggcaacttgcaaatcactagaa
gagaaactaaaagagaaagaatctgctttaaggttagcagaacaaaatatactgtcaagagacaaagtaatcaatgaactgaggc
ttcgattgcctgccactgcagaaagagaaaagctcatagctgagctaggcagaaaagagatggaaccaaaatctcaccacacattg
aaaattgctcatcaaaccattgcaaacatgcaagcaaggttaaatcaaaaagaagaagtattaaagaagtatcaacgtcttctaga
aaaagccagagaggagcaaagagaaattgtgaagaaacatgaggaagaccttcatattcttcatcacagattagaactacaggct
gatagttcactaaataaattcaaacaaacggcttgggatttaatgaaacagtctcccactccagttcctaccaacaagcattttattcg
tctggctgagatggaacagacagtagcagaacaagatgactctctttcctcactcttggtcaaactaaagaaagtatcacaagattt
ggagagacaaagagaaatcactgaattaaaagtaaaagaatttgaaaatatcaaattacagcttcaagaaaaccatgaagatga
agtgaaaaaagtaaaagcggaagtagaggatttaaagtatcttctggaccagtcacaaaaggagtcacagtgtttaaaatctgaac
ttcaggctcaaaaagaagcaaattcaagagctccaacaactacaatgagaaatctagtagaacggctaaagagccaattagccttg
aaggagaaacaacagaaagcacttagtcgggcacttttagaactccgggcagaaatgacagcagctgctgaagaacgtattatttc
tgcaacttctcaaaaagaggcccatctcaatgttcaacaaatcgttgatcgacatactagagagctaaagacacaagttgaagattt
aaatgaaaatcttttaaaattgaaagaagcacttaaaactagtaaaaacagagaaaactcactaactgataatttgaatgacttaa
ataatgaactgcaaaagaaacaaaaagcctataataaaatacttagagagaaagaggaaattgatcaagagaatgatgaactga
aaaggcaaattaaaagactaaccagtggattacagggcaaacccctgacagataataaacaaagtctaattgaagaactccaaag
gaaagttaaaaaactagagaaccaattagagggaaaggtggaggaagtagacctaaaacctatgaaagaaaagaatgctaaag
aagaattaattaggtgggaagaaggtaaaaagtggcaagccaaaatagaaggaattcgaaacaagttaaaagagaaagagggg
gaagtctttactttaacaaagcagttgaatactttgaaggatctttttgccaaagccgataaagagaaacttactttgcagaggaaac
taaaaacaactggcatgactgttgatcaggttttgggaatacgagctttggagtcagaaaaagaattggaagaattaaaaaagaga
aatcttgacttagaaaatgatatattgtatatgagggcccaccaagctcttcctcgagattctgttgtagaagatttacatttacaaaa
tagatacctccaagaaaaacttcatgctttagaaaaacagttttcaaaggatacatattctaagccttcaatttcaggaatagagtca
gatgatcattgtcagagagaacaggagcttcagaaggaaaacttgaagttgtcatctgaaaatattgaactgaaatttcagcttgaa
caagcaaataaagatttgccaagattaaagaatcaagtcagagatttgaaggaaatgtgtgaatttcttaagaaagaaaaagcag
aagttcagcggaaacttggccatgttagagggtctggtagaagtggaaagacaatcccagaactggaaaaaaccattggtttaatg
aaaaaagtagttgaaaaagtccagagagaaaatgaacagttgaaaaaagcatcaggaatattgactagtgaaaaaatggctaat
attgagcaggaaaatgaaaaattgaaggctgaattagaaaaacttaaagctcatcttgggcatcagttgagcatgcactatgaatcc
aagaccaaaggcacagaaaaaattattgctgaaaatgaaaggcttcgtaaagaacttaaaaaagaaactgatgctgcagagaaa
ttacggatagcaaagaataatttagagatattaaatgagaagatgacagttcaactagaagagactggtaagagattgcagtttgc
agaaagcagaggtccacagcttgaaggtgctgacagtaagagctggaaatccattgtggttacaagaatgtatgaaaccaagttaa
aagaattggaaactgatattgccaaaaaaaatcaaagcattactgaccttaaacagcttgtaaaagaagcaacagagagagaaca
aaaagttaacaaatacaatgaagaccttgaacaacagattaagattcttaaacatgttcctgaaggtgctgagacagagcaaggcc
ttaaacgggagcttcaagttcttagattagctaatcatcagctggataaagagaaagcagaattaatccatcagatagaagctaaca
aggaccaaagtggagctgaaagcaccatacctgatgctgatcaactaaaggaaaaaataaaagatctagagacacagctcaaaa
tgtcagatctagaaaagcagcatttgaaggaggaaataaagaagctgaaaaaagaactggaaaattttgatccttcattttttgaag
aaattgaagatcttaagtataattacaaggaagaagtgaagaagaatattctcttagaagagaaggtaaaaaaactttcagaaca
attgggagttgaattaactagccctgttgctgcttctgaagagtttgaagatgaagaagaaagtcctgttaatttccccatttac
C-intein DnaE (seq I)
3xflag (seq E)
shPolyA (seq V)
3′ ITR (seq H)
p1065 pAAV2.1-CMV260-5′ CEP290 intein (set 2)
5′ ITR (seq A)
CMV260 (seq U)
5′ CEP290: SEQ. ID No. 48
atgccacctaatataaactggaaagaaataatgaaagttgacccagatgacctgccccgtcaagaagaactggcagataatttatt
gatttccttatccaaggtggaagtaaatgagctaaaaagtgaaaagcaagaaaatgtgatacaccttttcagaattactcagtcact
aatgaagatgaaagctcaagaagtggagctggctttggaagaagtagaaaaagctggagaagaacaagcaaaatttgaaaatca
attaaaaactaaagtaatgaaactggaaaatgaactggagatggctcagcagtctgcaggtggacgagatactcggtttttacgta
atgaaatttgccaacttgaaaaacaattagaacaaaaagatagagaattggaggacatggaaaaggagttggagaaagagaaga
aagttaatgagcaattggctcttcgaaatgaggaggcagaaaatgaaaacagcaaattaagaagagagaacaaacgtctaaaga
aaaagaatgaacaactttgtcaggatattattgactaccagaaacaaatagattcacagaaagaaacacttttatcaagaagaggg
gaagacagtgactaccgatcacagttgtctaaaaaaaactatgagcttatccaatatcttgatgaaattcagactttaacagaagct
aatgagaaaattgaagttcagaatcaagaaatgagaaaaaatttagaagagtctgtacaggaaatggagaagatgactgatgaat
ataatagaatgaaagctattgtgcatcagacagataatgtaatagatcagttaaaaaaagaaaacgatcattatcaacttcaagtg
caggagcttacagatctcctgaaatcaaaaaatgaagaagatgatccaattatggtagctgtcaatgcaaaagtagaagaatgga
agctaattttgtcttctaaagatgatgaaattattgagtatcagcaaatgttacataacctaagggagaaacttaagaatgctcagct
tgatgctgataaaagtaatgttatggctctacagcagggtatacaggaacgagacagtcaaattaagatgctcaccgaacaagtag
aacaatatacaaaagaaatggaaaagaatacttgtattattgaagatttgaaaaatgagctccaaagaaacaaaggtgcttcaacc
ctttctcaacagactcatatgaaaattcagtcaacgttagacattttaaaagagaaaactaaagaggctgagagaacagctgaact
ggctgaggctgatgctagggaaaaggataaagagttagttgaggctctgaagaggttaaaagattatgaatcgggagtatatggtt
tagaagatgctgtcgttgaaataaagaattgtaaaaaccaaattaaaataagagatcgagagattgaaatattaacaaaggaaat
caataaacttgaattgaagatcagtgatttccttgatgaaaatgaggcacttagagagcgtgtgggccttgaaccaaagacaatgat
tgatttaactgaatttagaaatagcaaacacttaaaacagcagcagtacagagctgaaaaccagattcttttgaaagagattgaaa
gtctagaggaagaacgacttgatctgaaaaaaaaaattcgtcaaatggctcaagaaagaggaaaaagaagtgcaacttcaggatt
aaccactgaggacctgaacctaactgaaaacatttctcaaggagatagaataagtgaaagaaaattggatttattgagcctcaaaa
atatgagtgaagcacaatcaaagaatgaatttctttcaagagaactaattgaaaaagaaagagatttagaaaggagtaggacagt
gatagccaaatttcagaataaattaaaagaattagttgaagaaaataagcaacttgaagaaggtatgaaagaaatattgcaagca
attaaggaaatgcagaaagatcctgatgttaaaggaggagaaacatctctaattatccctagccttgaaagactagttaatgctata
gaatcaaagaatgcagaaggaatctttgatgcgagtctgcatttgaaagcccaagttgatcagcttaccggaagaaatgaagaatt
aagacaggagctcagggaatctcggaaagaggctataaattattcacagcagttggcaaaagctaatttaaagatagaccatcttg
aaaaagaaactagtcttttacgacaatcagaaggatcgaatgttgtttttaaaggaattgacttacctgatgggatagcaccatctag
tgccagtatcattaattctcagaatgaatatttaatacatttgttacaggaactagaaaataaagaaaaaaagttaaagaatttaga
agattctcttgaagattacaacagaaaatttgctgtaattcgtcatcaacaaagtttgttgtataaagaatacctaagtgaaaaggag
acctggaaaacagaatctaaaacaataaaagaggaaaagagaaaacttgaggatcaagtccaacaagatgctataaaagtaaaa
gaatataataatttgctcaatgctcttcagatggattcggatgaaatgaaaaaaatacttgcagaaaatagtaggaaaattactgttt
tgcaagtgaatgaaaaatcacttataaggcaatatacaaccttagtagaattggagcgacaacttagaaaagaaaatgagaagca
aaagaatgaattgttgtcaatggaggctgaagtttgtgaaaaaattgggtgtttgcaaagatttaaggaaatggccattttcaagatt
gcagctctccaaaaagttgtagataatagtgtttctttgtctgaactagaactggctaataaacagtacaatgaactgactgctaagt
acagggacatcttgcaaaaagataatatgcttgttcaaagaacaagtaacttggaacacctggagtgtgaaaacatctccttaaaa
gaacaagtggagtctataaataaagaactggagattaccaaggaaaaacttcacactattgaacaagcctgggaacaggaaacta
aattaggtaatgaatctagcatggataaggcaaagaaatcaataaccaacagtgacattgtttccatttcaaaaaaaataactatgc
tggaaatgaaggaattaaatgaaaggcagcgggctgaacattgtcaaaaaatgtatgaacacttacggacttcgttaaagcaaatg
gaggaacgtaattttgaattggaaaccaaatttgctgagcttaccaaaatcaatttggatgcacagaaggtggaacagatgttaaga
gatgaattagctgatagtgtgagcaaggcagtaagtgatgctgataggcaacggattctagaattagagaagaatgaaatggaact
aaaagttgaagtgtcaaaactgagagagatttctgatattgccagaagacaagttgaaattttgaatgcacaacaacaatctaggg
acaaggaagtagagtccctcagaatgcaactgctagactatcaggcacagtctgatgaaaagtcgctcattgccaagttgcaccaa
cataatgtctctcttcaactgagtgaggctactgctcttggtaagttggagtcaattacatctaaactgcagaagatggaggcctaca
acttgcgcttagagcagaaacttgatgaaaaagaacaggctctctattatgctcgtttggagggaagaaacagagcaaaacatctg
cgccaaacaattcagtctctacgacgacagttt
N-intein DnaE (seq D)
3xflag (seq E)
Bgh PolyA (seq G)
3′ ITR (seq H)
p1067 pAAV2.1-CMV260-3′ CEP290 intein (set 2)
5′ TR (seq A)
CMV260 (seq U)
3′ CEP290: SEQ. ID No. 49
agtggagctttacccttggcacaacaggaaaagttctccaaaacaatgattcaactacaaaatgacaaacttaagataatgcaaga
aatgaaaaattctcaacaagaacatagaaatatggagaacaaaacattggagatggaattaaaattaaagggcctggaagagtta
ataagcactttaaaggataccaaaggagcccaaaaggtaatcaactggcatatgaaaatagaagaacttcgtcttcaagaacttaa
actaaatcgggaattagtcaaggataaagaagaaataaaatatttgaataacataatttctgaatatgaacgtacaatcagcagtct
tgaagaagaaattgtgcaacagaacaagtttcatgaagaaagacaaatggcctgggatcaaagagaagttgacctggaacgcca
actagacatttttgaccgtcagcaaaatgaaatactaaatgcggcacaaaagtttgaagaagctacaggatcaatccctgaccctag
tttgccccttccaaatcaacttgagatcgctctaaggaaaattaaggagaacattcgaataattctagaaacacgggcaacttgcaa
atcactagaagagaaactaaaagagaaagaatctgctttaaggttagcagaacaaaatatactgtcaagagacaaagtaatcaat
gaactgaggcttcgattgcctgccactgcagaaagagaaaagctcatagctgagctaggcagaaaagagatggaaccaaaatctc
accacacattgaaaattgctcatcaaaccattgcaaacatgcaagcaaggttaaatcaaaaagaagaagtattaaagaagtatca
acgtcttctagaaaaagccagagaggagcaaagagaaattgtgaagaaacatgaggaagaccttcatattcttcatcacagattag
aactacaggctgatagttcactaaataaattcaaacaaacggcttgggatttaatgaaacagtctcccactccagttcctaccaaca
agcattttattcgtctggctgagatggaacagacagtagcagaacaagatgactctctttcctcactcttggtcaaactaaagaaagt
atcacaagatttggagagacaaagagaaatcactgaattaaaagtaaaagaatttgaaaatatcaaattacagcttcaagaaaac
catgaagatgaagtgaaaaaagtaaaagcggaagtagaggatttaaagtatcttctggaccagtcacaaaaggagtcacagtgttt
aaaatctgaacttcaggctcaaaaagaagcaaattcaagagctccaacaactacaatgagaaatctagtagaacggctaaagagc
caattagccttgaaggagaaacaacagaaagcacttagtcgggcacttttagaactccgggcagaaatgacagcagctgctgaag
aacgtattatttctgcaacttctcaaaaagaggcccatctcaatgttcaacaaatcgttgatcgacatactagagagctaaagacaca
agttgaagatttaaatgaaaatcttttaaaattgaaagaagcacttaaaactagtaaaaacagagaaaactcactaactgataattt
gaatgacttaaataatgaactgcaaaagaaacaaaaagcctataataaaatacttagagagaaagaggaaattgatcaagagaa
tgatgaactgaaaaggcaaattaaaagactaaccagtggattacagggcaaacccctgacagataataaacaaagtctaattgaa
gaactccaaaggaaagttaaaaaactagagaaccaattagagggaaaggtggaggaagtagacctaaaacctatgaaagaaaa
gaatgctaaagaagaattaattaggtgggaagaaggtaaaaagtggcaagccaaaatagaaggaattcgaaacaagttaaaaga
gaaagagggggaagtctttactttaacaaagcagttgaatactttgaaggatctttttgccaaagccgataaagagaaacttactttg
cagaggaaactaaaaacaactggcatgactgttgatcaggttttgggaatacgagctttggagtcagaaaaagaattggaagaatt
aaaaaagagaaatcttgacttagaaaatgatatattgtatatgagggcccaccaagctcttcctcgagattctgttgtagaagattta
catttacaaaatagatacctccaagaaaaacttcatgctttagaaaaacagttttcaaaggatacatattctaagccttcaatttcag
gaatagagtcagatgatcattgtcagagagaacaggagcttcagaaggaaaacttgaagttgtcatctgaaaatattgaactgaaa
tttcagcttgaacaagcaaataaagatttgccaagattaaagaatcaagtcagagatttgaaggaaatgtgtgaatttcttaagaaa
gaaaaagcagaagttcagcggaaacttggccatgttagagggtctggtagaagtggaaagacaatcccagaactggaaaaaacc
attggtttaatgaaaaaagtagttgaaaaagtccagagagaaaatgaacagttgaaaaaagcatcaggaatattgactagtgaaa
aaatggctaatattgagcaggaaaatgaaaaattgaaggctgaattagaaaaacttaaagctcatcttgggcatcagttgagcatg
cactatgaatccaagaccaaaggcacagaaaaaattattgctgaaaatgaaaggcttcgtaaagaacttaaaaaagaaactgatg
ctgcagagaaattacggatagcaaagaataatttagagatattaaatgagaagatgacagttcaactagaagagactggtaagag
attgcagtttgcagaaagcagaggtccacagcttgaaggtgctgacagtaagagctggaaatccattgtggttacaagaatgtatga
aaccaagttaaaagaattggaaactgatattgccaaaaaaaatcaaagcattactgaccttaaacagcttgtaaaagaagcaaca
gagagagaacaaaaagttaacaaatacaatgaagaccttgaacaacagattaagattcttaaacatgttcctgaaggtgctgagac
agagcaaggccttaaacgggagcttcaagttcttagattagctaatcatcagctggataaagagaaagcagaattaatccatcaga
tagaagctaacaaggaccaaagtggagctgaaagcaccatacctgatgctgatcaactaaaggaaaaaataaaagatctagaga
cacagctcaaaatgtcagatctagaaaagcagcatttgaaggaggaaataaagaagctgaaaaaagaactggaaaattttgatcc
ttcattttttgaagaaattgaagatcttaagtataattacaaggaagaagtgaagaagaatattctcttagaagagaaggtaaaaaa
actttcagaacaattgggagttgaattaactagccctgttgctgcttctgaagagtttgaagatgaagaagaaagtcctgttaatttcc
ccatttac
C-intein DnaE (seq I)
3xflag (seq E)
Bgh PolyA (seq G)
3′ ITR (seq H)
p1087 pAAV2.1-CMV260-5′ CEP290 intein (set 3)
5′ ITR (seq A)
CMV260 (seq U)
5′ CEP290: SEQ. ID No. 50
atgccacctaatataaactggaaagaaataatgaaagttgacccagatgacctgccccgtcaagaagaactggcagataatttatt
gatttccttatccaaggtggaagtaaatgagctaaaaagtgaaaagcaagaaaatgtgatacaccttttcagaattactcagtcact
aatgaagatgaaagctcaagaagtggagctggctttggaagaagtagaaaaagctggagaagaacaagcaaaatttgaaaatca
attaaaaactaaagtaatgaaactggaaaatgaactggagatggctcagcagtctgcaggtggacgagatactcggtttttacgta
atgaaatttgccaacttgaaaaacaattagaacaaaaagatagagaattggaggacatggaaaaggagttggagaaagagaaga
aagttaatgagcaattggctcttcgaaatgaggaggcagaaaatgaaaacagcaaattaagaagagagaacaaacgtctaaaga
aaaagaatgaacaactttgtcaggatattattgactaccagaaacaaatagattcacagaaagaaacacttttatcaagaagaggg
gaagacagtgactaccgatcacagttgtctaaaaaaaactatgagcttatccaatatcttgatgaaattcagactttaacagaagct
aatgagaaaattgaagttcagaatcaagaaatgagaaaaaatttagaagagtctgtacaggaaatggagaagatgactgatgaat
ataatagaatgaaagctattgtgcatcagacagataatgtaatagatcagttaaaaaaagaaaacgatcattatcaacttcaagtg
caggagcttacagatctcctgaaatcaaaaaatgaagaagatgatccaattatggtagctgtcaatgcaaaagtagaagaatgga
agctaattttgtcttctaaagatgatgaaattattgagtatcagcaaatgttacataacctaagggagaaacttaagaatgctcagct
tgatgctgataaaagtaatgttatggctctacagcagggtatacaggaacgagacagtcaaattaagatgctcaccgaacaagtag
aacaatatacaaaagaaatggaaaagaatacttgtattattgaagatttgaaaaatgagctccaaagaaacaaaggtgcttcaacc
ctttctcaacagactcatatgaaaattcagtcaacgttagacattttaaaagagaaaactaaagaggctgagagaacagctgaact
ggctgaggctgatgctagggaaaaggataaagagttagttgaggctctgaagaggttaaaagattatgaatcgggagtatatggtt
tagaagatgctgtcgttgaaataaagaattgtaaaaaccaaattaaaataagagatcgagagattgaaatattaacaaaggaaat
caataaacttgaattgaagatcagtgatttccttgatgaaaatgaggcacttagagagcgtgtgggccttgaaccaaagacaatgat
tgatttaactgaatttagaaatagcaaacacttaaaacagcagcagtacagagctgaaaaccagattcttttgaaagagattgaaa
gtctagaggaagaacgacttgatctgaaaaaaaaaattcgtcaaatggctcaagaaagaggaaaaagaagtgcaacttcaggatt
aaccactgaggacctgaacctaactgaaaacatttctcaaggagatagaataagtgaaagaaaattggatttattgagcctcaaaa
atatgagtgaagcacaatcaaagaatgaatttctttcaagagaactaattgaaaaagaaagagatttagaaaggagtaggacagt
gatagccaaatttcagaataaattaaaagaattagttgaagaaaataagcaacttgaagaaggtatgaaagaaatattgcaagca
attaaggaaatgcagaaagatcctgatgttaaaggaggagaaacatctctaattatccctagccttgaaagactagttaatgctata
gaatcaaagaatgcagaaggaatctttgatgcgagtctgcatttgaaagcccaagttgatcagcttaccggaagaaatgaagaatt
aagacaggagctcagggaatctcggaaagaggctataaattattcacagcagttggcaaaagctaatttaaagatagaccatcttg
aaaaagaaactagtcttttacgacaatcagaaggatcgaatgttgtttttaaaggaattgacttacctgatgggatagcaccatctag
tgccagtatcattaattctcagaatgaatatttaatacatttgttacaggaactagaaaataaagaaaaaaagttaaagaatttaga
agattctcttgaagattacaacagaaaatttgctgtaattcgtcatcaacaaagtttgttgtataaagaatacctaagtgaaaaggag
acctggaaaacagaatctaaaacaataaaagaggaaaagagaaaacttgaggatcaagtccaacaagatgctataaaagtaaaa
gaatataataatttgctcaatgctcttcagatggattcggatgaaatgaaaaaaatacttgcagaaaatagtaggaaaattactgttt
tgcaagtgaatgaaaaatcacttataaggcaatatacaaccttagtagaattggagcgacaacttagaaaagaaaatgagaagca
aaagaatgaattgttgtcaatggaggctgaagtttgtgaaaaaattgggtgtttgcaaagatttaaggaaatggccattttcaagatt
gcagctctccaaaaagttgtagataatagtgtttctttgtctgaactagaactggctaataaacagtacaatgaactgactgctaagt
acagggacatcttgcaaaaagataatatgcttgttcaaagaacaagtaacttggaacacctggagtgtgaaaacatctccttaaaa
gaacaagtggagtctataaataaagaactggagattaccaaggaaaaacttcacactattgaacaagcctgggaacaggaaacta
aattaggtaatgaatctagcatggataaggcaaagaaatcaataaccaacagtgacattgtttccatttcaaaaaaaataactatgc
tggaaatgaaggaattaaatgaaaggcagcgggctgaacattgtcaaaaaatgtatgaacacttacggacttcgttaaagcaaatg
gaggaacgtaattttgaattggaaaccaaatttgctgagcttaccaaaatcaatttggatgcacagaaggtggaacagatgttaaga
gatgaattagctgatagtgtgagcaaggcagtaagtgatgctgataggcaacggattctagaattagagaagaatgaaatggaact
aaaagttgaagtgtcaaaactgagagagatttctgatattgccagaagacaagttgaaattttgaatgcacaacaacaatctaggg
acaaggaagtagagtccctcagaatgcaactgctagactatcaggcacagtctgatgaaaagtcgctcattgccaagttgcaccaa
cataatgtctctcttcaactgagtgaggctactgctcttggtaagttggagtcaattacatctaaactgcagaagatggaggcctaca
acttgcgcttagagcagaaacttgatgaaaaagaacaggctctctattatgctcgtttggagggaagaaacagagcaaaacatctg
cgccaaacaattcagtctctacgacgacagttt
N-intein mDnaE (seq S)
3xflag (seq E)
Bgh PolyA (seq G)
3′ ITR (seq H)
p1088 pAAV2.1-CMV260-3′ CEP290 intein (set 3)
5′ ITR (seq A)
CMV260 (seq U)
3′ CEP290: SEQ. ID No. 51
agtggagctttacccttggcacaacaggaaaagttctccaaaacaatgattcaactacaaaatgacaaacttaagataatgcaaga
aatgaaaaattctcaacaagaacatagaaatatggagaacaaaacattggagatggaattaaaattaaagggcctggaagagtta
ataagcactttaaaggataccaaaggagcccaaaaggtaatcaactggcatatgaaaatagaagaacttcgtcttcaagaacttaa
actaaatcgggaattagtcaaggataaagaagaaataaaatatttgaataacataatttctgaatatgaacgtacaatcagcagtct
tgaagaagaaattgtgcaacagaacaagtttcatgaagaaagacaaatggcctgggatcaaagagaagttgacctggaacgcca
actagacatttttgaccgtcagcaaaatgaaatactaaatgcggcacaaaagtttgaagaagctacaggatcaatccctgaccctag
tttgccccttccaaatcaacttgagatcgctctaaggaaaattaaggagaacattcgaataattctagaaacacgggcaacttgcaa
atcactagaagagaaactaaaagagaaagaatctgctttaaggttagcagaacaaaatatactgtcaagagacaaagtaatcaat
gaactgaggcttcgattgcctgccactgcagaaagagaaaagctcatagctgagctaggcagaaaagagatggaaccaaaatctc
accacacattgaaaattgctcatcaaaccattgcaaacatgcaagcaaggttaaatcaaaaagaagaagtattaaagaagtatca
acgtcttctagaaaaagccagagaggagcaaagagaaattgtgaagaaacatgaggaagaccttcatattcttcatcacagattag
aactacaggctgatagttcactaaataaattcaaacaaacggcttgggatttaatgaaacagtctcccactccagttcctaccaaca
agcattttattcgtctggctgagatggaacagacagtagcagaacaagatgactctctttcctcactcttggtcaaactaaagaaagt
atcacaagatttggagagacaaagagaaatcactgaattaaaagtaaaagaatttgaaaatatcaaattacagcttcaagaaaac
catgaagatgaagtgaaaaaagtaaaagcggaagtagaggatttaaagtatcttctggaccagtcacaaaaggagtcacagtgttt
aaaatctgaacttcaggctcaaaaagaagcaaattcaagagctccaacaactacaatgagaaatctagtagaacggctaaagagc
caattagccttgaaggagaaacaacagaaagcacttagtcgggcacttttagaactccgggcagaaatgacagcagctgctgaag
aacgtattatttctgcaacttctcaaaaagaggcccatctcaatgttcaacaaatcgttgatcgacatactagagagctaaagacaca
agttgaagatttaaatgaaaatcttttaaaattgaaagaagcacttaaaactagtaaaaacagagaaaactcactaactgataattt
gaatgacttaaataatgaactgcaaaagaaacaaaaagcctataataaaatacttagagagaaagaggaaattgatcaagagaa
tgatgaactgaaaaggcaaattaaaagactaaccagtggattacagggcaaacccctgacagataataaacaaagtctaattgaa
gaactccaaaggaaagttaaaaaactagagaaccaattagagggaaaggtggaggaagtagacctaaaacctatgaaagaaaa
gaatgctaaagaagaattaattaggtgggaagaaggtaaaaagtggcaagccaaaatagaaggaattcgaaacaagttaaaaga
gaaagagggggaagtctttactttaacaaagcagttgaatactttgaaggatctttttgccaaagccgataaagagaaacttactttg
cagaggaaactaaaaacaactggcatgactgttgatcaggttttgggaatacgagctttggagtcagaaaaagaattggaagaatt
aaaaaagagaaatcttgacttagaaaatgatatattgtatatgagggcccaccaagctcttcctcgagattctgttgtagaagattta
catttacaaaatagatacctccaagaaaaacttcatgctttagaaaaacagttttcaaaggatacatattctaagccttcaatttcag
gaatagagtcagatgatcattgtcagagagaacaggagcttcagaaggaaaacttgaagttgtcatctgaaaatattgaactgaaa
tttcagcttgaacaagcaaataaagatttgccaagattaaagaatcaagtcagagatttgaaggaaatgtgtgaatttcttaagaaa
gaaaaagcagaagttcagcggaaacttggccatgttagagggtctggtagaagtggaaagacaatcccagaactggaaaaaacc
attggtttaatgaaaaaagtagttgaaaaagtccagagagaaaatgaacagttgaaaaaagcatcaggaatattgactagtgaaa
aaatggctaatattgagcaggaaaatgaaaaattgaaggctgaattagaaaaacttaaagctcatcttgggcatcagttgagcatg
cactatgaatccaagaccaaaggcacagaaaaaattattgctgaaaatgaaaggcttcgtaaagaacttaaaaaagaaactgatg
ctgcagagaaattacggatagcaaagaataatttagagatattaaatgagaagatgacagttcaactagaagagactggtaagag
attgcagtttgcagaaagcagaggtccacagcttgaaggtgctgacagtaagagctggaaatccattgtggttacaagaatgtatga
aaccaagttaaaagaattggaaactgatattgccaaaaaaaatcaaagcattactgaccttaaacagcttgtaaaagaagcaaca
gagagagaacaaaaagttaacaaatacaatgaagaccttgaacaacagattaagattcttaaacatgttcctgaaggtgctgagac
agagcaaggccttaaacgggagcttcaagttcttagattagctaatcatcagctggataaagagaaagcagaattaatccatcaga
tagaagctaacaaggaccaaagtggagctgaaagcaccatacctgatgctgatcaactaaaggaaaaaataaaagatctagaga
cacagctcaaaatgtcagatctagaaaagcagcatttgaaggaggaaataaagaagctgaaaaaagaactggaaaattttgatcc
ttcattttttgaagaaattgaagatcttaagtataattacaaggaagaagtgaagaagaatattctcttagaagagaaggtaaaaaa
actttcagaacaattgggagttgaattaactagccctgttgctgcttctgaagagtttgaagatgaagaagaaagtcctgttaatttcc
ccatttac
C-intein mDnaE (seq T)
3xflag (seq E)
Bgh PolyA (seq G)
3′ ITR (seq H)
p1182 pAAV2.1-CMV260-5′ CEP290 intein (set 4)
5′ TR (seq A)
CMV260 (seq U)
5′ CEP290: SEQ. ID No. 52
atgccacctaatataaactggaaagaaataatgaaagttgacccagatgacctgccccgtcaagaagaactggcagataatttatt
gatttccttatccaaggtggaagtaaatgagctaaaaagtgaaaagcaagaaaatgtgatacaccttttcagaattactcagtcact
aatgaagatgaaagctcaagaagtggagctggctttggaagaagtagaaaaagctggagaagaacaagcaaaatttgaaaatca
attaaaaactaaagtaatgaaactggaaaatgaactggagatggctcagcagtctgcaggtggacgagatactcggtttttacgta
atgaaatttgccaacttgaaaaacaattagaacaaaaagatagagaattggaggacatggaaaaggagttggagaaagagaaga
aagttaatgagcaattggctcttcgaaatgaggaggcagaaaatgaaaacagcaaattaagaagagagaacaaacgtctaaaga
aaaagaatgaacaactttgtcaggatattattgactaccagaaacaaatagattcacagaaagaaacacttttatcaagaagaggg
gaagacagtgactaccgatcacagttgtctaaaaaaaactatgagcttatccaatatcttgatgaaattcagactttaacagaagct
aatgagaaaattgaagttcagaatcaagaaatgagaaaaaatttagaagagtctgtacaggaaatggagaagatgactgatgaat
ataatagaatgaaagctattgtgcatcagacagataatgtaatagatcagttaaaaaaagaaaacgatcattatcaacttcaagtg
caggagcttacagatctcctgaaatcaaaaaatgaagaagatgatccaattatggtagctgtcaatgcaaaagtagaagaatgga
agctaattttgtcttctaaagatgatgaaattattgagtatcagcaaatgttacataacctaagggagaaacttaagaatgctcagct
tgatgctgataaaagtaatgttatggctctacagcagggtatacaggaacgagacagtcaaattaagatgctcaccgaacaagtag
aacaatatacaaaagaaatggaaaagaatacttgtattattgaagatttgaaaaatgagctccaaagaaacaaaggtgcttcaacc
ctttctcaacagactcatatgaaaattcagtcaacgttagacattttaaaagagaaaactaaagaggctgagagaacagctgaact
ggctgaggctgatgctagggaaaaggataaagagttagttgaggctctgaagaggttaaaagattatgaatcgggagtatatggtt
tagaagatgctgtcgttgaaataaagaattgtaaaaaccaaattaaaataagagatcgagagattgaaatattaacaaaggaaat
caataaacttgaattgaagatcagtgatttccttgatgaaaatgaggcacttagagagcgtgtgggccttgaaccaaagacaatgat
tgatttaactgaatttagaaatagcaaacacttaaaacagcagcagtacagagctgaaaaccagattcttttgaaagagattgaaa
gtctagaggaagaacgacttgatctgaaaaaaaaaattcgtcaaatggctcaagaaagaggaaaaagaagtgcaacttcaggatt
aaccactgaggacctgaacctaactgaaaacatttctcaaggagatagaataagtgaaagaaaattggatttattgagcctcaaaa
atatgagtgaagcacaatcaaagaatgaatttctttcaagagaactaattgaaaaagaaagagatttagaaaggagtaggacagt
gatagccaaatttcagaataaattaaaagaattagttgaagaaaataagcaacttgaagaaggtatgaaagaaatattgcaagca
attaaggaaatgcagaaagatcctgatgttaaaggaggagaaacatctctaattatccctagccttgaaagactagttaatgctata
gaatcaaagaatgcagaaggaatctttgatgcgagtctgcatttgaaagcccaagttgatcagcttaccggaagaaatgaagaatt
aagacaggagctcagggaatctcggaaagaggctataaattattcacagcagttggcaaaagctaatttaaagatagaccatcttg
aaaaagaaactagtcttttacgacaatcagaaggatcgaatgttgtttttaaaggaattgacttacctgatgggatagcaccatctag
tgccagtatcattaattctcagaatgaatatttaatacatttgttacaggaactagaaaataaagaaaaaaagttaaagaatttaga
agattctcttgaagattacaacagaaaatttgctgtaattcgtcatcaacaaagtttgttgtataaagaatacctaagtgaaaaggag
acctggaaaacagaatctaaaacaataaaagaggaaaagagaaaacttgaggatcaagtccaacaagatgctataaaagtaaaa
gaatataataatttgctcaatgctcttcagatggattcggatgaaatgaaaaaaatacttgcagaaaatagtaggaaaattactgttt
tgcaagtgaatgaaaaatcacttataaggcaatatacaaccttagtagaattggagcgacaacttagaaaagaaaatgagaagca
aaagaatgaattgttgtcaatggaggctgaagtt
N-intein DnaE (seq D)
3xflag (seq E)
WPRE (seq F)
Bgh PolyA (seq G)
3′ITR (seq H)
p1183 pAAV2.1-CMV260-CEP290 body intein (set 4)
5′ ITR (seq A)
CMV260 (seq U)
C-intein DnaE (seq I)
CEP290 body: SEQ. ID No. 53
tgtgaaaaaattgggtgtttgcaaagatttaaggaaatggccattttcaagattgcagctctccaaaaagttgtagataatagtgtttc
tttgtctgaactagaactggctaataaacagtacaatgaactgactgctaagtacagggacatcttgcaaaaagataatatgcttgtt
caaagaacaagtaacttggaacacctggagtgtgaaaacatctccttaaaagaacaagtggagtctataaataaagaactggaga
ttaccaaggaaaaacttcacactattgaacaagcctgggaacaggaaactaaattaggtaatgaatctagcatggataaggcaaa
gaaatcaataaccaacagtgacattgtttccatttcaaaaaaaataactatgctggaaatgaaggaattaaatgaaaggcagcggg
ctgaacattgtcaaaaaatgtatgaacacttacggacttcgttaaagcaaatggaggaacgtaattttgaattggaaaccaaatttg
ctgagcttaccaaaatcaatttggatgcacagaaggtggaacagatgttaagagatgaattagctgatagtgtgagcaaggcagta
agtgatgctgataggcaacggattctagaattagagaagaatgaaatggaactaaaagttgaagtgtcaaaactgagagagatttc
tgatattgccagaagacaagttgaaattttgaatgcacaacaacaatctagggacaaggaagtagagtccctcagaatgcaactgc
tagactatcaggcacagtctgatgaaaagtcgctcattgccaagttgcaccaacataatgtctctcttcaactgagtgaggctactgc
tcttggtaagttggagtcaattacatctaaactgcagaagatggaggcctacaacttgcgcttagagcagaaacttgatgaaaaaga
acaggctctctattatgctcgtttggagggaagaaacagagcaaaacatctgcgccaaacaattcagtctctacgacgacagtttag
tggagctttacccttggcacaacaggaaaagttctccaaaacaatgattcaactacaaaatgacaaacttaagataatgcaagaaa
tgaaaaattctcaacaagaacatagaaatatggagaacaaaacattggagatggaattaaaattaaagggcctggaagagttaat
aagcactttaaaggataccaaaggagcccaaaaggtaatcaactggcatatgaaaatagaagaacttcgtcttcaagaacttaaac
taaatcgggaattagtcaaggataaagaagaaataaaatatttgaataacataatttctgaatatgaacgtacaatcagcagtcttg
aagaagaaattgtgcaacagaacaagtttcatgaagaaagacaaatggcctgggatcaaagagaagttgacctggaacgccaact
agacatttttgaccgtcagcaaaatgaaatactaaatgcggcacaaaagtttgaagaagctacaggatcaatccctgaccctagttt
gccccttccaaatcaacttgagatcgctctaaggaaaattaaggagaacattcgaataattctagaaacacgggcaact
N-intein Rma DnaB (seq Q)
3xflag (seq E)
WPRE (seq F)
Bgh PolyA (seq G)
3′ITR (seq H)
p1181 pAAV2.1-CMV260-3′ CEP290 intein (set 4/set5)
5′ ITR (seq A)
CMV260 (seq U)
C-intein Rma DnaB (seq R)
3′ CEP290: SEQ ID No. 54
tgcaaatcactagaagagaaactaaaagagaaagaatctgctttaaggttagcagaacaaaatatactgtcaagagacaaagtaa
tcaatgaactgaggcttcgattgcctgccactgcagaaagagaaaagctcatagctgagctaggcagaaaagagatggaaccaaa
atctcaccacacattgaaaattgctcatcaaaccattgcaaacatgcaagcaaggttaaatcaaaaagaagaagtattaaagaagt
atcaacgtcttctagaaaaagccagagaggagcaaagagaaattgtgaagaaacatgaggaagaccttcatattcttcatcacaga
ttagaactacaggctgatagttcactaaataaattcaaacaaacggcttgggatttaatgaaacagtctcccactccagttcctacca
acaagcattttattcgtctggctgagatggaacagacagtagcagaacaagatgactctctttcctcactcttggtcaaactaaagaa
agtatcacaagatttggagagacaaagagaaatcactgaattaaaagtaaaagaatttgaaaatatcaaattacagcttcaagaa
aaccatgaagatgaagtgaaaaaagtaaaagcggaagtagaggatttaaagtatcttctggaccagtcacaaaaggagtcacagt
gtttaaaatctgaacttcaggctcaaaaagaagcaaattcaagagctccaacaactacaatgagaaatctagtagaacggctaaag
agccaattagccttgaaggagaaacaacagaaagcacttagtcgggcacttttagaactccgggcagaaatgacagcagctgctg
aagaacgtattatttctgcaacttctcaaaaagaggcccatctcaatgttcaacaaatcgttgatcgacatactagagagctaaaga
cacaagttgaagatttaaatgaaaatcttttaaaattgaaagaagcacttaaaactagtaaaaacagagaaaactcactaactgat
aatttgaatgacttaaataatgaactgcaaaagaaacaaaaagcctataataaaatacttagagagaaagaggaaattgatcaag
agaatgatgaactgaaaaggcaaattaaaagactaaccagtggattacagggcaaacccctgacagataataaacaaagtctaat
tgaagaactccaaaggaaagttaaaaaactagagaaccaattagagggaaaggtggaggaagtagacctaaaacctatgaaag
aaaagaatgctaaagaagaattaattaggtgggaagaaggtaaaaagtggcaagccaaaatagaaggaattcgaaacaagttaa
aagagaaagagggggaagtctttactttaacaaagcagttgaatactttgaaggatctttttgccaaagccgataaagagaaactta
ctttgcagaggaaactaaaaacaactggcatgactgttgatcaggttttgggaatacgagctttggagtcagaaaaagaattggaa
gaattaaaaaagagaaatcttgacttagaaaatgatatattgtatatgagggcccaccaagctcttcctcgagattctgttgtagaag
atttacatttacaaaatagatacctccaagaaaaacttcatgctttagaaaaacagttttcaaaggatacatattctaagccttcaatt
tcaggaatagagtcagatgatcattgtcagagagaacaggagcttcagaaggaaaacttgaagttgtcatctgaaaatattgaact
gaaatttcagcttgaacaagcaaataaagatttgccaagattaaagaatcaagtcagagatttgaaggaaatgtgtgaatttcttaa
gaaagaaaaagcagaagttcagcggaaacttggccatgttagagggtctggtagaagtggaaagacaatcccagaactggaaaa
aaccattggtttaatgaaaaaagtagttgaaaaagtccagagagaaaatgaacagttgaaaaaagcatcaggaatattgactagt
gaaaaaatggctaatattgagcaggaaaatgaaaaattgaaggctgaattagaaaaacttaaagctcatcttgggcatcagttgag
catgcactatgaatccaagaccaaaggcacagaaaaaattattgctgaaaatgaaaggcttcgtaaagaacttaaaaaagaaact
gatgctgcagagaaattacggatagcaaagaataatttagagatattaaatgagaagatgacagttcaactagaagagactggta
agagattgcagtttgcagaaagcagaggtccacagcttgaaggtgctgacagtaagagctggaaatccattgtggttacaagaatg
tatgaaaccaagttaaaagaattggaaactgatattgccaaaaaaaatcaaagcattactgaccttaaacagcttgtaaaagaagc
aacagagagagaacaaaaagttaacaaatacaatgaagaccttgaacaacagattaagattcttaaacatgttcctgaaggtgctg
agacagagcaaggccttaaacgggagcttcaagttcttagattagctaatcatcagctggataaagagaaagcagaattaatccat
cagatagaagctaacaaggaccaaagtggagctgaaagcaccatacctgatgctgatcaactaaaggaaaaaataaaagatcta
gagacacagctcaaaatgtcagatctagaaaagcagcatttgaaggaggaaataaagaagctgaaaaaagaactggaaaatttt
gatccttcattttttgaagaaattgaagatcttaagtataattacaaggaagaagtgaagaagaatattctcttagaagagaaggta
aaaaaactttcagaacaattgggagttgaattaactagccctgttgctgcttctgaagagtttgaagatgaagaagaaagtcctgtta
atttccccatttac
3xflag (seq E)
WPRE (seq F)
Bgh PolyA (seq G)
3′ITR (seq H)
p1179 pAAV2.1-CMV260-5′ CEP290 intein (set 5)
5′ ITR (seq A)
CMV260 (seq U)
5′ CEP290: SEQ. ID No. 55
atgccacctaatataaactggaaagaaataatgaaagttgacccagatgacctgccccgtcaagaagaactggcagataatttatt
gatttccttatccaaggtggaagtaaatgagctaaaaagtgaaaagcaagaaaatgtgatacaccttttcagaattactcagtcact
aatgaagatgaaagctcaagaagtggagctggctttggaagaagtagaaaaagctggagaagaacaagcaaaatttgaaaatca
attaaaaactaaagtaatgaaactggaaaatgaactggagatggctcagcagtctgcaggtggacgagatactcggtttttacgta
atgaaatttgccaacttgaaaaacaattagaacaaaaagatagagaattggaggacatggaaaaggagttggagaaagagaaga
aagttaatgagcaattggctcttcgaaatgaggaggcagaaaatgaaaacagcaaattaagaagagagaacaaacgtctaaaga
aaaagaatgaacaactttgtcaggatattattgactaccagaaacaaatagattcacagaaagaaacacttttatcaagaagaggg
gaagacagtgactaccgatcacagttgtctaaaaaaaactatgagcttatccaatatcttgatgaaattcagactttaacagaagct
aatgagaaaattgaagttcagaatcaagaaatgagaaaaaatttagaagagtctgtacaggaaatggagaagatgactgatgaat
ataatagaatgaaagctattgtgcatcagacagataatgtaatagatcagttaaaaaaagaaaacgatcattatcaacttcaagtg
caggagcttacagatctcctgaaatcaaaaaatgaagaagatgatccaattatggtagctgtcaatgcaaaagtagaagaatgga
agctaattttgtcttctaaagatgatgaaattattgagtatcagcaaatgttacataacctaagggagaaacttaagaatgctcagct
tgatgctgataaaagtaatgttatggctctacagcagggtatacaggaacgagacagtcaaattaagatgctcaccgaacaagtag
aacaatatacaaaagaaatggaaaagaatacttgtattattgaagatttgaaaaatgagctccaaagaaacaaaggtgcttcaacc
ctttctcaacagactcatatgaaaattcagtcaacgttagacattttaaaagagaaaactaaagaggctgagagaacagctgaact
ggctgaggctgatgctagggaaaaggataaagagttagttgaggctctgaagaggttaaaagattatgaa
N-intein mDnaE (seq S)
3xflag (seq E)
WPRE (seq F)
Bgh PolyA (seq G)
3′ITR (seq H)
p1180 pAAV2.1-CMV260-CEP290 body intein (set 5)
5′ ITR (seq A)
CMV260 (seq U)
C-intein mDnaE (seq T)
CEP290 body: SEQ. ID No. 56
tcgggagtatatggtttagaagatgctgtcgttgaaataaagaattgtaaaaaccaaattaaaataagagatcgagagattgaaat
attaacaaaggaaatcaataaacttgaattgaagatcagtgatttccttgatgaaaatgaggcacttagagagcgtgtgggccttga
accaaagacaatgattgatttaactgaatttagaaatagcaaacacttaaaacagcagcagtacagagctgaaaaccagattctttt
gaaagagattgaaagtctagaggaagaacgacttgatctgaaaaaaaaaattcgtcaaatggctcaagaaagaggaaaaagaag
tgcaacttcaggattaaccactgaggacctgaacctaactgaaaacatttctcaaggagatagaataagtgaaagaaaattggattt
attgagcctcaaaaatatgagtgaagcacaatcaaagaatgaatttctttcaagagaactaattgaaaaagaaagagatttagaaa
ggagtaggacagtgatagccaaatttcagaataaattaaaagaattagttgaagaaaataagcaacttgaagaaggtatgaaaga
aatattgcaagcaattaaggaaatgcagaaagatcctgatgttaaaggaggagaaacatctctaattatccctagccttgaaagact
agttaatgctatagaatcaaagaatgcagaaggaatctttgatgcgagtctgcatttgaaagcccaagttgatcagcttaccggaag
aaatgaagaattaagacaggagctcagggaatctcggaaagaggctataaattattcacagcagttggcaaaagctaatttaaag
atagaccatcttgaaaaagaaactagtcttttacgacaatcagaaggatcgaatgttgtttttaaaggaattgacttacctgatggga
tagcaccatctagtgccagtatcattaattctcagaatgaatatttaatacatttgttacaggaactagaaaataaagaaaaaaagtt
aaagaatttagaagattctcttgaagattacaacagaaaatttgctgtaattcgtcatcaacaaagtttgttgtataaagaataccta
agtgaaaaggagacctggaaaacagaatctaaaacaataaaagaggaaaagagaaaacttgaggatcaagtccaacaagatgc
tataaaagtaaaagaatataataatttgctcaatgctcttcagatggattcggatgaaatgaaaaaaatacttgcagaaaatagtag
gaaaattactgttttgcaagtgaatgaaaaatcacttataaggcaatatacaaccttagtagaattggagcgacaacttagaaaaga
aaatgagaagcaaaagaatgaattgttgtcaatggaggctgaagtttgtgaaaaaattgggtgtttgcaaagatttaaggaaatgg
ccattttcaagattgcagctctccaaaaagttgtagataatagtgtttctttgtctgaactagaactggctaataaacagtacaatgaa
ctgactgctaagtacagggacatcttgcaaaaagataatatgcttgttcaaagaacaagtaacttggaacacctggagtgtgaaaa
catctccttaaaagaacaagtggagtctataaataaagaactggagattaccaaggaaaaacttcacactattgaacaagcctggg
aacaggaaactaaattaggtaatgaatctagcatggataaggcaaagaaatcaataaccaacagtgacattgtttccatttcaaaa
aaaataactatgctggaaatgaaggaattaaatgaaaggcagcgggctgaacattgtcaaaaaatgtatgaacacttacggacttc
gttaaagcaaatggaggaacgtaattttgaattggaaaccaaatttgctgagcttaccaaaatcaatttggatgcacagaaggtgga
acagatgttaagagatgaattagctgatagtgtgagcaaggcagtaagtgatgctgataggcaacggattctagaattagagaaga
atgaaatggaactaaaagttgaagtgtcaaaactgagagagatttctgatattgccagaagacaagttgaaattttgaatgcacaa
caacaatctagggacaaggaagtagagtccctcagaatgcaactgctagactatcaggcacagtctgatgaaaagtcgctcattgc
caagttgcaccaacataatgtctctcttcaactgagtgaggctactgctcttggtaagttggagtcaattacatctaaactgcagaag
atggaggcctacaacttgcgcttagagcagaaacttgatgaaaaagaacaggctctctattatgctcgtttggagggaagaaacag
agcaaaacatctgcgccaaacaattcagtctctacgacgacagtttagtggagctttacccttggcacaacaggaaaagttctccaa
aacaatgattcaactacaaaatgacaaacttaagataatgcaagaaatgaaaaattctcaacaagaacatagaaatatggagaac
aaaacattggagatggaattaaaattaaagggcctggaagagttaataagcactttaaaggataccaaaggagcccaaaaggtaa
tcaactggcatatgaaaatagaagaacttcgtcttcaagaacttaaactaaatcgggaattagtcaaggataaagaagaaataaaa
tatttgaataacataatttctgaatatgaacgtacaatcagcagtcttgaagaagaaattgtgcaacagaacaagtttcatgaagaa
agacaaatggcctgggatcaaagagaagttgacctggaacgccaactagacatttttgaccgtcagcaaaatgaaatactaaatgc
ggcacaaaagtttgaagaagctacaggatcaatccctgaccctagtttgccccttccaaatcaacttgagatcgctctaaggaaaatt
aaggagaacattcgaataattctagaaacacgggcaact
N-intein RmaDnaB (seq Q)
3xflag (seq E)
WPRE (seq F)
BghPolyA (seq G)
3′ITR (seq H)
p1152 pAAV2.1-GRK1-5′ CEP290 intein (set 5)
5′ ITR (seq A)
GRK1 promoter (seq N)
5′ CEP290: SEQ. ID No. 57
atgccacctaatataaactggaaagaaataatgaaagttgacccagatgacctgccccgtcaagaagaactggcagataatttatt
gatttccttatccaaggtggaagtaaatgagctaaaaagtgaaaagcaagaaaatgtgatacaccttttcagaattactcagtcact
aatgaagatgaaagctcaagaagtggagctggctttggaagaagtagaaaaagctggagaagaacaagcaaaatttgaaaatca
attaaaaactaaagtaatgaaactggaaaatgaactggagatggctcagcagtctgcaggtggacgagatactcggtttttacgta
atgaaatttgccaacttgaaaaacaattagaacaaaaagatagagaattggaggacatggaaaaggagttggagaaagagaaga
aagttaatgagcaattggctcttcgaaatgaggaggcagaaaatgaaaacagcaaattaagaagagagaacaaacgtctaaaga
aaaagaatgaacaactttgtcaggatattattgactaccagaaacaaatagattcacagaaagaaacacttttatcaagaagaggg
gaagacagtgactaccgatcacagttgtctaaaaaaaactatgagcttatccaatatcttgatgaaattcagactttaacagaagct
aatgagaaaattgaagttcagaatcaagaaatgagaaaaaatttagaagagtctgtacaggaaatggagaagatgactgatgaat
ataatagaatgaaagctattgtgcatcagacagataatgtaatagatcagttaaaaaaagaaaacgatcattatcaacttcaagtg
caggagcttacagatctcctgaaatcaaaaaatgaagaagatgatccaattatggtagctgtcaatgcaaaagtagaagaatgga
agctaattttgtcttctaaagatgatgaaattattgagtatcagcaaatgttacataacctaagggagaaacttaagaatgctcagct
tgatgctgataaaagtaatgttatggctctacagcagggtatacaggaacgagacagtcaaattaagatgctcaccgaacaagtag
aacaatatacaaaagaaatggaaaagaatacttgtattattgaagatttgaaaaatgagctccaaagaaacaaaggtgcttcaacc
ctttctcaacagactcatatgaaaattcagtcaacgttagacattttaaaagagaaaactaaagaggctgagagaacagctgaact
ggctgaggctgatgctagggaaaaggataaagagttagttgaggctctgaagaggttaaaagattatgaa
N-intein mDnaE (seq S)
3xflag (seq E)
WPRE (seq F)
Bgh PolyA (seq G)
3′ ITR (seq H)
p1153 pAAV2.1-GRK1-CEP290 body intein (set 5)
5′ ITR (seq A)
GRK1 promoter (seq N)
C-intein mDnaE (seq T)
CEP290 body: SEQ. ID No. 58
tcgggagtatatggtttagaagatgctgtcgttgaaataaagaattgtaaaaaccaaattaaaataagagatcgagagattgaaat
attaacaaaggaaatcaataaacttgaattgaagatcagtgatttccttgatgaaaatgaggcacttagagagcgtgtgggccttga
accaaagacaatgattgatttaactgaatttagaaatagcaaacacttaaaacagcagcagtacagagctgaaaaccagattctttt
gaaagagattgaaagtctagaggaagaacgacttgatctgaaaaaaaaaattcgtcaaatggctcaagaaagaggaaaaagaag
tgcaacttcaggattaaccactgaggacctgaacctaactgaaaacatttctcaaggagatagaataagtgaaagaaaattggattt
attgagcctcaaaaatatgagtgaagcacaatcaaagaatgaatttctttcaagagaactaattgaaaaagaaagagatttagaaa
ggagtaggacagtgatagccaaatttcagaataaattaaaagaattagttgaagaaaataagcaacttgaagaaggtatgaaaga
aatattgcaagcaattaaggaaatgcagaaagatcctgatgttaaaggaggagaaacatctctaattatccctagccttgaaagact
agttaatgctatagaatcaaagaatgcagaaggaatctttgatgcgagtctgcatttgaaagcccaagttgatcagcttaccggaag
aaatgaagaattaagacaggagctcagggaatctcggaaagaggctataaattattcacagcagttggcaaaagctaatttaaag
atagaccatcttgaaaaagaaactagtcttttacgacaatcagaaggatcgaatgttgtttttaaaggaattgacttacctgatggga
tagcaccatctagtgccagtatcattaattctcagaatgaatatttaatacatttgttacaggaactagaaaataaagaaaaaaagtt
aaagaatttagaagattctcttgaagattacaacagaaaatttgctgtaattcgtcatcaacaaagtttgttgtataaagaataccta
agtgaaaaggagacctggaaaacagaatctaaaacaataaaagaggaaaagagaaaacttgaggatcaagtccaacaagatgc
tataaaagtaaaagaatataataatttgctcaatgctcttcagatggattcggatgaaatgaaaaaaatacttgcagaaaatagtag
gaaaattactgttttgcaagtgaatgaaaaatcacttataaggcaatatacaaccttagtagaattggagcgacaacttagaaaaga
aaatgagaagcaaaagaatgaattgttgtcaatggaggctgaagtttgtgaaaaaattgggtgtttgcaaagatttaaggaaatgg
ccattttcaagattgcagctctccaaaaagttgtagataatagtgtttctttgtctgaactagaactggctaataaacagtacaatgaa
ctgactgctaagtacagggacatcttgcaaaaagataatatgcttgttcaaagaacaagtaacttggaacacctggagtgtgaaaa
catctccttaaaagaacaagtggagtctataaataaagaactggagattaccaaggaaaaacttcacactattgaacaagcctggg
aacaggaaactaaattaggtaatgaatctagcatggataaggcaaagaaatcaataaccaacagtgacattgtttccatttcaaaa
aaaataactatgctggaaatgaaggaattaaatgaaaggcagcgggctgaacattgtcaaaaaatgtatgaacacttacggacttc
gttaaagcaaatggaggaacgtaattttgaattggaaaccaaatttgctgagcttaccaaaatcaatttggatgcacagaaggtgga
acagatgttaagagatgaattagctgatagtgtgagcaaggcagtaagtgatgctgataggcaacggattctagaattagagaaga
atgaaatggaactaaaagttgaagtgtcaaaactgagagagatttctgatattgccagaagacaagttgaaattttgaatgcacaa
caacaatctagggacaaggaagtagagtccctcagaatgcaactgctagactatcaggcacagtctgatgaaaagtcgctcattgc
caagttgcaccaacataatgtctctcttcaactgagtgaggctactgctcttggtaagttggagtcaattacatctaaactgcagaag
atggaggcctacaacttgcgcttagagcagaaacttgatgaaaaagaacaggctctctattatgctcgtttggagggaagaaacag
agcaaaacatctgcgccaaacaattcagtctctacgacgacagtttagtggagctttacccttggcacaacaggaaaagttctccaa
aacaatgattcaactacaaaatgacaaacttaagataatgcaagaaatgaaaaattctcaacaagaacatagaaatatggagaac
aaaacattggagatggaattaaaattaaagggcctggaagagttaataagcactttaaaggataccaaaggagcccaaaaggtaa
tcaactggcatatgaaaatagaagaacttcgtcttcaagaacttaaactaaatcgggaattagtcaaggataaagaagaaataaaa
tatttgaataacataatttctgaatatgaacgtacaatcagcagtcttgaagaagaaattgtgcaacagaacaagtttcatgaagaa
agacaaatggcctgggatcaaagagaagttgacctggaacgccaactagacatttttgaccgtcagcaaaatgaaatactaaatgc
ggcacaaaagtttgaagaagctacaggatcaatccctgaccctagtttgccccttccaaatcaacttgagatcgctctaaggaaaatt
aaggagaacattcgaataattctagaaacacgggcaact
N-intein RmaDnaB (seq Q)
3xflag (seq E)
WPRE (seq F)
Bgh PolyA (seq H)
p1156 pAAV2.1-GRK1-3′ CEP290 intein (set 5)
5′ ITR (seq A)
GRK1 promoter (seq N)
C-intein Rma DnaB (seq R)
3′ CEP290: SEQ. ID No. 59
tgcaaatcactagaagagaaactaaaagagaaagaatctgctttaaggttagcagaacaaaatatactgtcaagagacaaagtaa
tcaatgaactgaggcttcgattgcctgccactgcagaaagagaaaagctcatagctgagctaggcagaaaagagatggaaccaaa
atctcaccacacattgaaaattgctcatcaaaccattgcaaacatgcaagcaaggttaaatcaaaaagaagaagtattaaagaagt
atcaacgtcttctagaaaaagccagagaggagcaaagagaaattgtgaagaaacatgaggaagaccttcatattcttcatcacaga
ttagaactacaggctgatagttcactaaataaattcaaacaaacggcttgggatttaatgaaacagtctcccactccagttcctacca
acaagcattttattcgtctggctgagatggaacagacagtagcagaacaagatgactctctttcctcactcttggtcaaactaaagaa
agtatcacaagatttggagagacaaagagaaatcactgaattaaaagtaaaagaatttgaaaatatcaaattacagcttcaagaa
aaccatgaagatgaagtgaaaaaagtaaaagcggaagtagaggatttaaagtatcttctggaccagtcacaaaaggagtcacagt
gtttaaaatctgaacttcaggctcaaaaagaagcaaattcaagagctccaacaactacaatgagaaatctagtagaacggctaaag
agccaattagccttgaaggagaaacaacagaaagcacttagtcgggcacttttagaactccgggcagaaatgacagcagctgctg
aagaacgtattatttctgcaacttctcaaaaagaggcccatctcaatgttcaacaaatcgttgatcgacatactagagagctaaaga
cacaagttgaagatttaaatgaaaatcttttaaaattgaaagaagcacttaaaactagtaaaaacagagaaaactcactaactgat
aatttgaatgacttaaataatgaactgcaaaagaaacaaaaagcctataataaaatacttagagagaaagaggaaattgatcaag
agaatgatgaactgaaaaggcaaattaaaagactaaccagtggattacagggcaaacccctgacagataataaacaaagtctaat
tgaagaactccaaaggaaagttaaaaaactagagaaccaattagagggaaaggtggaggaagtagacctaaaacctatgaaag
aaaagaatgctaaagaagaattaattaggtgggaagaaggtaaaaagtggcaagccaaaatagaaggaattcgaaacaagttaa
aagagaaagagggggaagtctttactttaacaaagcagttgaatactttgaaggatctttttgccaaagccgataaagagaaactta
ctttgcagaggaaactaaaaacaactggcatgactgttgatcaggttttgggaatacgagctttggagtcagaaaaagaattggaa
gaattaaaaaagagaaatcttgacttagaaaatgatatattgtatatgagggcccaccaagctcttcctcgagattctgttgtagaag
atttacatttacaaaatagatacctccaagaaaaacttcatgctttagaaaaacagttttcaaaggatacatattctaagccttcaatt
tcaggaatagagtcagatgatcattgtcagagagaacaggagcttcagaaggaaaacttgaagttgtcatctgaaaatattgaact
gaaatttcagcttgaacaagcaaataaagatttgccaagattaaagaatcaagtcagagatttgaaggaaatgtgtgaatttcttaa
gaaagaaaaagcagaagttcagcggaaacttggccatgttagagggtctggtagaagtggaaagacaatcccagaactggaaaa
aaccattggtttaatgaaaaaagtagttgaaaaagtccagagagaaaatgaacagttgaaaaaagcatcaggaatattgactagt
gaaaaaatggctaatattgagcaggaaaatgaaaaattgaaggctgaattagaaaaacttaaagctcatcttgggcatcagttgag
catgcactatgaatccaagaccaaaggcacagaaaaaattattgctgaaaatgaaaggcttcgtaaagaacttaaaaaagaaact
gatgctgcagagaaattacggatagcaaagaataatttagagatattaaatgagaagatgacagttcaactagaagagactggta
agagattgcagtttgcagaaagcagaggtccacagcttgaaggtgctgacagtaagagctggaaatccattgtggttacaagaatg
tatgaaaccaagttaaaagaattggaaactgatattgccaaaaaaaatcaaagcattactgaccttaaacagcttgtaaaagaagc
aacagagagagaacaaaaagttaacaaatacaatgaagaccttgaacaacagattaagattcttaaacatgttcctgaaggtgctg
agacagagcaaggccttaaacgggagcttcaagttcttagattagctaatcatcagctggataaagagaaagcagaattaatccat
cagatagaagctaacaaggaccaaagtggagctgaaagcaccatacctgatgctgatcaactaaaggaaaaaataaaagatcta
gagacacagctcaaaatgtcagatctagaaaagcagcatttgaaggaggaaataaagaagctgaaaaaagaactggaaaatttt
gatccttcattttttgaagaaattgaagatcttaagtataattacaaggaagaagtgaagaagaatattctcttagaagagaaggta
aaaaaactttcagaacaattgggagttgaattaactagccctgttgctgcttctgaagagtttgaagatgaagaagaaagtcctgtta
atttccccatttac
3xflag (seq E)
WPRE (seq F)
Bgh PolyA (seq G)
3′ITR (seq H)
pzac-GRK1-5′ ABCA4 intein (set1) SEQ ID No. 60
5′ ITR (seq A)
GRK1: bold
5′ ABCA4: underline
N-intein Npu DnaE: double underline
3xflag: italic
SV40: bold underline
3′ ITR (seq H)
ctgcgcgctcgctcgctcactgaggccgcccgggcaaagcccgggcgtcgggcgacctttggtcgcccggcctcagtgagcgagcg
agcgcgcagagagggagtggccaactccatcactaggggttccttgtagttaatgattaacccgccatgctacttatctacgtagcca
tgctctaggaagatcttcaatattggccattagccatattattcattggttatatagcataaatcaatattggctattggccattgcata
cgttgtatctatatcataatatgtacatttatattggctcatgtccaatatgaccgccatgttggcattgattattgactagtgggcccca
gaagcctggtggttgtttgtccttctcaggggaaaagtgaggcggccccttggaggaaggggccgggcagaatgatctaatcgga
ttccaagcagctcaggggattgtctttttctagcaccttcttgccactcctaagcgtcctccgtgaccccggctgggatttagcctggt
gctgtgtcagccccgggctcccaggggcttcccagtggtccccaggaaccctcgacagggccagggcgtctctctcgtccagcaag
ggcagggacgggccacaggcaagggcgcggccgccatgggcttcgtgagacagatacagcttttgctctggaagaactggaccctg
cggaaaaaggcaaaagattcgctttgtggtggaactcgtgtggcctttatctttatttctggtcttgatctggttaaggaatgccaaccc
gctctacagccatcatgaatgccatttccccaacaaggcgatgccctcagcaggaatgctgccgtggctccaggggatcttctgcaat
gtgaacaatccctgttttcaaagccccaccccaggagaatctcctggaattgtgtcaaactataacaactccatcttggcaagggtat
atcgagattttcaagaactcctcatgaatgcaccagagagccagcaccttggccgtatttggacagagctacacatcttgtcccaatt
catggacaccctccggactcacccggagagaattgcaggaagaggaattcgaataagggatatcttgaaagatgaagaaacactg
acactatttctcattaaaaacatcggcctgtctgactcagtggtctaccttctgatcaactctcaagtccgtccagagcagttcgctcat
ggagtcccggacctggcgctgaaggacatcgcctgcagcgaggccctcctggagcgcttcatcatcttcagccagagacgcggggc
aaagacggtgcgctatgccctgtgctccctctcccagggcaccctacagtggatagaagacactctgtatgccaacgtggacttcttc
aagctcttccgtgtgcttcccacactcctagacagccgttctcaaggtatcaatctgagatcttggggaggaatattatctgatatgtc
accaagaattcaagagtttatccatcggccgagtatgcaggacttgctgtgggtgaccaggcccctcatgcagaatggtggtccaga
gacctttacaaagctgatgggcatcctgtctga cctcctgtgtggctaccccgagggaggtggctctcgggtgctctccttcaactggt
atgaagacaataactataaggcctttctggggattgactccacaaggaaggatcctatctattcttatgacagaagaacaacatcctt
ttgtaatgcattgatccagagcctggagtcaaatcctttaaccaaaatcgcttggagggcggcaaagcctttgctgatgggaaaaat
cctgtacactcctgattcacctgcagcacgaaggatactgaagaatgccaactcaacttttgaagaactggaacacgttaggaagtt
ggtcaaagcctgggaagaagtagggccccagatctggtacttctttgacaacagcacacagatgaacatgatcagagataccctgg
ggaacccaacagtaaaagactttttgaataggcagcttggtgaagaaggtattactgctgaagccatcctaaacttcctctacaagg
gccctcgggaaagccaggctgacgaatggccaacttcgactggagggacatatttaacatcactgatcgcaccctccgccttgtca
atcaatacctggagtgcttggtcctggataagtttgaaagctacaatgatgaaactcagctcacccaacgtgccctctctctactgga
ggaaaacatgttctgggccggagtggtattccctgacatgtatccctggaccagctctctaccaccccacgtgaagtataagatccga
atggacatagacgtggtggagaaaaccaataagattaaagacaggtattgggattctggtcccagagctgatcccgtggaagattt
ccggtacatctggggcgggtttgcctatctgcaggacatggttgaacaggggatcacaaggagccaggtgcaggcggaggctccag
ttggaatctacctccagcagatgccctacccctgcttcgtggacgattctttcatgatcatcctgaaccgctgtttccctatcttcatggt
gctggcatggatctactctgtctccatgactgtgaagagcatcgtcttggagaaggagttgcgactgaaggagaccttgaaaaatca
gggtgtctccaatgcagtgatttggtgtacctggttcctggacagcttctccatcatgtcgatgagcatcttcctcctgacgatattcatc
atgcatggaagaatcctacattacagcgacccattcatcctcttcctgttcttgttggctttctccactgccaccatcatgctgtgctttct
gctcagcaccttcttctccaaggccagtctggcagcagcctgtagtggtgtcatctatttcaccctctacctgccacacatcctgtgctt
cgcctggcaggaccgcatgaccgctgagctgaagaaggctgtgagcttactgtctccggtggcatttggatttggcactgagtacctg
gttcgctttgaagagcaaggcctggggctgcagtggagcaacatcgggaacagtcccacggaaggggacgaattcagcttcctgct
gtccatgcagatgatgctccttgatgctgctgtctatggcttactcgcttggtaccttgatcaggtgtttccaggagactatggaacccc
acttccttggtactttcttctacaagagtcgtattggcttggcggtgaagggtgttcaaccagagaagaaagagccctggaaaagac
cgagcccctaacagaggaaacggaggatccagagcacccagaaggaatacacgactccttctttgaacgtgagcatccagggtgg
gttcctggggtatgcgtgaagaatctggtaaagatttttgagccctgtggccggccagctgtggaccgtctgaacatcaccttctacg
agaaccagatcaccgcattcctgggccacaatggagctgggaaaaccaccaccttgtccatcctgacgggtctgttgccaccaacct
ctgggactgtgctcgttgggggaagggacattgaaaccagcctggatgcagtccggcagagccttggcatgtgtccacagcacaac
atcctgttccaccacctcacggtggctgagcacatgctgttctatgcccagctgaaaggaaagtcccaggaggaggcccagctggag
atggaagccatgttggaggacacaggcctccaccacaagcggaatgaagaggctcaggacctatcaggtggcatgcagagaaag
ctgtcggttgccattgcctttgtgggagatgccaaggtggtgattctggacgaacccacctctggggtggacccttactcgagacgctc
aatctgggatctgctcctgaagtatcgctcaggcagaaccatcatcatgtccactcaccacatggacgaggccgacctccttgggga
ccgcattgccatcattgcccagggaaggctctactgctcaggcaccccactcttcctgaagaactgcctgagctacgagaccgagat
cctgaccgtggagtacggcctgctgcccatcggcaagatcgtggagaagcggatcgagtgcaccgtgtacagcgtggacaacaacg
gcaacatctacacccagcccgtggcccagtggcacgaccggggcgagcaggaggtgttcgagtactgcctggaggacggcagcct
gatccgggccaccaaggaccacaagttcatgaccgtggacggccagatgctgcccatcgacgagatcttcgagcgggagctggacc
tgatgcgggtggacaacctgcccaacgactacaaagaccatgacggtgattataaagatcatgacatcgactacaaggatgac
gatgacaagtgagcggccgcttcgagcagacatgataagatacattgatgagtttggacaaaccacaactagaatgcagtgaaa
aaaatgctttatttgtgaaatttgtgatgctattgctttatttgtaaccattataagctgcaataaacaagttaacaacaacaattgc
attcattttatgtttcaggttcagggggagatgtgggaggttttttaaagcaagtaaaacctctacaaatgtggtaaaatcgataagg
atcttcctagagcatggctacgtagataagtagcatggcgggttaatcattaactacaaggaacccctagtgatggagttggccactc
cctctctgcgcgctcgctcgctcactgaggccgggcgaccaaaggtcgcccgacgcccgggctttgcccgggcggcctcagtgagcg
agcgagcgcgcag
pzac-GRK1-3′ ABCA4 intein (set1) SEQ ID No. 61
5′ ITR (seq A)
GRK1: bold
3′ ABCA4: underline
C-intein Npu DnaE: double underline
3xflag: italic
SV40: bold underline
3′ ITR (seq H)
ctgcgcgctcgctcgctcactgaggccgcccgggcaaagcccgggcgtcgggcgacctttggtcgcccggcctcagtgagcgagcg
agcgcgcagagagggagtggccaactccatcactaggggttccttgtagttaatgattaacccgccatgctacttatctacgtagcca
tgctctaggaagatcttcaatattggccattagccatattattcattggttatatagcataaatcaatattggctattggccattgcata
cgttgtatctatatcataatatgtacatttatattggctcatgtccaatatgaccgccatgttggcattgattattgactagtgggcccca
gaagcctggtggttgtttgtccttctcaggggaaaagtgaggcggccccttggaggaaggggccgggcagaatgatctaatcgga
ttccaagcagctcaggggattgtctttttctagcaccttcttgccactcctaagcgtcctccgtgaccccggctgggatttagcctggt
gctgtgtcagccccgggctcccaggggcttcccagtggtccccaggaaccctcgacagggccagggcgtctctctcgtccagcaag
ggcagggacgggccacaggcaagggcgcggccgcatgatcaagatcgccacccggaagtacctgggcaagcagaacgtgtacga
catcggcgtggagcgggaccacaacttcgccctgaaaacggcttcatcgccagcaattgctttggcacaggcttgtacttaaccttg
gtgcgcaagatgaaaaacatccagagccaaaggaaaggcagtgaggggacctgcagctgctcgtctaagggtttctccaccacgt
gtccagcccacgtcgatgacctaactccagaacaagtcctggatggggatgtaaatgagctgatggatgtagttctccaccatgttcc
agaggcaaagctggtggagtgcattggtcaagaacttatcttccttcttccaaataagaacttcaagcacagagcatatgccagcctt
ttcagagagctggaggagacgctggctgaccttggtctcagcagttttggaatttctgacactcccctggaagagatttttctgaaggt
cacggaggattctgattcaggacctctgtttgcgggtggcgctcagcagaaaagagaaaacgtcaacccccgacacccctgcttggg
tcccagagagaaggctggacagacaccccaggactccaatgtctgctccccaggggcgccggctgctcacccagagggccagcctc
ccccagagccagagtgcccaggcccgcagctcaacacggggacacagctggtcctccagcatgtgcaggcgctgctggtcaagag
attccaacacaccatccgcagccacaaacttcctggcgcagatcgtgctcccggctacctttgtgtttttggctctgatgctttctat
tgttatccctccttttggcgaataccccgctttgacccttcacccctggatatatgggcagcagtacaccttcttcagcatggatgaacc
aggcagtgagcagttcacggtacttgcagacgtcctcctgaataagccaggctttggcaccgctgcctgaaaagggtggcttcc
ggagtacccctgtggcaactcaacaccctggaaactccttctgtgtccccaaacatcacccagctgttccagaagcagaaatac
acaggtcaacccttcaccatcctgcaggtgcagcaccagggagaagctcaccatgctgccagagtgccccgagggtgccgggggcc
tcccgcccccccagagaacacagcgcagcacggaaattctacaagacctgacggacaggaacatctccgacttcttggtaaaaacg
tatcctgctcttataagaagcagcttaaagagcaaattctgggtcaatgaacagaggtatggaggaatttccattggaggaaagctc
ccagtcgtccccatcacgggggaagcacttgttgggtttttaagcgaccttggccggatcatgaatgtgagcgggggccctatcacta
gagaggcctctaaagaaatacctgatttccttaaacatctagaaactgaagacaacattaaggtgtggtttaataacaaaggctggc
atgccctggtcagctttctcaatgtggcccacaacgccatcttacgggccagcctgcctaaggacaggagccccgaggagtatggaa
tcaccgtcattagccaacccctgaacctgaccaaggagcagctctcagagattacagtgctgaccacttcagtggatgctgtggttgc
catctgcgtgattttctccatgtccttcgtcccagccagctttgtcctttatttgatccaggagcgggtgaacaaatccaagcacctcca
gtttatcagtggagtgagccccaccacctactgggtaaccaacttcctctgggacatcatgaattattccgtgagtgctgggctggtgg
tgggcatcttcatcgggtttcagaagaaagcctacacttctccagaaaaccttcctgcccttgtggcactgctcctgctgtatggatgg
gcggtcattcccatgatgtacccagcatccttcctgtttgatgtccccagcacagcctatgtggctttatcttgtgctaatctgttcatcg
gcatcaacagcagtgctattaccttcatcttggaattatttgagaataaccggacgctgctcaggttcaacgccgtgctgaggaagct
gctcattgtcttcccccacttctgcctgggccggggcctcattgaccttgcactgagccaggctgtgacagatgtctatgcccggtttgg
tgaggagcactctgcaaatccgttccactgggacctgattgggaagaacctgtttgccatggtggtggaaggggtggtgtacttcctc
ctgaccctgctggtccagcgccacttcttcctctcccaatggattgccgagcccactaaggagcccattgttgatgaagatgatgatgt
ggctgaagaaagacaaagaattattactggtggaaataaaactgacatcttaaggctacatgaactaaccaagatttatccaggca
cctccagcccagcagtggacaggctgtgtgtcggagttcgccctggagagtgctttggcctcctgggagtgaatggtgccggcaaaa
caaccacattcaagatgctcactggggacaccacagtgacctcaggggatgccaccgtagcaggcaagagtattttaaccaatattt
ctgaagtccatcaaaatatgggctactgtcctcagtttgatgcaatcgatgagctgctcacaggacgagaacatctttacctttatgcc
cggcttcgaggtgtaccagcagaagaaatcgaaaaggttgcaaactggagtattaagagcctgggcctgactgtctacgccgactg
cctggctggcacgtacagtgggggcaacaagcggaaactctccacagccatcgcactcattggctgcccaccgctggtgctgctgga
tgagcccaccacagggatggacccccaggcacgccgcatgctgtggaacgtcatcgtgagcatcatcagagaagggagggctgtg
gtcctcacatcccacagcatggaagaatgtgaggcactgtgtacccggctggccatcatggtaaagggcgcctttcgatgtatgggc
accattcagcatctcaagtccaaatttggagatggctatatcgtcacaatgaagatcaaatccccgaaggacgacctgcttcctgacc
tgaaccctgtggagcagttcttccaggggaacttcccaggcagtgtgcagagggagaggcactacaacatgctccagttccaggtct
cctcctcctccctggcgaggatcttccagctcctcctctcccacaaggacagcctgctcatcgaggagtactcagtcacacagaccac
actggaccaggtgtttgtaaattttgctaaacagcagactgaaagtcatgacctccctctgcaccctcgagctgctggagccagtcga
caagcccaggacgactacaaagaccatgacggtgattataaagatcatgacatcgactacaaggatgacgatgacaagtga
gcggccgcttcgagcagacatgataagatacattgatgagtttggacaaaccacaactagaatgcagtgaaaaaaatgctttatt
tgtgaaatttgtgatgctattgctttatttgtaaccattataagctgcaataaacaagttaacaacaacaattgcattcattttatgttt
caggttcagggggagatgtgggaggttttttaaagcaagtaaaacctctacaaatgtggtaaaatcgataaggatcttcctagagca
tggctacgtagataagtagcatggcgggttaatcattaactacaaggaacccctagtgatggagttggccactccctctctgcgcgct
cgctcgctcactgaggccgggcgaccaaaggtcgcccgacgcccgggctttgcccgggcggcctcagtgagcgagcgagcgcgca
g
pzac-CMV260-5′ ABCA4 intein (set1) SEQ ID No. 62
5′ ITR (seq A)
CMV260: bold
5′ ABCA4: underline
N-intein Npu DnaE: double underline
3xflag: italic
SV40: bold underline
3′ ITR (seq H)
ctgcgcgctcgctcgctcactgaggccgcccgggcaaagcccgggcgtcgggcgacctttggtcgcccggcctcagtgagcgagcg
agcgcgcagagagggagtggccaactccatcactaggggttccttgtagttaatgattaacccgccatgctacttatctacgtagcca
tgctctaggaagatcttcaatattggccattagccatattattcattggttatatagcataaatcaatattggctattggccattgcata
cgttgtatctatatcataatatgtacatttatattggctcatgtccaatatgaccgccatgttggcattgattattgactagcgttgacat
tgattattgactagtacggtaaatggcccgcctggctgatgactcacggggatttccaagtctccaccccattgacgtcaatgggag
tttgttttggcaccaaaatcaacgggactttccaaaatgtcgtaacaactccgccccattgacgcaaatgggcggtaggcgtgtac
ggtgggaggtctatataagcagagctggtttagtgaactagagaacccactgcttactggcttctcgagattccaccatggcggcc
gccatgggcttcgtgagacagatacagcttttgctctggaagaactggaccctgcggaaaaggcaaaagattcgctttgtggtggaa
ctcgtgtggcctttatctttatttctggtcttgatctggttaaggaatgccaacccgctctacagccatcatgaatgccatttccccaaca
aggcgatgccctcagcaggaatgctgccgtggctccaggggatcttctgcaatgtgaacaatccctgttttcaaagccccaccccag
gagaatctcctggaattgtgtcaaactataacaactccatcttggcaagggtatatcgagattttcaagaactcctcatgaatgcacc
agagagccagcaccttggccgtatttggacagagctacacatcttgtcccaattcatggacaccctccggactcacccggagagaat
tgcaggaagaggaattcgaataagggatatcttgaaagatgaagaaacactgacactatttctcattaaaaacatcggcctgtctga
ctcagtggtctaccttctgatcaactctcaagtccgtccagagcagttcgctcatggagtcccggacctggcgctgaaggacatcgcc
tgcagcgaggccctcctggagcgcttcatcatcttcagccagagacgcggggcaaagacggtgcgctatgccctgtgctccctctccc
agggcaccctacagtggatagaagacactctgtatgccaacgtggacttcttcaagctcttccgtgtgcttcccacactcctagacag
ccgttctcaaggtatcaatctgagatcttggggaggaatattatctgatatgtcaccaagaattcaagagtttatccatcggccgagta
tgcaggacttgctgtgggtgaccaggcccctcatgcagaatggtggtccagagacctttacaaagctgatgggcatcctgtctgacct
cctgtgtggctaccccgagggaggtggctctcgggtgctctccttcaactggtatgaagacaataactataaggcctttctggggatt
gactccacaaggaaggatcctatctattcttatgacagaagaacaacatccttttgtaatgcattgatccagagcctggagtcaaatc
ctttaaccaaaatcgcttggagggcggcaaagcctttgctgatgggaaaaatcctgtacactcctgattcacctgcagcacgaagga
tactgaagaatgccaactcaacttttgaagaactggaacacgttaggaagttggtcaaagcctgggaagaagtagggccccagatc
tggtacttctttgacaacagcacacagatgaacatgatcagagataccctggggaacccaacagtaaaagactttttgaataggca
gcttggtgaagaaggtattactgctgaagccatcctaaacttcctctacaagggccctcgggaaagccaggctgacgacatggccaa
cttcgactggagggacatatttaacatcactgatcgcaccctccgccttgtcaatcaatacctggagtgcttggtcctggataagtttg
aaagctacaatgatgaaactcagctcacccaacgtgccctctctctactggaggaaaacatgttctgggccggagtggtattccctga
catgtatccctggaccagctctctaccaccccacgtgaagtataagatccgaatggacatagacgtggtggagaaaaccaataaga
ttaaagacaggtattgggattctggtcccagagctgatcccgtggaagatttccggtacatctggggcgggtttgcctatctgcagga
catggttgaacaggggatcacaaggagccaggtgcaggcggaggctccagttggaatctacctccagcagatgccctacccctgctt
cgtggacgattctttcatgatcatcctgaaccgctgtttccctatcttcatggtgctggcatggatctactctgtctccatgactgtgaag
agcatcgtcttggagaaggagttgcgactgaaggagaccttgaaaaatcagggtgtctccaatgcagtgatttggtgtacctggttcc
tggacagcttctccatcatgtcgatgagcatcttcctcctgacgatattcatcatgcatggaagaatcctacattacagcgacccattc
atcctcttcctgttcttgttggctttctccactgccaccatcatgctgtgctttctgctcagcaccttcttctccaaggccagtctggcagc
agcctgtagtggtgtcatctatttcaccctctacctgccacacatcctgtgcttcgcctggcaggaccgcatgaccgctgagctgaaga
aggctgtgagcttactgtctccggtggcatttggatttggcactgagtacctggttcgctttgaagagcaaggcctggggctgcagtgg
agcaacatcgggaacagtcccacggaaggggacgaattcagcttcctgctgtccatgcagatgatgctccttgatgctgctgtctatg
gcttactcgcttggtaccttgatcaggtgtttccaggagactatggaaccccacttccttggtactttcttctacaagagtcgtattggct
tggcggtgaagggtgttcaaccagagaagaaagagccctggaaaagaccgagcccctaacagaggaaacggaggatccagagc
acccagaaggaatacacgactccttctttgaacgtgagcatccagggtgggttcctggggtatgcgtgaagaatctggtaaagatttt
tgagccctgtggccggccagctgtggaccgtctgaacatcaccttctacgagaaccagatcaccgcattcctgggccacaatggagc
tgggaaaaccaccaccttgtccatcctgacgggtctgttgccaccaacctctgggactgtgctcgttgggggaagggacattgaaac
cagcctggatgcagtccggcagagccttggcatgtgtccacagcacaacatcctgttccaccacctcacggtggctgagcacatgct
gttctatgcccagctgaaaggaaagtcccaggaggaggcccagctggagatggaagccatgttggaggacacaggcctccaccac
aagcggaatgaagaggctcaggacctatcaggtggcatgcagagaaagctgtcggttgccattgcctttgtgggagatgccaaggt
ggtgattctggacgaacccacctctggggtggacccttactcgagacgctcaatctgggatctgctcctgaagtatcgctcaggcaga
accatcatcatgtccactcaccacatggacgaggccgacctccttggggaccgcattgccatcattgcccagggaaggctctactgct
caggcaccccactcttcctgaagaactgcctgagctacgagaccgagatcctgaccgtggagtacggcctgctgcccatcggcaag
atcgtggagaagcggatcgagtgcaccgtgtacagcgtggacaacaacggcaacatctacacccagcccgtggcccagtggcacg
accggggcgagcaggaggtgttcgagtactgcctggaggacggcagcctgatccgggccaccaaggaccacaagttcatgacgt
ggacggccagatgctgcccatcgacgagatcttcgagcgggagctggacctgatgcgggtggacaacctgcccaacgactacaaa
gaccatgacggtgattataaagatcatgacatcgactacaaggatgacgatgacaagtgagcggccgcttcgagcagacatg
ataagatacattgatgagtttggacaaaccacaactagaatgcagtgaaaaaaatgctttatttgtgaatttgtgatgctattgct
ttatttgtaaccattataagctgcaataaacaagttaacaacaacaattgcattcattttatgtttcaggttcagggggagatgtggg
aggttttttaaagcaagtaaaacctctacaaatgtggtaaaatcgataaggatcttcctagagcatggctacgtagataagtagcat
ggcgggttaatcattaactacaaggaacccctagtgatggagttggccactccctctctgcgcgctcgctcgctcactgaggccgggc
gaccaaaggtcgcccgacgcccgggctttgcccgggcggcctcagtgagcgagcgagcgcgcag
pzac-CMV260-3′ ABCA4 intein (set1) SEQ ID No. 63
5′ ITR (seq A)
CMV260: bold
3′ ABCA4: underline
C-intein Npu DnaE: double underline
3xflag: italic
SV40: bold underline
3′ TR (seq H)
ctgcgcgctcgctcgctcactgaggccgcccgggcaaagcccgggcgtcgggcgacctttggtcgcccggcctcagtgagcgagcg
agcgcgcagagagggagtggccaactccatcactaggggttccttgtagttaatgattaacccgccatgctacttatctacgtagcca
tgctctaggaagatcttcaatattggccattagccatattattcattggttatatagcataaatcaatattggctattggccattgcata
cgttgtatctatatcataatatgtacatttatattggctcatgtccaatatgaccgccatgttggcattgattattgactagcgttgacat
tgattattgactagtacggtaaatggcccgcctggctgatgactcacggggatttccaagtctccaccccattgacgtcaatgggag
tttgttttggcaccaaaatcaacgggactttccaaaatgtcgtaacaactccgccccattgacgcaaatgggcggtaggcgtgtac
ggtgggaggtctatataagcagagctggtttagtgaactagagaacccactgcttactggcttctcgagattccaccatggcggcc
gccatgatcaagatcgccacccggaagtacctgggcaagcagaacgtgtacgacatcggcgtggagcgggaccacaacttcgccct
gaagaacggcttcatcgccagcaattgctttggcacaggcttgtacttaaccttggtgcgcaagatgaaaaacatccagagccaaag
gaaaggcagtgaggggacctgcagctgctcgtctaagggtttctccaccacgtgtccagcccacgtcgatgacctaactccagaaca
agtcctggatggggatgtaaatgagctgatggatgtagttctccaccatgttccagaggcaaagctggtggagtgcattggtcaaga
acttatcttccttcttccaaataagaacttcaagcacagagcatatgccagccttttcagagagctggaggagacgctggctgacctt
ggtctcagcagttttggaatttctgacactcccctggaagagatttttctgaaggtcacggaggattctgattcaggacctctgtttgcg
ggtggcgctcagcagaaaagagaaaacgtcaacccccgacacccctgcttgggtcccagagagaaggctggacagacaccccag
gactccaatgtctgctccccaggggcgccggctgctcacccagagggccagcctcccccagagccagagtgcccaggcccgcagct
caacacggggacacagctggtcctccagcatgtgcaggcgctgctggtcaagagattccaacacaccatccgcagccacaaggact
tcctggcgcagatcgtgctcccggctacctttgtgtttttggctctgatgctttctattgttatccctccttttggcgaataccccgctttga
cccttcacccctggatatatgggcagcagtacaccttcttcagcatggatgaaccaggcagtgagcagttcacggtacttgcagacgt
cctcctgaataagccaggctttggcaaccgctgcctgaaggaagggtggcttccggagtacccctgtggcaactcaacaccctggaa
gactccttctgtgtccccaaacatcacccagctgttccagaagcagaaatggacacaggtcaacccttcaccatcctgcaggtgcag
caccagggagaagctcaccatgctgccagagtgccccgagggtgccgggggcctcccgcccccccagagaacacagcgcagcacg
gaaattctacaagacctgacggacaggaacatctccgacttcttggtaaaaacgtatcctgctcttataagaagcagcttaaagagc
aaattctgggtcaatgaacagaggtatggaggaatttccattggaggaaagctcccagtcgtccccatcacgggggaagcacttgtt
gggtttttaagcgaccttggccggatcatgaatgtgagcgggggccctatcactagagaggcctctaaagaaatacctgatttcctta
aacatctagaaactgaagacaacattaaggtgtggtttaataacaaaggctggcatgccctggtcagctttctcaatgtggcccaca
acgccatcttacgggccagcctgcctaaggacaggagccccgaggagtatggaatcaccgtcattagccaacccctgaacctgacc
aaggagcagctctcagagattacagtgctgaccacttcagtggatgctgtggttgccatctgcgtgattttctccatgtccttcgtccca
gccagctttgtcctttatttgatccaggagcgggtgaacaaatccaagcacctccagtttatcagtggagtgagccccaccacctact
gggtaaccaacttcctctgggacatcatgaattattccgtgagtgctgggctggtggtgggcatcttcatcgggtttcagaagaaagc
ctacacttctccagaaaaccttcctgcccttgtggcactgctcctgctgtatggatgggcggtcattcccatgatgtacccagcatcctt
cctgtttgatgtccccagcacagcctatgtggctttatcttgtgctaatctgttcatcggcatcaacagcagtgctattaccttcatcttg
gaattatttgagaataaccggacgctgctcaggttcaacgccgtgctgaggaagctgctcattgtcttcccccacttctgcctgggccg
gggcctcattgaccttgcactgagccaggctgtgacagatgtctatgcccggtttggtgaggagcactctgcaaatccgttccactgg
gacctgattgggaagaacctgtttgccatggtggtggaaggggtggtgtacttcctcctgaccctgctggtccagcgccacttcttcct
ctcccaatggattgccgagcccactaaggagcccattgttgatgaagatgatgatgtggctgaagaaagacaaagaattattactgg
tggaaataaaactgacatcttaaggctacatgaactaaccaagatttatccaggcacctccagcccagcagtggacaggctgtgtgt
cggagttcgccctggagagtgctttggcctcctgggagtgaatggtgccggcaaaacaaccacattcaagatgctcactggggacac
cacagtgacctcaggggatgccaccgtagcaggcaagagtattttaaccaatatttctgaagtccatcaaaatatgggctactgtcct
cagtttgatgcaatcgatgagctgctcacaggacgagaacatctttacctttatgcccggcttcgaggtgtaccagcagaagaaatcg
aaaaggttgcaaactggagtattaagagcctgggcctgactgtctacgccgactgcctggctggcacgtacagtgggggcaacaag
cggaaactctccacagccatcgcactcattggctgcccaccgctggtgctgctggatgagcccaccacagggatggacccccaggca
cgccgcatgctgtggaacgtcatcgtgagcatcatcagagaagggagggctgtggtcctcacatcccacagcatggaagaatgtga
ggcactgtgtacccggctggccatcatggtaaagggcgcctttcgatgtatgggcaccattcagcatctcaagtccaaatttggagat
ggctatatcgtcacaatgaagatcaaatccccgaaggacgacctgcttcctgacctgaaccctgtggagcagttcttccaggggaac
ttcccaggcagtgtgcagagggagaggcactacaacatgctccagttccaggtctcctcctcctccctggcgaggatcttccagctcc
tcctctcccacaaacagcctgctcatcgaggagtactcagtcacacagaccacactaccaggtgtttgtaaattttgctaaaca
gcagactgaaagtcatgacctccctctgcaccctcgagctgctggagccagtcgacaagcccaggacgactacaaagaccatgac
ggtgattataaagatcatgacatcgactacaaggatgacgatgacaagtgagcggccgcttcgagcagacatgataagatac
attgatgagtttggacaaaccacaactagaatgcagtgaaaaaaatgctttatttgtgaaatttgtgatgctattgctttatttgtaa
ccattataagctgcaataaacaagttaacaacaacaattgcattcattttatgtttcaggttcagggggagatgtgggaggtttttta
aagcaagtaaaacctctacaaatgtggtaaaatcgataaggatcttcctagagcatggctacgtagataagtagcatggcgggtta
atcattaactacaaggaacccctagtgatggagttggccactccctctctgcgcgctcgctcgctcactgaggccgggcgaccaaag
gtcgcccgacgcccgggctttgcccgggcggcctcagtgagcgagcgagcgcgcag
p38 pAAV2.1-CMV260-5′ ABCA4 intein_ecDHFR (set1)
5′ ITR (seq A)
CMV260 (seq U)
5′ ABCA4 (from set 1)
N-intein Npu DnaE (seq D)
3xflag (seq E)
ecDHFR (seq O)
WPRE (seq F)
SV40 PolyA (seq W)
3′ ITR (seq H)
p39 pAAV2.1-CMV260-5′ ABCA4 intein_mini ecDHFR (set1)
5′ ITR (seq A)
CMV260 (seq U)
5′ ABCA4 (from set 1)
N-intein Npu DnaE (seq D)
3xflag (seq E)
mini ecDHFR (seq P)
WPRE (seq F)
SV40 PolyA (seq W)
3′ TR (seq H)
p40 pAAV2.1-GRK1-5′ ABCA4 intein_ecDHFR (set1)
5′ ITR (seq A)
GRK1 (seq N)
5′ ABCA4 (from set 1)
N-intein pu DnaE (seq D)
3xflag (seq E)
ecDHFR (seq O)
WPRE (seq F)
SV40 PolyA (seq W)
3′ ITR (seq H)
p41 pAAV2.1-GRK1-5′ ABCA4 intein_mini ecDHFR (set1) SEQ ID No. 64
5′ ITR (seq A)
GRK1: bold
5′ ABCA4: underline
N-intein Npu DnaE: double underline
3xf1ag: italic
Mini ecDHFR: thick underline
SV40: bold underline
3′ ITR (seq H)
ctgcgcgctcgctcgctcactgaggccgcccgggcaaagcccgggcgtcgggcgacctttggtcgcccggcctcagtgagcgagcg
agcgcgcagagagggagtggccaactccatcactaggggttcctgctagcctagtgggccccagaagcctggtggttgtttgtcctt
ctcaggggaaaagtgaggcggccccttggaggaaggggccgggcagaatgatctaatcggattccaagcagctcaggggattgt
ctttttctagcaccttcttgccactcctaagcgtcctccgtgaccccggctgggatttagcctggtgctgtgtcagccccgggctccca
ggggcttcccagtggtccccaggaaccctcgacagggccagggcgtctctctcgtccagcaagggcagggacgggccacaggcaa
gggcggccgccatgggcttcgtgagacagatacagcttttgctctggaagaactggaccctgcggaaaaggcaaaagattcgctttg
tggtggaactcgtgtggcctttatctttatttctggtcttgatctggttaaggaatgccaacccgctctacagccatcatgaatgccattt
ccccaacaaggcgatgccctcagcaggaatgctgccgtggctccaggggatcttctgcaatgtgaacaatccctgttttcaaagcccc
accccaggagaatctcctggaattgtgtcaaactataacaactccatcttggcaagggtatatcgagattttcaagaactcctcatga
atgcaccagagagccagcaccttggccgtatttggacagagctacacatcttgtcccaattcatggacaccctccggactcacccgg
agagaattgcaggaagaggaattcgaataagggatatcttgaaagatgaagaaacactgacactatttctcattaaaaacatcggc
ctgtctgactcagtggtctaccttctgatcaactctcaagtccgtccagagcagttcgctcatggagtcccggacctggcgctgaagga
catcgcctgcagcgaggccctcctggagcgcttcatcatcttcagccagagacgcggggcaaagacggtgcgctatgccctgtgctc
cctctcccagggcaccctacagtggatagaagacactctgtatgccaacgtggacttcttcaagctcttccgtgtgcttcccacactcc
tagacagccgttctcaaggtatcaatctgagatcttggggaggaatattatctgatatgtcaccaagaattcaagagtttatccatcg
gccgagtatgcaggacttgctgtgggtgaccaggcccctcatgcagaatggtggtccagagacctttacaaagctgatgggcatcct
gtctgacctcctgtgtggctaccccgagggaggtggctctcgggtgctctccttcaactggtatgaagacaataactataaggcctttc
tggggattgactccacaaggaaggatcctatctattcttatgacagaagaacaacatccttttgtaatgcattgatccagagcctgga
gtcaaatcctttaaccaaaatcgcttggagggcggcaaagcctttgctgatgggaaaaatcctgtacactcctgattcacctgcagca
cgaaggatactgaagaatgccaactcaacttttgaagaactggaacacgttaggaagttggtcaaagcctgggaagaagtagggc
cccagatctggtacttctttgacaacagcacacagatgaacatgatcagagataccctggggaacccaacagtaaaagactttttga
ataggcagcttggtgaagaaggtattactgctgaagccatcctaaacttcctctacaagggccctcgggaaagccaggctgacgac
atggccaacttcgactggagggacatatttaacatcactgatcgcaccctccgccttgtcaatcaatacctggagtgcttggtcctgga
taagtttgaaagctacaatgatgaaactcagctcacccaacgtgccctctctctactggaggaaaacatgttctgggccggagtggt
attccctgacatgtatccctggaccagctctctaccaccccacgtgaagtataagatccgaatggacatagacgtggtggagaaaac
caataagattaaagacaggtattgggattctggtcccagagctgatcccgtggaagatttccggtacatctggggcgggtttgcctat
ctgcaggacatggttgaacaggggatcacaaggagccaggtgcaggcggaggctccagttggaatctacctccagcagatgcccta
cccctgcttcgtggacgattctttcatgatcatcctgaaccgctgtttccctatcttcatggtgctggcatggatctactctgtctccatga
ctgtgaagagcatcgtcttggagaaggagttgcgactgaaggagaccttgaaaaatcagggtgtctccaatgcagtgatttggtgta
cctggttcctggacagcttctccatcatgtcgatgagcatcttcctcctgacgatattcatcatgcatggaagaatcctacattacagcg
acccattcatcctcttcctgttcttgttggctttctccactgccaccatcatgctgtgctttctgctcagcaccttcttctccaaggccagtc
tggcagcagcctgtagtggtgtcatctatttcaccctctacctgccacacatcctgtgcttcgcctggcaggaccgcatgaccgctgag
ctgaagaaggctgtgagcttactgtctccggtggcatttggatttggcactgagtacctggttcgctttgaagagcaaggcctggggc
tgcagtggagcaacatcgggaacagtcccacggaaggggacgaattcagcttcctgctgtccatgcagatgatgctccttgatgctg
ctgtctatggcttactcgcttggtaccttgatcaggtgtttccaggagactatggaaccccacttccttggtactttcttctacaagagtc
gtattggcttggcggtgaagggtgttcaaccagagaagaaagagccctggaaaagaccgagcccctaacagaggaaacggagga
tccagagcacccagaaggaatacacgactccttctttgaacgtgagcatccagggtgggttcctggggtatgcgtgaagaatctggt
aaagatttttgagccctgtggccggccagctgtggaccgtctgaacatcaccttctacgagaaccagatcaccgcattcctgggccac
aatggagctgggaaaaccaccaccttgtccatcctgacgggtctgttgccaccaacctctgggactgtgctcgttgggggaagggac
attgaaaccagcctggatgcagtccggcagagccttggcatgtgtccacagcacaacatcctgttccaccacctcacggtggctgag
cacatgctgttctatgcccagctgaaaggaaagtcccaggaggaggcccagctggagatggaagccatgttggaggacacaggcct
ccaccacaagcggaatgaagaggctcaggacctatcaggtggcatgcagagaaagctgtcggttgccattgcctttgtgggagatg
ccaaggtggtgattctggacgaacccacctctggggtggacccttactcgagacgctcaatctgggatctgctcctgaagtatcgctc
aggcagaaccatcatcatgtccactcaccacatggacgaggccgacctccttggggaccgcattgccatcattgcccagggaaggct
ctactgctcaggcaccccactcttcctgaagaactgcctgagctacgagaccgagatcctgaccgtggagtacggcctgctgcccatc
ggcaagatcgtggagaagcggatcgagtgcaccgtgtacagcgtggacaacaacggcaacatctacacccagcccgtggcccagt
ggcacgaccggggcgagcaggaggtgttcgagtactgcctggaggacggcagcctgatccgggccaccaaggaccacaagttcat
gaccgtggacggccagatgctgcccatcgacgagatcttcgagcgggagctggacctgatgcgggtggacaacctgcccaacgact
acaaagaccatgacggtgattataaagatcatgacatcgactacaaggatgacgatgacaagatcagcctgatcgccgccctg
gccgtggactacgtgatcggcatggagaacgccatgccctggaacctgcccgccgacctggcctggttcaagaggaacaccctgaa
caagcccgtgatcatgggcaggcacacctgggagagcatcggcaggcccctgcccggcaggaagaacatcatcctgagcagccag
cccagcaccgacgacagggtgacctgggtgaagagcgtggacgaggccatcgccgcctgcggcgacgtgcccgagatcatggtga
tcggcggcggcagggtgatcgagcagttcctgccctgattcgagcagacatgataagatacattgatgagtttggacaaaccacaa
ctagaatgcagtgaaaaaaatgctttatttgtgaaatttgtgatgctattgctttatttgtaaccattataagctgcaataaacaagt
taacaacaacaattgcattcattttatgtttcaggttcagggggagatgtgggaggttttttaaagcaagtaaaacctctacaaatg
tggtaaaatcgataaggatccaattgaggaacccctagtgatggagttggccactccctctctgcgcgctcgctcgctcactgaggc
cgggcgaccaaaggtcgcccgacgcccgggctttgcccgggcggcctcagtgagcgagcgagcgcgcag
pzac-CMV260-5′ ABCA4 intein (set2) SEQ ID No. 65
5′ ITR (seq A)
CMV260: bold
5′ ABCA4: underline
N-intein Npu DnaE: double underline
3xflag: italic
SV40: bold underline
3′ ITR (seq H)
ctgcgcgctcgctcgctcactgaggccgcccgggcaaagcccgggcgtcgggcgacctttggtcgcccggcctcagtgagcgagcg
agcgcgcagagagggagtggccaactccatcactaggggttccttgtagttaatgattaacccgccatgctacttatctacgtagcca
tgctctaggaagatcttcaatattggccattagccatattattcattggttatatagcataaatcaatattggctattggccattgcata
cgttgtatctatatcataatatgtacatttatattggctcatgtccaatatgaccgccatgttggcattgattattgactagcgttgacat
tgattattgactagtacggtaaatggcccgcctggctgatgactcacggggatttccaagtctccaccccattgacgtcaatgggag
tttgattggcaccaaaatcaacgggactuccaaaatgtcgtaacaactccgccccattgacgcaaatgggcggtaggcgtgtac
ggtgggaggtctatataagcagagctggtttagtgaactagagaacccactgcttactggcttctcgagattccaccatggcggcc
gccatgggcttcgtgagacagatacagcttttgctctggaagaactggaccctgcggaaaaggcaaaagattcgctttgtggtggaa
ctcgtgtggcctttatctttatttctggtcttgatctggttaaggaatgccaacccgctctacagccatcatgaatgccatttccccaaca
aggcgatgccctcagcaggaatgctgccgtggctccaggggatcttctgcaatgtgaacaatccctgttttcaaagccccaccccag
gagaatctcctggaattgtgtcaaactataacaactccatcttggcaagggtatatcgagattttcaagaactcctcatgaatgcacc
agagagccagcaccttggccgtatttggacagagctacacatcttgtcccaattcatggacaccctccggactcacccggagagaat
tgcaggaagaggaattcgaataagggatatcttgaaagatgaagaaacactgacactatttctcattaaaaacatcggcctgtctga
ctcagtggtctaccttctgatcaactctcaagtccgtccagagcagttcgctcatggagtcccggacctggcgctgaaggacatcgcc
tgcagcgaggccctcctggagcgcttcatcatcttcagccagagacgcggggcaaagacggtgcgctatgccctgtgctccctctccc
agggcaccctacagtggatagaagacactctgtatgccaacgtggacttcttcaagctcttccgtgtgcttcccacactcctagacag
ccgttctcaaggtatcaatctgagatcttggggaggaatattatctgatatgtcaccaagaattcaagagtttatccatcggccgagta
tgcaggacttgctgtgggtgaccaggcccctcatgcagaatggtggtccagagacctttacaaagctgatgggcatcctgtctgacct
cctgtgtggctaccccgagggaggtggctctcgggtgctctccttcaactggtatgaagacaataactataaggcctttctggggatt
gactccacaaggaaggatcctatctattcttatgacagaagaacaacatccttttgtaatgcattgatccagagcctggagtcaaatc
ctttaaccaaaatcgcttggagggcggcaaagcctttgctgatgggaaaaatcctgtacactcctgattcacctgcagcacgaagga
tactgaagaatgccaactcaacttttgaagaactggaacacgttaggaagttggtcaaagcctgggaagaagtagggccccagatc
tggtacttctttgacaacagcacacagatgaacatgatcagagataccctggggaacccaacagtaaaagactttttgaataggca
gcttggtgaagaaggtattactgctgaagccatcctaaacttcctctacaagggccctcgggaaagccaggctgacgacatggccaa
cttcgactggagggacatatttaacatcactgatcgcaccctccgccttgtcaatcaatacctggagtgcttggtcctggataagtttg
aaagctacaatgatgaaactcagctcacccaacgtgccctctctctactggaggaaaacatgttctgggccggagtggtattccctga
catgtatccctggaccagctctctaccaccccacgtgaagtataagatccgaatggacatagacgtggtggagaaaaccaataaga
ttaaagacaggtattgggattctggtcccagagctgatcccgtggaagatttccggtacatctggggcgggtttgcctatctgcagga
catggttgaacaggggatcacaaggagccaggtgcaggcggaggctccagttggaatctacctccagcagatgccctacccctgctt
cgtggacgattctttcatgatcatcctgaaccgctgtttccctatcttcatggtgctggcatggatctactctgtctccatgactgtgaag
agcatcgtcttggagaaggagttgcgactgaaggagaccttgaaaaatcagggtgtctccaatgcagtgatttggtgtacctggttcc
tggacagcttctccatcatgtcgatgagcatcttcctcctgacgatattcatcatgcatggaagaatcctacattacagcgacccattc
atcctcttcctgttcttgttggctttctccactgccaccatcatgctgtgctttctgctcagcaccttcttctccaaggccagtctggcagc
agcctgtagtggtgtcatctatttcaccctctacctgccacacatcctgtgcttcgcctggcaggaccgcatgaccgctgagctgaaga
aggctgtgagcttactgtctccggtggcatttggatttggcactgagtacctggttcgctttgaagagcaaggcctggggctgcagtgg
agcaacatcgggaacagtcccacggaaggggacgaattcagcttcctgctgtccatgcagatgatgctccttgatgctgctgtctatg
gcttactcgcttggtaccttgatcaggtgtttccaggagactatggaaccccacttccttggtactttcttctacaagagtcgtattggct
tggcggtgaagggtgttcaaccagagaagaaagagccctggaaaagaccgagcccctaacagaggaaacggaggatccagagc
acccagaaggaatacacgactccttctttgaacgtgagcatccagggtgggttcctggggtatgcgtgaagaatctggtaaagatttt
tgagccctgtggccggccagctgtggaccgtctgaacatcaccttctacgagaaccagatcaccgcattcctgggccacaatggagc
tgggaaaaccaccaccttgtccatcctgacgggtctgttgccaccaacctctgggactgtgctcgttgggggaagggacattgaaac
cagcctggatgcagtccggcagagccttggcatgtgtccacagcacaacatcctgttccaccacctcacggtggctgagcacatgct
gttctatgcccagctgaaaggaaagtcccaggaggaggcccagctggagatggaagccatgttggaggacacaggcctccaccac
aagcggaatgaagaggctcaggacctatcaggtggcatgcagagaaagctgtcggttgccattgcctttgtgggagatgccaaggt
ggtgattctggacgaacccacctctggggtggacccttactcgagacgctcaatctgggatctgctcctgaagtatcgctcaggcaga
accatcatcatgtccactcaccacatggacgaggccgacctccttggggaccgcattgccatcattgcccagggaaggctctactgct
caggcaccccactcttcctgaagaactgctttggcacaggcttgtacttaaccttggtgcgcaagatgaaaaacatccagtgcctgag
ctacgagaccgagatcctgaccgtggagtacggcctgctgcccatcggcaagatcgtggagaagcggatcgagtgcaccgtgtaca
gcgtggacaacaacggcaacatctacacccagcccgtggcccagtggcacgaccggggcgagcaggaggtgttcgagtactgcct
ggaggacggcagcctgatccgggccaccaaggaccacaagttcatgaccgtggacggccagatgctgcccatcgacgagatcttcg
agcgggagctggacctgatgcgggtggacaacctgcccaacgactacaaagaccatgacggtgattataaagatcatgacatc
gactacaaggatgacgatgacaagtgagcggccgcttcgagcagacatgataagatacattgatgagtttggacaaaccacaa
ctagaatgcagtgaaaaaaatgctttatttgtgaaatttgtgatgctattgctttatttgtaaccattataagctgcaataaacaagt
taacaacaacaattgcattcattttatgtttcaggttcagggggagatgtgggaggttttttaaagcaagtaaaacctctacaaatgt
ggtaaaatcgataaggatcttcctagagcatggctacgtagataagtagcatggcgggttaatcattaactacaaggaacccctagt
gatggagttggccactccctctctgcgcgctcgctcgctcactgaggccgggcgaccaaaggtcgcccgacgcccgggctttgcccg
ggcggcctcagtgagcgagcgagcgcgcag
pzac-CMV260-3′ ABCA4 intein (set2) SEQ ID No. 66
5′ ITR (seq A)
CMV260: bold
3′ ABCA4: underline
C-intein Npu DnaE: double underline
3xflag: italic
SV40: bold underline
3′ ITR (seq H)
ctgcgcgctcgctcgctcactgaggccgcccgggcaaagcccgggcgtcgggcgacctttggtcgcccggcctcagtgagcgagcg
agcgcgcagagagggagtggccaactccatcactaggggttccttgtagttaatgattaacccgccatgctacttatctacgtagcca
tgctctaggaagatcttcaatattggccattagccatattattcattggttatatagcataaatcaatattggctattggccattgcata
cgttgtatctatatcataatatgtacatttatattggctcatgtccaatatgaccgccatgttggcattgattattgactagcgttgacat
tgattattgactagtacggtaaatggcccgcctggctgatgactcacggggatttccaagtctccaccccattgacgtcaatgggag
tttgttttggcaccaaaatcaacgggactttccaaaatgtcgtaacaactccgccccattgacgcaaatgggcggtaggcgtgtac
ggtgggaggtctatataagcagagctggtttagtgaactagagaacccactgcttactggcttctcgagattccaccatggcggcc
gccatgatcaagatcgccacccggaagtacctgggcaagcagaacgtgtacgacatcggcgtggagcgggaccacaacttcgccct
gaagaacggcttcatcgccagcaatagccaaaggaaaggcagtgaggggacctgcagctgctcgtctaagggtttctccaccacgt
gtccagcccacgtcgatgacctaactccagaacaagtcctggatggggatgtaaatgagctgatggatgtagttctccaccatgttcc
agaggcaaagctggtggagtgcattggtcaagaacttatcttccttcttccaaataagaacttcaagcacagagcatatgccagcctt
ttcagagagctggaggagacgctggctgaccttggtctcagcagttttggaatttctgacactcccctggaagagatttttctgaaggt
cacggaggattctgattcaggacctctgtttgcgggtggcgctcagcagaaaagagaaaacgtcaacccccgacacccctgcttggg
tcccagagagaaggctggacagacaccccaggactccaatgtctgctccccaggggcgccggctgctcacccagagggccagcctc
ccccagagccagagtgcccaggcccgcagctcaacacggggacacagctggtcctccagcatgtgcaggcgctgctggtcaagag
attccaacacaccatccgcagccacaaggacttcctggcgcagatcgtgctcccggctacctttgtgtttttggctctgatgctttctat
tgttatccctccttttggcgaataccccgctttgacccttcacccctggatatatgggcagcagtacaccttcttcagcatggatgaacc
aggcagtgagcagttcacggtacttgcagacgtcctcctgaataagccaggctttggcaaccgctgcctgaaggaagggtggcttcc
ggagtacccctgtggcaactcaacaccctggaagactccttctgtgtccccaaacatcacccagctgttccagaagcagaaatggac
acaggtcaacccttcaccatcctgcaggtgcagcaccagggagaagctcaccatgctgccagagtgccccgagggtgccgggggcc
tcccgcccccccagagaacacagcgcagcacggaaattctacaagacctgacggacaggaacatctccgacttcttggtaaaaacg
tatcctgctcttataagaagcagcttaaagagcaaattctgggtcaatgaacagaggtatggaggaatttccattggaggaaagctc
ccagtcgtccccatcacgggggaagcacttgttgggtttttaagcgaccttggccggatcatgaatgtgagcgggggccctatcacta
gagaggcctctaaagaaatacctgatttccttaaacatctagaaactgaagacaacattaaggtgtggtttaataacaaaggctggc
atgccctggtcagctttctcaatgtggcccacaacgccatcttacmgccagcctgcctaaggacaggagccccgaggagtatggaa
tcaccgtcattagccaacccctgaacctgaccaaggagcagctctcagagattacagtgctgaccacttcagtggatgctgtggttgc
catctgcgtgattttctccatgtccttcgtcccagccagctttgtcctttatttgatccaggagcgggtgaacaaatccaagcacctcca
gtttatcagtggagtgagccccaccacctactgggtaaccaacttcctctgggacatcatgaattattccgtgagtgctgggctggtgg
tgggcatcttcatcgggtttcagaagaaagcctacacttctccagaaaaccttcctgcccttgtggcactgctcctgctgtatggatgg
gcggtcattcccatgatgtacccagcatccttcctgtttgatgtccccagcacagcctatgtggctttatcttgtgctaatctgttcatcg
gcatcaacagcagtgctattaccttcatcttggaattatttgagaataaccggacgctgctcaggttcaacgccgtgctgaggaagct
gctcattgtcttcccccacttctgcctgggccggggcctcattgaccttgcactgagccaggctgtgacagatgtctatgcccggtttgg
tgaggagcactctgcaaatccgttccactgggacctgattgggaagaacctgtttgccatggtggtggaaggggtggtgtacttcctc
ctgaccctgctggtccagcgccacttcttcctctcccaatggattgccgagcccactaaggagcccattgttgatgaagatgatgatgt
ggctgaagaaagacaaagaattattactggtggaaataaaactgacatcttaaggctacatgaactaaccaagatttatccaggca
cctccagcccagcagtggacaggctgtgtgtcggagttcgccctggagagtgctttggcctcctgggagtgaatggtgccggcaaaa
caaccacattcaagatgctcactggggacaccacagtgacctcaggggatgccaccgtagcaggcaagagtattttaaccaatattt
ctgaagtccatcaaaatatgggctactgtcctcagtttgatgcaatcgatgagctgctcacaggacgagaacatctttacctttatgcc
cggcttcgaggtgtaccagcagaagaaatcgaaaaggttgcaaactggagtattaagagcctgggcctgactgtctacgccgactg
cctggctggcacgtacagtgggggcaacaagcggaaactctccacagccatcgcactcattggctgcccaccgctggtgctgctgga
tgagcccaccacagggatggacccccaggcacgccgcatgctgtggaacgtcatcgtgagcatcatcagagaagggagggctgtg
gtcctcacatcccacagcatggaagaatgtgaggcactgtgtacccggctggccatcatggtaaagggcgcctttcgatgtatgggc
accattcagcatctcaagtccaaatttggagatggctatatcgtcacaatgaagatcaaatccccgaaggacgacctgcttcctgacc
tgaaccctgtggagcagttcttccaggggaacttcccaggcagtgtgcagagggagaggcactacaacatgctccagttccaggtct
cctcctcctccctggcgaggatcttccagctcctcctctcccacaaggacagcctgctcatcgaggagtactcagtcacacagaccac
actggaccaggtgtttgtaaattttgctaaacagcagactgaaagtcatgacctccctctgcaccctcgagctgctggagccagtcga
caagcccaggacgactacaaagaccatgacggtgattataaagatcatgacatcgactacaaggatgacgatgacaagtga
gcggccgcttcgagcagacatgataagatacattgatgagtttggacaaaccacaactagaatgcagtgaaaaaaatgctttatt
tgtgaaatttgtgatgctattgctttatttgtaaccattataagctgcaataaacaagttaacaacaacaattgcattcattttatgttt
caggttcagggggagatgtgggaggttttttaaagcaagtaaaacctctacaaatgtggtaaaatcgataaggatcttcctagagca
tggctacgtagataagtagcatggcgggttaatcattaactacaaggaacccctagtgatggagttggccactccctctctgcgcgct
cgctcgctcactgaggccgggcgaccaaaggtcgcccgacgcccgggctttgcccgggcggcctcagtgagcgagcgagcgcgca
g
pzac-CMV260-5′ ABCA4 intein (set3) SEQ ID No. 67
5′ ITR (seq A)
CMV260: bold
5′ ABCA4: underline
N-intein Npu DnaE: double underline
3xflag: italic
SV40: bold underline
3′ ITR (seq H)
ctgcgcgctcgctcgctcactgaggccgcccgggcaaagcccgggcgtcgggcgacctttggtcgcccggcctcagtgagcgagcg
agcgcgcagagagggagtggccaactccatcactaggggttccttgtagttaatgattaacccgccatgctacttatctacgtagcca
tgctctaggaagatcttcaatattggccattagccatattattcattggttatatagcataaatcaatattggctattggccattgcata
cgttgtatctatatcataatatgtacatttatattggctcatgtccaatatgaccgccatgttggcattgattattgactagcgttgacat
tgattattgactagtacggtaaatggcccgcctggctgatgactcacggggatttccaagtctccaccccattgacgtcaatgggag
tttgttttggcaccaaaatcaacgggactttccaaaatgtcgtaacaactccgccccattgacgcaaatgggcggtaggcgtgtac
ggtgggaggtctatataagcagagctggtttagtgaactagagaacccactgcttactggcttctcgagattccaccatggcggcc
gccatgggcttcgtgagacagatacagcttttgctctggaagaactggaccctgcggaaaaggcaaaagattcgctttgtggtggaa
ctcgtgtggcctttatctttatttctggtcttgatctggttaaggaatgccaacccgctctacagccatcatgaatgccatttccccaaca
aggcgatgccctcagcaggaatgctgccgtggctccaggggatcttctgcaatgtgaacaatccctgttttcaaagccccaccccag
gagaatctcctggaattgtgtcaaactataacaactccatcttggcaagggtatatcgagattttcaagaactcctcatgaatgcacc
agagagccagcaccttggccgtatttggacagagctacacatcttgtcccaattcatggacaccctccggactcacccggagagaat
tgcaggaagaggaattcgaataagggatatcttgaaagatgaagaaacactgacactatttctcattaaaaacatcggcctgtctga
ctcagtggtctaccttctgatcaactctcaagtccgtccagagcagttcgctcatggagtcccggacctggcgctgaaggacatcgcc
tgcagcgaggccctcctggagcgcttcatcatcttcagccagagacgcggggcaaagacggtgcgctatgccctgtgctccctctccc
agggcaccctacagtggatagaagacactctgtatgccaacgtggacttcttcaagctcttccgtgtgcttcccacactcctagacag
ccgttctcaaggtatcaatctgagatcttggggaggaatattatctgatatgtcaccaagaattcaagagtttatccatcggccgagta
tgcaggacttgctgtgggtgaccaggcccctcatgcagaatggtggtccagagacctttacaaagctgatgggcatcctgtctgacct
cctgtgtggctaccccgagggaggtggctctcgggtgctctccttcaactggtatgaagacaataactataaggcctttctggggatt
gactccacaaggaaggatcctatctattcttatgacagaagaacaacatccttttgtaatgcattgatccagagcctggagtcaaatc
ctttaaccaaaatcgcttggagggcggcaaagcctttgctgatgggaaaaatcctgtacactcctgattcacctgcagcacgaagga
tactgaagaatgccaactcaacttttgaagaactggaacacgttaggaagttggtcaaagcctgggaagaagtagggccccagatc
tggtacttctttgacaacagcacacagatgaacatgatcagagataccctggggaacccaacagtaaaagactttttgaataggca
gcttggtgaagaaggtattactgctgaagccatcctaaacttcctctacaagggccctcgggaaagccaggctgacgacatggccaa
cttcgactggagggacatatttaacatcactgatcgcaccctccgccttgtcaatcaatacctggagtgcttggtcctggataagtttg
aaagctacaatgatgaaactcagctcacccaacgtgccctctctctactggaggaaaacatgttctgggccggagtggtattccctga
catgtatccctggaccagctctctaccaccccacgtgaagtataagatccgaatggacatagacgtggtggagaaaaccaataaga
ttaaagacaggtattgggattctggtcccagagctgatcccgtggaagatttccggtacatctggggcgggtttgcctatctgcagga
catggttgaacaggggatcacaaggagccaggtgcaggcggaggctccagttggaatctacctccagcagatgccctacccctgctt
cgtggacgattctttcatgatcatcctgaaccgctgtttccctatcttcatggtgctggcatggatctactctgtctccatgactgtgaag
agcatcgtcttggagaaggagttgcgactgaaggagaccttgaaaaatcagggtgtctccaatgcagtgatttggtgtacctggttcc
tggacagcttctccatcatgtcgatgagcatcttcctcctgacgatattcatcatgcatggaagaatcctacattacagcgacccattc
atcctcttcctgttcttgttggctttctccactgccaccatcatgctgtgctttctgctcagcaccttcttctccaaggccagtctggcagc
agcctgtagtggtgtcatctatttcaccctctacctgccacacatcctgtgcttcgcctggcaggaccgcatgaccgctgagctgaaga
aggctgtgagcttactgtctccggtggcatttggatttggcactgagtacctggttcgctttgaagagcaaggcctggggctgcagtgg
agcaacatcgggaacagtcccacggaaggggacgaattcagcttcctgctgtccatgcagatgatgctccttgatgctgctgtctatg
gcttactcgcttggtaccttgatcaggtgtttccaggagactatggaaccccacttccttggtactttcttctacaagagtcgtattggct
tggcggtgaagggtgttcaaccagagaagaaagagccctggaaaagaccgagcccctaacagaggaaacggaggatccagagc
acccagaaggaatacacgactccttctttgaacgtgagcatccagggtgggttcctggggtatgcgtgaagaatctggtaaagatttt
tgagccctgtggccggccagctgtggaccgtctgaacatcaccttctacgagaaccagatcaccgcattcctgggccacaatggagc
tgggaaaaccaccaccttgtccatcctgacgggtctgttgccaccaacctctgggactgtgctcgttgggggaagggacattgaaac
cagcctggatgcagtccggcagagccttggcatgtgtccacagcacaacatcctgttccaccacctcacggtggctgagcacatgct
gttctatgcccagctgaaaggaaagtcccaggaggaggcccagctggagatggaagccatgttggaggacacaggcctccaccac
aagcggaatgaagaggctcaggacctatcaggtggcatgcagagaaagctgtcggttgccattgcctttgtgggagatgccaaggt
ggtgattctggacgaacccacctgcctgagctacgacaccgagatcctgaccgtggagtacggcatcctgcccatcggcaagatcgt
ggagaagaggatcgagtgcaccgtgtacagcgtggacaacaacggcaacatctacacccagcccgtggcccagtggcacgacag
gggcgagcaggaggtgttcgagtactgcctggaggacggcagcctgatcagggccaccaaggaccacaagttcatgaccgtggac
ggccagatgatgcccatcgacgagatcttcgagagggagctggacctgatgagggtggacaacctgcccaacgactacaaagacc
atgacggtgattataaagatcatgacatcgactacaaggatgacgatgacaagtgagcggccgcttcgagcagacatgataa
gatacattgatgagtttggacaaaccacaactagaatgcagtgaaaaaaatgctttatttgtgaaatttgtgatgctattgctttat
ttgtaaccattataagctgcaataaacaagttaacaacaacaattgcattcattttatgtttcaggttcagggggagatgtgggaggt
tttttaaagcaagtaaaacctctacaaatgtggtaaaatcgataaggatcttcctagagcatggctacgtagataagtagcatggcg
ggttaatcattaactacaaggaacccctagtgatggagttggccactccctctctgcgcgctcgctcgctcactgaggccgggcgacc
aaaggtcgcccgacgcccgggctttgcccgggcggcctcagtgagcgagcgagcgcgcag
pzac-CMV260-3′ ABCA4 intein (set3) SEQ ID No. 68
5′ ITR (seq A)
CMV260: bold
3′ ABCA4: underline
C-intein Npu DnaE: double underline
3xflag: italic
SV40: bold underline
3′ ITR (seq H)
ctgcgcgctcgctcgctcactgaggccgcccgggcaaagcccgggcgtcgggcgacctttggtcgcccggcctcagtgagcgagcg
agcgcgcagagagggagtggccaactccatcactaggggttccttgtagttaatgattaacccgccatgctacttatctacgtagcca
tgctctaggaagatcttcaatattggccattagccatattattcattggttatatagcataaatcaatattggctattggccattgcata
cgttgtatctatatcataatatgtacatttatattggctcatgtccaatatgaccgccatgttggcattgattattgactagcgttgacat
tgattattgactagtacggtaaatggcccgcctggctgatgactcacggggatttccaagtctccaccccattgacgtcaatgggag
tttgttttggcaccaaaatcaacgggactttccaaaatgtcgtaacaactccgccccattgacgcaaatgggcggtaggcgtgtac
ggtgggaggtctatataagcagagctggtttagtgaactagagaacccactgcttactggcttctcgagattccaccatggcggcc
gccatggtgaaggtgatcggcaggaggagcctgggcgtgcagaggatcttcgacatcggcctgccccagtaccacaacttcctgctg
gccaacggcgccatcgccgccaactctggggtggacccttactcgagacgctcaatctgggatctgctcctgaagtatcgctcaggc
agaaccatcatcatgtccactcaccacatggacgaggccgacctccttggggaccgcattgccatcattgcccagggaaggctctac
tgctcaggcaccccactcttcctgaagaactgctttggcacaggcttgtacttaaccttggtgcgcaagatgaaaaacatccagagcc
aaaggaaaggcagtgaggggacctgcagctgctcgtctaagggtttctccaccacgtgtccagcccacgtcgatgacctaactccag
aacaagtcctggatggggatgtaaatgagctgatggatgtagttctccaccatgttccagaggcaaagctggtggagtgcattggtc
aagaacttatcttccttcttccaaataagaacttcaagcacagagcatatgccagccttttcagagagctggaggagacgctggctg
accttggtctcagcagttttggaatttctgacactcccctggaagagatttttctgaaggtcacggaggattctgattcaggacctctgt
ttgcgggtggcgctcagcagaaaagagaaaacgtcaacccccgacacccctgcttgggtcccagagagaaggctggacagacacc
ccaggactccaatgtctgctccccaggggcgccggctgctcacccagagggccagcctcccccagagccagagtgcccaggcccgc
agctcaacacggggacacagctggtcctccagcatgtgcaggcgctgctggtcaagagattccaacacaccatccgcagccacaag
gacttcctggcgcagatcgtgctcccggctacctttgtgtttttggctctgatgctttctattgttatccctccttttggcgaataccccgc
tttgacccttcacccctggatatatgggcagcagtacaccttcttcagcatggatgaaccaggcagtgagcagttcacggtacttgca
gacgtcctcctgaataagccaggctttggcaaccgctgcctgaaggaagggtggcttccggagtacccctgtggcaactcaacaccc
tggaagactccttctgtgtccccaaacatcacccagctgttccagaagcagaaatggacacaggtcaacccttcaccatcctgcagg
tgcagcaccagggagaagctcaccatgctgccagagtgccccgagggtgccgggggcctcccgcccccccagagaacacagcgca
gcacggaaattctacaagacctgacggacaggaacatctccgacttcttggtaaaaacgtatcctgctcttataagaagcagcttaa
agagcaaattctgggtcaatgaacagaggtatggaggaatttccattggaggaaagctcccagtcgtccccatcacgggggaagca
cttgttgggtttttaagcgaccttggccggatcatgaatgtgagcgggggccctatcactagagaggcctctaaagaaatacctgatt
tccttaaacatctagaaactgaagacaacattaaggtgtggtttaataacaaaggctggcatgccctggtcagctttctcaatgtggc
ccacaacgccatcttacgggccagcctgcctaaggacaggagccccgaggagtatggaatcaccgtcattagccaacccctgaacc
tgaccaaggagcagctctcagagattacagtgctgaccacttcagtggatgctgtggttgccatctgcgtgattttctccatgtccttcg
tcccagccagctttgtcctttatttgatccaggagcgggtgaacaaatccaagcacctccagtttatcagtggagtgagccccaccac
ctactgggtaaccaacttcctctgggacatcatgaattattccgtgagtgctgggctggtggtgggcatcttcatcgggtttcagaaga
aagcctacacttctccagaaaaccttcctgcccttgtggcactgctcctgctgtatggatgggcggtcattcccatgatgtacccagca
tccttcctgtttgatgtccccagcacagcctatgtggctttatcttgtgctaatctgttcatcggcatcaacagcagtgctattaccttcat
cttggaattatttgagaataaccggacgctgctcaggttcaacgccgtgctgaggaagctgctcattgtcttcccccacttctgcctgg
gccggggcctcattgaccttgcactgagccaggctgtgacagatgtctatgcccggtttggtgaggagcactctgcaaatccgttcca
ctgggacctgattgggaagaacctgtttgccatggtggtggaaggggtggtgtacttcctcctgaccctgctggtccagcgccacttct
tcctctcccaatggattgccgagcccactaaggagcccattgttgatgaagatgatgatgtggctgaagaaagacaaagaattatta
ctggtggaaataaaactgacatcttaaggctacatgaactaaccaagatttatccaggcacctccagcccagcagtggacaggctgt
gtgtcggagttcgccctggagagtgctttggcctcctgggagtgaatggtgccggcaaaacaaccacattcaagatgctcactgggg
acaccacagtgacctcaggggatgccaccgtagcaggcaagagtattttaaccaatatttctgaagtccatcaaaatatgggctact
gtcctcagtttgatgcaatcgatgagctgctcacaggacgagaacatctttacctttatgcccggcttcgaggtgtaccagcagaaa
aatcgaaaaggttgcaaactggagtattaagagcctgggcctgactgtctacgccgactgcctggctggcacgtacagtgggggca
acaagcggaaactctccacagccatcgcactcattggctgcccaccgctggtgctgctggatgagcccaccacagggatggaccccc
aggcacgccgcatgctgtggaacgtcatcgtgagcatcatcagagaagggagggctgtggtcctcacatcccacagcatggaagaa
tgtgaggcactgtgtacccggctggccatcatggtaaagggcgcctttcgatgtatgggcaccattcagcatctcaagtccaaatttg
gagatggctatatcgtcacaatgaagatcaaatccccgaaggacgacctgcttcctgacctgaaccctgtggagcagttcttccagg
ggaacttcccaggcagtgtgcagagggagaggcactacaacatgctccagttccaggtctcctcctcctccctggcgaggatcttcca
gctcctcctctcccacaaacagcctgctcatcgaggagtactcagtcacacagaccacactaccaggtgtttgtaaattttgct
aaacagcagactgaaagtcatgacctccctctgcaccctcgagctgctggagccagtcgacaagcccaggacgactocaaagacc
atgacggtgattataaagatcatgacatcgactacaaggatgacgatgacaagtgagcggccgcttcgagcagacatgataa
gatacattgatgagtttggacaaaccacaactagaatgcagtgaaaaaaatgctttatttgtgaaatttgtgatgctattgctttat
ttgtaaccattataagctgcaataaacaagttaacaacaacaattgcattcattttatgtttcaggttcagggggagatgtgggaggt
tttttaaagcaagtaaaacctctacaaatgtggtaaaatcgataaggatcttcctagagcatggctacgtagataagtagcatggcg
ggttaatcattaactacaaggaacccctagtgatggagttggccactccctctctgcgcgctcgctcgctcactgaggccgggcgacc
aaaggtcgcccgacgcccgggctttgcccgggcggcctcagtgagcgagcgagcgcgcag
p836 (IRBP_DSRed) SEQ ID No. 69
5′ ITR (seq A)
IRBP bold
WPRE: italic underline
DsRed underline
BghpA: bold underline
3′ ITR (seq H)
ctgcgcgctcgctcgctcactgaggccgcccgggcaaagcccgggcgtcgggcgacctttggtcgcccggcctcagtgagcgagcg
agcgcgcagagagggagtggccaactccatcactaggggttcctctagtagcacagtgtctggcatgtagcaggaactaaaataa
tggcagtgattaatgttatgatatgcagacacaacacagcaagataagatgcaatgtaccttctgggtcaaaccaccctggccact
cctccccgatacccagggttgatgtgcttgaattagacaggattaaaggcttactggagctggaagccttgccccaactcaggagtt
tagccccagaccttctgtccaccagcgcggccgaccggccaagggcgaattctgcagatatccatcacactggcatggatagcact
gagaacgtcatcaagcccttcatgcgcttcaaggtgcacatggagggctccgtgaacggccacgagttcgagatcgagggcgaggg
cgagggcaagccctacgagggcacccagaccgccaagctgcaggtgaccaagggcggccccctgcccttcgcctgggacatcctgt
ccccccagttccagtacggctccaaggtgtacgtgaagcaccccgccgacatccccgactacaagaagctgtccttccccgagggct
tcaagtgggagcgcgtgatgaacttcgaggacggcggcgtggtgaccgtgacccaggactcctccctgcaggacggcaccttcatct
accacgtgaagttcatcggcgtgaacttcccctccgacggccccgtaatgcagaagaagactctgggctgggagccctccaccgag
cgcctgtacccccgcgacggcgtgctgaagggcgagatccacaaggcgctgaagctgaagggcggcggccactacctggtggagtt
caagtcaatctacatggccaagaagcccgtgaagctgcccggctactactacgtggactccaagctggacatcacctcccacaacg
aggactacaccgtggtggagcagtacgagcgcgccgaggcccgccaccacctgttccagtagaatcaacctctggattacaaaat
ttgtgaaagattgactggtattcttaactatgttgctccttttacgctatgtggatacgctgctttaatgctttgtatcatgctattg
cttcccgtatggctttcattttctcctccttgtataaatcctggttgctgtctctttatgaggagttgtggccgttgtcaggcaacgt
ggcgtggtgtgcactgtgtttgctgacgcaacccccactggttggggcattgccaccacctgtcagctcctttccgggactttcgct
ttccccctccctattgccacggcggaactcgtcgccgcctgccttgcccgctgctggacaggggctcggctgttaggcactacaa
ttccgtggtgttgtcggggaagctgacgtcctttccatggctgctcgcctgtgttgccacctggattctgcgcgggacgtccttcta
ctacgtccttcggccctcaatccagcggaccttcttcccgcggcctgctgccggctctgcggcctcttccgcgtcttcaggcctcga
ctgtgccttctagttgccagccatctgttgtttgcccctcccccgtgccttccttgaccctggaaggtgccactcccactgtcctttccta
ataaaatgaggaaattgcatcgcattgtctgagtaggtgtcattctattctggggggtggggtggggcaggacagcaagggggag
gattgggaagacaatagcaggcatgctggggaaggaacccctagtgatggagttggccactccctctctgcgcgctcgctcgctca
ctgaggccgggcgaccaaaggtcgcccgacgcccgggctttgcccgggcggcctcagtgagcgagcgagcgcgcag
p1232 pAAV2.1_HLP_5′ F8 intein (set 1)
5′ ITR (seq A)
HLP promoter (seq J) SEQ. ID No. 70
tgtttgctgcttgcaatgtttgcccattttagggtggacacaggacgctgtggtttctgagccagggggcgactcagatcccagccagt
ggacttagcccctgtttgctcctccgataactggggtgaccttggttaatattcaccagcagcctcccccgttgcccctctggatccact
gcttaaatacggacgaggacagggccctgtctcctcagcttcaggcaccaccactgacctgggacagtgat
F8 signal sequence (seq K) SEQ. ID No. 71
atgcaaatagagctctccacctgcttctttctgtgccttttgcgattctgctttagt
5′ F8: SEQ. ID No. 72
gccaccagaagatactacctgggtgcagtggaactgtcatgggactatatgcaaagtgatctcggtgagctgcctgtggacgcaag
atttcctcctagagtgccaaaatcttttccattcaacacctcagtcgtgtacaaaaagactctgtttgtagaattcacggatcacctttt
caacatcgctaagccaaggccaccctggatgggtctgctaggtcctaccatccaggctgaggtttatgatacagtggtcattacactt
aagaacatggcttcccatcctgtcagtcttcatgctgttggtgtatcctactggaaagcttctgagggagctgaatatgatgatcagac
cagtcaaagggagaaagaagatgataaagtcttccctggtggaagccatacatatgtctggcaggtcctgaaagagaatggtccaa
tggcctctgacccactgtgccttacctactcatatctttctcatgtggacctggtaaaagacttgaattcaggcctcattggagccctac
tagtatgtagagaagggagtctggccaaggaaaagacacagaccttgcacaaatttatactactttttgctgtatttgatgaaggga
aaagttggcactcagaaacaaagaactccttgatgcaggatagggatgctgcatctgctcgggcctggcctaaaatgcacacagtc
aatggttatgtaaacaggtctctgccaggtctgattggatgccacaggaaatcagtctattggcatgtgattggaatgggcaccactc
ctgaagtgcactcaatattcctcgaaggtcacacatttcttgtgaggaaccatcgccaggcgtccttggaaatctcgccaataactttc
cttactgctcaaacactcttgatggaccttggacagtttctactgttttgtcatatctcttcccaccaacatgatggcatggaagcttatg
tcaaagtagacagctgtccagaggaaccccaactacgaatgaaaaataatgaagaagcggaagactatgatgatgatcttactgat
tctgaaatggatgtggtcaggtttgatgatgacaactctccttcctttatccaaattcgctcagttgccaagaagcatcctaaaacttg
ggtacattacattgctgctgaagaggaggactgggactatgctcccttagtcctcgcccccgatgacagaagttataaaagtcaatat
ttgaacaatggccctcagcggattggtaggaagtacaaaaaagtccgatttatggcatacacagatgaaacctttaagactcgtgaa
gctattcagcatgaatcaggaatcttgggacctttactttatggggaagttggagacacactgttgattatatttaagaatcaagcaa
gcagaccatataacatctaccctcacggaatcactgatgtccgtcctttgtattcaaggagattaccaaaaggtgtaaaacatttgaa
ggattttccaattctgccaggagaaatattcaaatataaatggacagtgactgtagaagatgggccaactaaatcagatcctcggtg
cctgacccgctattactctagtttcgttaatatggagagagatctagcttcaggactcattggccctctcctcatctgctacaaagaatc
tgtagatcaaagaggaaaccagataatgtcagacaagaggaatgtcatcctgttttctgtatttgatgagaaccgaagctggtacct
cacagagaatatacaacgctttctccccaatccagctggagtgcagcttgaggatccagagttccaagcctccaacatcatgcacag
catcaatggctatgtttttgatagtttgcagttgtcagtttgtttgcatgaggtggcatactggtacattctaagcattggagcacagac
tgacttcctttctgtcttcttctctggatataccttcaaacacaaaatggtctatgaagacacactcaccctattcccattctcaggaga
aactgtcttcatgtcgatggaaaacccaggtctatggattctggggtgccacaactcagactttcggaacagaggcatgaccgcctta
ctgaaggtttctagttgtgacaagaacactggtgattattacgaggacagttatgaagatatttcagcatacttgctgagtaaaaaca
atgccattgaaccaagaagcttctcccagaattcaagacaccctagcactaggcaaaagcaatttaatgccaccacaattccagaa
aatgacatagagaagactgacccttggtttgcacacagaacacctatgcctaaaatacaaaatgtctcctctagtgatttgttgatgc
tcttgcgacagagtcctactccacatgggctatccttatctgatctccaagaagccaaatatgagactttttctgatgatccatcacctg
gagcaatagacagtaataacagcctgtctgaaatgacacacttcaggccacagctccatcacagtggggacatggtatttacccctg
agtcaggcctccaattaagattaaatgagaaactggggacaactgcagcaacagagttgaagaaacttgatttcaaagtttctagta
catcaaataatctgatttcaacaattccatcagacaatttggcagcaggtactgataatacaagttccttaggacccccaagtatgcc
agttcattatgatagtcaattagataccactctatttggcaaaaagtcatctccccttactgagtctggtggacctctgagcttgagtga
agaaaataatgattcaaagttgttagaatcaggtttaatgaatagccaagaaagttcatggggaaaaaatgtatcgtcaacagaga
gtggtaggttatttaaagggaaaagagctcatggacctgctttgttgactaaagataatgccttattcaaagttagcatctctttgtta
aagacaaacaaaacttccaataattcagcaactaatagaaagactcacattgatggcccatcattattaattgagaatagtccatca
gtctggcaaaatatattagaaagtgacactgagtttaaaaaagtgacacctttgattcatgacagaatgcttatggacaaaaatgct
acagctttgaggctaaatcatatgtcaaataaaactacttcatcaaaaaacatggaaatggtccaacagaaaaaagagggccccat
tccaccagatgcacaaaatccagatatgtcgttctttaagatgctattcttgccagaatcagcaaggtggatacaaaggactcatgg
aaagaactctctgaactctgggcaaggccccagtccaaagcaattagtatccttaggaccagaaaaatctgtggaaggtcagaattt
cttgtctgagaaaaacaaagtggtagtaggaaagggtgaatttacaaaggacgtaggactcaaagagatggtttttccaagcagca
gaaacctatttcttactaacttggataatttacatgaaaataatacacacaatcaagaaaaaaaaattcaggaagaaatagaaaag
aaggaaacattaatccaagagaatgtagttttgcctcagatacatacagtgactggcactaagaatttcatgaagaaccttttcttac
tgagcactaggcaaaatgtagaaggttcatatgacggggcatatgctccagtacttcaagattttaggtcattaaatgattcaacaaa
tagaacaaagaaacacacagctcatttctcaaaaaaaggggaggaagaaaacttggaaggcttgggaaatcaaaccaagcaaat
tgtagagaaatatgca
N-intein Npu DnaE (seq D)
3xflag (seq E)
shPolyA (seq V)
3′ ITR (seq H)
p1389 pAAV2.1_HLP_3′ F8 intein (set 1)
5′ ITR (seq A)
HLP promoter (seq J)
F8 signal sequence (seq K)
C-intein Npu DnaE (seq ()
3′ F8: SEQ ID No. 73
tgcaccacaaggatatctcctaatacaagccagcagaattttgtcacgcaacgtagtaagagagctttgaaacaattcagactccca
ctagaagaaacagaacttgaaaaaaggataattgtggatgacacctcaacccagtggtccaaaaacatgaaacatttgaccccga
gcaccctcacacagatagactacaatgagaaggagaaaggggccattactcagtctcccttatcagattgccttacgaggagtcata
gcatccctcaagcaaatagatctccattacccattgcaaaggtatcatcatttccatctattagacctatatatctgaccagggtcctat
tccaagacaactcttctcatcttccagcagcatcttatagaaagaaagattctggggtccaagaaagcagtcatttcttacaaggagc
caaaaaaaataacctttctttagccattctaaccttggagatgactggtgatcaaagagaggttggctccctggggacaagtgccac
aaattcagtcacatacaagaaagttgagaacactgttctcccgaaaccagacttgcccaaaacatctggcaaagttgaattgcttcc
aaaagttcacatttatcagaaggacctattccctacggaaactagcaatgggtctcctggccatctggatctcgtggaagggagcctt
cttcagggaacagagggagcgattaagtggaatgaagcaaacagacctggaaaagttccctttctgagagtagcaacagaaagct
ctgcaaagactccctccaagctattggatcctcttgcttgggataaccactatggtactcagataccaaaagaagagtggaaatccc
aagagaagtcaccagaaaaaacagcttttaagaaaaaggataccattttgtccctgaacgcttgtgaaagcaatcatgcaatagca
gcaataaatgagggacaaaataagcccgaaatagaagtcacctgggcaaagcaaggtaggactgaaaggctgtgctctcaaaac
ccaccagtcttgaaacgccatcaacgggaaataactcgtactactcttcagtcagatcaagaggaaattgactatgatgataccata
tcagttgaaatgaagaaggaagattttgacatttatgatgaggatgaaaatcagagcccccgcagctttcaaaagaaaacacgaca
ctattttattgctgcagtggagaggctctgggattatgggatgagtagctccccacatgttctaagaaacagggctcagagtggcagt
gtccctcagttcaagaaagttgttttccaggaatttactgatggctcctttactcagcccttataccgtggagaactaaatgaacatttg
ggactcctggggccatatataagagcagaagttgaagataatatcatggtaactttcagaaatcaggcctctcgtccctattccttcta
ttctagccttatttcttatgaggaagatcagaggcaaggagcagaacctagaaaaaactttgtcaagcctaatgaaaccaaaactta
cttttggaaagtgcaacatcatatggcacccactaaagatgagtttgactgcaaagcctgggcttatttctctgatgttgacctggaaa
aagatgtgcactcaggcctgattggaccccttctggtctgccacactaacacactgaaccctgctcatgggagacaagtgacagtac
aggaatttgctctgtttttcaccatctttgatgagaccaaaagctggtacttcactgaaaatatggaaagaaactgcagggctccctg
caatatccagatggaagatcccacttttaaagagaattatcgcttccatgcaatcaatggctacataatggatacactacctggctta
gtaatggctcaggatcaaaggattcgatggtatctgctcagcatgggcagcaatgaaaacatccattctattcatttcagtggacatg
tgttcactgtacgaaaaaaagaggagtataaaatggcactgtacaatctctatccaggtgtttttgagacagtggaaatgttaccatc
caaagctggaatttggcgggtggaatgccttattggcgagcatctacatgctgggatgagcacactttttctggtgtacagcaataag
tgtcagactcccctgggaatggcttctggacacattagagattttcagattacagcttcaggacaatatggacagtgggccccaaagc
tggccagacttcattattccggatcaatcaatgcctggagcaccaaggagcccttttcttggatcaaggtggatctgttggcaccaatg
attattcacggcatcaagacccagggtgcccgtcagaagttctccagcctctacatctctcagtttatcatcatgtatagtcttgatggg
aagaagtggcagacttatcgaggaaattccactggaaccttaatggtcttctttggcaatgtggattcatctgggataaaacacaata
tttttaaccctccaattattgctcgatacatccgtttgcacccaactcattatagcattcgcagcactcttcgcatggagttgatgggctg
tgatttaaatagttgcagcatgccattgggaatggagagtaaagcaatatcagatgcacagattactgcttcatcctactttaccaat
atgtttgccacctggtctccttcaaaagctcgacttcacctccaagggaggagtaatgcctggagacctcaggtgaataatccaaaa
gagtggctgcaagtggacttccagaagacaatgaaagtcacaggagtaactactcagggagtaaaatctctgcttaccagcatgta
tgtgaaggagttcctcatctccagcagtcaagatggccatcagtggactctcttttttcagaatggcaaagtaaaggtttttcagggaa
atcaagactccttcacacctgtggtgaactctctagacccaccgttactgactcgctaccttcgaattcacccccagagttgggtgcac
cagattgccctgaggatggaggttctgggctgcgaggcacaggacctctac
3xflag (seq E)
shPolyA (seq V)
3′ ITR (seq H)
p1207 pAAV2.1_HLP_5′ F8 intein (set 2)
5′ ITR (seq A)
HLP promoter (seq J)
F8 signal sequence (seq K)
5′ F8 (set 2): SEQ ID No. 74
gccaccagaagatactacctgggtgcagtggaactgtcatgggactatatgcaaagtgatctcggtgagctgcctgtggacgcaag
atttcctcctagagtgccaaaatcttttccattcaacacctcagtcgtgtacaaaaagactctgtttgtagaattcacggatcacctttt
caacatcgctaagccaaggccaccctggatgggtctgctaggtcctaccatccaggctgaggtttatgatacagtggtcattacactt
aagaacatggcttcccatcctgtcagtcttcatgctgttggtgtatcctactggaaagcttctgagggagctgaatatgatgatcagac
cagtcaaagggagaaagaagatgataaagtcttccctggtggaagccatacatatgtctggcaggtcctgaaagagaatggtccaa
tggcctctgacccactgtgccttacctactcatatctttctcatgtggacctggtaaaagacttgaattcaggcctcattggagccctac
tagtatgtagagaagggagtctggccaaggaaaagacacagaccttgcacaaatttatactactttttgctgtatttgatgaaggga
aaagttggcactcagaaacaaagaactccttgatgcaggatagggatgctgcatctgctcgggcctggcctaaaatgcacacagtc
aatggttatgtaaacaggtctctgccaggtctgattggatgccacaggaaatcagtctattggcatgtgattggaatgggcaccactc
ctgaagtgcactcaatattcctcgaaggtcacacatttcttgtgaggaaccatcgccaggcgtccttggaaatctcgccataactttc
cttactgctcaaacactcttgatggaccttggacagtttctactgttttgtcatatctcttcccaccacatgatggcatggaagcttatg
tcaaagtagacagctgtccagaggaaccccaactacgaatgaaaaataatgaagaagcggaagactatgatgatgatcttactgat
tctgaaatggatgtggtcaggtttgatgatgacaactctccttcctttatccaaattcgctcagttgccaagaagcatcctaaaacttg
ggtacattacattgctgctgaagaggaggactgggactatgctcccttagtcctcgcccccgatgacagaagttataaaagtcaatat
ttgaacaatggccctcagcggattggtaggaagtacaaaaaagtccgatttatggcatacacagatgaaacctttaagactcgtgaa
gctattcagcatgaatcaggaatcttgggacctttactttatggggaagttggagacacactgttgattatatttaagaatcaagcaa
gcagaccatataacatctaccctcacggaatcactgatgtccgtcctttgtattcaaggagattaccaaaaggtgtaaaacatttgaa
ggattttccaattctgccaggagaaatattcaaatataaatggacagtgactgtagaagatgggccaactaaatcagatcctcggtg
cctgacccgctattactctagtttcgttaatatggagagagatctagcttcaggactcattggccctctcctcatctgctacaaagaatc
tgtagatcaaagaggaaaccagataatgtcagacaagaggaatgtcatcctgttttctgtatttgatgagaaccgaagctggtacct
cacagagaatatacaacgctttctccccaatccagctggagtgcagcttgaggatccagagttccaagcctccaacatcatgcacag
catcaatggctatgtttttgatagtttgcagttgtcagtttgtttgcatgaggtggcatactggtacattctaagcattggagcacagac
tgacttcctttctgtcttcttctctggatataccttcaaacacaaaatggtctatgaagacacactcaccctattcccattctcaggaga
aactgtcttcatgtcgatggaaaacccaggtctatggattctggggtgccacaactcagactttcggaacagaggcatgaccgcctta
ctgaaggtttctagttgtgacaagaacactggtgattattacgaggacagttatgaagatatttcagcatacttgctgagtaaaaaca
atgccattgaaccaagaagcttctcccagaattcaagacaccctagcactaggcaaaagcaatttaatgccaccacaattccagaa
aatgacatagagaagactgacccttggtttgcacacagaacacctatgcctaaaatacaaaatgtctcctctagtgatttgttgatgc
tcttgcgacagagtcctactccacatgggctatccttatctgatctccaagaagccaaatatgagactttttctgatgatccatcacctg
gagcaatagacagtaataacagcctgtctgaaatgacacacttcaggccacagctccatcacagtggggacatggtatttacccctg
agtcaggcctccaattaagattaaatgagaaactggggacaactgcagcaacagagttgaagaaacttgatttcaaagtttctagta
catcaaataatctgatttcaacaattccatcagacaatttggcagcaggtactgataatacaagttccttaggacccccaagtatgcc
agttcattatgatagtcaattagataccactctatttggcaaaaagtcatctccccttactgagtctggtggacctctgagcttgagtga
agaaaataatgattcaaagttgttagaatcaggtttaatgaatagccaagaaagttcatggggaaaaaatgta
N-intein Npu DnaE (seq D)
3xflag (seq E)
shPolyA (seq V)
3′ ITR (seq H)
p1388 pAAV2.1_HLP_3′ F8 intein (set 2)
5′ ITR (seq A)
HLP promoter (seq J)
F8 signal sequence (seq K)
C-intein Npu DnaE (seq I)
3′ F8: SEQ ID No. 75
tcgtcaacagagagtggtaggttatttaaagggaaaagagctcatggacctgctttgttgactaaagataatgccttattcaaagtta
gcatctctttgttaaagacaaacaaaacttccaataattcagcaactaatagaaagactcacattgatggcccatcattattaattga
gaatagtccatcagtctggcaaaatatattagaaagtgacactgagtttaaaaaagtgacacctttgattcatgacagaatgcttatg
gacaaaaatgctacagctttgaggctaaatcatatgtcaaataaaactacttcatcaaaaaacatggaaatggtccaacagaaaaa
agagggccccattccaccagatgcacaaaatccagatatgtcgttctttaagatgctattcttgccagaatcagcaaggtggataca
aaggactcatggaaagaactctctgaactctgggcaaggccccagtccaaagcaattagtatccttaggaccagaaaaatctgtgg
aaggtcagaatttcttgtctgagaaaaacaaagtggtagtaggaaagggtgaatttacaaaggacgtaggactcaaagagatggtt
tttccaagcagcagaaacctatttcttactaacttggataatttacatgaaaataatacacacaatcaagaaaaaaaaattcaggaa
gaaatagaaaagaaggaaacattaatccaagagaatgtagttttgcctcagatacatacagtgactggcactaagaatttcatgaa
gaaccttttcttactgagcactaggcaaaatgtagaaggttcatatgacggggcatatgctccagtacttcaagattttaggtcattaa
atgattcaacaaatagaacaaagaaacacacagctcatttctcaaaaaaaggggaggaagaaaacttggaaggcttgggaaatc
aaaccaagcaaattgtagagaaatatgcatgcaccacaaggatatctcctaatacaagccagcagaattttgtcacgcaacgtagt
aagagagctttgaaacaattcagactcccactagaagaaacagaacttgaaaaaaggataattgtggatgacacctcaacccagt
ggtccaaaaacatgaaacatttgaccccgagcaccctcacacagatagactacaatgagaaggagaaaggggccattactcagtc
tcccttatcagattgccttacgaggagtcatagcatccctcaagcaaatagatctccattacccattgcaaaggtatcatcatttccat
ctattagacctatatatctgaccagggtcctattccaagacaactcttctcatcttccagcagcatcttatagaaagaaagattctggg
gtccaagaaagcagtcatttcttacaaggagccaaaaaaaataacctttctttagccattctaaccttggagatgactggtgatcaaa
gagaggttggctccctggggacaagtgccacaaattcagtcacatacaagaaagttgagaacactgttctcccgaaaccagacttg
cccaaaacatctggcaaagttgaattgcttccaaaagttcacatttatcagaaggacctattccctacggaaactagcaatgggtctc
ctggccatctggatctcgtggaagggagccttcttcagggaacagagggagcgattaagtggaatgaagcaaacagacctggaaa
agttccctttctgagagtagcaacagaaagctctgcaaagactccctccaagctattggatcctcttgcttgggataaccactatggta
ctcagataccaaaagaagagtggaaatcccaagagaagtcaccagaaaaaacagcttttaagaaaaaggataccattttgtccct
gaacgcttgtgaaagcaatcatgcaatagcagcaataaatgagggacaaaataagcccgaaatagaagtcacctgggcaaagca
aggtaggactgaaaggctgtgctctcaaaacccaccagtcttgaaacgccatcaacgggaaataactcgtactactcttcagtcaga
tcaagaggaaattgactatgatgataccatatcagttgaaatgaagaaggaagattttgacatttatgatgaggatgaaaatcaga
gcccccgcagctttcaaaagaaaacacgacactattttattgctgcagtggagaggctctgggattatgggatgagtagctccccac
atgttctaagaaacagggctcagagtggcagtgtccctcagttcaagaaagttgttttccaggaatttactgatggctcctttactcag
cccttataccgtggagaactaaatgaacatttgggactcctggggccatatataagagcagaagttgaagataatatcatggtaact
ttcagaaatcaggcctctcgtccctattccttctattctagccttatttcttatgaggaagatcagaggcaaggagcagaacctagaaa
aaactttgtcaagcctaatgaaaccaaaacttacttttggaaagtgcaacatcatatggcacccactaaagatgagtttgactgcaa
agcctgggcttatttctctgatgttgacctggaaaaagatgtgcactcaggcctgattggaccccttctggtctgccacactaacacac
tgaaccctgctcatgggagacaagtgacagtacaggaatttgctctgtttttcaccatctttgatgagaccaaaagctggtacttcact
gaaaatatggaaagaaactgcagggctccctgcaatatccagatggaagatcccacttttaaagagaattatcgcttccatgcaatc
aatggctacataatggatacactacctggcttagtaatggctcaggatcaaaggattcgatggtatctgctcagcatgggcagcaat
gaaaacatccattctattcatttcagtggacatgtgttcactgtacgaaaaaaagaggagtataaaatggcactgtacaatctctatc
caggtgtttttgagacagtggaaatgttaccatccaaagctggaatttggcgggtggaatgccttattggcgagcatctacatgctgg
gatgagcacactttttctggtgtacagcaataagtgtcagactcccctgggaatggcttctggacacattagagattttcagattacag
cttcaggacaatatggacagtgggccccaaagctggccagacttcattattccggatcaatcaatgcctggagcaccaaggagccct
tttcttggatcaaggtggatctgttggcaccaatgattattcacggcatcaagacccagggtgcccgtcagaagttctccagcctctac
atctctcagtttatcatcatgtatagtcttgatgggaagaagtggcagacttatcgaggaaattccactggaaccttaatggtcttcttt
ggcaatgtggattcatctgggataaaacacaatatttttaaccctccaattattgctcgatacatccgtttgcacccaactcattatagc
attcgcagcactcttcgcatggagttgatgggctgtgatttaaatagttgcagcatgccattgggaatggagagtaaagcaatatcag
atgcacagattactgcttcatcctactttaccaatatgtttgccacctggtctccttcaaaagctcgacttcacctccaagggaggagt
aatgcctggagacctcaggtgaataatccaaaagagtggctgcaagtggacttccagaagacaatgaaagtcacaggagtaacta
ctcagggagtaaaatctctgcttaccagcatgtatgtgaaggagttcctcatctccagcagtcaagatggccatcagtggactctcttt
tttcagaatggcaaagtaaaggtttttcagggaaatcaagactccttcacacctgtggtgaactctctagacccaccgttactgactc
gctaccttcgaattcacccccagagttgggtgcaccagattgccctgaggatggaggttctgggctgcgaggcacaggacctctac
3xflag (seq E)
shPolyA (seq V)
3′ ITR (seq H)

The present invention will now be illustrated by means of non-limiting examples.

Materials and Methods

Generation of AAV Vector Plasmids

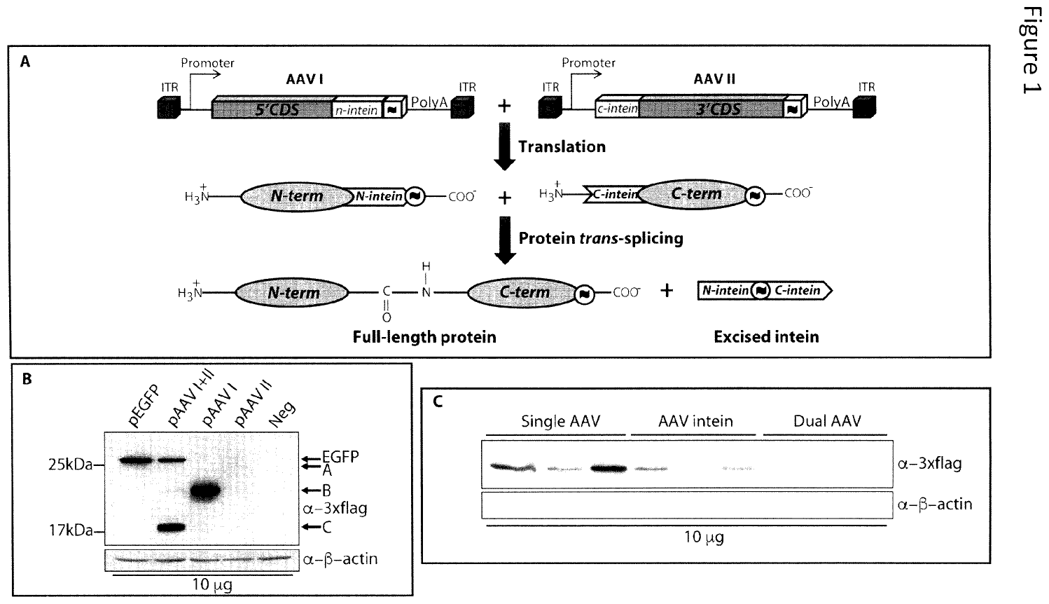

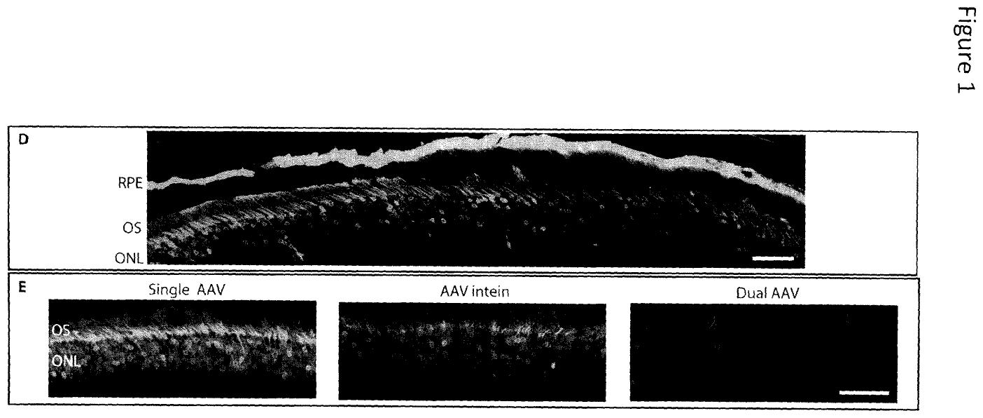

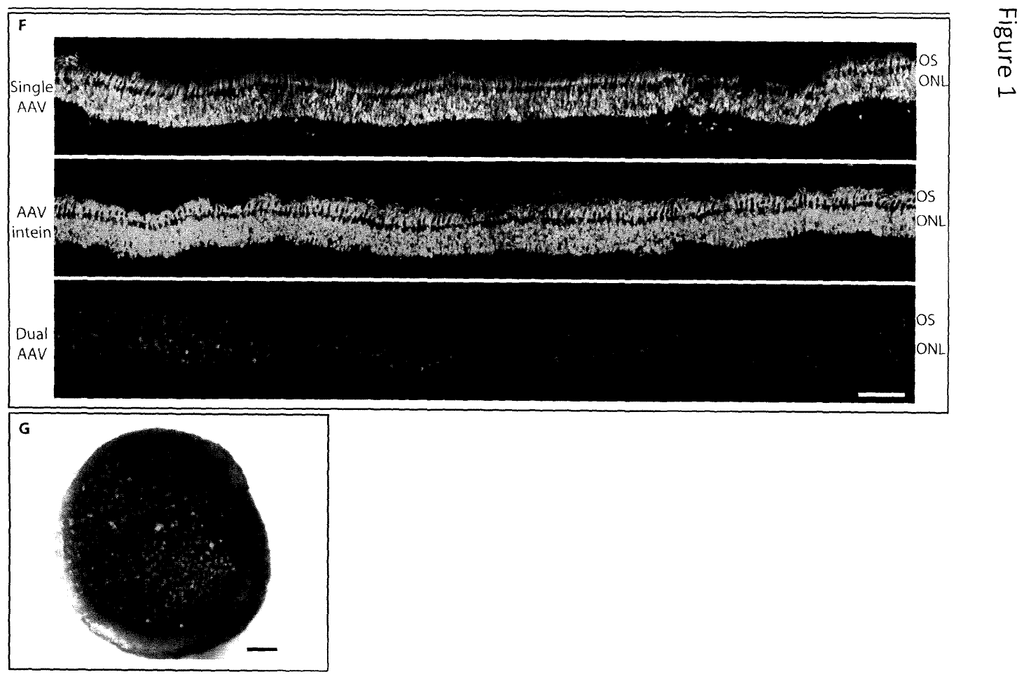

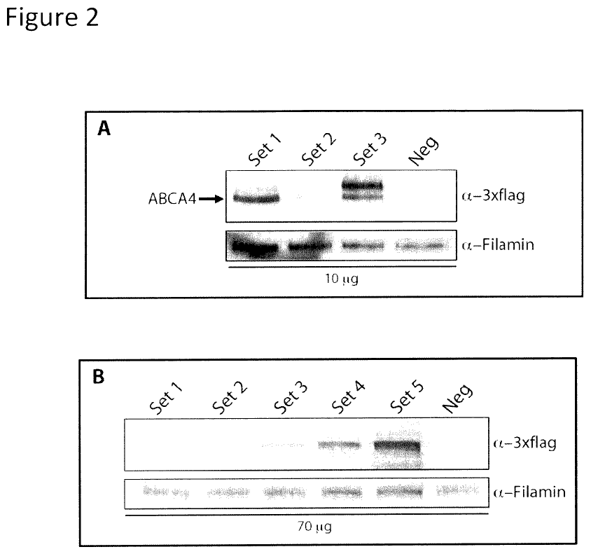

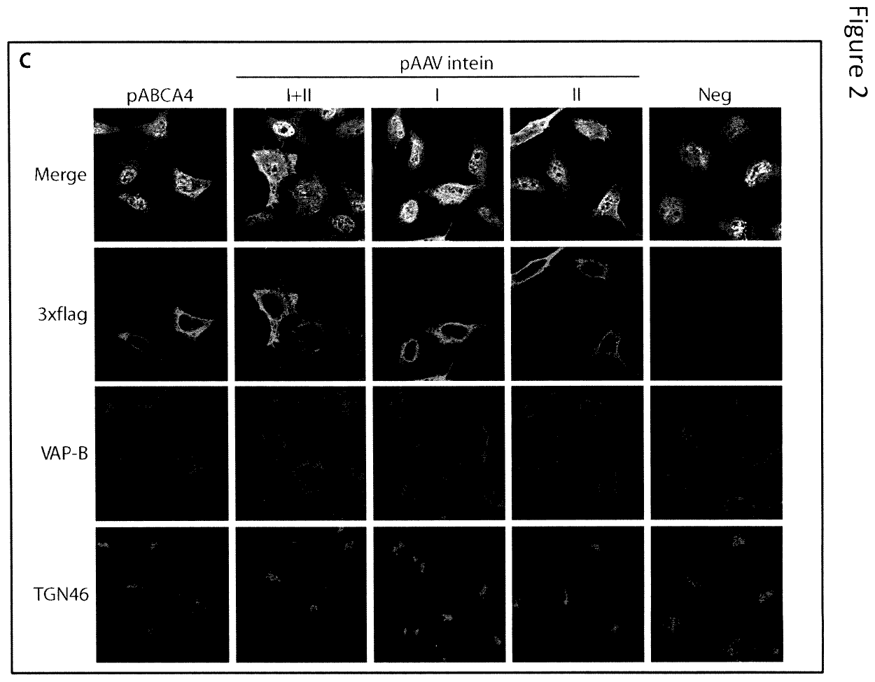

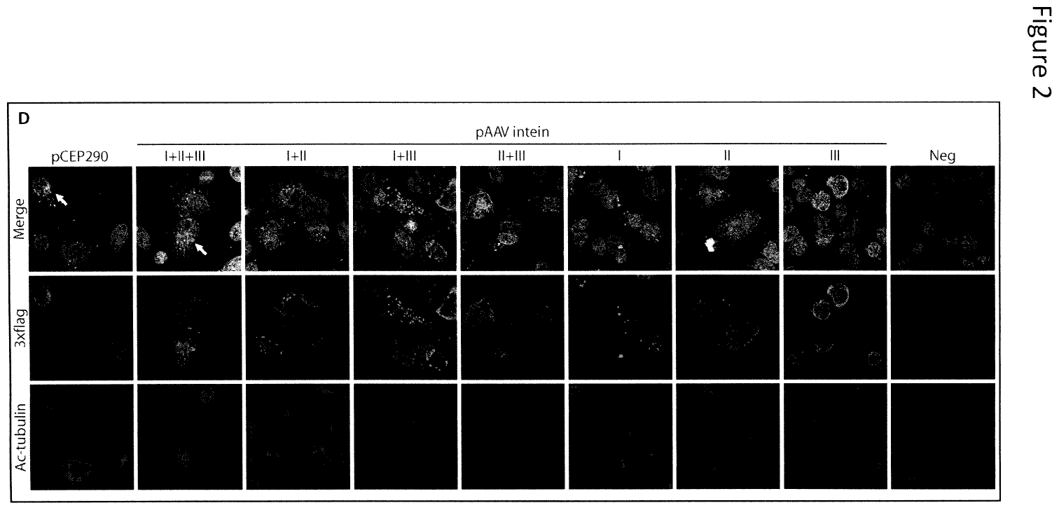

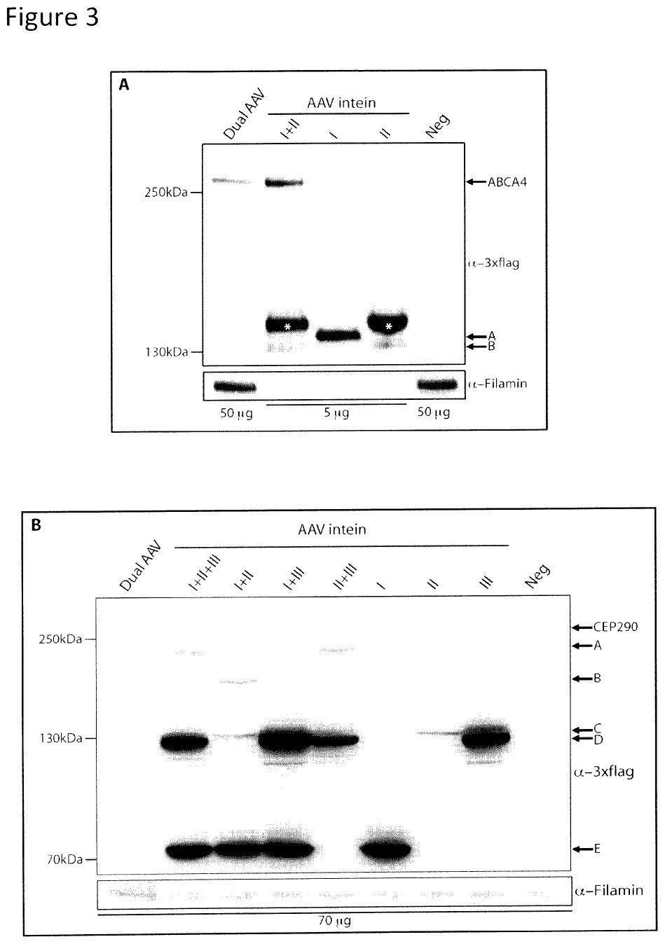

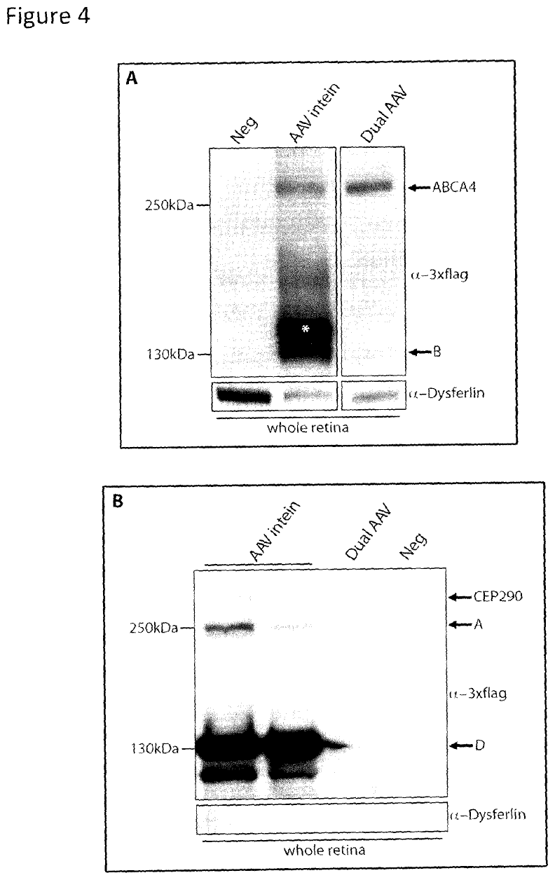

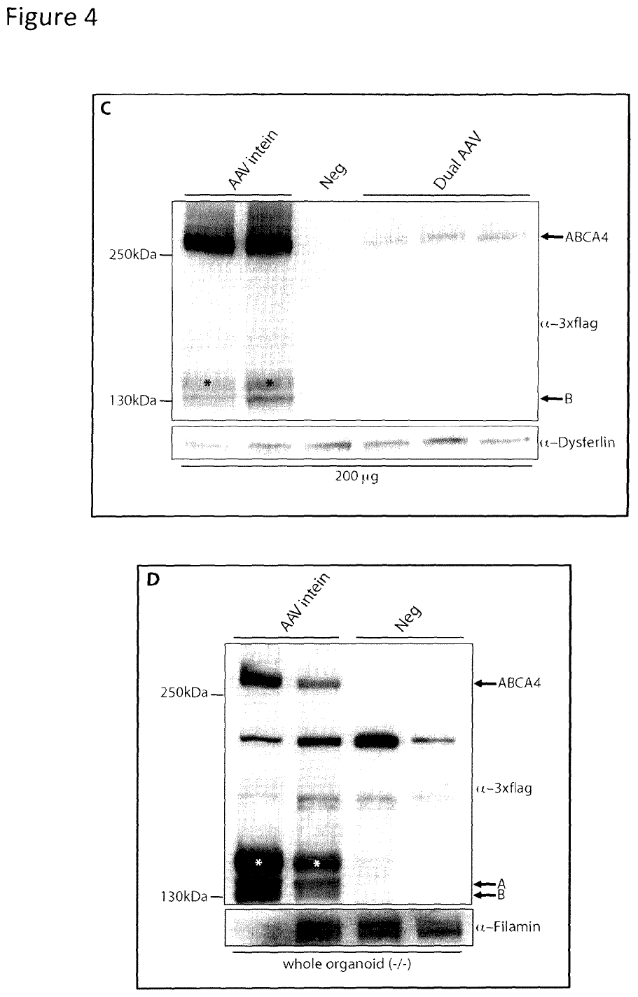

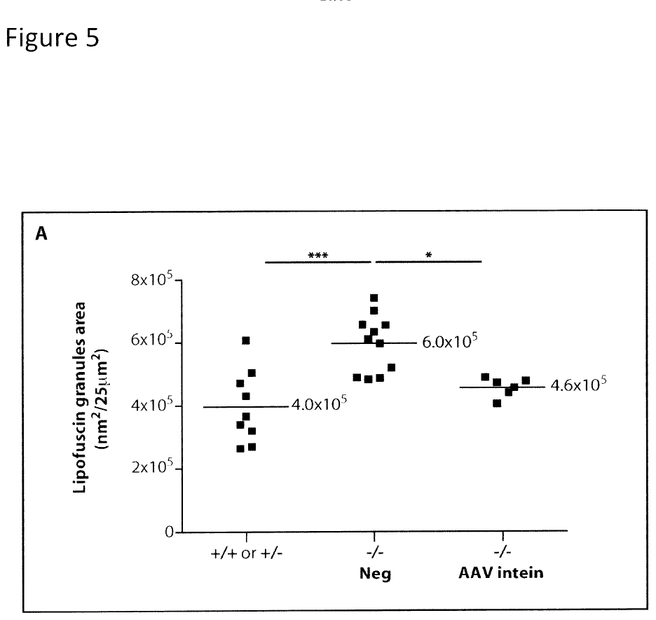

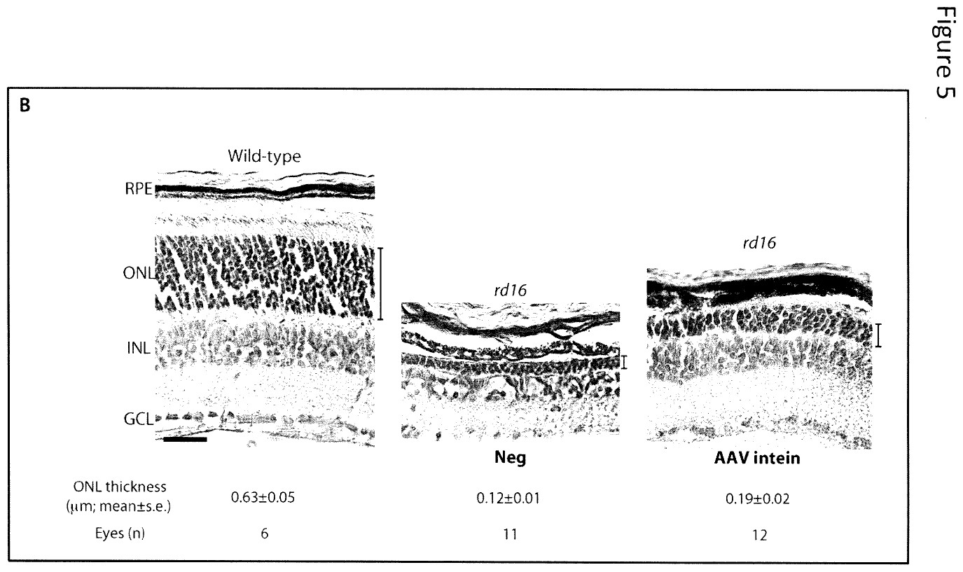

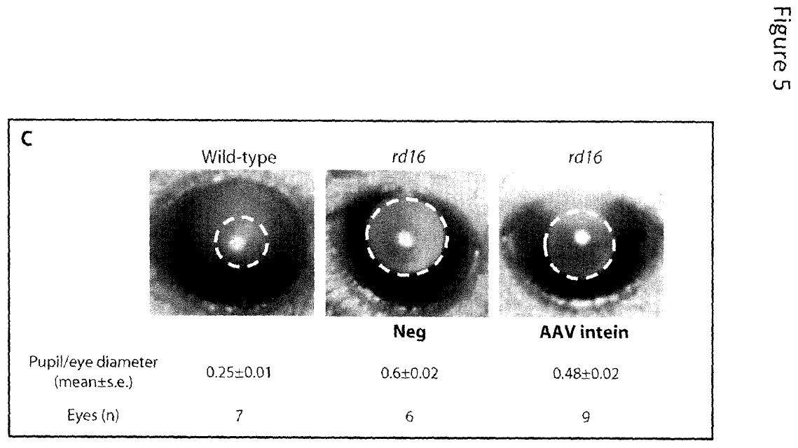

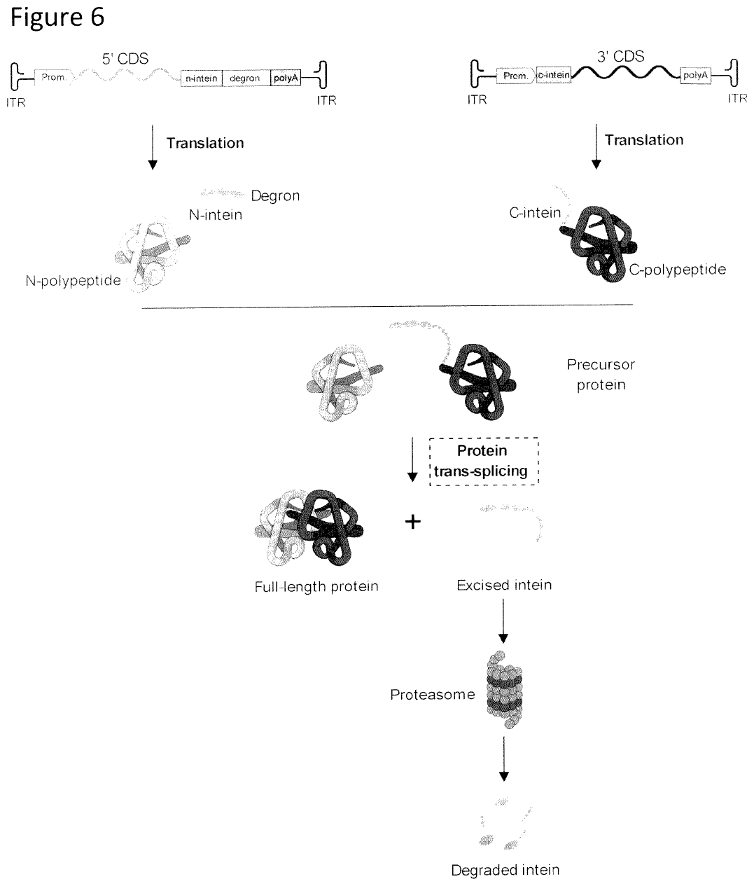

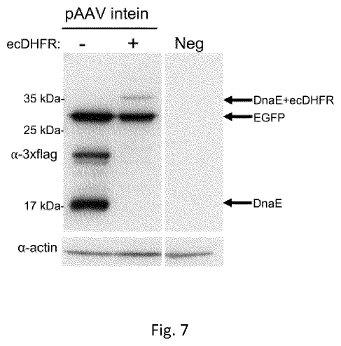

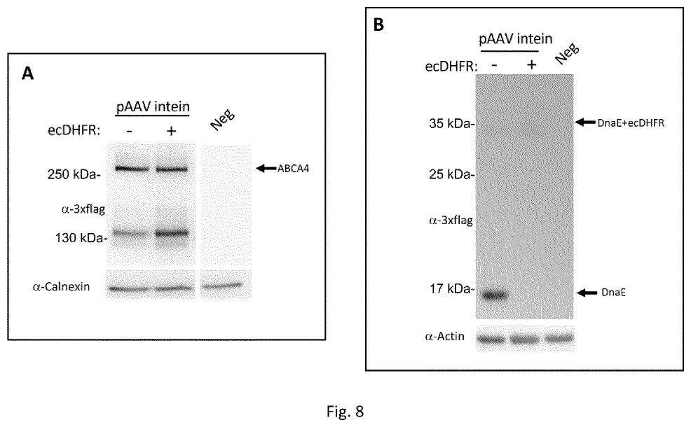

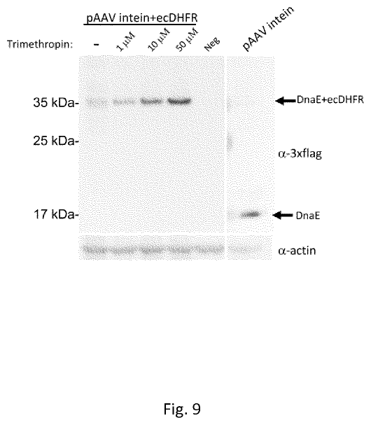

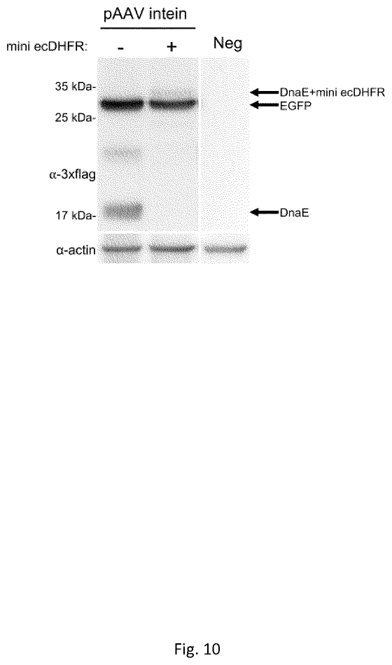

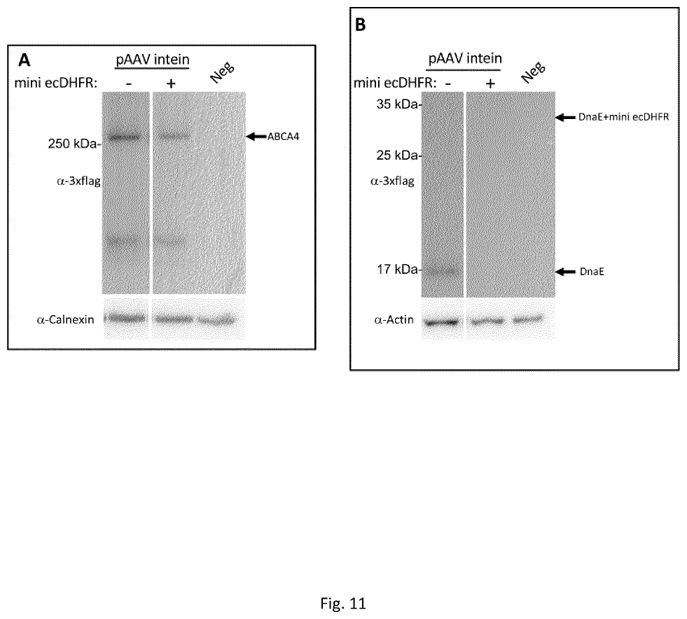

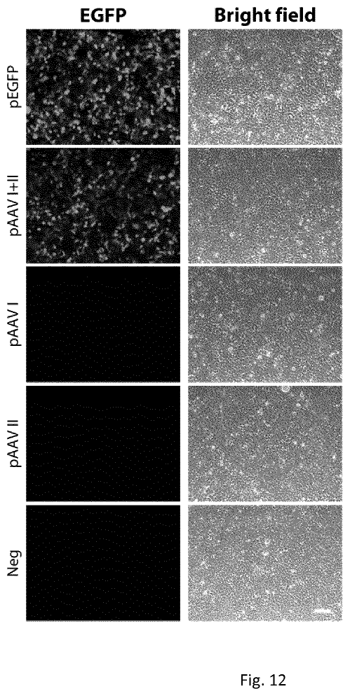

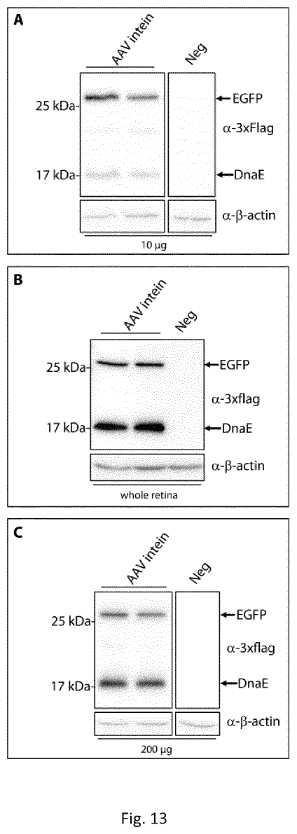

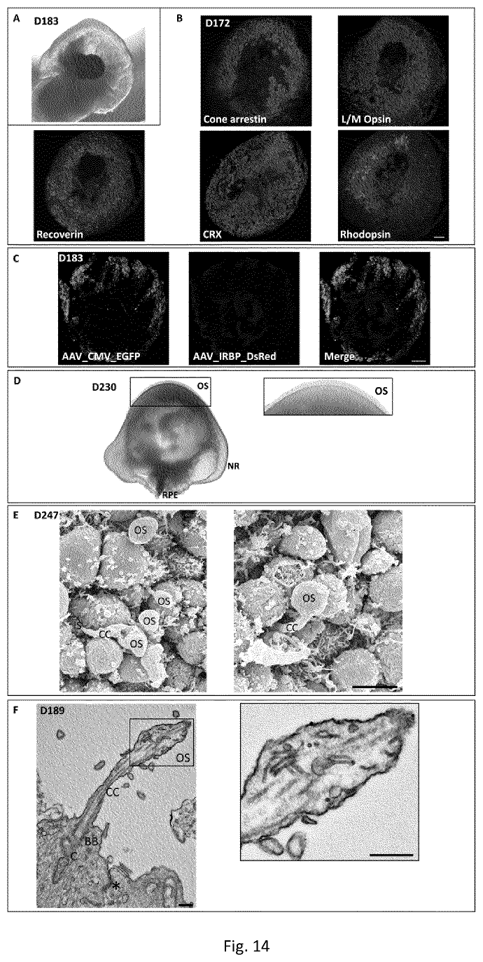

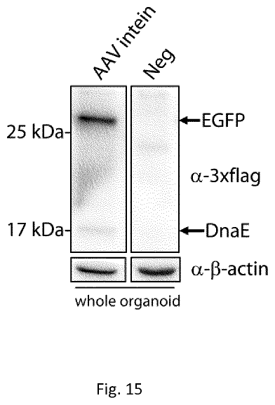

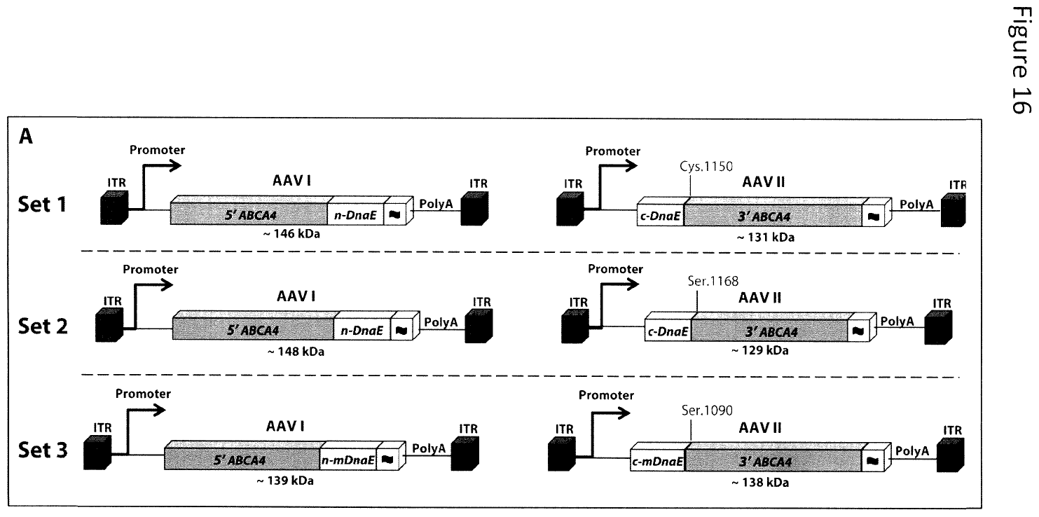

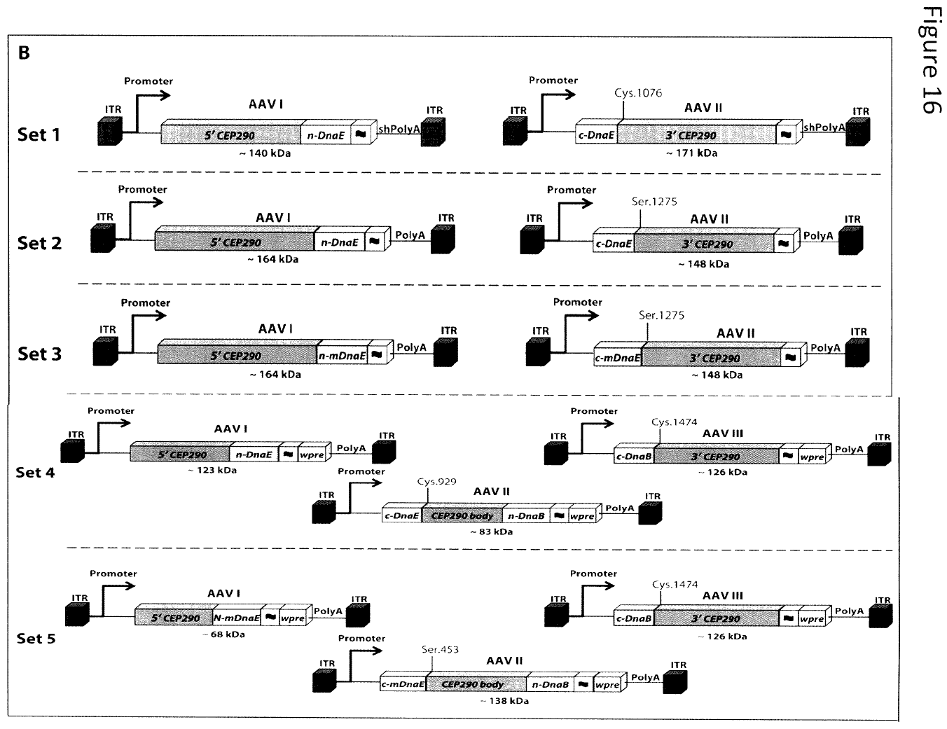

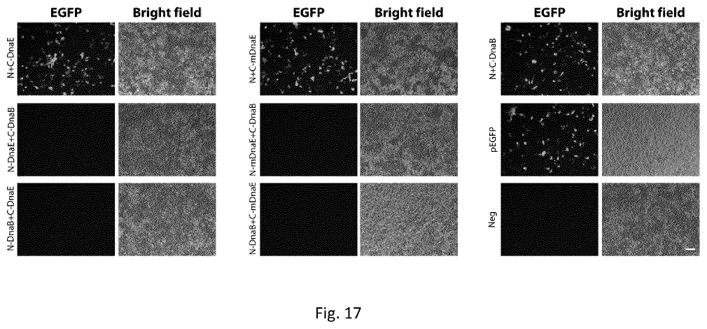

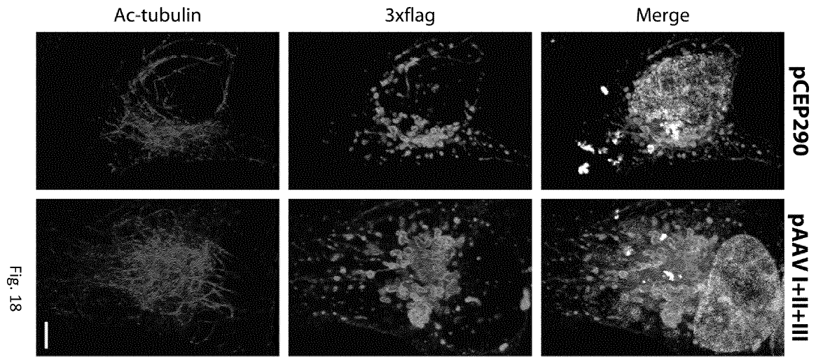

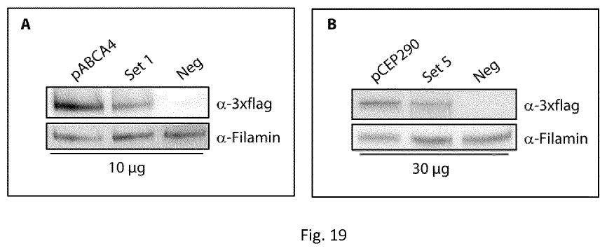

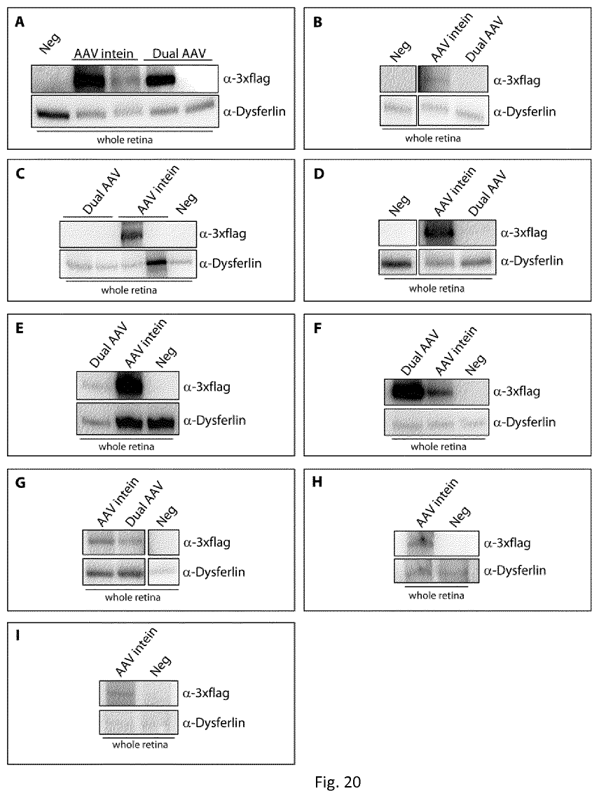

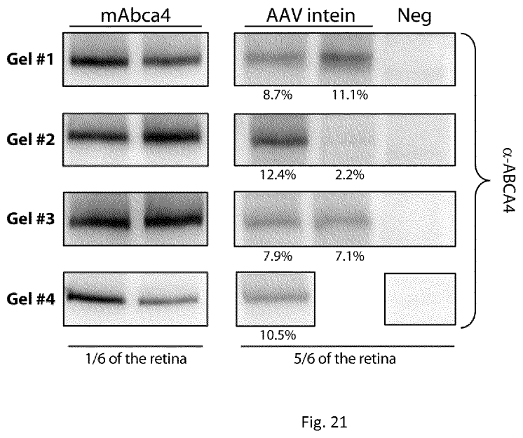

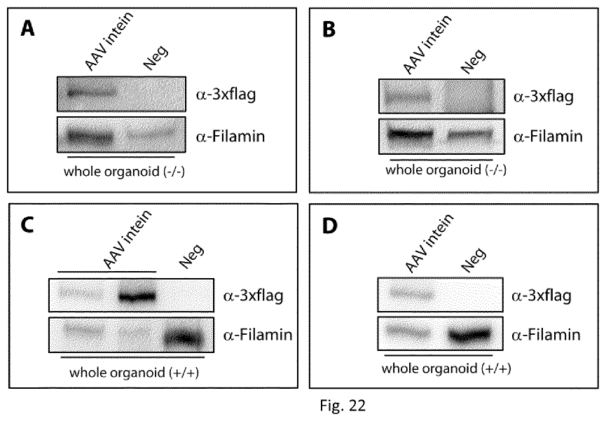

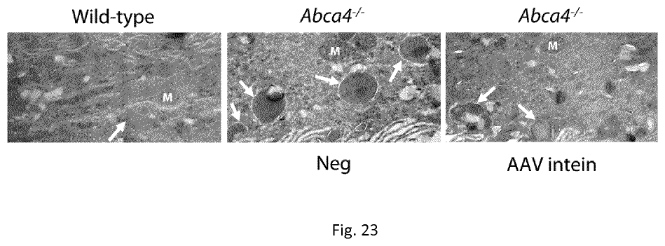

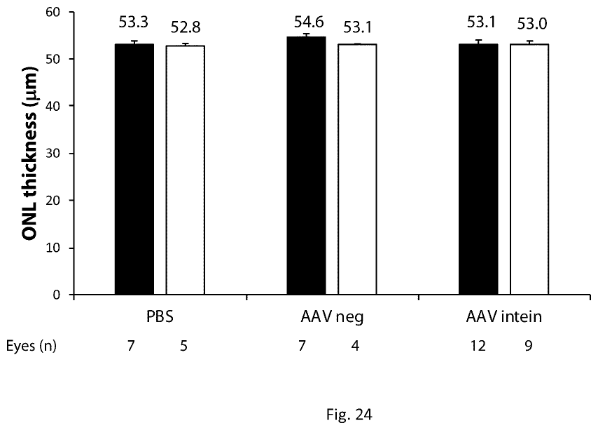

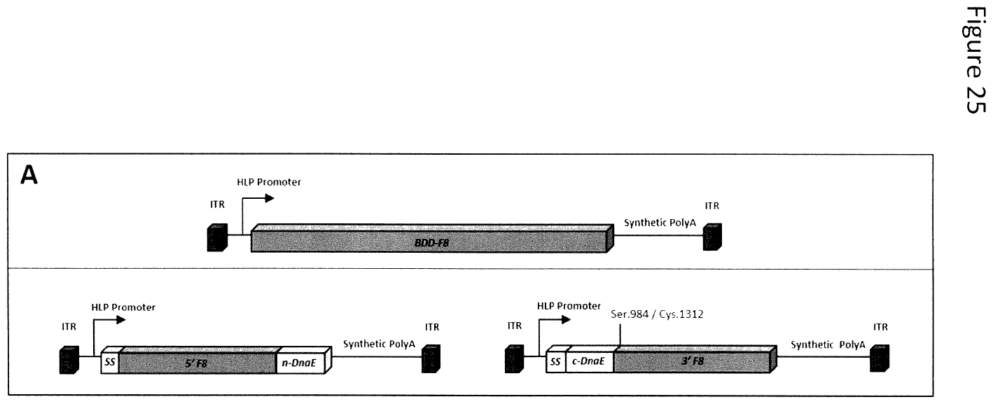

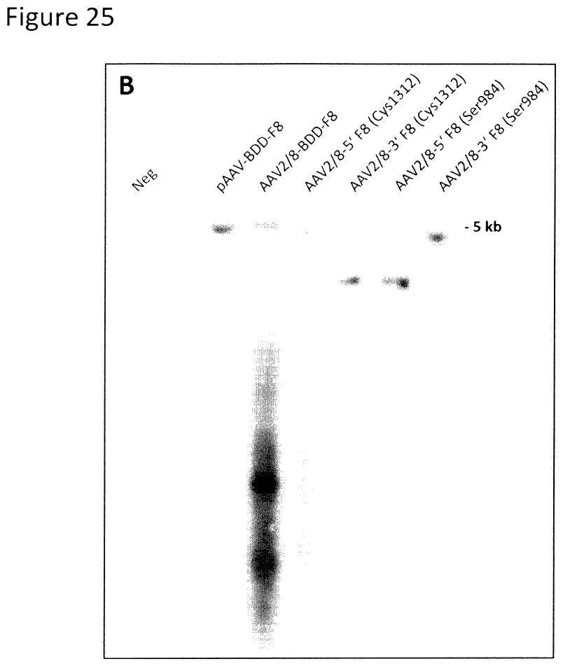

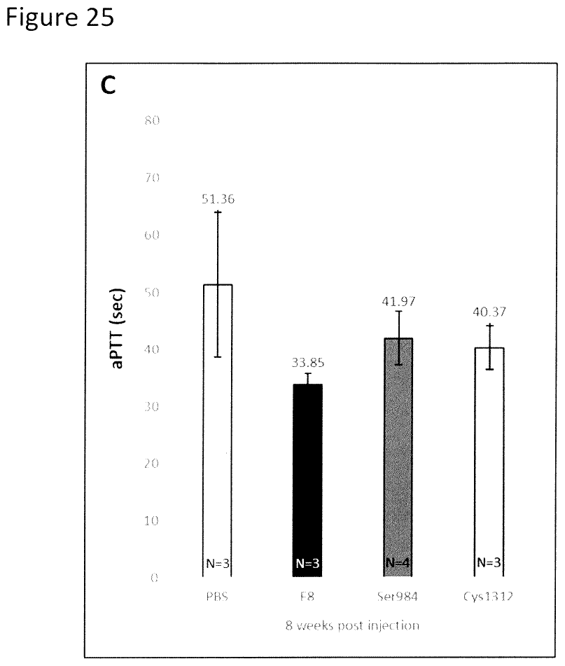

The plasmids used for AAV vector production derived from either the pAAV2.1 (36) or the pZac (37) plasmids that contain the ITRs of AAV serotype 2. The AAV intein plasmids were designed as detailed in FIG. 1A and in Figure S5. The EGFP protein was split at the amino acid (a.a.) C71. The ABCA4 protein was split in the large cytoplasmic domain CD1 (34, 35) at a.a. C1150 (Set 1), a.a. S1168 (Set 2) and a.a. C1090 (Set 3). While a.a. C1150 (Set 1) and S1168 (Set 2) fall within regions that are not associated with a known ABCA4 function, C1090 is included in the ABCA4 nucleotide binding domain which spans from a.a.929 to a.a.1148. All CEP290 splitting points fall in coiled-coil domains(36): when CEP290 was split in two polypeptides this occurred at either a.a. C1076 (Set 1) or S1275 (Set 2-3), when it was split in three polypeptides this was at either a.a. C929 and C1474 (Set 4) or a.a. S453 and C1474 (Set 5).

Inteins included in the plasmids were either the intein of DnaE from Nostoc punctiforme (Npu)(27, 28), or an intein composed of mutated N- and C-inteins from DnaE of Npu and Synechocystis sp. strain PCC6803 (Ssp), respectively(30), or the intein of DnaB from Rhodothermus marinus (Rma)(29). The plasmids used in the study were under the control of either the ubiquitous cytomegalovirus (CMV) (38) and short CMV (39) promoters or the photoreceptor-specific human G protein-coupled receptor kinase 1 (GRK1) 40 promoters. Plasmids encoding for EGFP and CEP290 included the bovine growth hormone polyadenylation signal (bGHpA) while plasmids encoding for ABCA4 included the simian virus 40 (SV40) polyadenylation signal.

AAV Vector Production and Characterization

AAV vectors were produced by the TIGEM AAV Vector Core by triple transfection of HEK293 cells as already described (14, 41). No differences in vector yields were observed between AAV vectors including or not intein sequences.

Transfection and AAV Infection of Cells

HEK293 cells were maintained and transfected using the calcium phosphate method (1 μg of each plasmid/well in 6-well plate format) as already described (14). For the experiments described in Figure S9, an amount of plasmid encoding for the full-length gene corresponding to the same number of molecules contained in 1 μg of AAV intein plasmids was used. The total amount of DNA transfected in each well was kept equal by addition of a scramble plasmid where needed.

HeLa cells used for experiments in FIGS. 2C and 2D, were transfected (either 1 or 0.5 μg of each plasmid/well in 24-well plate format) using Lipofectamine LTX (Invitrogen). AAV infections were performed as already described (14).

iPSCs and Retinal Differentiation Culture

Human induced pluripotent stem cells (iPSCs) were derived from fibroblasts which were cultured from skin biopsies using methods described in(42). The STGD1 cell lines carry either the ABCA4 compound heterozygous variants c.4892T>C and c.4539+2001G>A, also described in(43), or the compound heterozygous variants c.[2919-?_3328+?del; 4462T>C] and c.5196+1137G>A. c.[2919-?_3328+?del; 4462T>C] is an allele that consists of two variations. c.2919-? 3328+?del constitutes a deletion of exons 20, 21 and 22 as well as unknown segments of introns 19 and 22. This deletion was found in a cis configuration with c.4462T>C. iPSCs were maintained on matrigel (#354277, Corning® Matrigel® hESC-Qualified Matrix; Corning, N.Y.)-coated 6 well plates containing mTeSR™ medium (#85850; Stem cell technologies). Cells were passaged at around 80% confluence using 0.5 mM EDTA (#AM9260G; Ambion) for 2-6 minutes. Retinal differentiation was based on a combination of previously described protocols (44, 45). Briefly, iPSCs were plated in V-bottomed 96-well plates (9,000 cells/well) containing RevitaCell Supplement (#A-2644501; Gibco, ThermoFisher) and 1% matrigel to induce aggregates formation. Aggregates were then cultured to generates 3D retinal organoids as reported in (46).

Western Blot Analysis and ELISA

Samples (HEK293 cells, retinas and retinal organoids) were lysed in RIPA buffer to extract EGFP, ABCA4 and CEP290 proteins. Lysis buffers were supplemented with protease inhibitors (Complete Protease inhibitor cocktail tablets; Roche, Basel, Switzerland) and 1 mM phenylmethylsulfonyl. After lysis ABCA4 samples were denatured at 37° C. for 15 minutes in 1× Laemmli sample buffer supplemented with 2 M urea. EGFP and CEP290 samples were denatured at 99° C. for 5 minutes in 1× Laemmli sample buffer. Lysates were separated by either 12% (for EGFP sample) or 6% (for ABCA4 and CEP290 samples) SDS-polyacrylamide gel electrophoresis. The antibodies used for immuno-blotting are as follows: anti-3× flag (1:1000, A8592; Sigma-Aldrich, Saint Louis, Mo., USA) to detect the EGFP, ABCA4 and CEP290 proteins; anti-ABCA4 (1:500, LS-C87292; LifeSpan BioSciences, Inc. Seattle, USA) to detect ABCA4; anti-Filamin A (1:1000, #4762; Cell Signaling Technology, Danvers, Mass., USA); anti-β-Actin (1:1000, NB600-501; Novus Biological LLC, Littleton, Colo., USA) to detect Filamin A, β-Actin used as loading controls in the in vitro experiments; anti-Dysferlin (1:500, Dysferlin, clone Ham1/7B6, MONX10795; Tebu-bio, Le Perray-en-Yveline, France) to detect Dysferlin used as loading controls in in vivo experiments. The quantification of EGFP, ABCA4 and CEP290 bands detected by Western blot was performed using ImageJ software (free download is available at http://rsbweb.nih.gov/ij/).

For experiments shown in FIG. 21, retinal lysates from both Abca4−/− mice injected with AAV intein vectors and control littermate Abca4+/− mice were lysed in 30 [symbol] l of lysis buffer, as described above, and either 25 or 5 [symbol] [symbol] l of lysate, respectively, were used for Western blot using anti-ABCA4 antibodies (LS-C87292; epitope conservation: 100% for human ABCA4; 86% for murine Abca4). The amounts of ABCA4 in retinal lysates, measured by quantification of bands intensity using ImageJ software, was then normalized to the volume of retinal lysate loaded on the acrylamide gel. For experiments in FIG. 9, HEK293 cells were treated daily with increased dose of trimethoprim (T7883, Sigma-Aldrich) as reported in the figure.

The ELISA was performed either on cells or on mouse and pig retinal lysates using the Max Discovery Green Fluorescent Protein Kit ELISA (Bioo Scientific Corporation, Austin, Tex., USA).

Southern Blot Analyses of rAAV Vector DNA.

DNA was extracted from 1.5 to 6×10¹⁰ viral particles (measured as GC). To digest unpackaged genomes, the vector solution was incubated with 30 μl of DNase (Roche) in a total volume of 300 μl, containing 50 mM Tris, pH 7.5, and 1 mM MgCl₂ for 2 hour at 37° C. The DNase was then inactivated with 50 mM EDTA, followed by incubation at 50° C. for 1 hour with proteinase K and 2.5% N-lauryl-sarcosil solution to lyse the capsids. The DNA was extracted twice with phenol-chloroform and precipitated with 2 volumes of ethanol 100% and 10% sodium acetate (3 M) and 1 [symbol] l of Glycogen (20 [symbol] g). Alkaline agarose gel electrophoresis was performed as previously described (Sambrook, J., and Russell, D. W. 2001. Molecular cloning: a laboratory manual. Cold Spring Harbor Laboratory Press. Cold Spring Harbor, N.Y., USA. 999 pp). Markers were produced by double digestion of the pF8-V3 with SmaI, to produce a band of 5102 bp. A probe specific to the HLP promoter was used.

Activated Partial Thromboplastin Time (aPTT)

Nine parts of blood were collected by retro-orbital withdrawal into one part of buffered trisodium citrate 0.109M (BD, Franklin Lakes, N.J., USA). Blood plasma was isolated by centrifuging the samples at 13000 rpm for 15 minutes.

aPTT was measured on Coatron M4 (Teco, Binde, Germany) using the aPTT program following the manufacturer's manual.

Immunoprecipitation and Liquid Chromatography/Mass Spectrometry Analysis

Cells were plated in 100 mm plates (1×10⁷ cells/plates) and transfected in suspension with either AAV-EGFP or ABCA4 intein plasmids using the calcium phosphate method (20 μg of each plasmid/plate). Cells were harvested 72 hours post-transfection and both EGFP and ABCA4 proteins were immunoprecipitated using anti-flag M2 magnetic beads (M8823; Sigma-Aldrich), according to the manufacturer instructions. Proteins were eluted from the beads by incubation for 15 minutes in sample buffer supplemented with 4 M urea at 37° C. Proteins were then loaded on 12% (for EGFP) or 6% (for ABCA4) SDS-polyacrylamide gel electrophoresis. Twenty-six and thirty protein bands (from HEK293 cells transfected 2 and 3 times independently with AAV-EGFP and ABCA4 intein plasmids, respectively) cut after staining with Coomassie Blue were used for protein sequencing (Creative proteomics, Shirley, N.Y.). Briefly, 3 gel slides were used for digestion by each of the following enzymes: Trypsin, Chymotrypsin, Glu-C, Arg-C, Asp-N and Lys-N. Pepsin was additionally used to digest ABCA4. The resulting peptides were identified and quantified using nanoscale Liquid Chromatography coupled to tandem Mass Spectrometry (nano LC-MS/MS) analysis. Mass spectrometry data obtained were analyzed using PEAKS STUDIO 8.5. The inventors achieved 100% of protein sequence coverage for both EGFP and ABCA4 proteins.

Animal Models

Animal were housed at the TIGEM animal facility (Naples) and maintained under a 12 hours light/dark cycle. C57BL/6J mice were purchased from Envigo (Italy).

Albino Abca4^{-/-} mice were generated through successive crosses and backcrosses with BALB/c mice (homozygous for Rpe65 Leu450) and maintained inbred. BXD24/TyJ-Cep290^{rd16}/J (referred as rd16) mice were imported from The Jackson Laboratory (JAX stock #000031). The rd16 mouse carries an in-frame deletion of 897 bp encompassing exons 35-39 (46). The mice were maintained by crossing homozygous females with homozygous males. The hemophilic mice B6; 129S-F8^{tm1Kaz}/J (referred as F8tm1) were imported from The Jackson Laboratory (JAX stock #004424). The F8tm1 mouse has a neomycin resistance cassette that replaces 293 bp of sequence, including 7 bp at the 3′ end of exon 16 and 286 bp at the 5′ end of intron 16. The mice colony was maintained by crossing homozygous females with hemizygous males.

The Large White female pigs (Azienda Agricola Pasotti, Imola, Italy) used in this study were registered as purebred in the LWHerd Book of the Italian National Pig Breeders' Association and were housed at the Centro di Biotecnologie A.O.R.N. Antonio Cardarelli (Naples, Italy) and maintained under a 12 hours light/dark cycle.

Subretinal Injection of AAV Vectors in Mice and Pigs

This study was carried out in accordance with the Association for Research in Vision and Ophthalmology Statement for the Use of Animals in Ophthalmic and Vision Research and with the Italian Ministry of Health regulation for animal procedures. All procedures on mice were approved from the Italian Ministry of Health; Department of Public Health, Animal Health, Nutrition and Food Safety on Mar. 6, 2015.

Subretinal injections in mice and pigs were performed as previously described (for instance in 14). Mouse eyes were injected with either 1 μl or 0.5 μl (for rd16 pups) of vector solution. The AAV2/8 doses varied across different mouse experiments, as described in the Results section. Pig eyes were injected with 2 adjacent subretinal blebs of 100 μl of AAV2/8 vector solution. The AAV2/8 dose was 2×10\hat{ }11 GC of each vector/eye, thus co-injection of two AAV vectors resulted in a total dose of 4×10\hat{ }11 GC/eye.

Histology, Light and Fluorescence Microscopy

To evaluate EGFP expression in histological sections, retinal organoids, eyes from both C57BL/6J mice and Large White pigs were fixed and sectioned as already described. EGFP positive cryosections, mounted with Vectashield with DAPI (Vector Lab Inc., Peterborough, UK), were analyzed under the confocal LSM-700 microscope (Carl Zeiss, Oberkochen, Germany), using appropriate excitation and detection setting and acquired at 40× magnification. Due to the prevalence of red-green color blindness, to avoid the presence of red and green together colors of the original images have been modified in FIG. 14.

To evaluate the thickness of the outer nuclear layer in rd16 mice injected with AAV CEP290 intein vectors, eyes were fixed in 4% paraformaldehyde (PFA) overnight followed by dehydration in serial ethanols and then embedded in paraffin blocks. Serial cross-sections from rd16 mice (10 μm) were cut along the horizontal meridian, progressively distributed on slides, and stained with hematoxylin and eosin (H&E). Then, the sections were analyzed under the microscope (Leica Microsystems GmbH; DM5000) and acquired at 20× magnification. For each eye one image from the temporal injected side of a slice in the central region of the eye was used for the analysis. Three measurements of the ONL thickness were taken, in each image, by an operator masked to the genotype/treatment group, using the “freehand line” tool of the ImageJ software.

Immunofluorescence Analysis

HeLa cells transfected with either ABCA4 or CEP290 AAV intein plasmids were fixed 24 hours post-transfection in 4% PFA for 10 minutes. Cells were blocked in blocking buffer (0.05% Saponin, 0.5% BSA, 50 mM NH₄Cl, 0.02% NaN₃ in PBS, pH7.2) for 30 minutes and then incubated as follows:
for 1 hour with anti-FLAG M2 antibody (F1804, Sigma-Aldrich) to detect ABCA4 proteins; with anti-VAP-B antibody [produced in Antonella De Matteis lab ((47)], to stain the endoplasmic reticulum and with TGN46 (AHP-499, Serotech) to stain the Trans-Golgi network. After washing in PBS, cells were incubated with secondary antibodies for 30 min: goat anti-mouse Alexa Fluor 568; goat anti-rabbit Alexa Fluor 488, donkey anti-sheep Alexa Fluor 633 directed against anti-FLAG, -VAP-B and -TGN46 antibodies, respectively.
overnight with anti-FLAG antibody (F7425, Sigma-Aldrich) to detect CEP290 proteins, and with anti-Acetylated tubulin antibody (T6793, Sigma-Aldrich) to stain the microtubules. After washing in PBS, cells were incubated with appropriate secondary antibodies for 1 hour: goat anti-rabbit Alexa Fluor 594 and donkey anti-mouse Alexa Fluor 488, directed against anti-FLAG and —Ac-Tubulin antibody, respectively.

Nuclei were stained with DAPI. Due to the prevalence of red-green color blindness, to avoid the presence of red and green together colors of the original images have been modified in both FIGS. 2 C-D and FIG. 18.

The antibodies used for immunofluorescence of human retinal organoids are as follows: anti-human cone-arrestin (CAR) (50, 51) (1:10000, ‘Luminaire founders’ hCAR; gift from Dr Cheryl M. Craft, Doheny Eye Institute, Los Angeles, Calif., USA); anti-Opsin, Red/Green (1:200, AB5405; Merck Millipore, Darmstadt, Germania); anti-Recoverin (1:500, AB5585; Merck Millipore); anti-CRX (A-9, 1:250, sc377138; Santa Cruz Biotechnology, Dallas, Tex., USA); anti-Rhodopsin (1D4, 1:200, ab5417, Abcam, Cambridge, Mass., USA).

Transmission and Scanning Electron Microscopy Analyses

For electron microscopy (EM) analyzes Abca4^{-/-} mice at 3 months after AAV subretinal injection were dark-adapted overnight and then eyes were harvested. Eyes were fixed in 0.2% glutaraldehyde (GA)-2% PFA in 0.1 M PHEM buffer pH 6.9 for 18 hours and then rinsed in 0.1 M PHEM buffer. Eyes were then dissected under a light microscope to select the temporal injected area of the eyecups. This portion of the eyecups was subsequently embedded in 12% gelatin, infused with 2.3 M sucrose. Cryosections (60 nm) were frozen in liquid nitrogen and cut using a Leica Ultramicrotome EM FC7 (Leica Microsystems). To avoid bias in the attribution of data to the various experimental groups, measurements of the area occupied by lipofuscin granules in the retinal pigment epithelium were performed by an operator masked to the genotype/treatment group using the iTEM software (Olympus SYS, Hamburg, Germany). The area of each lipofuscin granule in each field was measured in at least 20 different images (25 μm² areas) using the ‘Free hand polygon’ tool of iTEM software. For scanning electron microscopy (SEM) analysis, retinal organoids were fixed in GA, stained with OsO4, dehydrated in ethanol and dried using critical point drying procedure. Dried specimens were then mounted on SEM specimen stub and coated with a thin layer of gold. Surface three-dimensional organization of the specimens was analyzed, and images were acquired using JEOL 6700F scanning electron microscope (JEOL Ltd., Tokyo, Japan).

For ultrastructure analysis, retinal organoids were fixed overnight with a mixture of 2% PFA and 1% GA in 0.2 M PHEM buffer pH 7.3. After fixation the specimens were post-fixed as previously described. Then they were dehydrated, embedded in epoxy resin and polymerized at 60° C. for 72 hours. Thin serial 60 nm sections were cut at the Leica EM UC7 microtome.

EM images were acquired using a FEI Tecnai-12 electron microscope equipped with a VELETTA CCD digital camera (FEI, Eindhoven, The Netherlands).

Electrophysiological Recordings and Spectral Domain Optical Coherence Tomography

Functional and morphological analysis were performed as already described (14).

Pupillary Light Response

Pupillary light responses from rd16 mice were recorded in dark condition using the TRC-501X retinal camera connected to a charge-coupled device NikonD1H digital camera (Topcon Biomedical Systems, Oakland, N.J.). Mice were exposed to 10 lux light-stimuli for approximately 10 seconds and one picture per eye was acquired using the IMAGEnet software (Topcon Biomedical Systems). For each eye, the pupil diameter was normalized to the eye diameter (from temporal to nasal side).

Statistical Analyses

One-way ANOVA test (parametric test) or Kruskal-Wallis rank sum test (non-parametric test) were performed to determine if there were statistically significant differences between two or more groups of an independent variable on a dependent variable. P-values are as follows: ELISA assay for EGFP protein quantification in vitro (p Kruskal-Wallis=0.006036), in the mouse retina (p ANOVA=0.00585), and in the pig retina (p Kruskal-Wallis=0.009005); FIG. 5A (p ANOVA=0.00585); FIG. 5B (p Kruskal-Wallis=5.547E-5); FIG. 5C (p ANOVA=5.81E-10); ERG analyses (p ANOVA or p Kruskal-Wallis >0.05 at all luminance analysed for both a- and b-wave amplitudes); OCT analysis in FIG. S14 (p ANOVA=0.52 for ABCA4 and p ANOVA=0.965 for CEP290). The statistically significant differences between groups determined with the multiple pairwise-comparison between the means of groups are the following: ELISA assay for EGFP protein quantification in vitro (single AAV versus dual AAV=0.012; AAV intein versus dual AAV=0.012; single AAV versus AAV intein=0.222), in the mouse retina (single AAV versus dual AAV=0.0044; AAV intein versus dual AAV=0.3754; single AAV versus AAV intein=0.0561) and in the pig retina (single AAV versus dual AAV=0.012; AAV intein versus dual AAV=0.012; single AAV versus AAV intein=0.841); FIG. 5A: +/+ versus −/− AAV intein=0.4530; +/+ versus −/−=0.0002; FIG. 5B: wild-type versus rd16 AAV intein=0.00131; FIG. 5C: wild-type versus rd16 AAV intein 1E-07; wild-type versus rd16 neg <1E-06.

### EXAMPLES

### Example 1. AAV-EGFP Intein Reconstitute Full-Length Protein In Vitro

The present inventors tested the efficiency of intein-mediated protein trans-splicing in the retina; two AAV vectors were generated, each encoding either the N- or the C-terminal half of the reporter EGFP protein fused to the N- and C-terminal halves of the DnaE split-intein from Nostoc punctiforme [Npu FIG. 1A], respectively. The EGFP protein was split at the amino acid (a.a.) C71. Each AAV vector included appropriate regulatory elements (i.e. promoter and the bovine growth hormone polyadenylation signal (bGHpA) and a triple flag tag (3× flag) to allow detection of both halves as well as of the full-length reconstituted EGFP protein (FIG. 1A).

AAV-EGFP Dna E intein plasmids were used to transfect human embryonic kidney 293 (HEK293) cells and evaluate the production of single N- and C-terminal halves as well as of the full-length EGFP protein. EGFP fluorescence, comparable to that observed in cells transfected with a single AAV plasmid that encodes full-length EGFP, was detected in cells co-transfected with the AAV-EGFP intein plasmids but not with the single N- and C-terminal AAV-EGFP intein plasmids, as shown in FIG. 12. The presence of trans-spliced EGFP protein of the expected size (˜28 kDa) along with DnaE intein (˜17 kDa) spliced out from the mature protein was confirmed by Western blot (WB) analysis of HEK293 cell lysates only following co-transfection of both AAV-EGFP intein plasmids, as shown in FIG. 1B. In addition, quantification of the intensity of the bands showed that EGFP protein amounts from AAV intein plasmids were 76±37% (n=3 independent experiments) of those observed from a single AAV plasmid. To define the accuracy of protein reconstitution, EGFP was immunopurified from HEK293 cells transfected with the AAV-EGFP intein plasmids and Liquid Chromatography-Mass Spectrometry (LC-MS) analysis was performed to define its protein sequence. The 3539 peptides obtained from proteolytic digestion of this sample, 7 of which included the splitting point (Table 5), covered the whole protein and confirmed that the amino acidic sequence of EGFP reconstituted by AAV intein plasmids precisely corresponds to that of wild-type EGFP.

TABLE 5
Peptides which include the EGFP
splitting point.

| Peptide sequence | Length |
|---|---|
| GVQCFSR | 7 |
| SEQ ID No. 76 | |
| LPVPWPTLVTTLTYGVQCFSRY | 22 |
| SEQ ID No. 77 | |
| PTLVTTLTYGVQCFSR | 16 |
| SEQ ID No. 78 | |
| TYGVQCFSR | 9 |
| SEQ ID No. 79 | |
| YGVQCFSR | 8 |
| SEQ ID No. 80 | |
| VQCFSR | 6 |
| SEQ ID No. 81 | |
| QCFSR | 5 |
| SEQ ID No. 82 | |

C = Cystein 71

### Example 2. AAV-EGFP Intein are More Efficient than Dual AAV Vectors In Vitro

To confirm EGFP protein reconstitution from the AAV intein vectors, HEK293 cells were infected with either AAV2/2-CMV-EGFP DnaE intein or with single and dual AAV vectors that included the same expression cassette. Multiplicity of infection (m.o.i), 5×10\hat{ }4 genome copies (GC)/cell of each vector, which means a similar dose between the 3 systems assuming that dual vectors undergo complete DNA or protein recombination. In order to quantify precisely EGFP amounts, cell lysates were harvested seventy-two hours after infection. EGFP expression was evaluated by both WB and enzyme-linked immunosorbent assay (ELISA): EGFP expression obtained with AAV intein vectors was around half of that achieved with a single AAV (single AAV=0.735±0.2 ng EGFP/μg total lysate, n=5 independent experiments; AAV intein=0.403±0.04 ng EGFP/μg total lysate, n=5 independent experiments) and 10-times higher than that obtained with dual AAV vectors, as shown in FIG. 1C (dual AAV=0.046±0.01 ng EGFP/μg total lysate, n=5 independent experiments). Further, the intensity of full-length EGFP relative to that of excised intein was quantified by WB; their relative abundance was found to be 1:0.2 (n=6 independent experiments, FIG. 13A).

### Example 3. Subretinal Administration of AAV-EGFP Intein Vectors Results in Efficient Full-Length Protein Reconstitution in Both Mouse and Pig Retina

To investigate whether AAV intein-mediated trans-splicing reconstitutes full-length protein expression in the retina, 4-week-old C57BL/6J mice were injected subretinally with AAV2/8-CMV-EGFP Dna E intein vectors (dose of each vector/eye: 5.8×10\hat{ }9 GC). Eyes were harvested 1 month later and analyzed by microscopy analysis. EGFP fluorescence was detected in all eyes in the retinal pigment epithelium and, most importantly, in photoreceptors (FIG. 1D). To compare transgene expression from AAV intein to that of single and dual AAV in photoreceptors, AAV2/8 vectors that encode EGFP under the control of the photoreceptor-specific human G protein-coupled receptor kinase 1 (GRK1) promoter were injected subretinally in 4-week-old C57BL/6J mice (dose of each vector/eye: 5×10\hat{ }9 GC). Eyes were harvested 1-month post-injection and analyzed by either fluorescence microscopy, ELISA or WB.

EGFP fluorescence was detected in the photoreceptor cell layer in eyes injected with all sets of vectors as seen in FIG. 1E. Precise quantification of EGFP protein amounts by ELISA confirmed that AAV intein reconstituted EGFP protein less efficiently than a single AAV and about 3-times more efficiently than dual AAV (single AAV=8.41±2.48 ng EGFP/retina, n=5 eyes; AAV intein=3.72±0.85 ng EGFP/retina, n=7 eyes; dual AAV=1.38±0.43 ng EGFP/retina, n=7 eyes). The relative amounts of full-length EGFP to excised intein following quantification of WB band intensities were 1:3 (n=14 eyes analyzed, FIG. 13B).

The inventors then evaluated the efficiency of AAV intein vectors at transducing photoreceptors in the pig retina, which is an excellent pre-clinical model to evaluate viral vector transduction, due to its size and architecture ((48). Thus, Large White pigs were injected subretinally with single, intein and dual AAV2/8-GRK1-EGFP vectors (dose of each vector/eye: 2×10\hat{ }11 GC, delivered through two adjacent subretinal blebs). Eyes were harvested 1 month post-injection and analyzed by either fluorescence microscopy, ELISA or WB. Notably, AAV intein-mediated EGFP protein reconstitution in the photoreceptor cell layer was higher than that mediated by dual AAV and indistinguishable from single AAV vectors, as assessed by EGFP fluorescence (FIG. 1F). Precise quantification of EGFP in retinal lysates confirmed that AAV intein reconstitutes the protein to quantities that are similar to those achieved with a single AAV and about 3-times higher than those obtained with dual AAV vectors (single AAV=247.5±45.1 ng EGFP/retina, n=5 eyes; AAV intein=227.0±15.7 ng EGFP/retina, n=5 eyes; dual AAV=82.3±9.6 ng EGFP/retina, n=5 eyes). The relative amount of full-length EGFP to excised intein following quantification of WB band intensities were 1:2 (n=8 eyes, FIG. 13C).

### Example 4. Full-Length EGFP is Reconstituted by AAV-Mediated Protein Trans-Splicing in 3D Human Retinal Organoids

As an additional pre-clinical model representative of the human retina, the inventors generated 3D retinal organoids((49, 50) from human induced pluripotent stem cells (iPSCs). Six month-old organoids (FIG. 14A) contained cells stained by mature photoreceptor markers, as shown in FIG. 14B; the organoids were successfully transduced by AAV2 vectors with a photoreceptor-specific promoter, namely AAV2/2 CMV EGFP and AAV2/2 IRBP DsRed vectors, as shown in FIG. 14C by fluorescence analysis. Light (FIG. 14D) and electron (FIG. 14E-F) microscopies show the presence of buds of photoreceptor outer segments. Nine-month old 3D human retinal organoids incubated for 30 days with AAV-GRK1-EGFP intein vectors (dose of each vector/organoid: 1×10\hat{ }12 GC) show EGFP fluorescence (FIG. 1G). WB analysis of retinal organoid lysates (FIG. 15) confirms full-length EGFP expression which was about 5-fold more abundant than excised intein following band intensity quantification (n=4 organoids).

### Example 5. Intein-Mediated Trans Splicing of Large Proteins (Identification of Optimal ABCA4 and CEP90 Splitting Points is Required for Efficient AAV Intein-Mediated Protein Trans-Splicing)

To test whether protein trans-splicing can be developed as a mechanism to reconstitute large therapeutic proteins, the inventors developed AAV-ABCA4 and -CEP290 intein vectors.

ABCA4 and CEP290 were split into either two (AAV I, AAV II) or three (AAV I, AAV II, AAV III) fragments whose coding sequences were separately cloned in single AAV vectors, fused to the coding sequences of the split-inteins N- and C-termini as shown in FIG. 16. The AAV intein vectors included either the ubiquitous short CMV [(shCMV), for all sets] or the GRK1 promoter (set 1 for ABCA4 and set 5 for CEP290).

Splitting points for each protein were selected taking into account both amino acid residue requirements at the junction points for efficient protein trans-splicing 18, 51), as well as preservation of the integrity of critical protein domains, which should favor proper folding and stability of each independent polypeptide, and thus, of the final reconstituted protein. Additional split-inteins were also considered. CEP290 sets in which the protein was split in 3 polypeptides (sets 4 and 5, FIG. 16B) were generated to allow the inclusion of the Woodchuck hepatitis virus Post-transcriptional Regulatory Element [WPRE, (52)] to increase transgene expression. To prevent unwanted trans-splicing between AAV I and AAV III which could reduce the amount of full-length protein generated, sets 4 and 5 included two different split-inteins at the two splitting junctions, specifically DnaB intein from Rhodothermus marinus and either wild-type or a mutated DnaE intein which the inventors show do not cross-react (FIG. 17).

The inventors compared the ability of each set of AAV intein plasmids to reconstitute ABCA4 and CEP290 following transfection of HEK293 cells. WB analysis of cell lysates 72 hours post-transfection showed that full-length ABCA4 and CEP290 proteins of the expected size (˜ 250 kDa and ˜ 290 kDa, respectively) were reconstituted from each set of AAV intein plasmids, although with variable efficiency (FIG. 2A-B). Sets 1 and 5 were found to be the most efficient for ABCA4 and CEP290 protein reconstitution, respectively, and thus used in all the subsequent experiments.

To define the accuracy of protein reconstitution, the inventors immunopurified ABCA4 from HEK293 cells transfected with set 1 and performed LC-MS analysis to define its protein sequence. The 3108 peptides obtained from proteolytic digestion of this sample, 22 of which included the splitting point (Table 6), covered the whole protein and confirmed that the amino acidic sequence of ABCA4 reconstituted by AAV intein plasmids precisely corresponds to that of wild-type ABCA4. The amino acid sequence of ABCA4 reconstituted by AAV intein matches that of wild-type ABCA4. Alignment between the wild-type ABCA4 sequence and peptides identified in the Liquid Chromatography-Mass Spectrometry analysis of ABCA4 reconstituted from AAV inteins was performed.

TABLE 6
Peptides which include the ABCA4
splitting point.

| Peptide sequence | Length |
|---|---|
| KNCFGT | 6 |
| SEQ ID No. 83 | |
| KNCFGTGL (x3) | 8 |
| SEQ ID No. 84 | |
| KNCFGTGLY (x2) | 9 |
| SEQ ID No. 85 | |
| FLKNCFGTGL | 10 |
| SEQ ID No. 86 | |
| KNCFGTGLYLT | 11 |
| SEQ ID No. 87 | |
| KNCFGTGLYLTL | 12 |
| SEQ ID No. 88 | |
| LYCSGTPLFLKNC | 13 |
| SEQ ID No. 89 | |
| YCSGTPLFLKNCF | 13 |
| SEQ ID No. 90 | |
| KNCFGTGLYLTLVR (x7) | 14 |
| SEQ ID No. 91 | |
| KNCFGTGLYLTLVRKM | 16 |
| SEQ ID No. 92 | |
| IAIIAQGRLYCSGTPLFLKNCFGTGLYLT | 29 |
| SEQ ID No. 93 | |
| QGRLYCSGTPLFLKNCFGTGLYLTLVRKMKNIQSQR | 36 |
| SEQ ID No. 94 | |
| GTPLFLKNCFGTGLYLTLVRKMKNIQSQRKGSEGTCSCSS | 40 |
| SEQ ID No. 95 | |

N.B.: C: Cystein 1150

The inventors then assessed the intracellular localization of the protein products of the different intein containing plasmids comparing them to the localization of the full-length protein. Full-length ABCA4 is known to localize at the endoplasmic reticulum (ER) when expressed in cultured cell lines (53, 54). The two ABCA4 polypeptides from set 1 were found to co-localize at the ER, while no-colocalization was found at the Trans-Golgi network (FIG. 2C). A similar localization was observed in cells co-transfected with both AAV intein plasmids, as well as in cells transfected with a plasmid encoding for the full-length ABCA4 protein, thus confirming the predominant localization in the ER of ABCA4 exogenously expressed in cell lines).

As for CEP290, it has been reported that the full-length protein shows a mixed distribution pattern with a predominant punctate and a minor fibrillar pattern (55). The dissection of the domains responsible for the subcellular targeting of CEP290 showed that N-terminal domain (a.a. 1-362) targets the protein to vesicular structures thanks to its ability to interact with membranes, while a region near the C-terminus of CEP290, encompassing much of the protein's myosin-tail homology domain, mediates microtubule binding (a.a. 580-2479) and when expressed as truncated form has a prominent fibrillar distribution coincident with acetylated tubulin (Ac-Tub)). In agreement with Drivas et al., immunofluorescence analysis on HeLa cells transfected with either AAV I, II or III intein plasmids singularly or co-transfected with AAV I+II, AAV I+III and AAV II+III showed that products from AAV I and AAV II have a predominant punctate pattern while that from AAV III (encompassing protein's myosin-tail homology domain) shows a fibrillar pattern and is the only one to completely colocalize to Ac-tub (FIG. 2D). Thus, products from AAV I+II have a predominant punctate pattern while those from AAV I+III and AAV II+III have a combined microtubule fibrillar and punctate pattern. Cells co-transfected with the three AAV CEP290 intein plasmids showed a predominant punctate signal partially aligned along microtubules which is comparable to the signal observed in cells transfected with a plasmid encoding for the full-length CEP290 protein (FIG. 2D and FIG. 18).

The present inventors then compared the amount of protein obtained with the best set of AAV-ABCA4 and -CEP290 intein plasmids to those obtained from a single AAV plasmid encoding the corresponding full-length protein. To this aim, HEK293 cells were transfected with same equimolar amounts of either the single or the AAV intein plasmids and 72 hours after transfection cell lysates were analyzed by WB (FIG. 19). Quantification of bands' intensity showed that ABCA4 and CEP290 expression from AAV intein plasmids was 61±4% (n=3 independent experiments) and 58±4% (n=3 independent experiments) of that observed with the corresponding single AAV plasmids, respectively.

### Example 6. AAV Intein Vectors Mediate Expression of Large Therapeutic Proteins In Vitro and in the Retina

The inventors compared the efficiency of AAV intein-mediated large protein reconstitution to that of dual AAV vectors both in vitro and in the mouse and pig retina. HEK293 cells were infected with either AAV2/2 dual or intein vectors encoding for either ABCA4 (set 1) or CEP290 (set 5) (m.o.i: 5\hat{ }10\hat{ }4 GC/cell of each vector) and cell lysates were analyzed 72 hours later by WB. As shown in FIGS. 3A and 3B, both AAV-ABCA4 and -CEP290 intein vectors mediated large protein reconstitution more efficiently than dual AAV vectors. As expected, in addition to full-length proteins, shorter polypeptides derived from either the single AAV intein vectors (in the case of both ABCA4 and CEP290) or from trans-splicing occurring between AAV II and AAV III (in the case of CEP290) were observed (FIGS. 3A and 3B).

Further, 4-week-old wild-type mice were injected subretinally with AAV-GRK1-ABCA4 or -CEP290 intein (set 1 and 5, respectively) compared to dual vectors (dose of each ABCA4 vector/eye: 3.3×10\hat{ }9 GC, dose of each CEP290 vector/eye: 1.1×10\hat{ }9 GC). Animals were sacrificed 4-7 weeks post-injection, and protein expression in retinal lysates was evaluated by WB. Full-length proteins were detected in 10/11 (91%) of AAV-ABCA4 intein-injected eyes (FIGS. 4A and 20) and in 5/10 (50%) of AAV-CEP290 intein-injected eyes (FIG. 4B). Conversely, full-length protein expression was evident in 5/9 (56%) and in 0/5 eyes injected with ABCA4 and CEP290 dual AAV vectors, respectively. Similarly to what observed in vitro, polypeptides derived from the single AAV intein vectors (in the case of both ABCA4 and CEP290) and from trans-splicing occurring between AAV II and AAV III (in the case of CEP290) were detected (FIGS. 4A and 4B).

To investigate the efficiency of protein reconstitution mediated by AAV intein relative to endogenous, 1-4-month-old Abca4^{-/-} mice were injected subretinally with AAV-GRK1-ABCA4 intein vectors (set 1) (dose of each ABCA4 vector/eye: 5.5×10\hat{ }9 GC). One month later, ABCA4 expression in retinal lysates from unaffected and AAV intein-injected Abca4^{-/-} mice was analyzed by WB using an antibody which recognizes both murine and human ABCA4 (FIG. 21). AAV intein ABCA4 expression was found to be 8.6±1.3% of endogenous ABCA4.

To confirm efficient large protein reconstitution in the clinically-relevant pig retina, Large White pigs were injected subretinally with either AAV2/8-GRK1-ABCA4 intein (set 1) or dual vectors (dose of each vector/eye: 2×10\hat{ }11 GC, delivered through two adjacent subretinal blebs) and 1 month post-injection protein expression was analyzed by WB. Notably, AAV intein was found to reconstitute full-length ABCA4 protein more efficiently than dual AAV vectors (FIG. 4C).

Lastly, human retinal organoids from iPSCs of either healthy individuals or STGD1 patients at 121 days of culture [when photoreceptor maturation starts (20)] were infected with AAV2/2-GRK1-ABCA4 intein vectors (set 1) (dose of each vector/organoid: 1×10\hat{ }12 GC). Organoids were lysed between 20 and 40 days after infection and analyzed by WB. ABCA4 of the expected size was detected in all infected organoids (FIG. 4D and FIG. 22; n=3 and n=4 from normal control and STGD1 organoids, respectively).

### Example 7. Subretinal Administration of AAV Intein Vectors Improves the Retinal Phenotype of STGD1 and LCA10 Mouse Models

To determine whether the photoreceptors transduction obtained with AAV intein vectors could be therapeutically relevant, they were tested in the retina of mouse models of STGD1 (Abca4−/−) and LCA10 (rd16).

One-month-old Abca4−/− mice were injected subretinally with AAV2/8-GRK1-ABCA4 intein vectors (set 1) (dose of each vector/eye: 4.3-4.8×10\hat{ }9 GC). Three months later the eyes were harvested, and transmission electron microscopy analysis of retinal ultrathin sections was performed to measure the amounts of lipofuscin, which accumulates in the retinal pigmented epithelium (RPE) of Abca4−/− mice (56, 57). Notably, RPE lipofuscin accumulation was significantly reduced in the Abca4−/− eyes injected with AAV intein vectors but not in negative control injected eyes (p value=0.0163; FIG. 5A and FIG. 23).

In parallel, 4-6-day-old rd16 mice were injected subretinally with AAV2/8-GRK1-CEP290 intein vectors (set 5) (dose of each vector/eye: 5.5×10\hat{ }8 GC). Microscopy analysis of retinal sections 1 month after injection showed that the thickness of the outer nuclear layer (ONL), which includes photoreceptors nuclei, was significantly reduced in rd16 mice compared to wild-type mice (p value=0.00048; FIG. 5B), as result of progressive retinal degeneration (55) Notably, the ONL thickness in the rd16 retinas injected with AAV intein vectors was significantly higher (about 60%, p value=0.00281) than that of negative control injected rd16 retinas (FIG. 5B). Accordingly, retinal function tests based on pupillary light responses (PLR) showed a significant higher pupil constriction (about 20%, p value=0.00073) in rd16 mice injected with AAV intein vectors than in negative control-injected rd16 eyes (FIG. 5C).

Further, the inventors investigated the safety of AAV intein vectors in the retina. To this aim, wild-type C57BL/6J mice were injected subretinally with either AAV2/8-GRK1-ABCA4 or -CEP290 intein vectors (set 1 and 5, respectively) (dose of each ABCA4 vector/eye: 4.3×10\hat{ }9 GC; dose of each CEP290 vector/eye: 1.1×10\hat{ }9 GC) and retinal electrical activity was measured by Ganzfeld electroretinogram (ERG) at 6 and 4.5 months post-injection, respectively. In both studies a- and b-wave amplitudes were similar between mouse eyes that were injected with AAV intein vectors (n=14-15 and n=11, for ABCA4 and CEP290, respectively) and eyes injected with either negative control AAV vectors (n=8 and n=5 for ABCA4 and CEP290, respectively) or PBS (n=6-7 and n=6, for ABCA4 and CEP290, respectively). Similarly, the thickness of the ONL measured by optical coherence tomography was similar between AAV intein-, negative control- and PBS-injected eyes (FIG. 24).

### Example 8. Safe AAV Intein-Mediated Large Gene Delivery

Although no evident signs of toxicity were observed in wild-type mice injected with AAV intein, the inventors have evaluated the inclusion in the trans-splicing system of a degron that, once embedded within the excised intein, leads fused protein to rapid ubiquitination and subsequent proteasomal destruction (FIG. 6). Most of the described degrons are functional at N- or C-terminal position (i.e CL1, SMN, CIITA, ODC), these degrons cannot be fused to N- or C-intein because will lead to the degradation of the single host protein thus subtracting polypeptides that need to be engaged in the Protein Trans-Splicing (PTS) reaction. Therefore the inventors chose the mutated form of the dihydrofolate reductase from E. coli (ecDHFR) which include three amino acidic mutations, R12Y, Y100I and G67S (69) that confer with functional activity only at N- or internal position.

To test the efficiency of the ecDHFR in reducing the amount of the excised intein, inventors generated an AAV vector encoding the N-terminal half of the EGFP fused to the N-terminal half of the Npu DnaE and ecDHFR (pAAV2.1-CMV-5′ EGFP intein_ecDHFR). Thus, the degron will be at the C-terminal end where it should be inactive. AAV-EGFP-ecDHFR intein plasmid in combination with vector II (encoding for the C-terminal half of the EGFP fused to the C-terminal half of the Npu DnaE (pAAV2.1-CMV-3′ EGFP intein)) were used to transfect HEK293 cells and evaluate the production of the full-length EGFP protein and excised intein. Trans-spliced EGFP protein with similar protein levels compared to AAV intein, was detected by WB analysis. In addition, the amount of the excised intein was considerably reduced in HEK293 cell lysates after cotransfection of AAV-EGFP-ecDHFR intein plasmids (FIG. 7). Then, inventors decided to apply the same strategy to the large ABCA4 protein (pAAV2.1-CMV260-5′ ABCA4 intein_ecDHFR). As for EGFP, they found similar amount of the full-length ABCA4 from AAV-ABCA4-ecDHFR intein plasmids compared to AAV-ABCA4-intein (FIG. 8A). Importantly, a complete abolishment of the excised intein was observed (FIG. 8B).

To prove that the inventors are observing an ecDHFR-mediated DnaE degradation, cells were treated with trimethoprim (TMP). The TMP is an antibiotic that can bind the ecDHFR preventing the protein from being degraded, which allows the fusion protein to escape degradation (69). HEK293 cells cotransfected with AAV-ABCA4-ecDHFR intein plasmids were treated with increased dose of TMP and found that the DnaE intein is not degraded anymore, the TMP stabilize the ecDHFR in a dose-dependent manner, meaning that the reduction of the DnaE intein is mediated by the ecDHFR (FIG. 9).

One limitation of including a degron in a vector (in addition to inteins) is that the cloning capacity of AAV is further reduced thus resulting in oversize AAV vectors for some application. Indeed, the ecDHFR is 159aa long. Thus, inventors designed a shorter ecDHFR variant of 105aa which retains the amino acid reported to be crucial for its activity at N- or internal position. The inventors tested this mini ecDHFR in both EGFP and ABCA4 intein plasmids (pAAV2.1-CMV-5′ EGFP intein_mini ecDHFR; pAAV2.1-CMV260-5′ ABCA4 intein_mini cDHFR). Upon cotransfection of either AAV-EGFP- or ABCA4-mini ecDHFR intein plasmids they found similar full-length protein expression compared to the AAV intein plasmids (FIGS. 10 and 11A) and a strong reduction of the DnaE intein (FIGS. 10 and 11B).

These results suggested that the inclusion of either ecDHFR or mini ecDHFR in the PTS system mediates selective intein degradation without affecting significantly the efficacy of protein trans-splicing and therapeutic protein production.

### Example 9. Intein-Mediated Protein Trans-Splicing in the Liver

To test the efficiency of intein-mediated protein trans-splicing in the liver two AAV vectors each encoding either the N- or the C-terminal half of the reporter EGFP protein fused to the N- and C-terminal halves of the DnaE split-intein from Nostoc punctiforme were generated. 5-weeks old C57/BL6 mice were injected retro-orbitally with AAV2/8 vectors with the liver-specific human thyroxine binding globulin (TBG) promoter (dose of each vector/kg: 5×10¹¹ GC). Livers were harvested 4 weeks post-injection and lysed for analysis by Western blot with anti-3× flag antibody to detect EGFP-3× flag and intein-3× flag. Quantification of EGFP bands' intensity showed that AAV intein transduce liver more efficiently than dual AAV with about 6-7-fold higher protein amount.

### Example 10. AAV Intein Vectors can be Used to Deliver the Large F8 Gene Affected in Hemophilia A

The F8 gene, mutated in haemophilia A, is too large (about 7 kb) to be delivered by a single AAV in its wild type conformation. Because of this, only B-domain deleted (BDD) conformations of the gene have been adapted in the context of AAV gene therapy. Recently a 5 kb expression cassette including a BDD-F8 and both short liver-specific promoter and a polyA signal has been packaged into AAV5 and shown to result in therapeutic levels of FVIII in mice and cynomolgus monkeys (70) as well as in HemA patients (71). However, the genome of this vector is slightly oversize and is packaged into AAV capsids as a library of heterogeneous truncated genomes, which upon reconstitution in target cells result in effective transduction. The efficiency of oversize AAV vectors is lower compared to normal size and the quality of such a product with heterogeneous truncated genomes may preclude its further development towards commercialization.

To overcome the limited AAV cargo capacity, a protein trans-splicing strategy involving two separate AAV vectors with regular size genomes, each encoding one of the 2 halves of the large FVIII protein flanked by the split Npu DnaE inteins was designed.

The wild type F8 gene was split into 2 different splitting points in the B domain, namely set 1 and set 2. The F8 intein vectors under the liver-specific hybrid liver promoter (HLP) together with a short synthetic polyA were produced (FIG. 25A). The vector genomes were properly packaged into AAV capsids unlike their oversize AAV BDD-F8 control as shown by Southern blot (FIG. 25B).

To determine the therapeutic relevance of the strategy, the AAV2/8 F8 intein vectors were injected systemically via retro-orbital infusion (dose of each vector/animal: 4-5×10¹¹ GC) into 7-8-week old hemophilia A knockout mice. aPTT (activated partial thromboplastin time) analysis of the blood plasma 8 weeks post injection showed slight correction of the bleeding phenotype albeit not at the same levels as the oversize single AAV BDD-F8 control (FIG. 25C).

### REFERENCES

1. M. M. Sohocki, et al. Hum. Mutat. 17, 42-51 (2001).
2. T. Dryja, in The Online Metabolic & Molecular Bases of Inherited Diseases C. Scriver, A. Beaudet, W. Sly, D. Valle, Eds. (McGraw-Hill, New York, N.Y., 2001), vol 4, pp. 5903-5933.
3. FDA approves hereditary blindness gene therapy. Nat Biotechnol 36, 6 (2018).
4. I. Trapani, A. Auricchio, Trends Mol Med, (2018).
5. A. Auricchio, A. J. Smith, R. R. Ali, Hum Gene Ther 28, 982-987 (2017).
6. I. Trapani et al., EMBO Mol Med 6, 194-211 (2014).
7. R. Allikmets, Nat. Genet. 17, 122 (1997).
8. J. M. Millan, et al. J. Ophthalmol. 2011, 417217 (2011).
9. T. Hasson, et al. Proc. Natl. Acad. Sci. USA 92, 9815-9819 (1995).
10. X. Liu, et al. Cell. Motil. Cytoskeleton 37, 240-252 (1997).
11. D. Gibbs, et al. Invest. Ophthalmol. Vis. Sci. 51, 1130-1135 (2010).
12. D. Duan, Y. Yue, J. F. Engelhardt, Mol Ther 4, 383-391 (2001).
13. Z. Yan, Y. et al., Proc Natl Acad Sci USA 97, 6716-6721 (2000).
14. A. Maddalena et al., Mol Ther 26, 524-541 (2018).
15. P. Colella et al., Gene Ther 21, 450-456 (2014).
16. O. Novikova, N. Topilina, M. Belfort, J Biol Chem 289, 14490-14497 (2014).
17. K. V. Mills, M. A. Johnson, F. B. Perler, J Biol Chem 289, 14498-14505 (2014).
18. N. H. Shah, et al., J Am Chem Soc 135, 5839-5847 (2013).
19. Y. Li, Biotechnol Lett 37, 2121-2137 (2015).
20. N. H. Shah, T. W. Muir, Chem Sci 5, 446-461 (2014).
21. C. Schmelas, D. Grimm, Biotechnol J 13, e1700432 (2018).
22. L. Villiger et al., Nat Med 24, 1519-1525 (2018).
23. F. Zhu et al, Sci China Life, 2010;
24. F. Zhu et al Sci China Life, 2013
25. Li at al., Hum Gene Ther, 2008
26 P. Subramanyam et al., Proc Natl Acad Sci, 2013
27. H. Iwai, S. Zuger, J. Jin, P. H. Tam, FEBS Lett 580, 1853-1858 (2006).
28. J. Zettler, V. Schutz, H. D. Mootz, FEBS Lett 583, 909-914 (2009).
29. J. Li, W. Sun, B. Wang, X. Xiao, X. Q. Liu, Hum Gene Ther 19, 958-964 (2008).
30. S. W. Lockless, T. W. Muir, Proc Natl Acad Sci USA 106, 10999-11004 (2009).
31. Stevens et al., J Am Chem Soc. 2016 Feb. 24; 138(7):2162-5
32. S. J. Reich, et al. Hum. Gene. Ther. 14, 37-44 (2003)
33. N. Esumi, et al. J. Biol. Chem. 279, 19064-19073 (2004).
34. Y. Tsybovsky, K. Palczewski, Protein Expr Purif 97, 50-60 (2014).
35. S. Bungert, L. L. Molday, R. S. Molday, J Biol Chem 276, 23539-23546 (2001).
36. T. G. Drivas, E. L. Holzbaur, J. Bennett, J Clin Invest 123, 4525-4539 (2013).
37. G. Gao et al., Hum Gene Ther 11, 2079-2091 (2000).
38. L. P. Pellissier et al., Mol Ther Methods Clin Dev 1, 14009 (2014).
39. L. P. Pellissier et al., Mol Ther Methods Clin Dev 1, 14009 (2014).
40. S. C. Khani et al., Invest Ophthalmol Vis Sci 48, 3954-3961 (2007).
41. M. Doria, A. Ferrara, A. Auricchio, Hum Gene Ther Methods 24, 392-398 (2013).
42. R. Sangermano et al., Ophthalmology 123, 1375-1385 (2016)
43. R. Sangermano et al., Ophthalmology 123, 1375-1385 (2016).
44. T. Nakano et al., ell Stem Cell 10, 771-785 (2012).
45. X. Zhong et al., Nat Commun 5, 4047 (2014).
46. X. Zhong et al., Nat Commun 5, 4047 (2014).
47. M. Jansen et al., Traffic 12, 218-231 (2011).
48. C. Mussolino et al., Gene Ther 18, 637-645 (2011).
49. T. Nakano et al., Cell Stem Cell 10, 771-785 (2012).
50. X. Zhong et al., Nat Commun 5, 4047 (2014).
51. M. Cheriyan, S. H. Chan, F. Perler, J Mol Biol 426, 4018-4029 (2014).
52. J. E. Donello, J. E. Loeb, T. J. Hope, J Virol 72, 5085-5092 (1998).
53. N. Zhang et al., Hum Mol Genet 24, 3220-3237 (2015).
54. H. Sun, P. M. SmaIlwood, J. Nathans, Nat Genet 26, 242-246 (2000).
55. T. G. Drivas, E. L. Holzbaur, J. Bennett, J Clin Invest 123, 4525-4539 (2013)
56. N. L. Mata et al., Invest Ophthalmol Vis Sci 42, 1685-1690 (2001).
57. J. Weng et al., Cell 98, 13-23 (1999).
58. Smith A J et al., Gene Ther. 2012 February; 19(2):154-61.
59. Liu X Q et al., Proc Natl Acad Sci USA. 1997 Jul. 22; 94(15):7851-6
60. Srivastava A, Curr Opin Virol. 2016 December; 21:75-80.
61. Auricchio et al. (2001) Hum. Mol. Genet. 10(26):3075-81
62. Dalkara D et al., Sci Transl Med. 2013 Jun. 12; 5(189):189ra76.
63. Petrs-Silva H et al., Mol Ther. 2011 February; 19(2):293-301.
64. Klimczak R R et al., PLoS One. 2009 Oct. 14; 4(10):e7467.
65. Hickey D G et al., Gene Ther. 2017 December; 24(12):787-800.
66. Perler, F. B. (2002). InBase, the Intein Database. Nucleic Acids Res. 30, 383-384
67. McIntosh J (2013).Blood 20 Feb. 2013, 121(17):3335-3344
68. Levitt N, (1989). Genes Dev. 1989 July; 3(7):1019-25
69. Iwamoto M et al., Chem Biol. 2010 Sep. 24; 17(9): 981-988.
70. Bunting, S., et al., Gene Therapy with BMN 270 Results in Therapeutic Levels of FVIII in Mice and Primates and Normalization of Bleeding in Hemophilic Mice. Mol Ther, 2018. 26(2): p. 496-509.
71. Rangarajan, S., et al., AAV5-Factor VIII Gene Transfer in Severe Hemophilia A. N Engl J Med, 2017. 377(26): p. 2519-2530.

1- A vector system to express a coding sequence in a cell, said coding sequence consisting of a first portion (CDS1), a second portion (CDS2) and optionally a third portion (CDS3), said vector system comprising:
a) a first vector comprising:
said first portion of said coding sequence (CDS1),
a first intein nucleotide sequence coding for a N-Intein, said sequence being located at the 3′ end of CDS1; and
b) a second vector comprising:
said second portion of said coding sequence (CDS2),
a second intein nucleotide sequence coding for a C-Intein, said sequence being located at the 5′ end of CDS2;
wherein when the first vector and the second vector are inserted in a cell, the protein product of the coding sequence is produced by protein splicing;
or said vector system comprising:
a′) a first vector comprising:
said first portion of said coding sequence (CDS1),
a first intein nucleotide sequence coding for a first N-Intein, said sequence being located at the 3′ end of CDS1; and
b′) a second vector comprising:
said second portion of said coding sequence (CDS2),
a second intein nucleotide sequence coding for a first C-Intein, said sequence being located at the 5′ end of CDS2;
a third intein nucleotide sequence coding for a second N-Intein, said sequence being located at the 3′ end of CDS2; and
c′) a third vector comprising:
said third portion of said coding sequence (CDS3)
a fourth intein nucleotide sequence coding for a second C-Intein, said sequence being located at the 5′ end of CDS3
wherein the first intein nucleotide sequence is different from the third intein nucleotide sequence and the second intein sequence is different from the fourth intein nucleotide sequence, wherein when the first vector, the second vector, the third vector are inserted in a cell, the protein product of the coding sequence is produced by protein splicing.

2- The vector system according to claim 1, wherein the first intein, the second intein, the third intein and the fourth intein encodes for a split intein, preferably said split intein has a maximum length of 150 amino acids, more preferably said split intein is a DnaE or DnaB intein.

3- The vector system according to claim 1 or 2, wherein
the first intein nucleotide sequence encodes for an intein selected from the group consisting of: SEQ ID No 1, 3, 5, 7, 9, 11, 13 or a variant thereof or a fragment thereof or an homolog thereof;
the second intein nucleotide sequence encodes for an intein selected from the group consisting of: SEQ ID No 2, 4, 6, 8, 10, 12, 14 or a variant thereof or a fragment thereof or an homolog thereof;
the third intein nucleotide sequence encodes for an intein selected from the group consisting of: SEQ ID No1, 3, 5, 7, 9, 11, 13 or a variant thereof or a fragment thereof or an homolog thereof;
the fourth intein nucleotide sequence encodes for an intein selected from the group consisting of: SEQ ID No2, 4, 6, 8, 10, 12, 14 or a variant thereof or a fragment thereof or an homolog thereof.

4- The vector system according to any one of previous claims, wherein the first vector, the second vector and the third vector further comprise a promoter sequence operably linked to the 5′end portion of said first portion of the coding sequence (CDS1) or of said second portion of the coding sequence (CDS2) or of said third portion of the coding sequence (CDS3).

5- The vector system according to any one of previous claims, wherein the first vector, the second vector and the third vector further comprise a 5′-terminal repeat (5′-TR) nucleotide sequence and a 3′-terminal repeat (3′-TR) nucleotide sequence, preferably the 5′-TR is a 5′-inverted terminal repeat (5′-ITR) nucleotide sequence and the 3′-TR is a 3′-inverted terminal repeat (3′-ITR) nucleotide sequence.

6- The vector system according to any one of previous claims, wherein the first vector, the second vector and the third vector further comprise a poly-adenylation signal nucleotide sequence and/or wherein at least one of the first vector or the second vector or the third vector further comprises a nucleotide sequence coding for a degradation signal.

7- The vector system according to claim 6 wherein the degradation signal is selected from the group consisting of CL1, PB29, SMN, CITTA, ODc, ecDHFR or a fragment thereof.

8- The vector system according to any one of previous claims, wherein the coding sequence is split into the first portion, the second portion and optionally the third portion, at a position consisting of a nucleophile amino acid which does not fall within a structural domain or a functional domain of the encoded protein product, wherein the nucleophile aminoacid is selected from serine, threonine, or cysteine.

9- The vector system according to any one of previous claims, wherein at least one of the first vector, the second vector and the third vector further comprises at least one enhancer or regulatory nucleotide sequence, operably linked to the coding sequence.

10- The vector system according to any one of previous claims, wherein the coding sequence encodes a protein able to correct a pathological state or disorder, preferably the disorder is a retinal degeneration, a metabolic disorder, a blood disorder, a neurodegenerative disorder, hearing loss, channellopathy, lung disease, myopathy, heart disease.

11- The vector system according to any one of previous claims, wherein the coding sequence encodes a protein able to correct a pathological state or disorder, preferably the disorder is a retinal degeneration, preferably the retinal degeneration is inherited, preferably the pathology or disease is selected from the group consisting of: retinitis pigmentosa (RP), Leber congenital amaurosis (LCA), Stargardt disease (STGD), Usher disease (USH), Alstrom syndrome, congenital stationary night blindness (CSNB), macular dystrophy, occult macular dystrophy, a disease caused by a mutation in the ABCA4 gene.

12- The vector system according to any one of claims 1 to 10, wherein the coding sequence encodes a protein able to correct Duchenne muscular dystrophy, cystic fibrosis, hemophilia A, Wilson disease, Phenylketonuria, dysferlinopathies, Rett's syndrome, Polycystic kidney disease, Niemann-Pick type C, Huntington's disease.

13- The vector system according to any one of claims 1 to 11, wherein the coding sequence is the coding sequence of a gene selected from the group consisting of: ABCA4, MYO7A, CEP290, CDH23, EYS, PCDH15, CACNA1, SNRNP200, RP1, PRPF8, RP1L1, ALMS1, USH2A, GPR98, HMCN1.

14- The vector system according to any one of claims 1 to 12, wherein the coding sequence is the coding sequence of a gene selected from the group consisting of: DMD, CFTR, F8, ATP7B, PAH, DYSF, MECP2, PKD, NPC1 HTT.

15- The vector system according to any one of previous claims comprising:
a) a first vector comprising in a 5′-3′ direction:
a 5′-inverted terminal repeat (5′-ITR) sequence;
a promoter sequence;
a 5′ end portion of a coding sequence (CDS1), said 5′end portion being operably linked to and under control of said promoter;
a first intein nucleotide sequence coding for a N-Intein; and
a 3′-inverted terminal repeat (3′-ITR) sequence; and
b) a second vector comprising in a 5′-3′ direction:
a 5′-inverted terminal repeat (5′-ITR) sequence;
a promoter sequence;
a second intein nucleotide sequence coding for a C-Intein;
a 3′end portion of the coding sequence (CDS2); and
a 3′-inverted terminal repeat (3′-ITR) sequence;
or comprising:
a′) a first vector comprising in a 5′-3′ direction:
a 5′-inverted terminal repeat (5′-ITR) sequence;
a promoter sequence;
a 5′ end portion of a coding sequence (CDS1′), said 5′end portion being operably linked to and under control of said promoter;
a first intein nucleotide sequence coding for a first N-Intein; and
a 3′-inverted terminal repeat (3′-ITR) sequence; and
b′) a second vector comprising in a 5′-3′ direction:
a 5′-inverted terminal repeat (5′-ITR) sequence;
a promoter sequence;
a second intein nucleotide sequence coding for a first C-Intein;
the second portion of the coding sequence (CDS2′); and
a third intein nucleotide sequence coding for a second N-intein;
a 3′-inverted terminal repeat (3′-ITR) sequence; and
c′) a third vector comprising in a 5′-3′ direction:
a 5′-inverted terminal repeat (5′-ITR) sequence;
a promoter sequence;
a fourth intein nucleotide sequence coding for a second C-Intein;
the third portion of the coding sequence (CDS3′); and
a 3′-inverted terminal repeat (3′-ITR) sequence.

16. The vector system according to any one of previous claims wherein the coding sequence encodes the ABCA4 gene, preferably, said coding sequence is split at a nucleotide corresponding to aa Cys1150, Ser1168, Ser 1090 of the ABCA4 protein, and a split intein is inserted at the split point or the coding sequence encodes the CEP290 gene, preferably, said coding sequence is split at a nucleotide corresponding to aa Cys1076; Ser1275 of the CEP290 protein, preferably, the coding sequence encoding the CEP290 gene is split at a nucleotide sequence corresponding to aa Cys 929 and 1474; Ser 453 and Cys 1474 of said CEP290 protein, and two split inteins are inserted at the split points.

17- The vector system according to any one of previous claims wherein said first, second and third vector are independently a viral vector, preferably an adeno viral vector or adeno-associated viral (AAV) vector, preferably said first, second and third adeno-associated viral (AAV) vectors are selected from the same or different AAV serotypes, preferably the serotype is selected from the serotype 2, the serotype 8, the serotype 5, the serotype 7 or the serotype 9, serotype 7m8, serotype sh10; serotype 2(quad Y-F).

18- A host cell transformed with the vector system according to any one of previous claims.

19- The vector system according to any one of claims 1 to 17 or the host cell according to claim 18 for medical use.

20- The vector system according to any one of claims 1 to 19 or the host cell according to claim 18 for use in gene therapy, preferably for use in the treatment and/or prevention of a pathology or disease characterized by a retinal degeneration, a metabolic disorder, a blood disorder, a neurodegenerative disorder, hearing loss, channellopathy, lung disease, myopathy, heart disease.

21- The vector system or the host cell for use according to claim 20 wherein the retinal degeneration is inherited, preferably the pathology or disease is selected from the group consisting of: retinitis pigmentosa (RP), Leber congenital amaurosis (LCA), Stargardt disease (STGD), Usher disease (USH), Alstrom syndrome, congenital stationary night blindness (CSNB), macular dystrophy, occult macular dystrophy, a disease caused by a mutation in the ABCA4 gene.

22- The vector system or the host cell for use according to claim 20 for use in the prevention and/or treatment of Duchenne muscular dystrophy, cystic fibrosis, hemophilia A, Wilson disease, Phenylketonuria, dysferlinopathies, Rett's syndrome, Polycystic kidney disease, Niemann-Pick type C, Huntington's disease.

23- A pharmaceutical composition comprising the vector system according to any one of claims 1 to 17 or the host cell according to claim 18 and pharmaceutically acceptable vehicle.
